(19) United States
(12) Patent Application Publication
(10) Pub. No.: US 2024/0156880 A1
(43) Pub. Date: May 16, 2024

(54) BACTERIOTHERAPY

(71) Applicant: Genome Research Limited, Hinxton (GB)

(72) Inventors: Anne NEVILLE, Hinxton (GB); Hilary BROWNE, Hinxton (GB); Sam FORSTER, Hinxton (GB); Trevor LAWLEY, Hinxton (GB)

(21) Appl. No.: 18/446,994

(22) Filed: Aug. 9, 2023

Related U.S. Application Data
(63) Continuation of application No. 16/094,856, filed on Oct. 18, 2018, now Pat. No. 11,786,562.

(30) Foreign Application Priority Data
Apr. 19, 2016 (GB) 1606801.7

(51) Int. Cl. A61K 35/742; A61K 9/00; A61P 1/04; A61P 1/06; A61P 1/14; A61P 1/16; A61P 31/04

(52) U.S. Cl. CPC A61K 35/742; A61K 9/0031; A61K 9/0053; A61P 1/04; A61P 1/06; A61P 1/14; A61P 1/16; A61P 31/04

(57) ABSTRACT

The invention relates to therapeutic compositions comprising at least one isolated bacterium and a pharmaceutically acceptable excipient, as well as methods of preparing such therapeutic compositions. The therapeutic compositions find application in the treatment of dysbiosis, in particular dysbiosis of the gastrointestinal tract.

### REFERENCE TO SEQUENCE LISTING SUBMITTED ELECTRONICALLY

The content of the electronically submitted sequence listing (Name: P34640USN1 SEQ LIST updated_ST25.txt; Size: 100,657 bytes; and Date of Creation: Aug. 16, 2021) is herein incorporated by reference in its entirety.

### FIELD OF THE INVENTION

The present invention relates to therapeutic compositions comprising at least one isolated bacterium as defined herein and a pharmaceutically acceptable excipient, as well as methods of preparing such therapeutic compositions. The therapeutic compositions find application in the treatment of dysbiosis, in particular dysbiosis of the gastrointestinal tract.

The dysbiosis may be a dysbiosis associated with an enteric bacterial infection, inflammatory bowel disease, pouchitis, irritable bowel syndrome, a metabolic disease, a neuropsychiatric disorder, an autoimmune disease, an allergic disorder, a cancer, or hepatic encephalopathy.

### BACKGROUND TO THE INVENTION

A typical human intestinal microbiota contains 100-1000 bacterial species. There is extensive compositional diversity between individuals, such that each individual's microbiota is as unique as a fingerprint (Qin, Li et al. 2010; Nielsen, Almeida et al. 2014). The majority of the bacterial species within the adult human microbiota are derived from four high level taxonomic classifications or phyla, the Firmicutes, Bacteroidetes, Actinobacteria and Proteobacteria. These groups change in abundance from birth to adulthood to old age, reflecting changing environmental influences such as initial mode of delivery, diet, insults such as pathogen infection and in many cases antibiotic usage (Dominguez-Bello, Costello et al. 2010, Koenig, Spor et al. 2011, Ottman, Smidt et al. 2012). In adulthood, the intestinal microbiota is dominated by the Firmicutes and the Bacteroidetes, both of which are strict anaerobes.

The intestinal microbiota plays a key role in digesting food inaccessible to the human gastrointestinal tract, such as metabolizing carbohydrates into short chain fatty acids (Sekirov, Russell et al. 2010), interacting with the immune system to maintain homeostasis (Hooper, Littman et al. 2012), promoting maturation of the gut (Hooper, Wong et al. 2001) and development of the immune system. The intestinal microbiota also plays an important role in resisting pathogen invasion, termed ‘colonisation resistance’. This functions through the diversity and abundance of commensal species present and through the occupation of key niches and utilization of nutrients (Lawley and Walker 2013; Britton and Young 2014). If microbial homeostasis is disturbed, for example through use of antibiotics, a shift towards dysbiosis can occur.

Dysbiosis provides the opportunity for pathogens to establish themselves and cause disease to the individual in question. This has been best studied in terms of a single implicated pathogen such as Clostridium difficile (Lawley et al. 2012; Britton and Young 2014; Buffie et al. 2015), but dysbiosis has also been linked with other more complex, multi-factorial diseases such as Inflammatory Bowel Disease (IBD), pouchitis (Angeriman et al. 2014), Irritable Bowel Syndrome (IBS), hepatic encephalopathy (Bajaj 2014; Bajaj et al. 2012) metabolic diseases (including metabolic syndrome, malnutrition, and obesity), neuropsychiatric disorders such as Parkinson's and Alzheimer's disease, autoimmune diseases, allergic disorders, and cancer (Jostins, Ripke et al. 2012, Collins 2014, Hold, Smith et al. 2014, Perez Martinez, Bauerl et al. 2014, Scheperjans, Aho et al. 2015; Blanton et al. 2016, Xu et al. 2015).

Faecal microbiota transplantation (FMT) has proved successful in resolving C. difficile associated dysbiosis (Petrof et al. 2013, van Nood et al. 2013), and the administration of specific bacteria has also proved effective for this purpose (Lawley et al. 2012, Buffie et al. 2015). FMT has also showed promising results in the treatment of other intestinal diseases, as well as the management of extra-intestinal disorders associated with gut microbiota, including metabolic diseases, neuropsychiatric disorders, autoimmune diseases, allergic disorders, and tumours (Xu et al. 2015).

Recent years have seen great advances in understanding the role the intestinal microbiota plays in health and disease and how it can be manipulated for the benefit of the host. The majority of our understanding has to date been derived by culture-independent studies, i.e. by studying the compositional components of the microbiota and how they change during disease using molecular and genomic techniques. This process allows identification of potential therapeutic candidates that can resolve disease. However, the isolation, purification and acquisition of such candidate therapeutic bacteria has proven difficult.

There is therefore a need in the art to identify and isolate specific bacteria, as well as combinations of bacteria, which can be used to treat dysbiosis. Therapeutic compositions based on known, defined, bacteria or bacterial mixtures are advantageous as they improve patient safety because they comprise only defined and well characterised bacteria that are known to promote, and not harm, human health, and eliminate the possibility of inadvertently transferring pathogenic material to a recipient by FMT. In addition, such therapeutic compositions can be prepared in vitro in a large-scale manner using standardised, reproducible procedures, thereby providing batch consistency, and do not rely on regular donations from healthy human donors. Therapeutic compositions comprising known, defined, bacteria or bacterial mixtures can also be therapeutically delivered e.g. in a capsule, as a tablet, or as an enema, which is more acceptable to patients and health care professionals than suspensions of faecal material used in the case of FMT. The bacteria included in such therapeutic compositions can further be tailored to the treatment of specific dysbiotic states and diseases associated therewith by specifically altering the bacterial composition to optimally resolve the dysbiotic state in question and thus improve efficacy.

However, in order to isolate such candidate therapeutic bacteria for the treatment of dysbiosis, a thorough understanding of the biology of the candidates in question is required, as well as a large initial panel of candidates to select from. This poses a problem as the majority of the bacteria in the intestinal microbiota are considered to be unculturable and have never been isolated in the laboratory (Eckburg, Bik et al. 2005, Hattori and Taylor 2009, Stewart 2012). Thus, gaining a basic understanding of the functional attributes of the microbiota and developing a multi-species bacteria-based therapeutic with fastidious, anaerobic commensal isolates presents a formidable challenge. While recent efforts have made progress in resolving this issue (Goodman, Kallstrom et al. 2011, Lagier, Hugon et al. 2015), there remains a need in the art to identify and isolate bacteria capable of treating dysbiosis.

### STATEMENTS OF INVENTION

The present invention relates to therapeutic compositions, in particular therapeutic compositions for use in the treatment of dysbiosis in an individual. Dysbiosis can occur in any part of the human or animal body which is normally colonized by bacteria and other microbes. The present invention particularly concerns dysbiosis of the gastrointestinal tract in humans.

The present inventors have surprisingly found that the majority of bacteria present in the human intestinal microbiota can be cultured, contrary to the prevailing view in the art which was that the majority of the human intestinal microbiota is unculturable. This major breakthrough now allows the majority of bacteria present in the human microbiota to be isolated and characterised, and evaluated for their activity in treating dysbiosis. This is possible not only for individual bacterial isolates but also for combinations of bacteria isolated from the intestinal microbiota. In addition, isolation of these bacteria allows the bacteria to be screened, for example, for the absence of virulence factors and antibiotic resistance prior to their inclusion in a therapeutic composition, thereby improving safety. In addition, the bacteria included in a therapeutic composition can be tailored to the treatment of a specific dysbiotic state and/or disease associated therewith by optimising the bacterial composition to resolve the dysbiosis in question, thereby improving efficacy. None of this is possible in FMT where undefined mixes of bacteria are used, usually obtained from a faecal sample of a healthy human donor. The use of isolated bacteria for the treatment of dysbiosis has the further advantage that it allows the bacteriotherapy treatment to be standardised, making patient outcomes more predictable, as well as facilitating evaluation of the therapeutic potential of bacteriotherapy in the context of particular diseases by removing the variability in bacterial composition associated with the use of FMT.

Through surprisingly being able to culture the majority of bacteria present in the human intestinal microbiota, the present inventors were able to prepare libraries of intestinal bacteria which were then subjected to whole-genome sequencing and screened using both in silico analysis and in vitro experiments to identify bacteria which are expected to be useful in treating dysbiosis, in particular dysbiosis of the gastrointestinal tract. Using this approach, the present inventors identified 51 bacteria which are expected to be useful for this purpose, including several families, genera, and species of bacteria which have not previously been described, let alone isolated or employed in the treatment of dysbiosis. As already explained above, the majority of the human microbiota was thought in the art to be unculturable, so the mere disclosure of a 16S ribosomal RNA sequence of one of these bacteria does not in itself enable the isolation of such a bacterium from its natural environment. Nor does the disclosure of such a 16S ribosomal RNA sequence suggest that a bacterium with such a sequence has previously been isolated, as 16S ribosomal RNA sequence information can be obtained from bacterial populations, including faecal samples, without the need to isolate individual bacteria. However, the ability to isolate bacteria in pure form from their natural environment is a prerequisite for their inclusion in therapeutic compositions according to the present invention.

Thus, in a first aspect, the present invention provides a therapeutic composition comprising at least one isolated bacterium and a pharmaceutically acceptable excipient. The bacterium preferably comprises a gene encoding a 16S ribosomal RNA (rRNA), wherein said gene comprises a sequence with at least 90% sequence identity with the sequence set forth in any one of SEQ ID NOs 1 to 51.

The therapeutic composition may comprise more than one isolated bacterium, in addition to the pharmaceutically acceptable excipient. Where more than one bacterium is included in the therapeutic composition, the bacteria are preferably distinct, wherein each bacterium comprises a gene encoding a 16S rRNA, wherein said gene comprises a sequence with at least 90% sequence identity with the sequence set forth in any one of SEQ ID NOs 1 to 51. Thus, for example, the therapeutic composition may comprise two distinct isolated bacteria, wherein the first bacterium comprises a gene encoding a 16S rRNA, wherein said gene comprises a sequence with at least 90% sequence identity with the sequence set forth in SEQ ID NO: 1 and the second bacterium comprises a gene encoding a 16S rRNA, wherein said gene comprises a sequence with at least 90% sequence identity with the sequence set forth in SEQ ID NO: 2.

As mentioned above, the therapeutic compositions of the present invention find application in the treatment of dysbiosis, in particular the treatment of a dysbiosis of the intestinal tract. Thus, in a second aspect, the present invention provides a therapeutic composition according to the invention for use in a method of treating a dysbiosis, preferably a dysbiosis of the gastrointestinal tract, in an individual. Also provided is a method of treating a dysbiosis in an individual, the method comprising administering a therapeutically effective amount of a therapeutic composition according to the invention to an individual in need thereof, as well as the use of a therapeutic composition according to the present invention for the manufacture of a medicament for the treatment of a dysbiosis in an individual. Also provided is the use of at least one isolated bacterium, as described herein, and optionally a pharmaceutically acceptable excipient, for the manufacture of a medicament for the treatment of a dysbiosis in an individual, the bacterium preferably comprising a gene encoding a 16S rRNA and said gene comprises a sequence with at least 90% sequence identity with the sequence set forth in any one of SEQ ID NOs 1 to 51.

Methods of preparing or manufacturing a therapeutic composition according to the present invention also form part of the invention. Thus, in a third aspect, the present invention provides a method of preparing or manufacturing a therapeutic composition according to the present invention, wherein the method preferably comprises the steps of:
(i) culturing an isolated bacterium as described herein; and
(ii) mixing the bacteria obtained in (i) with a pharmaceutically acceptable excipient.

As mentioned above, the therapeutic compositions of the present invention may comprise at least two distinct isolated bacteria as described herein. Where the therapeutic composition comprise more than one distinct isolated bacteria, the method of preparing or manufacturing a therapeutic composition preferably comprises steps of:
(i) culturing a first isolated bacterium as described herein;
(ii) culturing a second isolated bacterium as described herein; and
(ii) mixing the bacteria obtained in (i) and (ii) with a pharmaceutically acceptable excipient. The bacteria cultured in steps (i) and (ii) preferably have distinct 16S rRNA sequences. Steps (i) and (ii) are preferably performed independently. The above method can be adapted to include further steps to allow the culturing of more than two distinct isolated bacteria, preferably bacteria with distinct 16S rRNA sequences, by including an additional step or steps for the culturing of a third or further isolated bacterium as disclosed herein. In this case, all bacteria cultured in the method are mixed with a pharmaceutically acceptable excipient.

A therapeutic composition obtainable by a method of preparing or manufacturing a therapeutic composition, as disclosed herein also forms part of the present invention.

### BRIEF DESCRIPTION OF THE FIGURES

FIG. 1 shows a schematic diagram of the workflow used to culture, archive and characterise the intestinal microbiota. The process incorporates several steps which are: culture, re-streak, archive and phenotype. (A) Fresh faecal samples were left untreated or were treated to select for bacteria with a desired phenotype (such as sporulation). The stool was homogenised and then serially diluted before aliquots of the homogenate were inoculated on YCFA agar to culture the bacteria present in the faecal samples. (B) Bacterial isolates were identified by selecting single colonies that were then streaked to purity before full-length 16S rRNA gene amplification and sequencing was performed. (C) Each unique, novel and desired bacterial isolate was archived frozen in a culture collection and a whole genome sequence was generated for each. (D) Phenotypic characterisation and functional validation of metagenomics studies was then performed using in vitro and in vivo methods.

FIG. 2: Targeted phenotypic culturing facilitates bacterial discovery from healthy human faecal microbiota. FIG. 2 shows the relative abundance of bacteria in faecal samples (x axis) compared to the relative abundance of bacteria growing on YCFA agar plates (y axis) as determined by metagenomic sequencing. The results demonstrate that the bacteria grown on YCFA agar are representative of the bacteria present in complete faecal samples as indicated by Spearman Rho=0.75.

FIG. 3 shows a principal component analysis (PCoA) plot of 16S rRNA gene sequences detected from 6 donor faecal samples representing bacteria in the complete faecal samples (unfilled circles), faecal bacterial colonies recovered from YCFA agar plates without ethanol pre-treatment (filled black squares) or with ethanol pre-treatment to select for ethanol-resistant spore-forming bacteria (filled black circles). These results demonstrate that culturing without ethanol selection is representative of the complete faecal sample, while ethanol treatment shifts the profile, enriching for ethanol-resistant spore-forming bacteria and allowing their subsequent isolation.

FIG. 4: Archiving of bacterial diversity and novelty through anaerobic culturing. FIG. 4A shows that the culture conditions employed by the present inventors enabled isolation of representatives from 21 of the 25 most abundant faecal bacterial genera as determined by metagenomic sequencing. A black dot indicates the number of species cultured and archived from each genus. Lachnospiraceae incertae sedis, unclassified Lachnospiraceae, Clostridium IV and Clostridium XI are not strict genera and represent currently unclassified species. FIG. 4B shows the 24 most abundant bacterial species (comprising 90% of the total bacterial abundance at the species level) as determined by metagenomic sequencing. All were cultured and archived except for Odoribacter splanchnicus. FIG. 4C shows that intestinal microbiota members present at low abundance were also cultured. At least one representative species from each of the genera presented was cultured. Genera are listed in order of decreasing abundance.

FIG. 5: Phylogeny of cultured and archived bacteria from healthy human faecal microbiota. FIG. 5 shows a phylogenetic tree of bacteria cultured from the 6 donors constructed from full length 16S rRNA gene sequences. Novel candidate species (filled black circles), genera (grey filled circles) and families (filled stars) are shown by dot colours. Major phyla and family names are indicated. Proteobacteria were not cultured, but are included for context.

FIG. 6: Faecal Microbiota Transplant (FMT) restores the intestinal microbiota of patients with recurrent C. difficile infection to a healthy state. FIG. 6 shows the results of a principal component analysis of the donors, recipients and controls at 2-3 months after FMT. The clustering of the faecal samples indicates a similar microbial community structure. Antibiotic use and exposure to C. difficile likely leads to a shift from the healthy state, as seen in the metronidazole-treated control samples. Treatment of C. difficile infection (CDI) with vancomycin, an antibiotic that targets Gram-positive organisms would presumably lead to further disruption of the intestinal microbiota. FMT led to a shift from a diseased microbiota to a healthy one with most of the post-FMT samples clustering with the donors and healthy control samples.

FIG. 7: Taxonomic summary of the bacteria isolated from faecal samples of the donors, recipients and controls from a study to treat C. difficile using FMT. These bacterial isolates represent a large cross-section of intestinal microbiota diversity.

FIG. 8: Average relative abundance of bacteriotherapy candidates in healthy individuals. The bacteriotherapy candidates occur at an average abundance of greater than 0.001% within the gastrointestinal microbiota of 1883 healthy individuals (3218 samples).

FIG. 9: The bacteriotherapy candidates are depleted in dysbiotic and disease states. The average fold change in the relative abundance of each bacteriotherapy candidate in diseased and dysbiotic states in comparison to its relative abundance in a healthy microbiota is plotted. Escherichia coli, a marker of dysbiosis is also included for comparison. The relative abundance of a bacterium refers to the proportion of the total microbiota represented by the bacterium in question.

FIG. 10 demonstrates how the zone of clearing around bacteriotherapy candidates was measured in the overlay assays. FIG. 10 shows part of a YCFA agar plate on which a bacteriotherapy candidate was streaked in an X-shape and allowed to grow. Following growth of the bacteriotherapy candidate, the plate was covered with overlay agar comprising C. difficile or E. coli. Inhibition of C. difficile or E. coli growth by a bacteriotherapy candidate was measured by determining the width of the zone of clearing around the bacteriotherapy candidate strain grown on the plate. The black diagonal line in FIG. 10 indicates the distance measured and recorded as the width of the zone of clearing for an exemplary bacteriotherapy candidate. Four such measurements were taken per plate.

FIG. 11 shows the results of C. difficile and E. coli growth overlay assays to determine the anti-pathogen activity of bacteriotherapy candidates. The zones of clearing were measured with a ruler as described in FIG. 10. Millimetre (mm) measurements were taken. FIG. 11 shows the mean measurement ±standard deviation from a representative experiment.

FIG. 12 shows the results of C. difficile and E. coli growth inhibition assays to determine the anti-pathogen activity of bacteriotherapy candidates. FIG. 12A shows the reduced relative C. difficile growth in Cell Free Supernatant (CFS) of bacteriotherapy candidate cultures at the 18.17 h time-point, while FIG. 12B shows reduced relative E. coli growth in CFS from the candidate bacteriotherapy isolates. The relative growth of either pathogen in the control YCFA medium was high (C. difficile=8.96±0.39 rel. growth units; E. coli 11.61±2.55 rel. growth units, at the 18.17 h time-point). When the mean and two standard deviations of relative growth of either pathogen in CFS derived from a bacteriotherapy candidate culture was more than two standard deviations below its mean growth in YCFA at the 18.17 h time-point, growth of the pathogen was considered to be inhibited. Where only one relative growth value was available for a particular CFS (vs C. difficile, FIG. 12A: HMI 15, HMI 26, HMI 27, HMI 28), the bacteriotherapy candidate was considered to be inhibitory if the relative growth of C. difficile was two standard deviations below the mean growth of C. difficile in YCFA broth. Only the results from inhibitory CFS are shown.

FIG. 13 shows a summary of the results obtained in the growth overlay and growth inhibition assays. Bacterial isolates shown to have inhibitory activity in the E. coli (AIEC) overlay assay, C. difficile overlay assay and C. difficile and E. coli growth assays are shown. Bacterial isolates showing inhibitory activity in two or more assays are shown in the overlapping regions. Bacterial isolates are referred to by their isolate number. See FIG. 15 for details of the bacterial isolates listed.

FIG. 14: Dendrogram and bar plots showing the relative abundance of each genus indicated in FIG. 14 at 3 months post-FMT in both donors and recipients. The dendrogram clusters samples based on the phylogenetic relationship of the microbial community present within the samples. The composition of donor and recipient profiles were similarwhen assessed at the genus level post-FMT. FIG. 15 shows characteristics of deposited bacteriotherapy candidates.

### DETAILED DESCRIPTION

The bacteria disclosed herein have been obtained from human stool samples, and thus are naturally present in the gastrointestinal tract of at least some healthy human individuals. However, these bacteria have been cultured in vitro for the first time by the present inventors, thereby isolating them from their environment in pure form, and making it possible to include them as defined active ingredients in therapeutic compositions. The bacterium present in the therapeutic composition of the present invention is thus isolated. In other words, the bacterium present in the therapeutic composition is provided in isolated and/or purified form, e.g. isolated and/or purified from the environment in which it is normally present, such as the gastrointestinal tract and/or stool samples. The isolated bacterium present in the therapeutic composition may be in substantially pure, or in homogeneous form. For example, the bacterium may be free, or substantially free, of material with which it is found in the environment in which it is normally present (e.g. the gastrointestinal tract and/or stool samples).

The bacterium present in the therapeutic composition of the present invention is preferably a human intestinal bacterium, i.e. a bacterium found in the human intestine. The bacteria whose 16S rRNA gene sequences are set out in SEQ ID NOs 1 to 51 are all intestinal bacteria.

The bacterium is preferably a non-pathogenic bacterium. In other words, the bacterium preferably does not cause disease in a healthy human individual when administered to said individual, in particular the gastrointestinal tract of said individual. The therapeutic composition can be administered to an individual in a variety of ways as described in more detailed elsewhere herein, including in the form of a tablet or enema.

The bacterium present in the therapeutic composition of the present invention is preferably susceptible to treatment with one or more antibiotics. In other words, the bacterium is preferably not resistant to treatment with at least one antibiotic. This allows antibiotic treatment of an individual in the event that one or more of the bacteria included in a therapeutic composition administered to the individual causes disease in the individual, contrary to expectations. All of the 51 bacteria disclosed herein where found to carry no known genes conferring resistance to the following antibiotics: beta-lactams, fusidic acid, elfamycin, aminoglycoside, fosfomycin, and tunicamycin. Thus, in a preferred embodiment, the bacterium is susceptible to treatment with one or more antibiotics selected from the group consisting of: a beta-lactam, fusidic acid, elfamycin, aminoglycoside, fosfomycin, and tunicamycin. In vitro and in silico methods for screening bacteria for antibiotic resistance are known in the art. Exemplary in silico methods are also described in Example 1.

The bacterium included in the therapeutic composition of the present invention preferably does not comprise one or more genes encoding one or more virulence factors and/or preferably does not produce one or more virulence factors. Virulence factors in this context are properties which enhance the potential of a bacterium to cause disease in an individual. Virulence factors include the production of bacterial toxins, such as endotoxins and exotoxins by a bacterium, as well as the production of hydrolytic enzymes that may contribute to the pathogenicity of the bacterium. Methods for screening bacteria for genes encoding virulence factors are known in the art and include the in silico methods described in Example 1. The 51 bacteria disclosed herein were found not to carry any known virulence factors using in silico analysis. Methods for screening bacteria for the production of virulence factors are similarly known in the art.

Bacteria can be taxonomically classified based on the sequence of the gene encoding the 16S ribosomal RNA (rRNA) in the bacterium. This gene sequence is also referred to as the ribosomal DNA sequence (rDNA). A bacterium comprising a gene which encodes a 16S rRNA which has 90% or more sequence identity with the 16S rRNA encoded by a second bacterium belongs to the same family as said second bacterium. A bacterium comprising a gene which encodes a 16S rRNA which has 95% or more sequence identity with the 16S rRNA encoded by a second bacterium belongs to the same genus as said second bacterium. A bacterium comprising a gene which encodes a 16S rRNA which has 97% or more, or 98.7% or more sequence identity with the 16S rRNA encoded by a second bacterium belongs to the same species as said second bacterium. A bacterium included in the therapeutic composition of the present invention may be a bacterium which belongs to the same family, genus, and/or species as a bacterium disclosed herein.

A bacterium which belongs to the same family, genus, and/or species as a bacterium disclosed herein is expected to retain one or more properties of the disclosed bacterium. Thus, in a preferred embodiment, a bacterium present in the therapeutic composition of the present invention belongs to the same family, genus, and/or species as a bacterium disclosed herein and retains at least one property of the bacterium disclosed herein. Various properties of the bacteria disclosed herein are described and include, for example, a lack of production of one or more virulence factors, susceptibility to treatment with one or more antibiotics, and a lack of pathogenicity.

The therapeutic composition of the present invention may comprise at least one isolated bacterium, wherein the bacterium comprises a gene encoding a 16S rRNA, wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence set forth in any one of SEQ ID NOs 1 to 51.

For example, the therapeutic composition of the present invention may comprise at least one isolated bacterium, wherein the bacterium comprises a gene encoding a 16S rRNA, wherein said gene comprises a sequence with at least 90% sequence identity with the sequence set forth in any one of SEQ ID NOs 1 to 51. In a preferred embodiment, the therapeutic composition comprises an isolated bacterium, wherein the bacterium comprises a gene encoding a 16S rRNA, and wherein said gene comprises a sequence with at least 90% sequence identity with the sequence set forth in SEQ ID NO: 1. In addition, or alternatively, the therapeutic composition may comprise an isolated bacterium, wherein the bacterium comprises a gene encoding a 16S rRNA, and wherein said gene comprises a sequence with at least 90% sequence identity with the sequence set forth in SEQ ID NO: 21.

In addition, or alternatively, the therapeutic composition may comprise at least one isolated bacterium, wherein the bacterium comprises a gene encoding a 16S rRNA, wherein said gene comprises a sequence with at least 95% sequence identity with the sequence set forth in any one of SEQ ID NOs 1 to 51. In a preferred embodiment, the therapeutic composition comprises at least one isolated bacterium, wherein said bacterium comprises a gene encoding a 16S rRNA, and wherein said gene comprises a sequence with at least 95% sequence identity with the sequence set forth in any one of SEQ ID NOs 2 to 20, or 22 to 51, more preferably any one of SEQ ID NOs 5, 6, 11, 13, 14, 15, 17, 18, 19, 20, 22, 23, 24, 26, 29, 33, 35, 41, 43, 45, 46, 47, 49, or 50, yet more preferably any one of SEQ ID NOs 5, 6, 11, 13, 15, 19, 22, 23, 29, 33, 35, 41, 43, 45, 46, or 50.

In addition, or alternatively, the therapeutic composition may comprise at least one isolated bacterium, wherein the bacterium comprises a gene encoding a 16S rRNA, wherein said gene comprises a sequence with at least 97%, or at least 98.7%, sequence identity with the sequence set forth in any one of SEQ ID NOs 1 to 51. In a preferred embodiment, the therapeutic composition comprises at least one isolated bacterium, wherein said bacterium comprises a gene encoding a 16S rRNA, and wherein said gene comprises a sequence with at least 97% sequence identity with the sequence set forth in any one of SEQ ID NOs 2 to 20, or 22 to 51, more preferably any one of SEQ ID NOs 2 to 3, 5 to 8, 10 to 20, 22 to 26, 29 to 37, or 39 to 50, yet more preferably any one of SEQ ID NOs 3, 5 to 8, 10 to 13, 15, 16, 19, 22, 23, 29, 32 to 37, 39 to 46, or 48 to 50. In an alternative preferred embodiment, the therapeutic composition may comprise at least one isolated bacterium, wherein said bacterium comprises a gene encoding a 16S rRNA, and wherein said gene comprises a sequence with at least 98.7% sequence identity with the sequence set forth in any one of SEQ ID NOs 2 to 20, or 22 to 51, more preferably any one of SEQ ID NOs 2 to 4, 5 to 20, 22 to 26, 29 to 37 to 51, yet more preferably any one of SEQ ID NOs 2 to 8, 10 to 13, 15, 16, 17, 19, 20, 22, 23, 29, 31, 32 to 37 to 46, or 48 to 51.

As mentioned above, in a preferred embodiment, the therapeutic composition of the present invention may comprise at least one isolated bacterium, wherein the bacterium comprises a gene encoding a 16S rRNA, wherein said gene comprises a sequence with at least 90% sequence identity with the sequence set forth in SEQ ID NO: 1 or SEQ ID NO: 21. In addition, or alternatively, the therapeutic composition may comprise at least one isolated bacterium, wherein the bacterium comprises a gene encoding a 16S rRNA, wherein said gene comprises a sequence with at least 91% sequence identity with the sequence set forth in SEQ ID NO: 29. In addition, or alternatively, the therapeutic composition may comprise at least one isolated bacterium, wherein the bacterium comprises a gene encoding a 16S rRNA, wherein said gene comprises a sequence with at least 92% sequence identity with the sequence set forth in any one of SEQ ID NOs 6, 11, 19 or 24. In addition, or alternatively, the therapeutic composition may comprise at least one isolated bacterium, wherein the bacterium comprises a gene encoding a 16S rRNA, wherein said gene comprises a sequence with at least 93% sequence identity with the sequence set forth in any one of SEQ ID NOs 13, 22, 26 or 35. In addition, or alternatively, the therapeutic composition may comprise at least one isolated bacterium, wherein the bacterium comprises a gene encoding a 16S rRNA, wherein said gene comprises a sequence with at least 94% sequence identity with the sequence set forth in any one of SEQ ID NOs 5, 14, 15, 17, 18, 23, or 50. In addition, or alternatively, the therapeutic composition may comprise at least one isolated bacterium, wherein the bacterium comprises a gene encoding a 16S rRNA, wherein said gene comprises a sequence with at least 95% sequence identity with the sequence set forth in any one of SEQ ID NOs 20, 33, 41, 43, 45, 46, 47, or 49. In addition, or alternatively, the therapeutic composition may comprise at least one isolated bacterium, wherein the bacterium comprises a gene encoding a 16S rRNA, wherein said gene comprises a sequence with at least 96% sequence identity with the sequence set forth in any one of SEQ ID NOs 2, 7, 8, 10, 12, 30, 32, 39, 42, 44, or 48. In addition, or alternatively, the therapeutic composition may comprise at least one isolated bacterium, wherein the bacterium comprises a gene encoding a 16S rRNA, wherein said gene comprises a sequence with at least 97% sequence identity with the sequence set forth in any one of SEQ ID NOs 3, 16, 25, 31, 34, 36, 37, or 40. In addition, or alternatively, the therapeutic composition may comprise at least one isolated bacterium, wherein the bacterium comprises a gene encoding a 16S rRNA, wherein said gene comprises a sequence with at least 98% sequence identity with the sequence set forth in any one of SEQ ID NOs 4 or 9. In addition, or alternatively, the therapeutic composition may comprise at least one isolated bacterium, wherein the bacterium comprises a gene encoding a 16S rRNA, wherein said gene comprises a sequence with at least 99% sequence identity with the sequence set forth in any one of SEQ ID NOs 27, 28, 38, or 51.

More preferably, the therapeutic composition of the present invention may comprise at least one isolated bacterium, wherein the bacterium comprises a gene encoding a 16S rRNA, wherein said gene comprises a sequence with at least 90% sequence identity with the sequence set forth in SEQ ID NO: 1 or SEQ ID NO: 21. In addition, or alternatively, the therapeutic composition may comprise at least one isolated bacterium, wherein the bacterium comprises a gene encoding a 16S rRNA, wherein said gene comprises a sequence with at least 92% sequence identity with the sequence set forth in any one of SEQ ID NOs 6, or 11. In addition, or alternatively, the therapeutic composition may comprise at least one isolated bacterium, wherein the bacterium comprises a gene encoding a 16S rRNA, wherein said gene comprises a sequence with at least 93% sequence identity with the sequence set forth in SEQ ID NO: 35. In addition, or alternatively, the therapeutic composition may comprise at least one isolated bacterium, wherein the bacterium comprises a gene encoding a 16S rRNA, wherein said gene comprises a sequence with at least 94% sequence identity with the sequence set forth in any one of SEQ ID NOs 5, 19, 22, 23, or 50.

In addition, or alternatively, the therapeutic composition may comprise at least one isolated bacterium, wherein the bacterium comprises a gene encoding a 16S rRNA, wherein said gene comprises a sequence with at least 95% sequence identity with the sequence set forth in any one of SEQ ID NOs 13, 15, 29, 33, 41, 43, 45, or 46. In addition, or alternatively, the therapeutic composition may comprise at least one isolated bacterium, wherein the bacterium comprises a gene encoding a 16S rRNA, wherein said gene comprises a sequence with at least 96% sequence identity with the sequence set forth in any one of SEQ ID NOs 7, 12, 32, 39, 42, or 44, at least 97% sequence identity with the sequence set forth in any one of SEQ ID NOs 3, 8, 10, 16, 34, 36, 37, 40, 48, or 49. In addition, or alternatively, the therapeutic composition may comprise at least one isolated bacterium, wherein the bacterium comprises a gene encoding a 16S rRNA, wherein said gene comprises a sequence with at least 98% sequence identity with the sequence set forth in any one of SEQ ID NOs 4, 9, 17 or 31. In addition, or alternatively, the therapeutic composition may comprise at least one isolated bacterium, wherein the bacterium comprises a gene encoding a 16S rRNA, wherein said gene comprises a sequence with at least 99% sequence identity with the sequence set forth in any one of SEQ ID NOs 2, 20, 38, or 51. In addition, or alternatively, the therapeutic composition may comprise at least one isolated bacterium, wherein the bacterium comprises a gene encoding a 16S rRNA, wherein said gene comprises a sequence with 100% sequence identity with the sequence set forth in any one of SEQ ID NOs 14, 18, 24, 25, 26, 27, 28, 30, or 47.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Other algorithms suitable for the alignment of nucleotide sequences may be used instead of GAP, e.g. BLAST (Basic Local Alignment Search Tool) (which uses the method of Altschul et al. (1990) J. Mol. Biol. 215: 405-410), FASTA (which uses the method of Pearson and Lipman (1988) PNAS USA 85: 2444-2448), the Smith-Waterman algorithm (Smith and Waterman (1981) J. Mol Biol. 147: 195-197), the TBLASTN program, of Altschul et al. (1990) supra, or the psi-Blast algorithm (Nucl. Acids Res. (1997) 25 3389-3402), generally employing default parameters. In particular, BLAST may be used, preferably employing default parameters.

Sequence alignment algorithms, such as BLAST, calculate the similarity score between a query sequence and a subject sequence. The sequence identity of the query sequence to the subject sequence may be dependent on the percentage of the query sequence that is required to overlap with the subject sequence. This is also referred to as query coverage. In a preferred embodiment, the isolated bacterium present in the therapeutic composition of the present invention comprises a gene encoding a 16S rRNA, wherein said gene comprises a sequence, which (in addition to the specified sequence identity) has a query coverage of at least 98%, at least 99%, or 100%, preferably at least 98%. The query coverage refers to the percentage of said sequence which overlaps with the sequence with which it has the specified sequence identity, e.g. SEQ ID NO: 1. For example, the bacterium present in the therapeutic composition may comprise a gene encoding a 16S rRNA, wherein said gene comprises a sequence with at least 90% sequence identity with the sequence set forth in any one of SEQ ID NOs 1 to 51 and at least 98% query coverage.

Yet more preferably, the therapeutic composition of the present invention may comprise at least one isolated bacterium, wherein the bacterium is a bacterium as deposited under the Budapest Treaty on the International Recognition of the Deposit of Microorganisms for the Purposes of Patent Procedures at the Leibniz-Institut DSMZ—Deutsche Sammlung von Mikroorganismen und Zellkulturen GmbH (DSMZ), Inhoffenstr. 7B, 38124 Braunschweig by Genome Research Limited under an accession number as listed in FIG. 15. Specifically, the therapeutic composition of the present invention may comprise at least one isolated bacterium, wherein the bacterium is a bacterium as deposited under the Budapest Treaty at DSMZ under one of the following accession numbers (the date of deposit with DSMZ for each bacterium deposited is indicated in brackets after the accession number): DSM32191 (27 Oct. 2015), DSM32147 (23 Sep. 2015), DSM32149 (23 Sep. 2015), DSM32175 (6 Oct. 2015), DSM32153 (27 Oct. 2015), DSM32152 (23 Sep. 2015), DSM32158 (23 Sep. 2015), DSM32192 (27 Oct. 2015), DSM32148 (23 Sep. 2015), DSM32166 (6 Oct. 2015), DSM32151 (23 Sep. 2015), DSM32150 (23 Sep. 2015), DSM32193 (27 Oct. 2015), DSM32162 (6 Oct. 2015), DSM32194 (27 Oct. 2015), DSM32163 (6 Oct. 2015), DSM32205 (1 Mar. 2016), DSM32195 (27 Oct. 2015), DSM32164 (6 Oct. 2015), DSM32177 (13 Oct. 2015), DSM32167 (6 Oct. 2015), DSM32165 (6 Oct. 2015), DSM32169 (6 Oct. 2015), DSM32168 (6 Oct. 2015), DSM32178 (13 Oct. 2015), DSM32182 (13 Oct. 2015), DSM32179 (13 Oct. 2015), DSM32180 (13 Oct. 2015), DSM32184 (13 Oct. 2015), DSM32181 (13 Oct. 2015), DSM32183 (13 Oct. 2015), DSM 32262 (2 Feb. 2016), DSM32211 (2 Dec. 2015), DSM 32219 (8 Dec. 2015), DSM 32222 (8 Dec. 2015), DSM 32261 (2 Feb. 2016), DSM32212 (2 Dec. 2015), DSM32220 (8 Dec. 2015), DSM32213 (2 Dec. 2015), DSM 32226 (8 Dec. 2015), DSM32215 (2 Dec. 2015), DSM32216 (2 Dec. 2015), DSM 32217 (2 Feb. 2016), DSM32221 (8 Dec. 2015), DSM32218 (2 Dec. 2015), DSM 32224 (8 Dec. 2015), DSM 32214 (2 Dec. 2015), DSM 32263 (2 Feb. 2016), DSM 32223 (8 Dec. 2015), DSM 32225 (8 Dec. 2015), and DSM 32265 (10 Feb. 2016). The putative genus and species names of the deposited bacteria, as well as their known characteristics, are listed in FIG. 15.

Yet more preferably, the therapeutic composition of the present invention comprises at least one isolated bacterium, wherein the bacterium is a bacterium as deposited under the Budapest Treaty at DSMZ under one of the following accession numbers: DSM32191 and DSM32177. In addition, or alternatively, the therapeutic composition may comprise at least one isolated bacterium, wherein the bacterium is a bacterium as deposited under the Budapest Treaty at DSMZ under one of the following accession numbers: DSM32153, DSM32152, DSM32151, DSM32193, DSM32162, DSM32194, DSM32205, DSM32195, DSM32164, DSM32177, DSM32165, DSM32169, DSM32168, DSM32182, DSM32184, DSM32211, DSM32222, DSM32215, DSM32217, DSM32218, DSM32224, DSM32214, DSM32223, and DSM32225; more preferably a bacterium as deposited under one of the following accession numbers: DSM32153, DSM32152, DSM32151, DSM32193, DSM32194, DSM32164, DSM32165, DSM32169, DSM32184, DSM32211, DSM32222, DSM32215, DSM32217, DSM32218, DSM32224, and DSM32225.

Alternatively, the therapeutic composition of the present invention may comprise at least one isolated bacterium, said bacterium comprising a gene encoding a 16S rRNA, wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence of the gene encoding the 16S rRNA in a deposited bacterium as described above.

The therapeutic composition of the invention may comprise at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least nineteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-one, at least thirty-two, at least thirty-three, at least thirty-four, at least thirty-five, at least thirty-six, at least thirty-seven, at least thirty-eight, at least thirty-nine, at least forty, at least forty-one, at least forty-two, at least forty-three, at least forty-four, at least forty-five, at least forty-six, at least forty-seven, at least forty-eight, at least forty-nine, at least fifty, or at least fifty-one bacteria as disclosed herein.

The therapeutic composition of the invention may comprise one, up to two, up to three, up to four, up to five, up to six, up to seven, up to eight, up to nine, up to ten, up to eleven, up to twelve, up to thirteen, up to fourteen, up to fifteen, up to sixteen, up to seventeen, up to eighteen, up to nineteen, up to nineteen, up to twenty, up to twenty-one, up to twenty-two, up to twenty-three, up to twenty-four, up to twenty-five, up to twenty-six, up to twenty-seven, up to twenty-eight, up to twenty-nine, up to thirty, up to thirty-one, up to thirty-two, up to thirty-three, up to thirty-four, up to thirty-five, up to thirty-six, up to thirty-seven, up to thirty-eight, up to thirty-nine, up to forty, up to forty-one, up to forty-two, up to forty-three, up to forty-four, up to forty-five, up to forty-six, up to forty-seven, up to forty-eight, up to forty-nine, up to fifty, or up to fifty-one bacteria as disclosed herein. Preferably, the therapeutic composition of the invention comprise up to twenty, preferably up to ten, bacteria as disclosed herein.

Where a therapeutic composition comprises more than one isolated bacterium, the isolated bacteria are preferably distinct. “Distinct” may refer to the isolated bacteria encoding distinct 16S rRNA sequences.

The therapeutic composition of the invention may comprise at least one isolated bacterium which forms spores. Such a bacterium is also referred to as a spore-forming bacterium. Spores are metabolically dormant structures that are resilient to environmental insults and are used by certain bacteria as a survival strategy upon encountering adverse conditions. Bacteriotherapy candidates HMI_1, HMI_2, HMI_4, HMI_6, HMI_10, HMI_15, HMI_17, HMI_21, HMI_22, HMI_33, HMI_36, HMI_37, HMI_38, HMI_44, HMI_47, HMI_48, HMI_50, HMI_51, and HMI_52 were isolated from ethanol-treated samples and are thus expected to be capable of forming spores. In addition, HMI_3, HMI_7, HMI_8, HMI_16, HMI_18, HMI_19, HMI_24, HMI_25, HMI_26, HMI_27, HMI_28, HMI_29, HMI_30, HMI_34, HMI_41, and HMI_46 are expected to be spore formers based on phylogenetic analysis.

Thus, the therapeutic composition of the present invention may comprise at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-one, at least thirty-two, at least thirty-three, at least thirty-four, or at least thirty-five isolated spore-forming bacteria. In one embodiment, the bacteria in the therapeutic composition may consist of spore forming bacteria.

The spore-forming bacterium may thus be a bacterium comprising a gene encoding a 16S ribosomal RNA (rRNA), wherein said gene comprises a sequence with at least 90% sequence identity with the sequence set forth in any one of SEQ ID NOs 1, 2, 3, 4, 6, 7, 8, 10, 14, 15, 16, 17, 18, 20, 21, 23, 24, 25, 26, 27, 28, 29, 32, 33, 35, 36, 37, 40, 43, 45, 46, 47, 49, 50, or 51. Alternatively, the spore forming bacterium may be a bacterium comprising a gene encoding a 16S ribosomal RNA (rRNA), wherein said gene comprises a sequence with at least 90% sequence identity with the sequence set forth in any one of SEQ ID NOs 1, 2, 4, 6, 10, 14, 16, 20, 21, 32, 35, 36, 37, 43, 46, 47, 49, 50, or 51.

The spore-forming bacterium may be a bacterium comprising a gene encoding a 16S ribosomal RNA (rRNA), wherein said gene comprises a sequence with at least 91%, at least 92%, at least 93%, at least 94%, at least 95%, at least 96%, at least 97%, at least 98%, at least 98.7%, at least 99%, or 100% sequence identity with the sequence set forth in any one of SEQ ID NOs 1, 2, 3, 4, 6, 7, 8, 10, 14, 15, 16, 17, 18, 20, 21, 23, 24, 25, 26, 27, 28, 29, 32, 33, 35, 36, 37, 40, 43, 45, 46, 47, 49, 50, or 51. Alternatively, the spore forming bacterium may be a bacterium comprising a gene encoding a 16S ribosomal RNA (rRNA), wherein said gene comprises a sequence with at least 91%, at least 92%, at least 93%, at least 94%, at least 95%, at least 96%, at least 97%, at least 98%, at least 98.7%, at least 99%, or 100% sequence identity with the sequence set forth in any one of SEQ ID NOs 1, 2, 4, 6, 10, 14, 16, 20, 21, 32, 35, 36, 37, 43, 46, 47, 49, 50, or 51.

Preferably, the spore forming bacterium is a bacterium comprising a gene encoding a 16S ribosomal RNA (rRNA), wherein said gene comprises a sequence with at least 90% sequence identity with the sequence set forth in SEQ ID NOs 1 or SEQ ID NO: 21, and/or at least 91%, at least 92%, at least 93%, at least 94%, at least 95%, at least 96%, at least 97%, at least 98%, at least 98.7%, at least 99%, or 100% sequence identity with the sequence set forth in any one of SEQ ID NOs 2, 3, 4, 6, 7, 8, 10, 14, 15, 16, 17, 18, 20, 23, 24, 25, 26, 27, 28, 29, 32, 33, 35, 36, 37, 40, 43, 45, 46, 47, 49, 50, or 51. More preferably, the spore forming bacterium may be a bacterium comprising a gene encoding a 16S ribosomal RNA (rRNA), wherein said gene comprises a sequence with at least 90% sequence identity with the sequence set forth in SEQ ID NOs 1 or SEQ ID NO: 21, and/or at least 91%, at least 92%, at least 93%, at least 94%, at least 95%, at least 96%, at least 97%, at least 98%, at least 98.7%, at least 99%, or 100% sequence identity with the sequence set forth in any one of SEQ ID Nos 2, 4, 6, 10, 14, 16, 20, 32, 35, 36, 37, 43, 46, 47, 49, 50, or 51.

More preferably, the spore forming bacterium may be a bacterium comprising a gene encoding a 16S ribosomal RNA (rRNA), wherein said gene comprises a sequence with at least 90% sequence identity with the sequence set forth SEQ ID NO: 1 or SEQ ID NO: 21, at least 92% sequence identity with the sequence set forth in SEQ ID NO: 29, at least 92% sequence identity with the sequence set forth in SEQ ID NOs 6, or 24 at least 93% sequence identity with the sequence set forth in SEQ ID NOs 35 or 26, at least 94% sequence identity with the sequence set forth in any one of SEQ ID NOs 14, 15, 17, 18, 23, or 50, at least 95% sequence identity with the sequence set forth in any one of SEQ ID NOs 20, 33, 43, 45, 46, 47, or 49, at least 96% sequence identity with the sequence set forth in any one of SEQ ID NOs 2, 7, 8, 10, or 32, at least 97% sequence identity with the sequence set forth in any one of SEQ ID NOs 3, 16, 25, 36, 37 or 40, at least 98% sequence identity with the sequence set forth in SEQ ID NO: 4, or at least 99% sequence identity with the sequence set forth in SEQ ID NOs 27, 28, or 51. Yet more preferably, the spore forming bacterium may be a bacterium comprising a gene encoding a 16S ribosomal RNA (rRNA), wherein said gene comprises a sequence with at least 90% sequence identity with the sequence set forth SEQ ID NO: 1 or SEQ ID NO: 21, at least 92% sequence identity with the sequence set forth in SEQ ID NO: 6, at least 93% sequence identity with the sequence set forth in SEQ ID NO: 35, at least 94% sequence identity with the sequence set forth in any one of SEQ ID NOs 14, or 50, at least 95% sequence identity with the sequence set forth in any one of SEQ ID NOs 20, 43, 46, 47, or 49, at least 96% sequence identity with the sequence set forth in any one of SEQ ID NOs 2, 10, or 32, at least 97% sequence identity with the sequence set forth in any one of SEQ ID NOs 16, 36, or 37, at least 98% sequence identity with the sequence set forth in SEQ ID NO: 4, or at least 99% sequence identity with the sequence set forth in SEQ ID NO: 51.

More preferably, the spore forming bacterium may be a bacterium comprising a gene encoding a 16S ribosomal RNA (rRNA), wherein said gene comprises a sequence with at least 90% sequence identity with the sequence set forth in SEQ ID NO: 1 or SEQ ID NO: 21, at least 92% sequence identity with the sequence set forth SEQ ID NO: 6, at least 93% sequence identity with the sequence set forth in SEQ ID NO: 35, at least 94% sequence identity with the sequence set forth in SEQ ID NOs 23 or 50, at least 95% sequence identity with the sequence set forth in any one of SEQ ID NOs 15, 29, 33, 43, 45 or 46, at least 96% sequence identity with the sequence set forth in SEQ ID NOs 7 or 32, at least 97% sequence identity with the sequence set forth in any one of SEQ ID NOs 3, 10, 16, 36, 37, 40 or 49, at least 98% sequence identity with the sequence set forth in SEQ ID NOs 4, 8 or 17, at least 99% sequence identity with the sequence set forth in any one of SEQ ID NOs 2, 20, or 51, or 100% sequence identity with the sequence set forth in any one of SEQ ID NOs 14, 18, 25, 26, 27, 28, or 47. Even more preferably, the spore forming bacterium may be a bacterium comprising a gene encoding a 16S ribosomal RNA (rRNA), wherein said gene comprises a sequence with at least 90% sequence identity with the sequence set forth in SEQ ID NO: 1 or SEQ ID NO: 21, at least 92% sequence identity with the sequence set forth SEQ ID NO: 6, at least 93% sequence identity with the sequence set forth in SEQ ID NO: 35, at least 94% sequence identity with the sequence set forth in SEQ ID NO: 50, at least 95% sequence identity with the sequence set forth in any one of SEQ ID NOs 43, or 46, at least 96% sequence identity with the sequence set forth in SEQ ID NO: 32, at least 97% sequence identity with the sequence set forth in any one of SEQ ID NOs 10, 16, 36, 37, or 49, at least 98% sequence identity with the sequence set forth in SEQ ID NO: 4, at least 99% sequence identity with the sequence set forth in any one of SEQ ID NOs 2, 20, or 51, or 100% sequence identity with the sequence set forth in any one of SEQ ID NOs 14, or 47.

Yet more preferably, the spore forming bacterium may be a bacterium as deposited at DSMZ under accession number DSM32191, DSM32147, DSM32175, DSM32152, DSM32166, DSM32162, DSM32163, DSM32177, DSM32167, DSM 32262, DSM 32222, DSM 32261, DSM32212, DSM32217, DSM32224, DSM32214, DSM32223, DSM32225, DSM32265, DSM32149, DSM32158, DSM32192, DSM32194, DSM32205, DSM32195, DSM32169, DSM32168, DSM32178, DSM32182, DSM32179, DSM32180, DSM32211, DSM32226, or DSM32218. Most preferably, the spore forming bacterium is a bacterium as deposited at DSMZ under accession number DSM32191, DSM32147, DSM32175, DSM32152, DSM32166, DSM32162, DSM32163, DSM32177, DSM32167, DSM 32262, DSM 32222, DSM 32261, DSM32212, DSM32217, DSM32224, DSM32214, DSM32223, DSM32225, or DSM32265. Alternatively, the therapeutic composition of the present invention may comprise at least one isolated spore-forming bacterium, said bacterium comprising a gene encoding a 16S rRNA, wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence of the gene encoding the 16S rRNA in a deposited bacterium as described above.

The isolated bacterium present in the therapeutic composition may be antagonistic towards an intestinal bacterium, inhibit or prevent the growth or sporulation of an intestinal bacterium, and/or neutralize or protect against a toxin produced by an intestinal bacterium. Preferably the bacterium inhibits or prevents the growth of an intestinal bacterium. The intestinal bacterium may be a pathogenic or non-pathogenic intestinal bacterium. Preferably, the intestinal bacterium is a pathogenic bacterium. This is particularly preferred in the context of a therapeutic composition for use in the treatment of a dysbiosis associated with an enteric bacterial infection. However, other diseases are also known to be characterised by an increase in certain types of bacteria in the gastrointestinal tract. For example, inflammatory bowel disease is known to be characterised by an increase in bacteria from the Proteobacteria phylum, such as Escherichia coli, in the intestinal microbiota. Similarly, irritable bowel syndrome, obesity and malnutrition are known to be characterised by an increase in certain types of bacteria in the gastrointestinal tract. A bacterial composition comprising at least one bacterium which is antagonistic towards an intestinal bacterium, inhibits or prevents the growth or sporulation of an intestinal bacterium, and/or neutralizes or protects against a toxin produced by an intestinal bacterium thus also finds application in the treatment of dysbiosis associated with inflammatory bowel disease, irritable bowel syndrome, obesity, or malnutrition.

The pathogenic bacterium may be a Gram positive bacterium, or a Gram negative bacterium. Exemplary pathogenic bacteria include pathogenic bacteria of the genera Clostridium, Escherichia, Enterococcus, Klebsiella, Enterobacter, Proteus, Salmonella, Shigella, Staphylococcus, Vibrio, Aeromonas, Campylobacter, Bacillus, Helicobacter, Listeria, Plesiomonas, or Yersinia. In a preferred embodiment, the pathogenic bacterium is a pathogenic bacterium of the genera Clostridium or Escherichia, such as Clostridium difficile or Escherichia coli.

Examples of pathogenic Escherichia coli include adherent-invasive Escherichia coli (AIEC), enteroaggregative Escherichia coli, enterohemorrhagic Escherichia coli, enteroinvasive Escherichia coli, enterotoxigenic Escherichia coli, and Escherichia coli 0157:H7. An enterotoxigenic Escherichia coli may produce a heat-labile enterotoxin, or heat-stable enterotoxin.

For example, the pathogenic bacterium may be Clostridium difficile or adherent-invasive E. coli (AIEC).

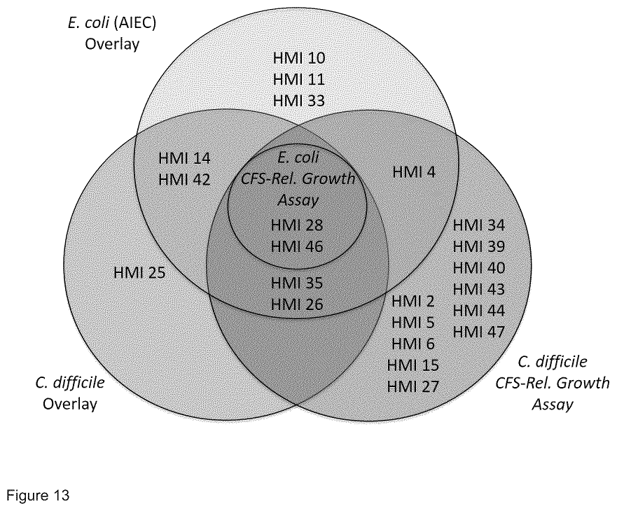

Bacteriotherapy candidates HMI_14, HMI_25, HMI_42, HMI_26, HMI_28, HMI_35 and HMI_46 have been shown to inhibit growth of Clostridium difficile in an overlay assay. In addition, HMI_2, HMI_4, HMI_5, HMI_6, HMI_15, HMI_26, HMI_27, HMI_28, HMI_34, HMI_35, HMI_39, HMI_40, HMI_43, HMI_44, HMI_46 and HMI_47 have been shown to inhibit growth of Clostridium difficile in a CFS-relative growth inhibition assay (see Example 2, FIG. 13, and Table 1).

Bacteriotherapy candidates HMI_4, HMI_10, HMI_11, HMI_14, HMI_26, HMI_28, HMI_33, HMI 35. HMI_42 and HMI_46 have been shown to inhibit growth of Escherichia coli in an overlay assay. In addition, HMI_46 and HMI_28, have been shown to inhibit growth of Escherichia coli in a CFS-relative growth inhibition assay (see Example 2, FIG. 13, and FIG. 15).

It is expected that a bacterium which inhibits the growth of Escherichia coli also inhibits the growth of other Proteobacteria. Thus, the pathogenic bacterium may be a proteobacterium. Proteobacteria include (apart from Escherichia species), Salmonella species, Campylobacter species, Vibrio species, Helicobacter species, and Yersinia species.

It is expected that a bacterium which inhibits the growth of Clostridium difficile also inhibits the growth of other bacteria of the genus Clostridium. Thus, the pathogenic bacterium may be a bacterium of the genus Clostridium. Pathogenic bacteria of the genus Clostridium (apart from Clostridium difficile), include Clostridium perfringens, Clostridium botulinum, and Clostridium tetani.

The therapeutic composition may thus comprise at least one isolated bacterium which inhibits the growth of Clostridium difficile and/or Escherichia coli. For example, the therapeutic composition may comprise at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, or at least twenty-two isolated bacteria which inhibit the growth of Clostridium difficile and/or Escherichia coli. In one embodiment, the bacteria in the therapeutic composition may consist of one or more isolated bacteria which have been shown to inhibit the growth of Clostridium difficile and/or Escherichia coli.

In a preferred embodiment, the therapeutic composition may comprise at least one isolated bacterium which has been shown to inhibit the growth of Clostridium difficile. This is preferred in the context of a therapeutic composition for use in the treatment of a dysbiosis associated with an enteric infection, in particular a dysbiosis associated with an infection with a pathogenic Clostridium-related species, such as Clostridium difficile, Clostridium perfringens, Clostridium botulinum, or Clostridium tetani, most preferably Clostridium difficile.

For example, the therapeutic composition may comprise at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or at least nineteen isolated bacteria which inhibit the growth of Clostridium difficile. In one embodiment, the bacteria in the therapeutic composition may consist of bacteria which inhibit the growth of Clostridium difficile.

A bacterium inhibits the growth of Clostridium difficile may be a bacterium comprising a gene encoding a 16S ribosomal RNA (rRNA), wherein said gene comprises a sequence with at least 90% sequence identity with the sequence set forth in any one of SEQ ID NOs 2, 4, 5, 6, 13, 14, 24, 25, 26, 27, 33, 34, 38, 39, 41, 42, 43, 45 and 46.

Alternatively, a bacterium which inhibits the growth of Clostridium difficile may be a bacterium comprising a gene encoding a 16S ribosomal RNA (rRNA), wherein said gene comprises a sequence with at least 91%, at least 92%, at least 93%, at least 94%, at least 95%, at least 96%, at least 97%, at least 98%, at least 99%, or 100% sequence identity with the sequence set forth in any one of SEQ ID NOs 2, 4, 5, 6, 13, 14, 24, 25, 26, 27, 33, 34, 38, 39, 41,42, 43, 45 and 46.

More preferably, a bacterium which inhibits the growth of Clostridium difficile may be a bacterium comprising a gene encoding a 16S ribosomal RNA (rRNA), wherein said gene comprises a sequence with at least 92% sequence identity with the sequence set forth in any one of SEQ ID NOs 6, or 24, at least 93% sequence identity with the sequence set forth in any one of SEQ ID NOs 13, or 26, at least 94% sequence identity with the sequence set forth in any one of SEQ ID NOs 5, or 14, at least 95% sequence identity with the sequence set forth in any one of SEQ ID NOs 33, 41, 43, 45, or 46, at least 96% sequence identity with the sequence set forth in any one of SEQ ID NOs 2, 39, or 42, at least 97% sequence identity with the sequence set forth in any one of SEQ ID NOs 25, or 34, at least 98% sequence identity with the sequence set forth in SEQ ID NO: 4, or at least 99% sequence identity with the sequence set forth in any one of SEQ ID NOs 27, or 38.

Even more preferably, a bacterium which inhibits the growth of Clostridium difficile may be a bacterium comprising a gene encoding a 16S ribosomal RNA (rRNA), wherein said gene comprises a sequence with at least 92% sequence identity with the sequence set forth SEQ ID NO: 6, at least 94% sequence identity with the sequence set forth in SEQ ID NO: 5, at least 95% sequence identity with the sequence set forth in any one of SEQ ID NOs 13, 33, 41, 43, 45, or 46, at least 96% sequence identity with the sequence set forth in any one of SEQ ID NOs 39, or 42, at least 97% sequence identity with the sequence set forth in SEQ ID NO: 34, at least 98% sequence identity with the sequence set forth in SEQ ID NO: 4, at least 99% sequence identity with the sequence set forth in any one of SEQ ID NOs 2, or 38, or 100% sequence identity with the sequence set forth in any one of SEQ ID NOs 14, 24, 25, 26, or 27.

Most preferably, the bacterium which inhibits the growth of Clostridium difficile may be a bacterium as deposited at DSMZ under accession number DSM32147, DSM32175, DSM32153, DSM32152, DSM32193, DSM32162, DSM32168, DSM32178, DSM32182, DSM32179, DSM32211, DSM 32219, DSM32220, DSM32213, DSM32215, DSM32216, DSM 32217, DSM32218, DSM 32224. Alternatively, the therapeutic composition of the present invention may comprise at least one isolated bacterium which inhibits the growth of Clostridium difficile, wherein said bacterium comprising a gene encoding a 16S rRNA, wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence of the gene encoding the 16S rRNA in a deposited bacterium as described above.

In an alternative preferred embodiment, the therapeutic composition may comprise at least one isolated bacterium which inhibits the growth of Escherichia coli. This is preferred in the context of a therapeutic composition for use in the treatment of a dysbiosis associated with an enteric infection, in particular a dysbiosis associated with an infection with a Proteobacterium, such as Escherichia species, Salmonella species, Campylobacter species, Vibrio species, Helicobacter species, and Yersinia species, most preferably a dysbiosis associated with an infection with Escherichia coli.

For example, the therapeutic composition may comprise at least one, at least two, at least three, at least four, at least five, at least six, at least seven, at least eight, at least nine, or at least ten, isolated bacteria which inhibit the growth of Escherichia coli. In one embodiment, the bacteria in the therapeutic composition may consist of bacteria which inhibit the growth of Escherichia coli.

A bacterium which inhibits the growth of Escherichia coli may be a bacterium comprising a gene encoding a 16S ribosomal RNA (rRNA), wherein said gene comprises a sequence with at least 90% sequence identity with the sequence set forth in any one of SEQ ID NOs 4, 10, 11, 13, 25, 27, 32, 34, 41, and 45.

Alternatively, a bacterium which inhibits the growth of Escherichia coli may be a bacterium comprising a gene encoding a 16S ribosomal RNA (rRNA), wherein said gene comprises a sequence with at least 91%, at least 92%, at least 93%, at least 94%, at least 95%, at least 96%, at least 97%, at least 98%, at least 99%, or 100% sequence identity with the sequence set forth in any one of SEQ ID NOs 4, 10, 11, 13, 25, 27, 32, 34, 41, and 45.

More preferably, a bacterium which inhibits the growth of Escherichia coli may be a bacterium comprising a gene encoding a 16S ribosomal RNA (rRNA), wherein said gene comprises a sequence with at least 92% sequence identity with the sequence set forth in SEQ ID NO: 11, at least 93% sequence identity with the sequence set forth in SEQ ID NO: 13, at least 95% sequence identity with the sequence set forth in any one of SEQ ID NOs 41, or 45, at least 96% sequence identity with the sequence set forth in any one of SEQ ID NOs 10, or 32, at least 97% sequence identity with the sequence set forth in any one of SEQ ID NOs 25, or 34, at least 98% sequence identity with the sequence set forth in SEQ ID NO: 4, or at least 99% sequence identity with the sequence set forth in SEQ ID NO: 27.

Even more preferably, a bacterium which inhibits the growth of Escherichia coli may be a bacterium comprising a gene encoding a 16S ribosomal RNA (rRNA), wherein said gene comprises a sequence with at least 92% sequence identity with the sequence set forth in SEQ ID NO: 11, at least 95% sequence identity with the sequence set forth in any one of SEQ ID NOs 13, 41, or 45, at least 96% sequence identity with the sequence set forth in SEQ ID NO: 32, at least 97% sequence identity with the sequence set forth in any one of SEQ ID NOs 10, or 34, at least 98% sequence identity with the sequence set forth in SEQ ID NO: 4, or 100% sequence identity with the sequence set forth in any one of SEQ ID NOs 25, or 27.

Most preferably, the bacterium which inhibits the growth of Escherichia coli may be a bacterium as deposited at DSMZ under accession number DSM32175, DSM32166, DSM32151, DSM32193, DSM32178, DSM32179, DSM 32262, DSM 32219, DSM32215, DSM32218. Alternatively, the therapeutic composition of the present invention may comprise at least one isolated bacterium which inhibits the growth of Escherichia coli, wherein said bacterium comprising a gene encoding a 16S rRNA, wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence of the gene encoding the 16S rRNA in a deposited bacterium as described above.

In addition to an isolated bacterium which inhibits the growth of Clostridium difficile and/or Escherichia coli, the therapeutic composition may comprise at least one isolated bacterium which co-occurs with a bacterium which inhibits the growth of Clostridium difficile and/or Escherichia coli as disclosed herein. Bacteriotherapy candidates which have been shown to co-occur with a bacterium which inhibits the growth of Clostridium difficile and/or Escherichia coli as disclosed herein are HMI_2, HMI_5, HMI_6, HMI_7, HMI_8, HMI_9, HMI_10, HMI_11, HMI_12, HMI_14, HMI_15, HMI_16, HMI_17, HMI_18, HMI_19, HMI_20, HMI_26, HMI_27, HMI_31, HMI_33, HMI_34, HMI_35, HMI_37, HMI_38, HMI_39, HMI_41, HMI_42, HMI_43, HMI_44, HMI_46, HMI_47, HMI_48, HMI_50, HMI_51, and HMI_52 (see FIG. 15 for details).

Thus, the therapeutic composition may comprise at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least nineteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-one, at least thirty-two, at least thirty-three, at least thirty-four, or thirty-five isolated bacteria which co-occur with a bacterium which inhibits the growth of Clostridium difficile and/or Escherichia coli as disclosed herein.

The bacterium which co-occurs with a bacterium which inhibits the growth of Clostridium difficile and/or Escherichia coli as disclosed herein, may be a bacterium comprising a gene encoding a 16S ribosomal RNA (rRNA), wherein said gene comprises a sequence with at least 90% sequence identity with the sequence set forth in any one of SEQ ID NOs 2, 5, 6, 7, 8, 9, 10, 11, 12, 13, 14, 15, 16, 17, 18, 19, 25, 26, 30, 32, 33, 34, 36, 37, 38, 40, 41, 42, 43, 45, 46, 47, 49, 50, or 51.

Alternatively, the bacterium which co-occurs with a bacterium which inhibits the growth of Clostridium difficile and/or Escherichia coli as disclosed herein, may be a bacterium comprising a gene encoding a 16S ribosomal RNA (rRNA), wherein said gene comprises a sequence with at least 91%, at least 92%, at least 93%, at least 94%, at least 95%, at least 96%, at least 97%, at least 98%, at least 98.7%, at least 99%, or 100% sequence identity with the sequence set forth in any one of SEQ ID NOs 2, 5, 6, 7, 8, 9, 10, 11, 12, 13, 14, 15, 16, 17, 18, 19, 25, 26, 30, 32, 33, 34, 36, 37, 38, 40, 41,42, 43, 45, 46, 47, 49, 50, or 51.

Preferably, the bacterium which co-occurs with a bacterium which inhibits the growth of Clostridium difficile and/or Escherichia coli as disclosed herein, is a bacterium comprising a gene encoding a 16S ribosomal RNA (rRNA), wherein said gene comprises a sequence with at least 92% sequence identity with the sequence set forth in any one of SEQ ID NOs 6, 11, or 19, at least 93% sequence identity with the sequence set forth in any one of SEQ ID NOs 13 or 26, at least 94% sequence identity with the sequence set forth in any one of SEQ ID NOs 5, 14, 15, 17, 18, or 50, at least 95% sequence identity with the sequence set forth in any one of SEQ ID NOs 33, 41, 43, 45, 46, 47, or 49, at least 96% sequence identity with the sequence set forth in any one of SEQ ID NOs 2, 7, 8, 10, 12, 30, 32, or 42, at least 97% sequence identity with the sequence set forth in any one of SEQ ID NOs 16, 25, 34, 36, 37, or 40, at least 98% sequence identity with the sequence set forth in SEQ ID NO: 9, or at least 99% sequence identity with the sequence set forth in any one of SEQ ID NOs 38, or 51.

More preferably, the bacterium which co-occurs with a bacterium which inhibits the growth of Clostridium difficile and/or Escherichia coli as disclosed herein, is a bacterium comprising a gene encoding a 16S ribosomal RNA (rRNA), wherein said gene comprises a sequence with at least 92% sequence identity with the sequence set forth in any one of SEQ ID NOs 6, or 11, at least 94% sequence identity with the sequence set forth in any one of SEQ ID NOs 5, 19, or 50, at least 95% sequence identity with the sequence set forth in any one of SEQ ID NOs 13, 15, 33, 41, 43, 45, or 46, at least 96% sequence identity with the sequence set forth in any one of SEQ ID NOs 7, 12, 32, or 42, at least 97% sequence identity with the sequence set forth in any one of SEQ ID NOs 8, 10, 16, 34, 36, 37, 40, or 49, at least 98% sequence identity with the sequence set forth in any one of SEQ ID NOs 9 or 17, at least 99% sequence identity with the sequence set forth in any one of SEQ ID NOs 2, 38, 51, or at least 100% sequence identity with the sequence set forth in any one of SEQ ID NOs 14, 18, 25, 26, 30, or 47.

Most preferably, the bacterium which co-occurs with a bacterium which inhibits the growth of Clostridium difficile and/or Escherichia coli as disclosed herein, is a bacterium as deposited at DSMZ under accession number DSM32147, DSM32153, DSM32152, DSM32158, DSM32192, DSM32148, DSM32166, DSM32151, DSM32150, DSM32193, DSM32162, DSM32194, DSM32163, DSM32205, DSM32195, DSM32164, DSM32178, DSM32182, DSM32181, DSM32262, DSM32211, DSM32219, DSM32261, DSM32212, DSM32220, DSM32226, DSM32215, DSM32216, DSM32217, DSM32218, DSM32224, DSM32214, DSM32223, DSM32225, or DSM32265. Alternatively, the therapeutic composition of the present invention may comprise at least one isolated bacterium which co-occurs with a bacterium which inhibits the growth of Clostridium difficile and/or Escherichia coli as disclosed herein, wherein said bacterium comprises a gene encoding a 16S rRNA, wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence of the gene encoding the 16S rRNA in a deposited bacterium as described above.

Bacteria related to the genus Clostridium have been shown to be beneficial in reducing inflammation through interactions with the host immune system (Atarashi, Tanoue et al. 2013). The isolated bacterium present in the therapeutic composition may thus be a bacterium which has immunomodulatory activity. For example, the bacterium may reduce inflammation in the individual, e.g. in the gastrointestinal tract of the individual. Bacteriotherapy candidates which are in the same genus as bacteria which have been shown to be beneficial in reducing inflammation through interactions with the host immune system are HMI_4, HMI_9, HMI_10, HMI_15, HMI_27, HMI_28 and HMI_38. The bacteria ar therefore expected to have immunomodulatory activity, such as reducing inflammation in the individual, e.g. in the gastrointestinal tract of the individual.

Thus, the therapeutic composition may comprise at least one, at least two, at least three, at least four, at least five, at least six, or at least seven isolated bacteria which have immunomodulatory activity. In one embodiment, the bacteria in the therapeutic composition may consist of bacteria which reduce inflammation in the individual.

The bacterium which has immunomodulatory activity may be a bacterium comprising a gene encoding a 16S ribosomal RNA (rRNA), wherein said gene comprises a sequence with at least 90% sequence identity with the sequence set forth in any one of SEQ ID NOs 4, 9, 10, 14, 26, 27, or 37.

Alternatively, a bacterium which has immunomodulatory activity may be a bacterium comprising a gene encoding a 16S ribosomal RNA (rRNA), wherein said gene comprises a sequence with at least 91%, at least 92%, at least 93%, at least 94%, at least 95%, at least 96%, at least 97%, at least 98%, at least 99%, or 100% sequence identity with the sequence set forth in any one of SEQ ID NOs 4, 9, 10, 14, 26, 27, or 37.

Preferably, a bacterium which has immunomodulatory activity may be a bacterium comprising a gene encoding a 16S ribosomal RNA (rRNA), wherein said gene comprises a sequence with at least 93% sequence identity with the sequence set forth in SEQ ID NO: 26, at least 94% sequence identity with the sequence set forth in SEQ ID NO: 14, at least 96% sequence identity with the sequence set forth in SEQ ID NO:10, at least 97% sequence identity with the sequence set forth in SEQ ID NO: 37, at least 98% sequence identity with the sequence set forth in any one of SEQ ID NOs 4 or 9, or at least 99% sequence identity with the sequence set forth in SEQ ID NO: 27. More preferably, a bacterium which has immunomodulatory activity may be a bacterium comprising a gene encoding a 16S ribosomal RNA (rRNA), wherein said gene comprises a sequence with at least 97% sequence identity with the sequence set forth in in any of SEQ ID NOs 10 or 37, at least 98% sequence identity with the sequence set forth in any one of SEQ ID NOs 4 or 9, or at least 100% sequence identity with the sequence set forth in any one of SEQ ID NOs 14, 26, or 27.

Most preferably, the bacterium which has immunomodulatory activity may be a bacterium as deposited at DSMZ under accession number DSM32175, DSM32148, DSM32166, DSM32162, DSM32182, DSM32179, or DSM32212. Alternatively, the therapeutic composition of the present invention may comprise at least one isolated bacterium which has immunomodulatory activity, wherein said bacterium comprising a gene encoding a 16S rRNA, wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence of the gene encoding the 16S rRNA in a deposited bacterium as described above.

The therapeutic composition of the invention may comprise at least one isolated bacterium which is on the Human Microbiome Project's (HMP) “most wanted” list. Bacteriotherapy candidates HMI_1, HMI_2, HMI_4, HMI_5, HMI_7, HMI_11, HMI_12, HMI_15, HMI_16, HMI_17, HMI_18, HMI_19, HMI_35, HMI_37, HMI_38, HMI_39, HMI_45, HMI_50, and HMI_51 are on HMP's “most wanted” list.

Thus, the therapeutic composition of the present invention may comprise at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, or nineteen bacteria which are on HMP's “most wanted” list. In one embodiment, the bacteria in the therapeutic composition may consist of bacteria which are on HMP's “most wanted” list.

The bacterium which is on HMP's “most wanted” list may be a bacterium comprising a gene encoding a 16S ribosomal RNA (rRNA), wherein said gene comprises a sequence with at least 90% sequence identity with the sequence set forth in any one of SEQ ID NOs 1, 2, 4, 5, 7, 11, 12, 14, 15, 16, 17, 18, 34, 36, 37, 38, 44, 49, or 50.

Alternatively, the bacterium which is on HMP's “most wanted” list may be a bacterium comprising a gene encoding a 16S ribosomal RNA (rRNA), wherein said gene comprises a sequence with at least 91%, at least 92%, at least 93%, at least 94%, at least 95%, at least 96%, at least 97%, at least 98%, at least 98.7%, at least 99%, or 100% sequence identity with the sequence set forth in any one of SEQ ID NOs 1, 2, 4, 5, 7, 11, 12, 14, 15, 16, 17, 18, 34, 36, 37, 38, 44, 49, or 50.

Preferably, the bacterium which is on HMP's “most wanted” list is a bacterium comprising a gene encoding a 16S ribosomal RNA (rRNA), wherein said gene comprises a sequence with at least 90% sequence identity with the sequence set forth SEQ ID NO: 1, at least 92% sequence identity with the sequence set forth SEQ ID NO: 11, at least 94% sequence identity with the sequence set forth in any one of SEQ ID NOs 5, 14, 15, 17, 18, or 50, at least 95% sequence identity with the sequence set forth SEQ ID NO: 49, at least 96% sequence identity with the sequence set forth in any one of SEQ ID NOs 2, 7, 12, or 44, at least 97% sequence identity with the sequence set forth in any one of SEQ ID NOs 16, 34, 36, or 37, at least 98% sequence identity with the sequence set forth in SEQ ID NO: 4, or at least 99% sequence identity with the sequence set forth in SEQ ID NO: 38.

More preferably, the bacterium which is on HMP's “most wanted” list is a bacterium comprising a gene encoding a 16S ribosomal RNA (rRNA), wherein said gene comprises a sequence with at least 90% sequence identity with the sequence set forth SEQ ID NO: 1, at least 92% sequence identity with the sequence set forth SEQ ID NO: 11, at least 94% sequence identity with the sequence set forth in any one of SEQ ID NOs 5 or 50, at least 95% sequence identity with the sequence set forth SEQ ID NO: 15, at least 96% sequence identity with the sequence set forth in any one of SEQ ID NOs 7, 12, or 44, at least 97% sequence identity with the sequence set forth in any one of SEQ ID NOs 16, 34, 36, 37, 49, at least 98% sequence identity with the sequence set forth in any one of SEQ ID NOs 4, or 17, at least 99% sequence identity with the sequence set forth in any one of SEQ ID NOs 2, or 38, or 100% sequence identity with the sequence set forth in any one of SEQ ID NOs 14, or 18.

Most preferably, the bacterium which is on HMP's “most wanted” list is a bacterium as deposited at DSMZ under accession number DSM32191, DSM32147, DSM32175, DSM32153, DSM32158, DSM32151, DSM32150, DSM32162, DSM32194, DSM32163, DSM32205, DSM32195, DSM32219, DSM32261, DSM32212, DSM32220, DSM32221, DSM32223, or DSM32225. Alternatively, the therapeutic composition of the present invention may comprise at least one bacterium which is on HMP's “most wanted” list, wherein said bacterium comprises a gene encoding a 16S rRNA, wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence of the gene encoding the 16S rRNA in a deposited bacterium as described above.

The therapeutic composition of the invention may comprise at least one isolated bacterium which is a keystone species. Bacteriotherapy candidates HMI_17, HMI_23, HMI_24, HMI_25, HMI_26, HMI_27, HMI_28, HMI_29, HMI_30, HMI_31, HMI_32, HMI_45, HMI_49, HMI_51, and HMI_52 are expected to be keystone species.

Thus, the therapeutic composition of the present invention may comprise at least one, at least two, at least three, at least four, at least five, at least six, at least seven, at least eight, at least nine, at least ten, at least eleven, at least twelve, at least thirteen, at least fourteen, or fifteen bacteria which are keystone species. In one embodiment, the bacteria in the therapeutic composition may consist of bacteria which are keystone species.

The bacterium which is a keystone species may be a bacterium comprising a gene encoding a 16S ribosomal RNA (rRNA), wherein said gene comprises a sequence with at least 90% sequence identity with the sequence set forth in any one of SEQ ID NOs 16, 22, 23, 24, 25, 26, 27, 28, 29, 30, 31, 44, 48, 50, or 51.

Alternatively, the bacterium which is a keystone species may be a bacterium comprising a gene encoding a 16S ribosomal RNA (rRNA) wherein said gene comprises a sequence with at least 91%, at least 92%, at least 93%, at least 94%, at least 95%, at least 96%, at least 97%, at least 98%, at least 98.7%, at least 99%, or 100% sequence identity with the sequence set forth in any one of SEQ ID NOs 16, 22, 23, 24, 25, 26, 27, 28, 29, 30, 31, 44, 48, 50, or 51.

Preferably, the bacterium which is a keystone species is a bacterium comprising a gene encoding a 16S ribosomal RNA (rRNA) wherein said gene comprises a sequence with at least 91% sequence identity with the sequence set forth in SEQ ID NO: 29, at least 92% sequence identity with the sequence set forth in SEQ ID NO: 24, at least 93% sequence identity with the sequence set forth in any one of SEQ ID NOs 22, or 26, at least 94% sequence identity with the sequence set forth in any one of SEQ ID NOs 23, or 50, at least 96% sequence identity with the sequence set forth in any one of SEQ ID NOs 30, 44, or 48, at least 97% sequence identity with the sequence set forth in any one of SEQ ID NOs 16, 25, or 31, or at least 99% sequence identity with the sequence set forth in any one of SEQ ID NOs 27, 28, or 51.

More preferably, the bacterium which is a keystone species is a bacterium comprising a gene encoding a 16S ribosomal RNA (rRNA) wherein said gene comprises a sequence with at least 94% sequence identity with the sequence set forth in any one of SEQ ID NOs 22, 23, or 50, at least 95% sequence identity with the sequence set forth in SEQ ID NO: 29, at least 96% sequence identity with the sequence set forth in SEQ ID NO: 44, at least 97% sequence identity with the sequence set forth in any one of SEQ ID NOs 16, or 48, at least 98% sequence identity with the sequence set forth in SEQ ID NO: 31, at least 99% sequence identity with the sequence set forth in SEQ ID NO: 51, or 100% sequence identity with the sequence set forth in any one of SEQ ID NOs 24, 25, 26, 27, 28, or 30.

Most preferably, the bacterium which is a keystone species is a bacterium as deposited at DSMZ under accession number DSM32163, DSM32165, DSM32169, DSM32168, DSM32178, DSM32182, DSM32179, DSM32180, DSM32184, DSM32181, DSM32183, DSM32221, DSM32263, DSM32225, or DSM32265. Alternatively, the therapeutic composition of the present invention may comprise at least one bacterium which is a keystone species, wherein said bacterium comprises a gene encoding a 16S rRNA, wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence of the gene encoding the 16S rRNA in a deposited bacterium as described above.

In addition, or alternatively, the therapeutic composition of the invention may comprise at least one isolated bacterium which has been shown to be present post-FMT. The bacteriotherapy candidates to which this applies are set out in FIG. 15. For example, the therapeutic composition may comprise at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one or twenty two bacteria which has been shown to be present post-FMT. For example the bacterium which has been shown to be present post-FMT may comprise a gene encoding a 16S ribosomal RNA (rRNA), wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence set forth in any one of SEQ ID NOs 22, 23, 24, 25, 26, 27, 28, 29, 30, 31, 32, 33, 34, 36, 38, 40, 43, 44, 45, 46, 48, or 51. Preferably, the bacterium which has been shown to be present post-FMT is a bacterium as deposited at DSMZ under accession number DSM32165, DSM32169, DSM32168, DSM32178, DSM32182, DSM32179, DSM32180, DSM32184, DSM32181, DSM32183, DSM32262, DSM32211, DSM32219, DSM32261, DSM32220, DSM32226, DSM32217, DSM32221, DSM32218, DSM32224, DSM32263, or DSM32265. Alternatively, the therapeutic composition of the present invention may comprise at least one bacterium which has been shown to be present post-FMT, wherein said bacterium comprises a gene encoding a 16S rRNA, wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence of the gene encoding the 16S rRNA in a deposited bacterium as described above.

In addition, or alternatively, the therapeutic composition of the invention may comprise at least one isolated bacterium which is expected to produce one or more beneficial metabolites, such as short chain fatty acids (SCFA). The bacteriotherapy candidates to which this applies are set out in FIG. 15. For example, the therapeutic composition may comprise at least one, at least two, at least three, at least four, at least five, at least six, at least seven, at least eight, at least nine, at least ten, at least eleven, at least twelve, at least thirteen, or fourteen bacteria which produce one or more beneficial metabolites. For example the bacterium which produces one or more beneficial metabolites may comprise a gene encoding a 16S ribosomal RNA (rRNA), wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence set forth in any one of SEQ ID NOs 9, 12, 19, 20, 22, 23, 24, 25, 26, 27, 28, 29, 30, or 31. Preferably, the bacterium which is expected to produce one or more beneficial metabolites is a bacterium as deposited at DSMZ under accession number DSM32148, DSM32150, DSM32164, DSM32177, DSM32165, DSM32169, DSM32168, DSM32178, DSM32182, DSM32179, DSM32180, DSM32184, DSM32181, or DSM32183. Alternatively, the therapeutic composition of the present invention may comprise at least one bacterium which produces one or more beneficial metabolites, wherein said bacterium comprises a gene encoding a 16S rRNA, wherein said gene comprises a sequence with at least 90%, at least 91%, at least 92%, at least 93%, at least 94%, at least 95%, at least 96%, at least 97%, at least 98%, at least 98.7%, at least 99%, or 100% sequence identity with the sequence of the gene encoding the 16S rRNA in a deposited bacterium as described above.

The isolated bacterium or isolated bacteria present in a therapeutic composition may make up at least 1%, 5%, 10%, 15%, 20%, 25%, 30%, 35%, 40%, 45%, 50%, 55%, 60%, 65%, 7%, 75%, 80%, 85%, or 90% of the therapeutic composition by volume or weight.

The therapeutic composition may comprise no other active ingredient other than the isolated bacterium or isolated bacteria in question, and optionally a prebiotic. Thus, the active ingredient of the therapeutic composition may consist of one or more isolated bacteria disclosed herein, and optionally a prebiotic. This may also be referred to as a defined active ingredient.

The therapeutic composition of the present invention is not a faecal microbiota transplant (FMT). FMTs usually consist of a stool sample from a healthy human donor which is administered directly to the recipient, e.g. in the form of an enema, without bacteria present in the stool sample being isolated prior to the administration of the FMT to the recipient. An advantage of the therapeutic composition of the invention is that it may comprise no undefined components, which are present in FMTs, thereby allowing the therapeutic composition to be standardised and increasing safety.

The therapeutic composition of the present invention may be prepared by a method comprising culturing the one or more isolated bacteria present in the therapeutic composition in a suitable medium or media. Media and conditions suitable for culturing the bacteria to be included in the therapeutic composition of the present invention are described in detail elsewhere herein. For example, a method of preparing a therapeutic composition according to the present invention may comprise the steps of:
(i) culturing a first isolated bacterium;
(ii) optionally culturing a second isolated bacterium; and
(iii) mixing the bacteria obtained in (i) and optionally (ii) to prepare the therapeutic composition. The isolated bacteria to be included in the therapeutic composition are preferably cultured in separate steps. In other words, a separate culture of each bacterium to be included in the therapeutic composition is preferably prepared. This allows the growth of each bacterium to be evaluated and the amount of each bacterium to be included in the pharmaceutical composition to be controlled as desired. The bacteria cultured in steps (i) and (ii) preferably have distinct 16S rRNA sequences.

The above method may include steps of culturing each isolated bacterium which is to be included in the therapeutic composition. Thus, the method may e.g. further include steps of culturing a third, fourth, fifth, sixth, seventh, eighth, ninth, and/or tenth distinct isolated bacterium, as required. In this way, the method comprise steps of culturing up to 51 distinct isolated bacteria. The bacterium or bacteria cultured by said method may be any bacterium as disclosed herein.

The method may optionally comprise one or more further steps in which the bacteria are mixed with one or more additional ingredients, such as a pharmaceutically acceptable excipient, prebiotic, carrier, insoluble fibre, buffer, osmotic agent, antifoaming agent, and/or preservative. In addition, or alternatively, the method may comprise suspending the bacteria obtained in (i) and optionally (ii) in a chemostat medium, or saline, e.g. 0.9% saline. The bacteria obtained in (i) and optionally (ii) may be provided under a reduced atmosphere, such as N₂, CO₂, H₂, or a mixture thereof, e.g. N₂:CO₂:H₂. The gases may be present in appropriate ratios for the preservation of the bacteria present in the therapeutic composition. For Example, the reduced atmosphere may comprise 80% N₂, 10% CO₂ and 10% H₂. In addition, or alternatively, the method may comprise a step of lyophilising the bacteria obtained in (i) and optionally (ii), optionally in the presence of a stabiliser and/or cryprotectant. The method may also comprise a step of preparing a capsule, tablet, or enema comprising the bacteria obtained in (i) and optionally (ii). The capsule or tablet may be enteric-coated, pH dependant, slow-release, and/or gastro-resistant.

The present invention also encompasses a therapeutic composition obtainable by, or obtained by, a method as disclosed herein. Such a therapeutic composition may further be used for a therapeutic purpose, in a therapeutic method, or for the manufacture of a medicament, as described herein, such as treatment of a dysbiosis, in particular a dysbiosis of the gastrointestinal tract.

It is expected that the bacteria disclosed herein will be suitable for the treatment of a dysbiosis, in particular a dysbiosis of the gastrointestinal tract. Without wishing to be limited by theory, it is expected that administration of one or more of the bacteria disclosed herein to an in individual will resolve a gastrointestinal dysbiosis, where present, and/or prevent the occurrence of gastrointestinal dysbiosis, in the individual. “Individual”, as used herein, refers to a human individual or human patient.

Treatment of a dysbiosis may refer to the cure, prevention, or amelioration of a dysbiosis or the amelioration of at least one symptom associated with dysbiosis. Where the dysbiosis is associated with a disease, such as inflammatory bowel disease, treatment of the dysbiosis may refer to the cure, prevention, or amelioration of said disease, or the amelioration of at least one symptom associated with said disease.

The therapeutic compositions of the present invention thus find application in the treatment of dysbiosis, in particular dysbiosis of the gastrointestinal tract. Accordingly, the invention provides a method of treating a dysbiosis comprising administering a therapeutically effective amount of a therapeutic composition of the invention to an individual in need thereof, a therapeutic composition according to the invention for use in a method of treating a dysbiosis in an individual, and the use of a therapeutic composition of the invention for the manufacture of a medicament for the treatment of a dysbiosis in an individual.

“Dysbiosis” in the context of the present invention refers to a state in which the normal diversity and/or function of the microbiota or microbiome, in particular the human gastrointestinal microbiota, is disrupted. Any disruption from the normal state of the microbiota in a healthy individual can be considered a dysbiosis, even if the dysbiosis does not result in a detectable decrease in health in the individual. In a preferred embodiment, the dysbiosis may be associated with one or more pathological symptoms. For example, “dysbiosis” may refer to a decrease in the microbial diversity of the microbiota. In addition, or alternatively, “dysbiosis” may refer to an increase in the abundance of one or more bacteria, e.g. one or more pathogenic bacteria, in the microbiota of an individual relative to the abundance of said bacterium or bacteria in the microbiota of a healthy individual, i.e. an individual without a dysbiosis. The pathogenic bacteria present during dysbiosis are often Proteobacteria and resistant to one or more antibiotics. Examples of Proteobacteria include Escherichia, Salmonella, Campylobacter, Vibrio, Helicobacter, and Yersinia species.

The dysbiosis may be a dysbiosis associated with an enteric bacterial infection, such as an infection of the gastrointestinal tract with a pathogenic bacterium. Many bacteria capable of causing infections of the gastrointestinal tract in humans are known and include: gram positive bacteria, and gram negative bacteria. The pathogenic bacterium is preferably a pathogenic species of the genus Clostridium, Escherichia, Enterococcus, Klebsiella, Enterobacter, Proteus, Salmonella, Shigella, Staphylococcus, Vibrio, Aeromonas, Campylobacter, Plesiomonas, Bacillus, Helicobacter, Listeria, or Yersinia. Preferred examples of such pathogenic bacteria include Clostridium difficile, Clostridium perfringens, Clostridium botulinum, Escherichia coli, Salmonella typhi, Staphylococcus aureus, Vibrio cholerae, Vibrio parahaemolyticus, Vibrio vulnificus, Campylobacter fetus, Campylobacter jejuni, Aeromonas hydrophila, Plesiomonas shigelloides, Bacillus cereus, Helicobacter pylori, Listeria monocytogenes, and Yersinia enterocolitica. More preferably, the pathogenic bacterium is a pathogenic species of the genus Clostridium or Escherichia. Most preferably, the pathogenic bacterium is Clostridium difficile or Escherichia coli.

The pathogenic bacterium may resistant to one or more antibiotics. For example, the pathogenic bacterium, e.g. Clostridium difficile, may be resistant to fluoroquinolones. In addition, or alternatively, the pathogenic bacterium may be resistant to one or more carbapenems. Carbapenems are antibiotics used for the treatment of infections by multidrug-resistant (MDR) bacteria, and examples include imipenem, meropenem, ertapenem, doripenem, panipenem, and biapenem.

Treatment of a dysbiosis associated with an infection with a pathogenic bacterium may comprise reducing the abundance of the pathogenic bacterium, e.g. in the gastrointestinal tract of the individual, relative to the abundance of the pathogenic bacterium prior to treatment.

The dysbiosis may be a recurrent or chronic dysbiosis. For example, Clostridium difficile is known to result in recurrent infections in some individuals, with the infection reoccurring once antibiotic treatment is stopped. This may be referred to as a recurrent or chronic dysbiosis.

Dysbiosis of the gastrointestinal tract is known to be associated with, and is thought to play a causal role in, a number of diverse diseases, including inflammatory bowel disease, irritable bowel syndrome, metabolic disease, a neuropsychiatric disorder, an autoimmune disease, an allergic disorder, or a cancer. Thus the dysbiosis may be a dysbiosis associated with inflammatory bowel disease, irritable bowel syndrome, a metabolic disease, a neuropsychiatric disorder, an autoimmune disease, an allergic disorder, a cancer, or hepatic encephalopathy. Examples of inflammatory bowel disease include ulcerative colitis and Crohn's disease.

Metabolic disease in which dysbiosis of the gastrointestinal tract has been shown to play a role include metabolic syndrome, obesity, type 2 diabetes mellitus, a cardiovascular disease, and non-alcoholic fatty liver.

Neuropsychiatric disorder in which dysbiosis of the gastrointestinal tract has been shown to play a role include Parkinson's disease, Alzheimer's disease, multiple sclerosis, myoclonus dystonia, autism and chronic fatigue syndrome.

Autoimmune diseases in which dysbiosis of the gastrointestinal tract has been shown to play a role include idiopathic thrombocytopenic purpura, arthritis, Sjögren's syndrome, systemic lupus erythematosus, and Hashimoto's thyroiditis.

Allergic disorder in which dysbiosis of the gastrointestinal tract has been shown to play a role include atopy, and asthma.

Cancers in which dysbiosis of the gastrointestinal tract has been shown to play a role include colorectal cancer, extra-intestinal tumours, mammary tumours, hepatocellular carcinoma, lymphoma, melanoma, and lung cancer.

The therapeutic composition of the invention may comprise a pharmaceutically acceptable excipient, carrier, buffer, stabilizer or other materials well known to those skilled in the art. Such materials should be non-toxic and should not interfere with the efficacy of the isolated bacteria present in the therapeutic composition. The precise nature of the pharmaceutically acceptable excipient or other material will depend on the route of administration, which may be oral or rectal. Many methods for the preparation of therapeutic compositions are known to those skilled in the art. See e.g. Robinson ed., Sustained and Controlled Release Drug Delivery Systems, Marcel Dekker, Inc., New York, 1978.

The therapeutic composition of the invention may comprise a prebiotic, a carrier, insoluble fibre, a buffer, an osmotic agent, an anti-foaming agent and/or a preservative.

Prebiotics may provide nutrients for the isolated bacteria present in the therapeutic composition to assist their early growth and colonisation after administration to the individual. Any prebiotic known in the art may be used. Non-limiting examples of prebiotics include oligosaccharides, e.g., fructooligosaccharides such as oligofructose and inulin, mannan oligosaccharides and galactooligosaccharides, soluble, oligofructose-enriched inulin and soluble fiber. Insoluble fiber may be included in the therapeutic composition as a carrier, e.g., to provide protection during transit or storage. A buffer may be included in the therapeutic composition to promote the viability of the isolated bacteria present. An anti-fungal agent may included in the therapeutic composition as a preservative.

The therapeutic composition may be made or provided in chemostat medium. Alternatively, the therapeutic composition may be made or provided in saline, e.g., 0.9% saline. It will be understood that any carrier or solution which does not impair viability of the bacteria present in the therapeutic composition and is compatible with administration to an individual may be used.

The therapeutic composition may be made or provided under reduced atmosphere, i.e., in the absence of oxygen. The synthetic stool preparation may be made or provided under N₂, CO₂, H₂, or a mixture thereof, optionally with controlled levels of partial pressure of N₂:CO₂:H₂.

The therapeutic composition may be for oral or rectal administration to the individual. Where the therapeutic composition is for oral administration, the therapeutic composition may be in the form of a capsule, or a tablet. Where the therapeutic composition is for rectal administration, the therapeutic composition may be in the form of an enema. The preparation of suitable capsules, tablets and enema is well-known in the art. The capsule or tablet may comprise a coating to protect the capsule or tablet from stomach acid. For example, the capsule or tablet may be enteric-coated, pH dependant, slow-release, and/or gastro-resistant. Such capsules and tablets are used, for example, to minimize dissolution of the capsule or tablet in the stomach but allow dissolution in the small intestine.

The therapeutic composition may be lyophilized. The lyophilized therapeutic composition may comprise one or more stabilisers and/or cryoprotectants. The lyophilized therapeutic composition may be reconstituted using a suitable diluent prior to administration to the individual.

A therapeutic composition according to the present invention may be administered alone or in combination with other treatments, concurrently or sequentially or as a combined preparation with another therapeutic agent or agents, for the treatment of dysbiosis, or a disease associated with dysbiosis as described herein. For example, a conjugate of the invention may be used in combination with an existing therapeutic agent for inflammatory bowel disease, irritable bowel syndrome, a metabolic disease, a neuropsychiatric disorder, an autoimmune disease, an allergic disorder, a cancer, or hepatic encephalopathy.

For example, where the therapeutic composition is for the treatment of a dysbiosis associated with cancer, the therapeutic composition may optionally be administered in combination a cancer immunotherapy, such as an immune check-point inhibitor, to the individual. Examples of check-point inhibitors which may be employed in this context include Programmed cell death protein 1 (PD-1) inhibitors, Programmed death-ligand 1 (PD-L1) inhibitors, cytotoxic T-lymphocyte-associated protein 4 (CTLA-4) inhibitors. Manipulation of the gut microbiota in combination with immune check-point inhibitor treatment has been shown to improve efficacy of immune check-point inhibitors in treating cancer (Snyder et al. 2015). In a preferred embodiment, the cancer in this context is lung cancer or melanoma. Immune check-point inhibitors have been approved for the treatment of these cancers and bacteriotherapy has been shown to improve efficacy of check-point inhibitors in the treatment of melanoma (Snyder et al. 2015).

The therapeutic compositions of the invention may be administered to an individual, preferably a human individual. Administration may be in a “therapeutically effective amount” this being sufficient to show benefit to the individual. Such benefit may be at least amelioration of at least one symptom. Thus “treatment” of a specified disease refers to amelioration of at least one symptom. The actual amount administered, and rate and time-course of administration, will depend on the nature and severity of what is being treated, the particular patient being treated, the clinical condition of the individual patient, the cause of the dysbiosis, the site of delivery of the composition, the type of therapeutic composition, the method of administration, the scheduling of administration and other factors known to medical practitioners. Prescription of treatment, e.g. decisions on dosage etc., is within the responsibility of general practitioners and other medical doctors, and may depend on the severity of the symptoms and/or progression of a disease being treated. A therapeutically effective amount or suitable dose of a therapeutic composition of the invention can be determined by comparing its in vitro activity and in vivo activity in an animal model. Methods for extrapolation of effective dosages in mice and other test animals to humans are known.

The precise dose will depend upon a number of factors, including whether the therapeutic composition is for prevention or for treatment.

Further aspects and embodiments of the invention will be apparent to those skilled in the art given the present disclosure including the following experimental exemplification.

All documents mentioned in this specification are incorporated herein by reference in their entirety.

Unless the context dictates otherwise, the singular includes the plural.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### EXAMPLES

### Example 1—Identification and Isolation of Bacteriotherapy Candidates

Materials and Methods

Two different approaches were used to isolate bacterial species for inclusion in a therapeutic composition for treating dysbiosis. The first relied on a broad culturing approach from healthy adult donors to establish a culture collection which is as representative as possible of the bacterial component of the intestinal microbiota of healthy individuals. This process also incorporated a targeted culturing approach to preferentially select bacteria displaying a particular phenotype or function e.g. spore formation. The second approach was more targeted in nature and aimed to isolate bacterial species specifically associated with resolving gastrointestinal dysbiosis by comparing the microbiota of individuals before and after Faecal Microbiota Transplantation (FMT) administered to resolve dysbiosis associated with C. difficile infection. These two approaches are respectively referred to as Candidate Isolation Process 1 (CIP1) and Candidate Isolation Process 2 (CIP2) below.

Sample Collection and Culturing

For CIP1, fresh faecal samples were obtained from six consenting healthy adult human donors (one faecal sample per donor—minimum 0.5 g). The samples were placed in anaerobic conditions within one hour of passing to preserve the viability of anaerobic bacteria. All sample processing and culturing took place under anaerobic conditions in a Whitley DG250 workstation (Don Whitley, West Yorkshire, UK) at 37° C. Culture media, phosphate-buffered saline (PBS) and all other materials that were used for culturing were placed in the anaerobic cabinet 24 hours before use. The faecal samples were divided into two portions. One portion was homogenised in reduced PBS (0.1 g stool/ml PBS) and was serially diluted and plated directly onto YCFA (Duncan, Hold et al. 2002) agar supplemented with 0.002 g/ml each of glucose, maltose and cellobiose in large (13.5 cm diameter) petri dishes. This sample was also subjected to metagenomic sequencing to profile the entire community. The other portion was treated with an equal volume of 70% (v/v) ethanol for 4 hours at room temperature under ambient aerobic conditions to kill vegetative cells. Then, the solid material was washed 3 times with PBS and it was eventually resuspended in PBS. Plating was performed in same manner as described for the non-ethanol treated samples above.

For the ethanol-treated samples of CIP1, the medium was supplemented with 0.1% sodium taurocholate to stimulate spore germination. Colonies were picked 72 hours after plating from petri dishes of both ethanol-treated and non-ethanol-treated conditions harbouring non-confluent growth, (i.e. plates on which the colonies were distinct and not touching). The colonies that were picked were re-streaked to confirm purity.

For CIP2, twelve individuals who had each experienced more than three recurrences of Clostridium difficile infection (CDI), having failed treatment with metronidazole and vancomycin were selected for Faecal Microbiota Transplantation (FMT). The donors were screened for pathogens and other viral infections as previously described (Landy, Al-Hassi et al. 2011). The patients discontinued oral vancomycin 1-2 days before FMT. FMT was administered to recipients by enema (n=3), pills (n=6), a combination of both (n=2, R8 and R10), or by nasogastric infusion (n=1, R7). Faecal samples were collected from the patients 1-2 days after stopping vancomycin treatment (pre-FMT) and at different times post-FMT. Stool samples used for FMT were also collected from the donors. Samples from healthy individuals and individuals infected with C. difficile treated with antibiotics were also included as controls. Faecal samples were collected in sterile containers and were frozen at −80° C. DNA was extracted from all samples for 454 sequencing and subsequent analysis as described below.

For the culturing of samples from the faecal microbiota transplant (FMT) recipients (CIP2), 50 mg of each faecal sample was mixed thoroughly in 0.5 ml sterile, reduced phosphate buffered saline (PBS). The homogenate was serially diluted to 10⁻⁶ and aliquots of this dilution were plated on a panel of media under anaerobic conditions. The following media were used: fastidious anaerobic agar (FAA, Lab M Ltd, Lancashire, UK) containing 2% defibrinated horse blood, Brain Heart Infusion (BHI, Oxoid UK), de Man Rogosa Sharpe and CCEY (Bioconnections, UK) agars with and without the addition of 10 μg/ml vancomycin (AppliChem, Germany). All plated media were incubated anaerobically at 37° C. for 48-72 hours except for the BHI agar, which was incubated aerobically at 37° C. for 24-48 hours.

Microbiota Profiling and Sequencing

Identification of each cultured isolate was performed by PCR amplification of the full length 16S rRNA gene (using 7F (5-AGAGTTTGATYMTGGCTCAG-3) (SEQ ID NO: 52) forward primer and 1510R (5-ACGGYTACCTTGTTACGACTT-3) (SEQ ID NO: 53) reverse primer followed by capillary sequencing. For both CIP1 and CIP2, 16S rRNA gene sequence reads were aligned in the Ribosomal Database Project (RDP) and manually curated in ARB (Ludwig, Strunk et al. 2004). For CIP1, the R package seqinr version 3.1 was used to determine sequence similarity between 16S rRNA gene sequences and, as full-length 16S rRNA gene sequence reads were generated, 98.7% was used as the species-level cut-off to classify reads to Operational Taxonomic Units (OTUs) (Bosshard, Abels et al. 2003, Clarridge 2004). As only partial length 16S rRNA gene sequence reads were generated for candidate bacteria from CIP2, 97% was used as the species-level cut-off (Bosshard, Abels et al. 2003, Clarridge 2004) and the OTUs at this cut-off were determined using mothur (Schloss, Westcott et al. 2009). For both CIP1 and CIP2, the 16S rRNA gene sequence of each species-level OTU was then compared to the Ribosomal Database Project (RDP) reference database to assign taxonomic designations down to the genus level (Wang, Garrity et al. 2007). A BLASTn search was then performed with the 16S rRNA gene sequences to determine whether the OTU represented either a previously characterised or a novel species (Altschul, Gish et al. 1990).

Comparisons of the OTUs with the Human Microbiome Project (HMP) “Most Wanted” list and reference genomes database were carried out using 97% sequence identity of the 16S rRNA gene sequences to define a bacterial species because only partial 16S rRNA gene sequences were available for the bacteria on the HMP “Most Wanted” list and reference genomes database. HMP data regarding the most wanted taxa and the completed sequencing projects were downloaded from the NIH Human Microbiome Project's “Most Wanted” Taxa from the Human Microbiome for Whole Genome Sequencing (Web 8 Mar. 2016 hmpdacc.org/most_wanted/#data) and the NIH Human Microbiome Project's Reference Genomes Data (Web 8 Mar. 2016 hmpdacc.org/HMRGD/), respectively. Genomic DNA was extracted from at least one representative of each unique OTU using a phenol-chloroform based DNA isolation procedure. DNA was sequenced on the Illumina HiSeq platform generating read lengths of 100 bp and these were assembled and annotated for further analysis.

DNA was also extracted directly from each faecal sample for whole community metagenomic and 16S rRNA gene amplicon sequencing using the MP Biomedical FastDNA SPIN Kit for soil. To enable comparisons with the complete community samples, non-confluent cultures were scraped from agar plates 72 hours after inoculation with the initial faecal sample and DNA was extracted from this community using the same DNA isolation process. 16S rRNA gene amplicon libraries were made by PCR amplification of variable regions 1 and 2 of the 16S rRNA gene using the Q5 High-Fidelity Polymerase Kit supplied by New England Biolabs. Primers 27F AATGATACGGCGACCACCGAGATCTACAC TATGGTAATT CC AGMGTTYGATYMTGGCTCAG (SEQ ID NO: 54) (1^{st} part=Illumina adapter, 2^{nd}=forward primer pad, 3^{rd}=Forward primer linker and 4^{th}=Forward primer) and 338R CAAGCAGAAGACGGCATACGAGAT ACGAGACTGATT AGTCAGTCAG AA GCTGCCTCCCGTAGGAGT (SEQ ID NO: 55) (1^{st} part=reverse complement of 3′ lllumina adapter, 2^{nd}=golay barcode, 3^{rd}=reverse primer pad, 4^{th}=reverse primer linker and 5^{th}=reverse primer) were used. Four PCR amplification reactions per sample were carried out; products were pooled and combined in equimolar amounts for sequencing using the Illumina MiSeq platform, generating 150 bp reads.

For 454 amplicon sequencing of the CIP2 derived faecal samples, DNA was extracted directly from the faecal samples (70 mg) using the FastDNA Spin Kit for Soil on a Fastprep instrument (MP Biomedicals, USA) following the manufacturer's instructions. The V3-V5 regions of the 16S rRNA gene were amplified using barcoded primers 338F (5′-ACTCCTACGGGAGGCAGCAG-3′) (SEQ ID NO: 56) and 926R (5′-CCG TCA ATT CMT TTR AGT-3′) (SEQ ID NO: 57) adapted with linkers. Thermocycling involved an initial 2-min denaturation step at 94° C. followed by 20 cycles of denaturation (94° C. for 30 s), annealing (53° C. for 30 s) and elongation (68° C. for 2 min). The PCR products were purified using the Wizard SV Gel and PCR Clean-Up System (Promega, UK) following the manufacturer's protocol and quantified using the Qubit® dsDNA HS Assay Kit (Life_Technologies, UK). Equimolar volumes of each cleaned-up products of each PCR reaction were sequenced on the Roche 454 FLX-Titanium platform.

Microbiota Analysis

A maximum likelihood phylogeny of the culture derived bacteria from CIP1 was generated from the aligned RDP sequence using FastTree version 2.1.3 (Price, Dehal et al. 2010) with the following settings: a Generalised Time-Reversible (GTR) model of nucleotide substitution and CAT approximation of the variation in rates across sites with 20 rate categories. The ethanol resistant phylogeny was derived directly from the entire culture phylogeny. All phylogenetic trees were edited in ITOL (Letunic and Bork 2011).

Analysis of the partial 16S rRNA gene sequence generated from the 16S rRNA gene amplicon libraries from the CIP1 derived faecal samples was carried out using the mothur MiSeq SOP (Kozich, Westcott et al. 2013) on Aug. 29, 2014, identifying 7549 OTUs across all samples. A sequence identity threshold of >97% was again used to define an OTU.

For the 454 sequence analysis from CIP2 derived faecal samples the sequence reads were trimmed, filtered and pre-processed using the mothur software 454 SOP accessed in November 2012 (Schloss, Westcott et al. 2009, Schloss, Gevers et al. 2011). To ensure high quality sequence data for analysis, the sequences were trimmed using a window size of 50 bp (average quality score of 35 bp), homopolymers >8 bp were removed and no ambiguous bases or mismatches in the primer sequence were allowed. Redundant sequence reads were removed to generate unique sequences, which were aligned to the SILVA alignment database (Pruesse, Quast et al. 2007). These aligned sequences were screened to ensure that sequences overlapped in the same alignment space using the screen.seqs command in mothur. Unique sequences were again generated and the sequences were preclustered to remove sequences that were likely due to pyrosequencing errors (Huse, Dethlefsen et al. 2008). Chimeric sequences were removed using Perseus (Quince, Lanzen et al. 2011) and other contaminants such as chloroplast and mitochrondria were also removed. Sequences with 97% sequence identity and their assigned taxonomy from phylum to genus level according to the Ribosomal Database Project (RDP) (Cole, Wang et al. 2014) and SILVA (Pruesse, Quast et al. 2007) databases were regarded as belonging to the same operational taxonomic units (OTUs). The species diversity in each sample was measured by calculating the Shannon diversity Index (SDI), which takes into account both species richness and relative proportional abundance (Schloss, Westcott et al. 2009). The OTUs were then used to cluster dendrograms, using the Bray Curtis calculator in the mothur package. Other analyses, such as Invsimpson index, principal component analysis (PCA) and the UniFrac method of comparing microbial communities were performed as described previously using the mothur software (Lozupone and Knight 2005, Lawley, Clare et al. 2012).

Metagenomic Analysis

Microbial abundance was calculated using the Human Pan-Microbe Community Database (Forster, Browne et al. 2015) against 1883 healthy individuals (3218 samples) and 458 diseased individuals (628 samples). Occurrence was calculated as greater than 1000, independent, normalised reads with abundance calculated relative to total high quality reads within the sample. Antimicrobial resistance and virulence factor identification were performed using automated sequence homology search against protein sequences annotated in the complete genome sequence. The antimicrobial resistance reference list was defined based on the comprehensive antimicrobial CARD database (McArthur, Waglechner et al. 2013) while toxins were identified by occurrence in the Database of Bacterial Exotoxins for Humans (DBETH) (Chakraborty, Ghosh et al. 2012).

Experimental Set-Up and Results

The inventors established methods to isolate and identify bacteria for incorporation into a therapeutic composition tailored to the treatment of dysbiosis of the gastrointestinal tract, as well as e.g. enteric infections, such as, but not limited to, those caused by Clostridium difficile. As mentioned above, two different approaches for acquiring bacterial candidates for inclusion in a therapeutic were employed. The first (CIP1) relied on a broad culturing approach from healthy adult donors to establish a culture collection which is as representative as possible of the bacterial component of the healthy human intestinal microbiota. This process also incorporated a targeted culturing approach to preferentially select bacteria displaying a particular phenotype or function e.g. spore formation. The second process (CIP2) was more targeted in nature and aimed to acquire bacterial species specifically associated with resolving gastrointestinal dysbiosis by comparing individuals before and FMT to resolve C. difficile associated dysbiosis. These two approaches are described in more detail below.

CIP1—Broad Culturing Approach to Identify Therapeutic Candidates:

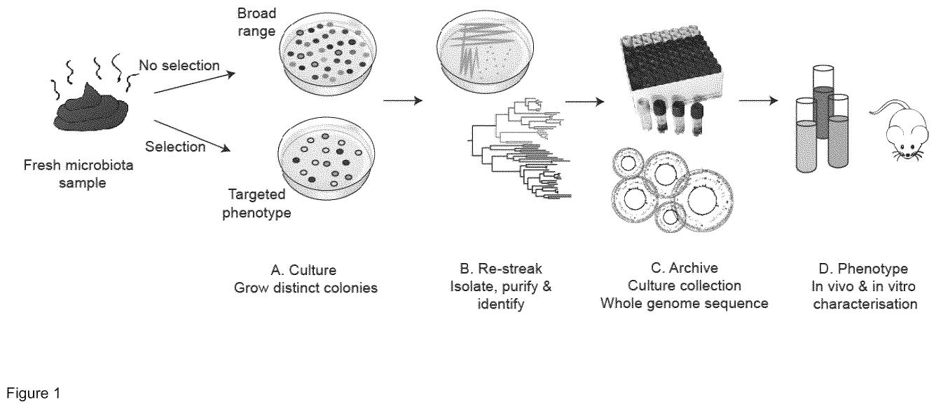

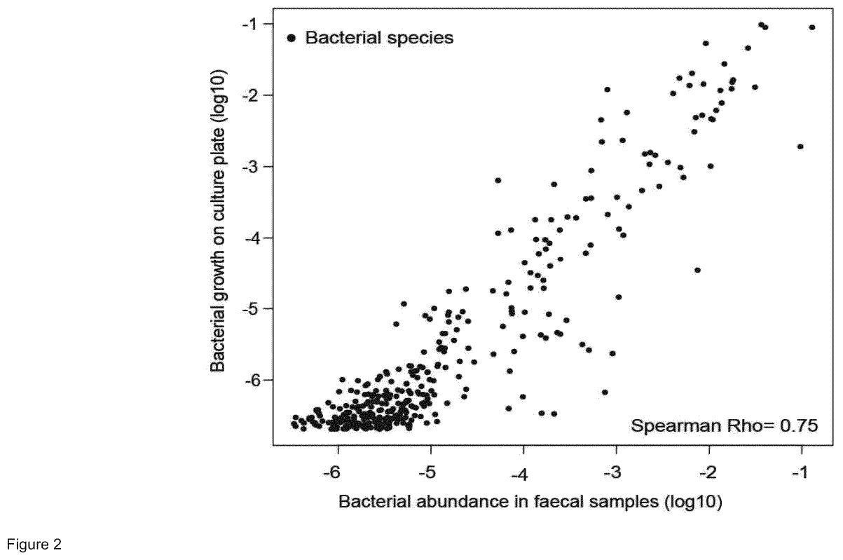

The inventors first sought to establish a genomic-based workflow that could be used as a platform for targeted culturing of specific bacterial phenotypes (FIG. 1). Fresh faecal samples were collected from 6 healthy humans and defined the resident bacterial communities with a combined metagenomic sequencing and bacterial culturing approach. Applying shotgun metagenomic sequencing, the inventors profiled and compared the bacterial species present in the original faecal samples to those that grew as distinct colonies on agar plates containing the complex, broad range bacteriological medium, YCFA (Duncan, Hold et al. 2002) supplemented with glucose, maltose and cellobiose. Importantly, a strong correlation was observed between the two samples at the species level (Spearman Rho=0.75, p<0.01) (FIG. 2). When sequenced, the original faecal sample and the cultured bacterial community shared an average of 93% of raw reads across the 6 donors.

These results demonstrate that surprisingly, and contrary to the established view in the art, a significant proportion of the bacteria within the faecal microbiota can be cultured with a single growth medium. Thus, a broad range culturing method was established that, when combined with high throughput archiving or specific phenotypic selection, can be utilised to isolate and identify novel bacteria from the human gastrointestinal tract.

The human intestinal microbiota is dominated by strict anaerobic bacteria that are extremely sensitive to ambient oxygen. Certain members of the Firmicutes, including Clostridium difficile, produce metabolically dormant and highly resistant spores during colonisation that facilitate both persistence within the host and environmental transmission (Lawley, Clare et al. 2009, Francis, Allen et al. 2013, Janoir, Deneve et al. 2013). Relatively few intestinal spore-forming bacteria have been cultured to date and while metagenomic studies suggest that other unexpected members of the intestinal microbiota possess potential sporulation genes, they remain poorly characterised (Galperin, Mekhedov et al. 2012, Abecasis, Serrano et al. 2013, Meehan and Beiko 2014, Rajilic-Stojanovic and de Vos 2014).

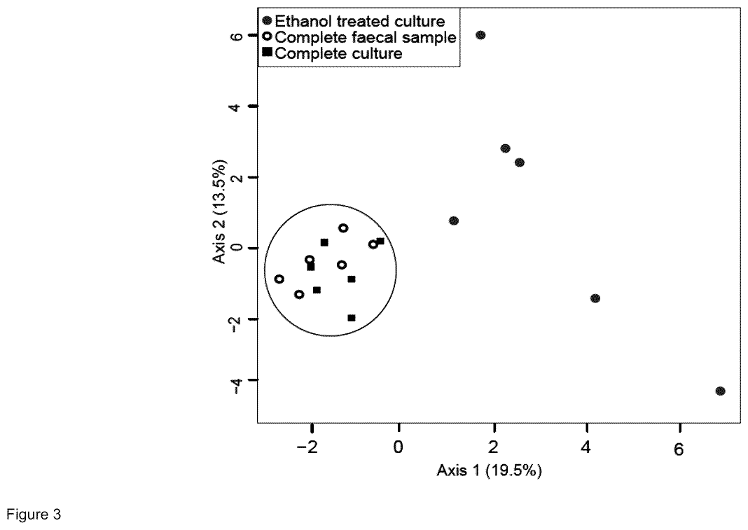

The inventors hypothesized that sporulation might be an unappreciated basic phenotype of the human intestinal microbiota that may have a profound impact on microbiota persistence and spread between humans. Spore-formation is also viewed as desirable for bacteriotherapy formulations since the resistant nature of the spore structure would promote survival of the medicine during production and subsequent storage. Spores from C. difficile are resistant to ethanol and this phenotype can be used to select for spores from a mixed population of spores and ethanol-sensitive vegetative cells (Riley, Brazier et al. 1987). Faecal samples with or without ethanol treatment were processed using our combined culture and metagenomics workflow (FIG. 1). Principle component analysis demonstrated that ethanol treatment profoundly altered the culturable bacterial composition and when compared to the original profile, efficiently enriched for ethanol-resistant bacteria, facilitating their isolation (FIG. 3). ˜2,000 individual bacterial colonies were picked from both ethanol-treated and non-ethanol-treated conditions, re-streaked them to purity and performed full-length 16S rRNA gene sequencing to enable taxonomic characterisation. Unique taxa were then archived as frozen stocks for future phenotypic analysis.

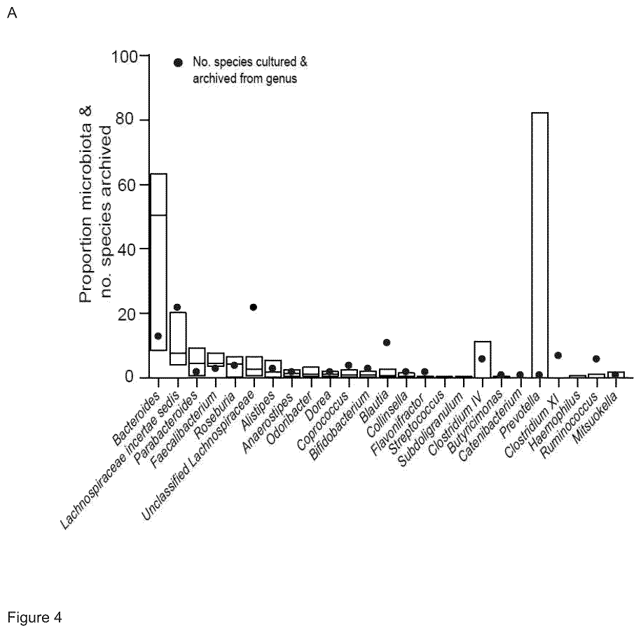

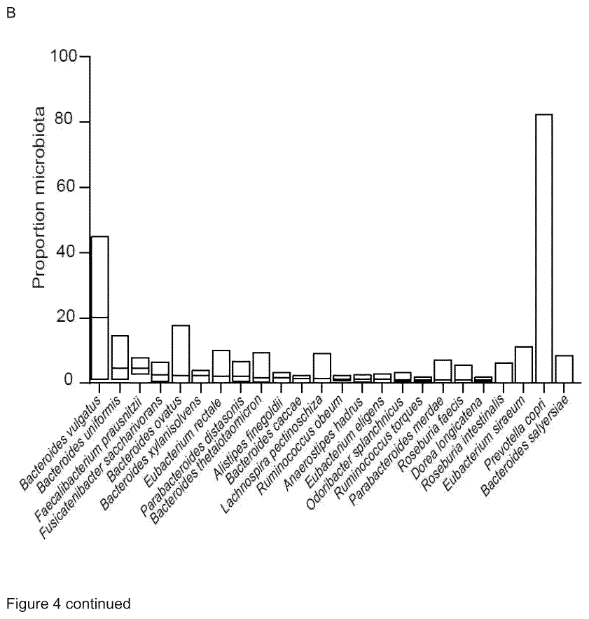

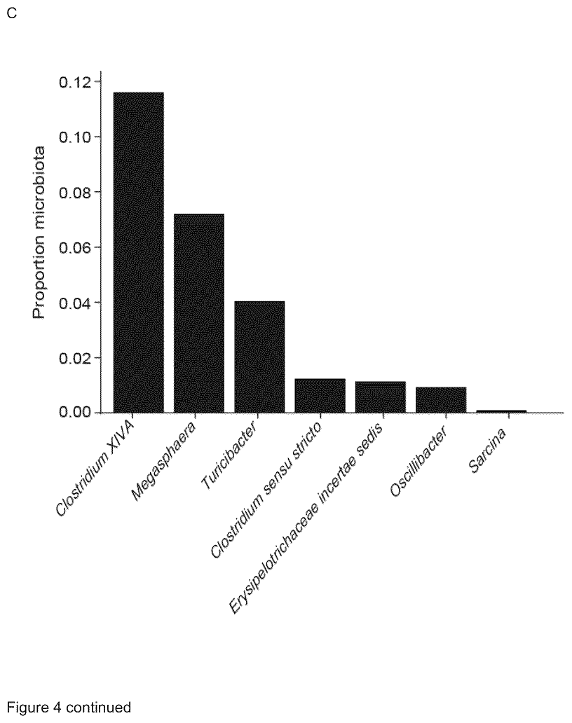

In total, bacteria representing 96% of the bacterial abundance at the genus level and 90% of the bacterial abundance at the species level based on average relative abundance across the 6 donors (FIGS. 4A and 48) were archived. Even genera that were present at low average relative abundance (<0.2%) were isolated and purified (FIG. 4C). Ethanol-resistant species were isolated from 5 known families (Clostridiaceae, Peptostreptococcaceae, Lachnospiraceae, Ruminococcaceae and Erysipelotrichaceae) and 2 newly identified candidate families (bacterial isolates HMI_1 and HMI_22) (see FIG. 15 for details). The identification of these new and unexpected spore-formers highlights the broad taxonomic distribution of this phenotype among the enteric species of the Firmicutes.

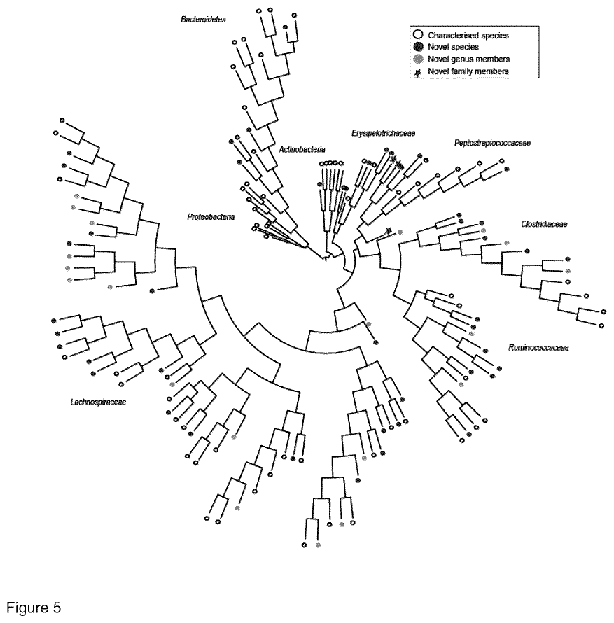

Overall, 137 distinct bacterial species including 45 candidate novel species and isolates representing 20 candidate novel genera and 2 candidate novel families were archived (FIG. 5). Our collection contains 90 species from the Human Microbiome Project's (HMP) ‘most wanted’ list of previously uncultured and unsequenced microbes (Fodor, Desantis et al. 2012). 19 of the deposited bacterial isolates listed in FIG. 15 are included in the HMP's “Most Wanted” list, namely: HMI_1, HMI_2, HMI_4, HMI_5, HMI_7, HMI_11, HMI_12, HMI_15, HMI_16, HMI_17, HMI_18, HMI_19, HMI_35, HMI_37, HMI_38, HMI_39, HMI_45, HMI_50 and HMI_51 (see FIG. 15 for details). Thus, our broad-range YCFA based culturing approach led to the discovery of large numbers of novel bacteria (including novel families, genera, species, and isolates) and challenges the prevailing perception in the art that the majority of the intestinal microbiota is “unculturable”.

CIP2: Targeted Identification of Candidates to Resolve Gastrointestinal Dysbiosis

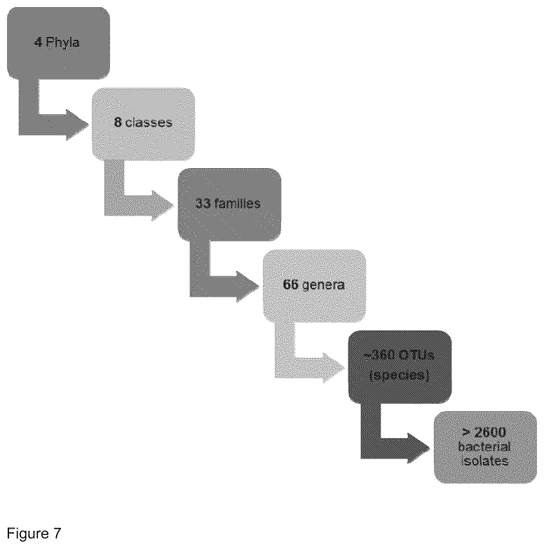

As described above, FMT has proven effective in resolving CDI. The inventors therefore sought to culture from faecal samples from FMT donors and recipients to isolate candidate bacteria that could be used in therapy. A panel of different microbiological media were tested to recover the broadest range of bacterial species from the faecal samples (see Methods). This approach allowed culturing and archival of bacterial candidates. Over 2600 bacterial isolates were cultured and using 16S rRNA gene sequencing these were taxonomically classified (FIG. 7). These bacterial isolates were members of the 4 major phyla (Actinobacteria, Bacteroidetes, Proteobacteria and Firmicutes) in the intestinal microbiota. These bacterial isolates represented more than 350 different OTUs based on alignments of the partial length 16S rRNA gene.

In silico analysis of candidate bacteria:

Having established a culture collection through the two approaches described above (CIP1 and CIP2), the inventors next sought to screen these bacteria to identify bacterial candidates for bacteriotherapy.

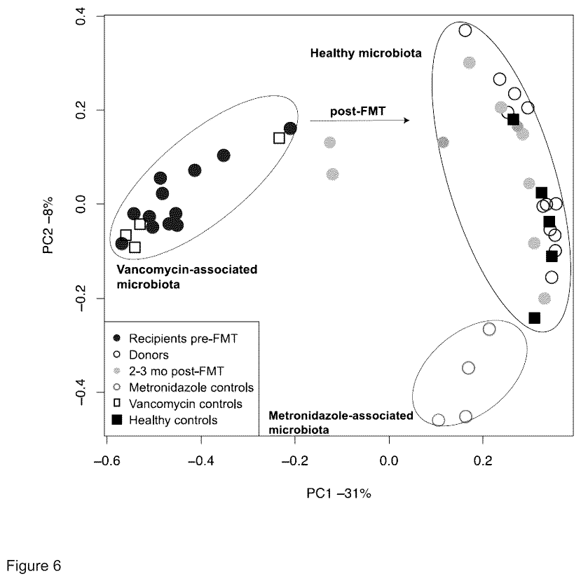

The inventors first sought to analyse the isolates cultured from FMT donors and recipients. At one to three months post-FMT, the faecal microbiota profiles of the recipients were similar to those of the donors and the healthy controls. In particular, the relative abundances of the four major bacterial phyla present in the gut microbiota were also similar across these groups. The microbiota community structure in the donors and recipients (before and after FMT) was visualised for evaluation using principal component analysis (PCA) (FIG. 6).

The PCA plot demonstrates the presence of two distinct compositional profiles representing a “health-associated” microbiota, and a “vancomycin” microbiota. The health-associated profile contained samples from FMT donors, healthy controls and FMT recipients at 2-3 months post treatment. The “vancomycin” microbiota profile was separated from the health-associated microbiota along Principal Component 1 and only contained individuals treated with vancomycin. These vancomycin control individuals became infected with C. difficile while taking antibiotics to treat other disease conditions. Additionally, the “metronidazole-associated” profile was separated from the “health-associated” profile along Principal Component 2 and contained samples from C. difficile infected patients treated with metronidazole.

The donor-recipient profiles of each pair were compared before and after FMT to identify taxa that were present in the donor sample, and which increased in relative abundance in the recipient's profile after FMT. A total of 786 OTUs from all recipient samples were detected after FMT but singleton OTUs present at the different time points were removed. This resulted in 375 OTUs for further analysis. Given that recurrence of CDI typically occurs 3-4 weeks after the withdrawal of antibiotic treatment, (Comely, Miller et al. 2012, Abujamel, Cadnum et al. 2013) OTUs that were increased in relative proportional abundances at 2-3 months post-FMT were analysed further.

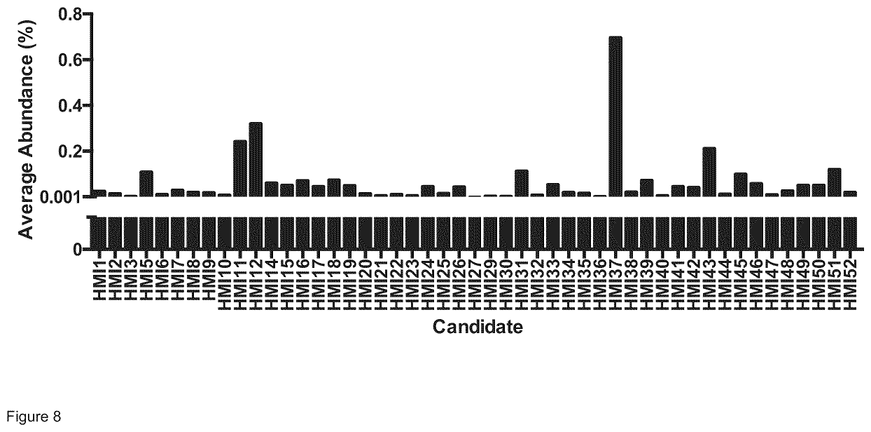

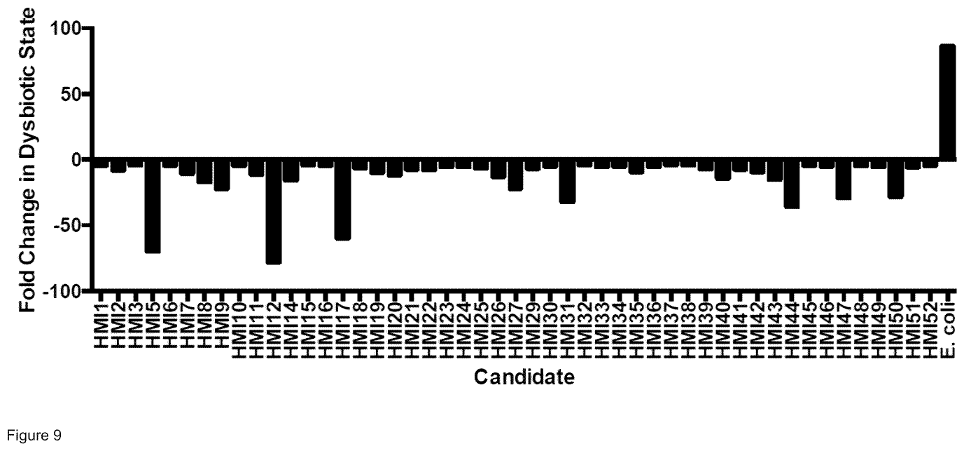

Next, the inventors undertook in silico analysis to further screen the bacteriotherapy candidates from both of our culturing approaches (CIP1 and CIP2). As described above a healthy intestinal microbiota is based on a diverse and abundant microbial community. Using the whole genome sequences that the inventors generated from bacterial isolates from CIP1 and CIP2, the inventors computationally assessed their prevalence in healthy and diseased individuals in public metagenomic data-sets using the HPMC database tool (Forster, Browne et al. 2015). Candidate bacteria were first filtered to include only those isolates with greater than 0.001% average abundance within the bacterial community across all healthy individuals in which they were detected (FIG. 8). All of the bacteria deposited with DSMZ thus had greater than 0.001% average abundance within the bacterial community across all healthy individuals in which they were detected (see FIG. 15). In addition to being health-associated, preferred candidates for bacteriotherapy applications are expected to ameliorate gastrointestinal dysbiosis. To identify such candidates, the distribution of each of our isolates in publicly available metagenomics datasets was examined. Bacterial species whose total average abundance was substantially decreased (greater than four-fold decrease) in individuals with gastrointestinal dysbiosis relative to healthy individuals were selected and subjected to further analysis as described below (FIG. 9). All of the bacteria deposited with DSMZ thus showed a decreased total average abundance (greater than four-fold decrease) in individuals with gastrointestinal dysbiosis relative to healthy individuals (see FIG. 15).

The list of bacteriotherapy candidates was further analysed on the basis of computationally predicted antimicrobial resistance (AMR) and virulence factors. Bacteriotherapy candidates with overall predicted resistance scores below 20% of the overall predicted resistance scores of the known pathogens C. difficile, Enterococcus faecalis and Escherichia coliwere included. Candidates were also selected for the absence of in-silico predicted resistance to beta-lactams, fusidic acid, elfamycin, aminoglycoside, fosfomycin and tunicamycin and by the absence of known toxins as listed in Chakrabory A. et al, 2012, A Database of Bacterial Exotoxins for Humans (DBETH). Based on this analysis the inventors identified 51 candidates for use in bacteriotherapy from CIP1 and CIP2 (see FIG. 15). 10 of these bacteriotherapy candidates were identified using CIP2, namely: HMI_23, HMI_24, HMI_25, HMI_26, HMI_27, HMI_28, HMI_29, HMI_30, HMI_31 and HMI_32 (see FIG. 15 for details). All of these 10 isolates were cultured from healthy donors. The remaining bacteriotherapy candidates were identified using CIP1.

The bacteriotherapy candidates identified using CIP1 and CIP2, with the exception of HMI_17, were then subjected to in vitro analysis to establish their therapeutic efficacy in treating C. difficile and E. coli infection as described in Example 2 below.

### Example 2—In Vitro Analysis of Bacteriotherapy Candidates

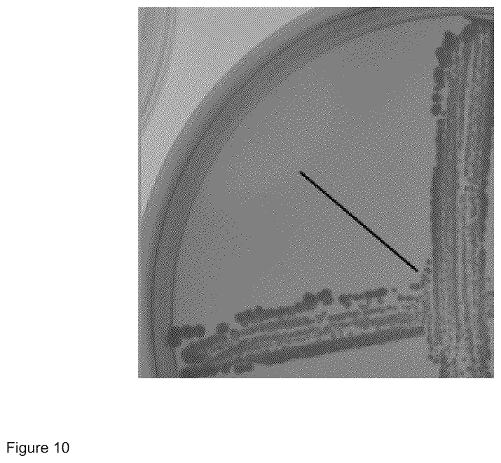

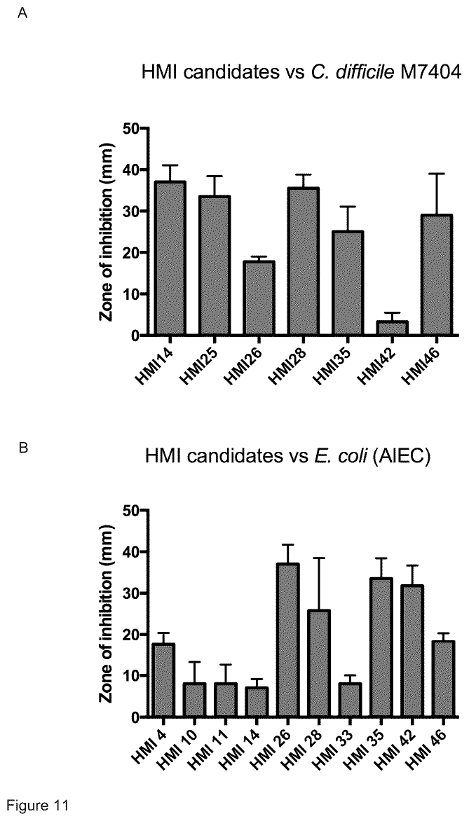

Detection of anti-pathogen activities of the bacteriotherapy candidates by an overlay assay The bacterial isolates of interest identified in Example 1 were streaked in an “X” shape over the surface of a standard Petri dish containing warmed and reduced YCFA agar. These inoculated plates were then incubated anaerobically at 37° C. for 3 to 6 days, until bacterial growth was clearly visible. Overlay agar was prepared by adding 0.8% agar to an appropriate broth. For C. difficile, BHI broth+0.8% agar was used. For E. coli, LB+0.8% agar was prepared. The overlay agar was held molten at 50° C. before use. The overlay agar was inoculated (1% inoculum) with an aliquot of a turbid culture of the pathogen of interest, in this case either C. difficile M7404 or E. coli (AIEC). A 10 ml aliquot of the inoculated overlay agar was added to the surface of the agar plates bearing each commensal strain of interest. The overlay agar was allowed to set and the plates were incubated anaerobically at 37° C. for one to two days. Following incubation, zones of clearing could be observed if the commensal strain of interest was capable of inhibiting the growth of the pathogen in the overlay layer. The width of each zone of clearing was measured with a ruler as shown in FIG. 10. The results are shown in FIG. 11.

Detection of Anti-Pathogen Activity by CFS-Relative Growth Inhibition Assay.

Bacteriotherapy candidates were grown in 1 ml aliquots of reduced YCFA broth at 37° C. under anaerobic conditions for two days. Cell free supernatant (CFS) was prepared by centrifuging each culture to remove the bacteria and by passing the resulting supernatant through a 0.22 μm filter to sterilise it. Uninoculated YCFA broth was also filter sterilised. The CFSs and filtered YCFA broth aliquots were frozen at −20° C. until they were required. These filtrates were thawed under anaerobic conditions at 37° C. and a 100 μl aliquot of each CFS was added to one well of a flat-bottomed 96-well plate. Several wells were filled with filter-sterilised YCFA broth to serve as positive controls for pathogen growth. Each well was inoculated (2-5% inoculum) with a turbid, early-mid exponential phase C. difficile M7404 culture. Alternatively, a 5% inoculum of a stationary phase E. coli culture adjusted to OD₆₀₀ ≈1 was used. The 96-well plate was sealed with an optically clear film and it was transferred to a FLUOstar Omega microplate-reader (BMG Labtech). The plate was incubated static at 37° C. in the plate-reader and ODeoo readings were taken every 10 min for 18.17 hours. The plate was shaken for 10 seconds before each OD reading was taken. All isolates, except HM117 were tested.

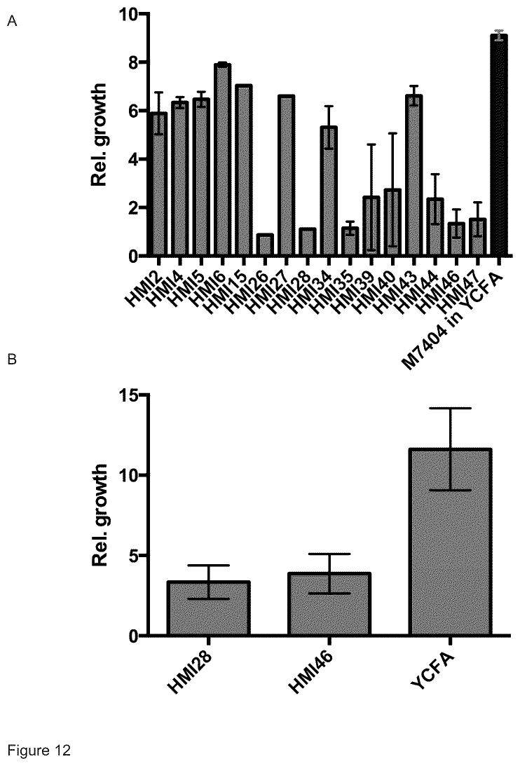

The relative growth of the pathogen of interest in each of the CFSs tested was then calculated as follows: For each CFS tested, every raw-data value was expressed relative to its OD₆₀₀ reading that was taken at the ten minute time-point. Such data normalisation permitted direct comparison of C. difficile or E. coli growth in the various CFSs by eliminating the initial inherent variation in the optical density of the CFSs (due to the pre-fermentation of the media) from consideration. The relative growth achieved at the 18.17 h time-point by C. difficile or E. coli in each of the CFSs was compared to the relative growth of the pathogen of interest that was achieved in YCFA broth. A commensal strain was considered a potential inhibitor of C. difficile or E. coli if the relative growth plus two standard deviations of the pathogen of interest in the CFS derived from that same commensal isolate, was less than the mean minus two standard deviations of the relative growth of the pathogen in YCFA broth. Where only one relative growth value was available, a CFS was considered as potentially inhibitory if the relative pathogen growth was more than two standard deviations below the mean relative growth in YCFA broth. The results for bacteriotherapy candidates found to have inhibitory activity are shown in FIG. 12.

Results

A summary of the results obtained in the growth overlay and growth inhibition assays is shown in FIG. 13 and FIG. 15. The bacteriotherapy candidates which showed activity in each of the in vitro assays are indicated in this figure.

Of the 50 bacteriotherapy candidates tested, 22 demonstrate growth inhibition of at least one of C. difficile M7404 or E. coli (AIEC) in one of the assays performed. 11 of the bacteriotherapy candidates inhibited the growth of at least one of either C. difficile or E. coli in overlay assays, suggesting that the inhibition conferred by these bacteriotherapy candidates is direct. According to the overlay assay data, 5 of the bacteriotherapy candidates inhibit only the growth of either C. difficile or E. coli, suggesting that the inhibitory activity of these bacteriotherapy candidates is not generic, i.e. that the inhibitory activity is specific for one or more pathogenic bacteria.

Of the 50 bacteriotherapy candidates tested, 6 inhibited the growth of both C. difficile and E. coli in overlay assays, suggesting that they have a broad-spectrum of inhibitory activity and are likely to also have inhibitory activity against other pathogenic bacteria.

The results from the CFS-relative growth inhibition assay demonstrate that the CFS from 16 of the 50 bacteriotherapy candidates tested, only supported the relative growth of C. difficile to levels more than two standard deviations below the mean relative growth in YCFA broth at the 18.17 h time-point. These bacteriotherapy candidates are thus considered to inhibit C. difficile growth. 5 of these bacteriotherapy candidates were also shown to directly inhibit C. difficile and/or E. coli growth in the overlay assays. This suggests that these 5 bacteriotherapy candidates secrete one or more substances which inhibit the growth of these pathogenic bacteria. The remaining 11 bacteriotherapy candidates which showed inhibitory activity in the CFS-relative growth inhibition assay are likely to compete with C. difficile for nutrients. CFS from two of the bacteriotherapy candidates did not support growth of E. coli to within two standard deviations of the mean growth observed for E. coli in YCFA broth. These isolates are therefore considered to inhibit the growth of E. coli.

### Example 3—in Silico Co-Abundance Network Analysis

To identify bacteria that, while not capable of directly inhibiting pathogen growth as tested in Example 2, may support the growth or survival of those bacteria that exhibited direct inhibition of pathogen growth in Example 2, co-abundance network analysis was performed. This analysis was performed as described previously using the complete list of healthy datasets in the HPMC database tool (Forster, Browne et al. 2015). For each candidate bacterium that demonstrated inhibition of pathogen growth in Example 2, a complete list of first degree neighbour species that exhibited co-occurrence with the candidate bacterium across at least 95% of faecal samples with an average abundance greater than 0.001% and a minimum of 100 reads was generated. Bacteria that exhibited extensive co-occurrence with candidate bacteria exhibiting direct inhibitory activity of pathogen growth are predicted to provide a metabolic, environmental and/or immunomodulatory support function required for colonization of the gastrointestinal tract by the candidate bacteria. The deposited bacteria demonstrating such co-occurrence is indicated in FIG. 15.

### DISCUSSION

Bacterial isolates that inhibited the growth of one or more pathogenic bacteria as shown in Example 2 are expected to be suitable for treating gastrointestinal dysbiosis in humans.

However, bacterial isolates that did not show evidence of pathogen inhibition in Example 2 are still expected to be useful for the treatment of gastrointestinal dysbiosis.

Firstly, based on the co-occurrence data obtained in Example 3, a large number of the deposited bacteria are expected to support the colonization of the gastrointestinal tract by the inhibitory bacteria identified in Example 2 through direct or indirect interaction. Metabolic networks in which consortia of bacteria thrive by cross-feeding, structural networks, such as biofilms, or the interactions of ‘keystone species’, allow the microbiota to establish and stabilise (Ze and Mougen et al. 2013). Co-occurrence analysis identified 35 candidates that formed first degree co-occurrence neighbours with direct inhibitors at a rate above 95% (HMI_2, HMI_5, HMI_6, HMI_7, HMI_8, HMI_9, HMI_10, HMI_11, HMI_12, HMI_14, HMI_15, HMI_16, HMI_17, HMI_18, HMI_19, HMI_20, HMI_26, HMI_27, HMI_31, HMI_33, HMI_34, HMI_35, HMI_37, HMI_38, HMI_39, HMI_41, HMI_42, HMI_43, HMI_44, HMI_46, HMI_47, HMI_48, HMI_50, HMI_51, HMI_52, see FIG. 15 for details). In addition, several of the bacterial isolates listed in FIG. 15 reside within the same genera as known keystone species (HMI_17, HMI_23 to HMI_32, HMI_45, HMI_49, HMI_51 and HMI_52; see FIG. 15 for details) and thus are expected to represent keystone species themselves.

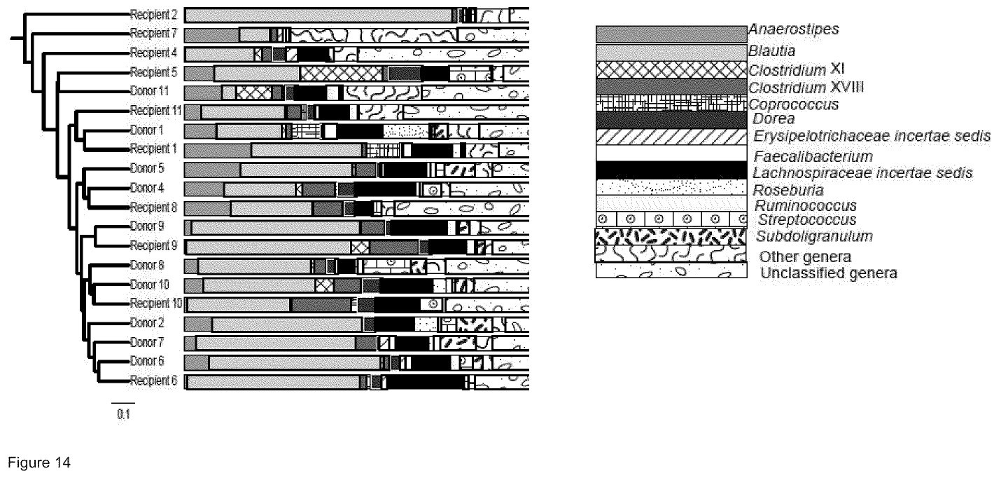

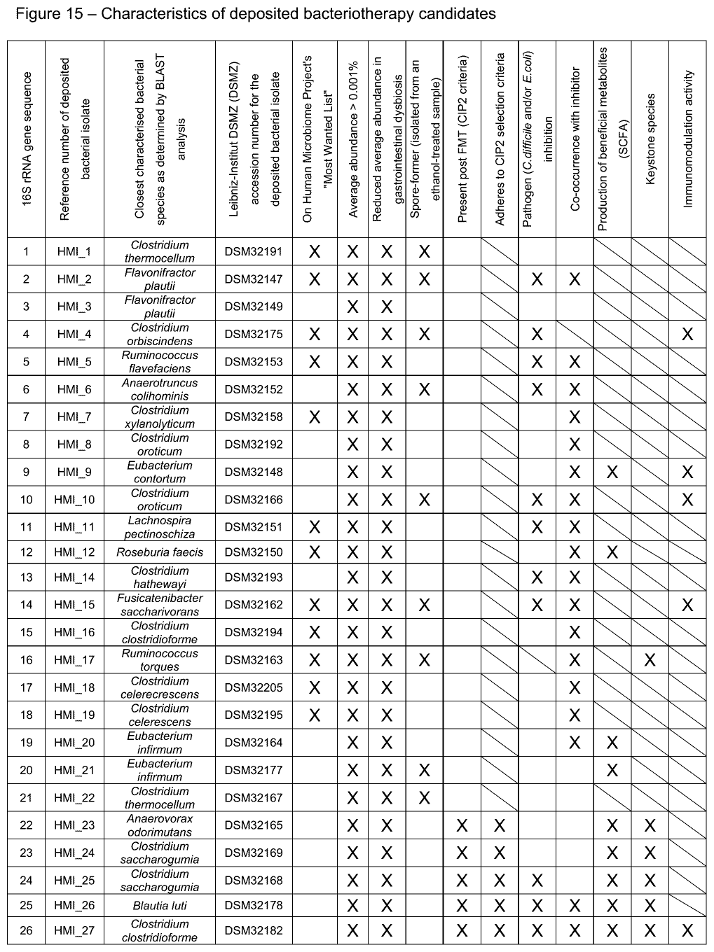

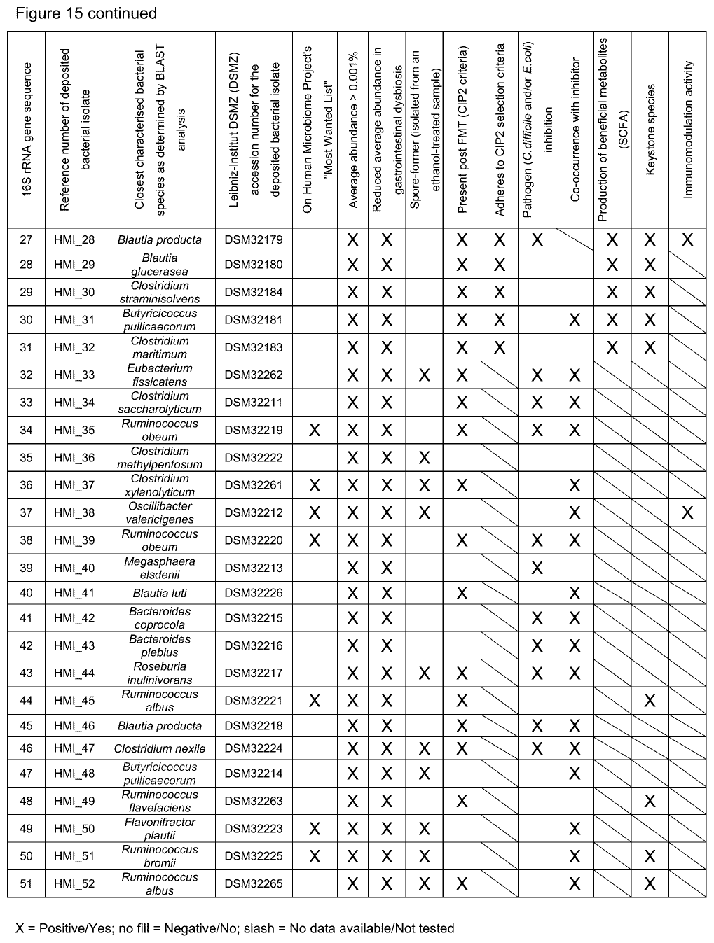

Secondly, the bacterial isolates listed in FIG. 15 are shown in Example 1 to contribute to the overall diversity of the gastrointestinal microbiota, which is low during dysbiosis. Specifically, a number of these bacteria (HMI_23 to HMI_32 inclusive) were recovered from the intestinal microbiota of FMT donors as part of CIP2. When the microbiota of a healthy donor was transferred to an individual with dysbiosis due to antibiotic treatment for recurrent C. difficile infection, all were restored to health (FIG. 6), which was determined as the absence of C. difficile at 2-3 months post-FMT. The criteria for identifying bacteriotherapy candidates by the CIP2 process required that a certain candidate bacterial species was present in the microbiota of half the recipients at more than >0.6% average relative abundance at 2-3 months post-FMT. Moreover, genera representing several of the 51 bacteria listed in FIG. 15 were also identified in healthy donors and in cured recipients post-FMT (FIG. 14). Furthermore, the in silico analysis presented herein (FIGS. 8 and 9) revealed that the 51 candidate bacteriotherapy isolates are prevalent in healthy individuals, in whom they occur at an average relative abundance >0.001% and these bacteria tend to be depleted under conditions of dysbiosis (FIG. 9). Together, these data strongly suggest that the 51 bacterial isolates listed in FIG. 15 are suitable in the treatment of gastrointestinal dysbiosis.

Thirdly, the bacterial isolates listed in FIG. 15 are expected to compete with enteric pathogens in the gastrointestinal tract, and thus find application in the treatment of gastrointestinal dysbiosis. Specifically, the widespread occurrence of these bacteria in healthy individuals implies that they efficiently colonise the gastrointestinal tract. When the microbiota is populated by these health-associated bacteria, the likelihood of enteric infection with any pathogenic bacterium is known to be low, as such infections usually do not occur in individuals with a healthy gastrointestinal microbiota. Indeed, following FMT, during which genera representing many of the 51 bacteria listed in FIG. 15 were identified in individuals treated for a dysbiosis of the gastrointestinal tract following antibiotic treatment for C. difficile infection (FIG. 14), a healthy microbiota profile was restored (FIG. 6 and FIG. 14) and C. difficile infection did not recur within 3 months. This indicates that these bacteria promote health according to the principles of colonisation resistance, in which pathogens are excluded or suppressed by competition with the resident health-associated bacteria for nutrients and attachment sites (Britton & Young 2014; Lawley & Walker 2013).

Fourthly, several of the bacterial isolates listed in FIG. 15 are expected, on the basis of extrapolation from investigations of other species in the same genera or clades (Louis & Flint, 2009), to produce metabolites, such as short chain fatty acids, which have known benefits for gastrointestinal health (e.g. HMI_9, HMI_12, HMI_20, HMI_21 and HMI_23-HMI_32; see FIG. 15 for details).

Finally some Clostridium related species have been shown to be immunomodulatory and can be beneficial in reducing inflammation (Atarashi, Tanoue et al. 2013). Based on a comparison of 16S rRNA gene sequences using 95% sequence identity as a cut-off to define a genus (Bosshard, Abels et al. 2003), examples in this context that are in the same genus as these bacteria are HMI 4. HMI 9. HMI 10. HMI 15. HMI 27. HMI_28 and HMI_38.

### SEQUENCE LISTING

16S rRNA gene sequences of the 51 deposited bacterial isolates listed in FIG. 15 are set out below. For each bacteriotherapy candidate a putative genus and species name is give. The genus was and species names were assigned based on the Ribosomal Database Project (RDP) reference database and BLASTn analysis as explained in Example 1. The genus and species names assigned to each of the bacteriotherapy candidates are thus that of the most closely related known bacterium and hence subject to change.

HMI_1 Clostridium thermocellum 16S rDNA sequence
(SEQ ID NO: 1)
CAGGACGAACGCTGGCGGCGTGCCTAACACATGCAAGTCGAACGAGAATCTTTGAACAGA
TCTTTTCGGAGTGACGTTCAAAGAGGAAAGTGGCGGACGGGCGAGTAACGCGTGAGTAAC
CTGCCCATAAGAGGGGGATAATCCATGGAAACGTGGACTAATACCGCATATTGTAGTCAA
GTCGCATGACTAGATTATGAAAGATTTATCGCTTATGGATGGACTCGCGTCAGATTAGAT
AGTTGGTGAGGTAACGGCTCACCAAGTCAACGATCTGTAGCCGAACTGAGAGGTTGATCG
GCCGCATTGGGACTGAGACACGGCCCAGACTCCTACGGGAGGCAGCAGTGGGGAATATTG
CGCAATGGGGGCAACCCTGACGCAGCAACGCCGCGTGCAGGAAGAAGGTCTTCGGATTGT
AAACTGTTGTCGCAAGGGAAGAAGACAGTGACGGTACCTTGTGAGAAAGTCACGGCTAAC
TACGTGCCAGCAGCCGCGGTAATACGTAGGTGACAAGCGTTGTCCGGATTTACTGGGTGT
AAAGGGCGCGTAGGCGGACTGTCAAGTCAGTCGTGAAATACCGGGGCTTAACCCCGGGGC
TGCGATTGAAACTGACAGCCTTGAGTATCGGAGAGGAAAGCGGAATTCCTAGTGTAGCGG
TGAAATGCGTAGATATTAGGAGGAACACCAGTGGCGAAGGCGGCTTTCTGGACGACAACT
GACGCTGAGGCGCGAAAGTGTGGGGAGCAAACAGGATTAGATACCCTGGTAGTCCACACC
GTAAACGATGGATACTAGGTGTAGGAGGTATCGACCCCTTCTGTGCCGCAGTTAACACAA
TAAGTATCCCACCTGGGGAGTACGACCGCAAGGTTGAAACTCAAAGGAATTGACGGGGGC
CCGCACAAGCAGTGGAGTATGTGGTTTAATTCGAAGCAACGCGAAGAACCTTACCTGGGC
TTGACATCCCTGGAATCGAGTAGAGATACTTGAGTGCCTTCGGGAATCAGGTGACAGGTG
GTGCATGGTTGTCGTCAGCTCGTGTCGTGAGATGTTGGGTTAAGTCCCGCAACGAGCGCA
ACCCCTATTGTCAGTTGCCATCATTAAGTTGGGCACTCTGGCGAGACTGCCGGTGACAAA
TCGGAGGAAGGTGGGGACGACGTCAAATCATCATGCCCCTTATGCCCAGGGCTACACACG
TACTACAATGGCCGATAACAAAGTGCAGCGAAACCGTGAGGTGGAGCGAATCACAAAACT
CGGTCTCAGTTCAGATTGCAGGCTGCAACTCGCCTGCATGAAGTTGGAATTGCTAGTAAT
CGCGGATCAGAATGCCGCGGTGAATACGTTCCCGGGCCTTGTACACACCGCCCGTCACAC
CATGAGAGTCGATAACACCCGAAGCCTGTGAGCTAACCTATTAGGAGGCAGCAGTCGAAG
GTGGGGTTGATGATTGGGGTGAAGTCG
HMI_2 Flavonifractor plautii 16S rDNA sequence
(SEQ ID NO: 2)
GAGTGCTCATGACAGAGGATTCGTCCAATGGAGTGAGTTACTTAGTGGCGGACGGGTGAGTAACGCGTGAGTAAC
CTGCCTTGGAGTGGGGAATAACAGGTGGAAACATCTGCTAATACCGCATGATGCAGTTGGGTCGCATGGCTCTGA
CTGCCAAAGATTTATCGCTCTGAGATGGACTCGCGTCTGATTAGCTGGTTGGCGGGGTAACGGCCCACCAAGGCG
ACGATCAGTAGCCGGACTGAGAGGTTGGCCGGCCACATTGGGACTGAGACACGGCCCAGACTCCTACGGGAGGCA
GCAGTGGGGAATATTGGGCAATGGGCGCAAGCCTGACCCAGCAACGCCGCGTGAAGGAAGAAGGCTTTCGGGTTG
TAAACTTCTTTTCTCAGGGACGAAGCAAGTGACGGTACCTGAGGAATAAGCCACGGCTAACTACGTGCCAGCAGC
CGCGGTAATACGTAGGTGGCGAGCGTTATCCGGATTTACTGGGTGTAAAGGGCGTGTAGGCGGGACTGCAAGTCA
GATGTGAAAACCATGGGCTCAACCTGTGGCCTGCATTTGAAACTGTAGTTCTTGAGTACTGGAGAGGCAGACGGA
ATTCCTAGTGTAGCGGTGAAATGCGTAGATATTAGGAGGAACACCAGTGGCGAAGGCGGTCTGCTGGACAGCAAC
TGACGCTGAGGCGCGAAAGCGTGGGGAGCAAACAGGATTAGATACCCTGGTAGTCCACGCTGTAAACGATGGATA
CTAGGTGTGGGGGGTCTGACCCCCTCCGTGCCGCAGTTAACACAATAAGTATCCCACCTGGGGAGTACGATCGCA
AGGTTGAAACTCAAAGGAATTGACGGGGGCCCGCACAAGCGGTGGAGTATGTGGTTTAATTCGAAGCAACGCGAA
GAACCTTACCAGGGCTTGACATCCCGGTGACCGGTGTAGAGATACACCTTCTTCTTCGGAAGCGCCGGTGACAGG
TGGTGCATGGTTGTCGTCAGCTCGTGTCGTGAGATGTTGGGTTAAGTCCCGCAACGAGCGCAACCCTTATTGTTA
GTTGCTACGCAAGAGCACTCTAGCGAGACTGCCGTTGACAAAACGGAGGAAGGTGGGGACGACGTCAAATCATCA
TGCCCCTTATGTCCTGGGCCACACACGTACTACAATGGTGGTCAACAGAGGGAAGCAAGACCGCGAGGTGGAGCA
AACCCCTAAAAGCCATCCCAGTTCGGATTGCAGGCTGCAACTCGCCTGTATGAAGTTGGAATCGCTAGTAATCGC
GGATCAGCATGCCGCGGTGAATACGTTCCCGGGCCTTGTACACACCGCCCGTCACACCATGAGAGTCGGGAACAC
CCGAAGTCCGTAGCCTAACCGCAAGGGGGGCGCGGCCGAAGGTGGGTTCGATAATTGGGGTGAAGTCGT
HMI_3 Flavonifractor plautii 16S rDNA sequence
(SEQ ID NO: 3)
TGGCTGTTTAGTGGCGGACGGGTGAGTAACGCGTGAGTAACCTGCCTTGGAGTGGGGAATAACACAGTGAAAACT
GTGCTAATACCGCATGACATATTGGTGTCGCATGGCACTGATATCAAAGATTTATCGCTCTGAGATGGACTCGCG
TCTGATTAGATAGTTGGCGGGGTAACGGCCCACCAAGTCGACGATCAGTAGCCGGACTGAGAGGTTGGCCGGCCA
CATTGGGACTGAGACACGGCCCAGACTCCTACGGGAGGCAGCAGTGGGGAATATTGGGCAATGGGCGCAAGCCTG
ACCCAGCAACGCCGCGTGAAGGAAGAAGGCTTTCGGGTTGTAAACTTCTTTTAACAGGGACGAAGTAAGTGACGG
TACCTGTTGAATAAGCCACGGCTAACTACGTGCCAGCAGCCGCGGTAATACGTAGGTGGCAAGCGTTATCCGGAT
TTACTGGGTGTAAAGGGCGTGTAGGCGGGACTGCAAGTCAGATGTGAAAACTATGGGCTCAACCCATAGCCTGCA
TTTGAAACTGTAGTTCTTGAGTGTCGGAGAGGCAATCGGAATTCCGTGTGTAGCGGTGAAATGCGTAGATATACG
GAGGAACACCAGTGGCGAAGGCGGATTGCTGGACGATAACTGACGCTGAGGCGCGAAAGCGTGGGGAGCAAACAG
GATTAGATACCCTGGTAGTCCACGCCGTAAACGATGGATACTAGGTGTGGGGGGTCTGACCCCCTCCGTGCCGCA
GCTAACGCAATAAGTATCCCACCTGGGGAGTACGATCGCAAGGTTGAAACTCAAAGGAATTGACGGGGGCCCGCA
CAAGCGGTGGAGTATGTGGTTTAATTCGAAGCAACGCGAAGAACCTTACCAGGGCTTGACATCCTACTAACGAAC
CAGAGATGGATTAGGTGCCCTTCGGGGAAAGTAGAGACAGGTGGTGCATGGTTGTCGTCAGCTCGTGTCGTGAGA
TGTTGGGTTAAGTCCCGCAACGAGCGCAACCCTTATTGTTAGTTGCTACGCAAGAGCACTCTAGCGAGACTGCCG
TTGACAAAACGGAGGAAGGTGGGGACGACGTCAAATCATCATGCCCCTTATGTCCTGGGCCACACACGTACTACA
ATGGCGGTTAACAGAGGGAGGCAAAGCCGCGAGGCAGAGCAAACCCCTAAAAGCCGTCCCAGTTCGGATTGCAGG
CTGAAACCCGCCTGTATGAAGTCGGAATCGCTAGTAATCGCGGATCAGCATGCCGCGGTGAATACGTTCCCGGGC
CTTGTACACACCGCCCGTCACACCATGAGAGTCGGGAACACCCGAAGTCCGTAGCCTAACTGCAAAGGGGGCGCG
GCCGAAGGTGGGTTCGATAATTGGGGTGAAGTCGTAACAGGGTAACCG
HMI_4 Clostridium orbiscindens 16S rDNA sequence
(SEQ ID NO: 4)
TGGCGGACGGGTGAGTAACGCGTGAGGAACCTGCCTCGGAGTGGGGAATAACAGACCGAAAGGCCTGCTAATACC
GCATGATGCAGTTGGACCGCATGGTCCTGACTGCCAAAGATTTATCGCTCTGAGATGGCCTCGCGTCTGATTAGC
TTGTTGGCGGGGTAATGGCCCACCAAGGCGACGATCAGTAGCCGGACTGAGAGGTTGGCCGGCCACATTGGGACT
GAGACACGGCCCAGACTCCTACGGGAGGCAGCAGTGGGGAATATTGGGCAATGGGCGCAAGCCTGACCCAGCAAC
GCCGCGTGAAGGAAGAAGGCTTTCGGGTTGTAAACTTCTTTTCTCAGGGACGAACAAATGACGGTACCTGAGGAA
TAAGCCACGGCTAACTACGTGCCAGCAGCCGCGGTAATACGTAGGTGGCAAGCGTTATCCGGATTTACTGGGTGT
AAAGGGCGTGTAGGCGGGAAGGCAAGTCAGATGTGAAAACTATGGGCTCAACCCATAGCCTGCATTTGAAACTGT
TTTTCTTGAGTGCTGGAGAGGCAATCGGAATTCCGTGTGTAGCGGTGAAATGCGTAGATATACGGAGGAACACCA
GTGGCGAAGGCGGATTGCTGGACAGTAACTGACGCTGAGGCGCGAAAGCGTGGGGAGCAAACAGGATTAGATACC
CTGGTAGTCCACGCTGTAAACGATGGATACTAGGTGTGGGGGGTCTGACCCCCTCCGTGCCGCAGTTAACACAAT
AAGTATCCCACCTGGGGAGTACGATCGCAAGGTTGAAACTCAAAGGAATTGACGGGGGCCCGCACAAGCGGTGGA
GTATGTGGTTTAATTCGAAGCAACGCGAAGAACCTTACCAGGGCTTGACATCCTACTAACGAAGCAGAGATGCAT
TAGGTGCCCTTCGGGGAAAGTAGAGACAGGTGGTGCATGGTTGTCGTCAGCTCGTGTCGTGAGATGTTGGGTTAA
GTCCCGCAACGAGCGCAACCCTTATTGTTAGTTGCTACGCAAGAGCACTCTAGCGAGACTGCCGTTGACAAAACG
GAGGAAGGCGGGGACGACGTCAAATCATCATGCCCCTTATGTCCTGGGCTACACACGTACTACAATGGTGGTAAA
CAGAGGGAAGCAAGACCGCGAGGTGGAGCAAATCCCTAAAAGCCATCCCAGTTCGGATTGCAGGCTGAAACCCGC
CTGTATGAAGTTGGAATCGCTAGTAATCGCGGATCAGCATGCCGCGGTGAATACGTTCCCGGGCCTTGTACACAC
CGCCCGTCACACCATGAGAGTCGGGAACACCCGAAGTCCGTAGTCTAACCGCAAGGGGGACGCGGCCGAAGGTGG
GTTCGATAATTGGGGTGAAGTCGTAACAGGGTAACC
HMI_5 Ruminococcus flavefaciens 16S rDNA sequence
(SEQ ID NO: 5)
CGGATCAGTGGCGGACGGGTGAGTAACACGTGAGCAACCTGCCTTTAAGAGGGGGATAACGTTTGGAAACGAACG
CTAATACCGCATAACATAGAAGATTCACATGTTTCTTCTATCAAAGATTTATCGCTTAAAGATGGGCTCGCGTCT
GATTAGCTAGTTGGTGAGGTAACGGCTCACCAAGGCGACGATCAGTAGCCGTACTGAGAGGTAGAACGGCCACAT
TGGGACTGAGACACGGCCCAGACTCCTACGGGAGGCAGCAGTGGGGAATATTGCACAATGGAGGAAACTCTGATG
CAGCGATGCCGCGTGAGGGAAGAAGGTTTTCGGATTGTAAACCTCTGTCTTCAGGGACGATAATGACGGTACCTG
AGGAGGAAGCTCCGGCTAACTACGTGCCAGCAGCCGCGGTAATACGTAGGGAGCGAGCGTTGTCCGGAATTACTG
GGTGTAAAGGGAGCGTAGGCGGGATCTTAAGTCAGGTGTGAAAACTATGGGCTCAACCCATAGACTGCACTTGAA
ACTGAGGTTCTTGAGTGAAGTAGAGGCAGGCGGAATTCCTAGTGTAGCGGTGAAATGCGTAGATATTAGGAGGAA
CATCAGTGGCGAAGGCGGCCTGCTGGGCTTTTACTGACGCTGAGGCTCGAAAGCGTGGGGAGCAAACAGGATTAG
ATACCCTGGTAGTCCACGCTGTAAACGATGATTACTAGGTGTGGGGGGACTGACCCCTTCCGTGCCGCAGTTAAC
ACAATAAGTAATCCACCTGGGGAGTACGGCCGCAAGGTTGAAACTCAAAGGAATTGACGGGGGCCCGCACAAGCA
GTGGAGTATGTGGTTTAATTCGAAGCACGCGAAGAACCTTACCGGGTCTTGACATCTACAGAATCCTTTAGAGAT
AAGGGAGTGCCCTTCGGGGAACTGTAAGACAGGTGGTGCATGGTTGTCGTCAGCTCGTGTCGTGAGATGTTGGGT
TAAGTCCCGCAACGAGCGCAACCCCTATCATTAGTTGCTACGCAAGAGCACTCTAATGAGACTGCCGTTGACAAA
ACGGAGGAAGGTGGGGATGACGTCAAATCATCATGCCCCTTATGACCCGGGCTACACACGTACTACAATGGCGTA
ACAGAGGGAAGCAACATCGCGAGGTGAAGCAAATCTCTAAAAAACGTCCCAGTTCAGATTGCAGGCTGCAACTCG
CCTGCATGAAGACGGAATTGCTAGTAATCGCAGATCAGCATGCTGCGGTGAATACGTTCCCGGGCCTTGTACACA
CCGCCCGTCACACCATGGGAGTCGGTAACACCCGAAGTCGCTTGTCTAA
HMI_6 Anaerotruncus colihominis 16S rDNA sequence
(SEQ ID NO: 6)
AGTCGACGGACACATCCGACGGAATAGCTTGCTAGGAAGATGGATGTTGTTAGTGGCGGACGGGTGAGTAACACG
TGAGCAACCTACCTCAGAGTGGGGGACAACAGTTGGAAACGACTGCTAATACCGCATAAGATGGCAGGGTCGCAT
GGCCTGGTCATAAAAGGAGCAATTCGCTCTGAGATGGGCTCGCGTCTGATTAGCTAGTTGGTGAGGTAACGGCTC
ACCAAGGCAACGATCAGTAGCCGGACTGAGAGGTTGAACGGCCACATTGGGACTGAGACACGGCCCAGACTCCTA
CGGGAGGCAGCAGTGGGGAATATTGCACAATGGGGGAAACCCTGATGCAGCGACGCCGCGTGAGGGAAGACGGTT
TTCGGATTGTAAACCTCTGTCTTGTGGGACGATAGTGACGGTACCACAGGAGGAAGCCATGGCTAACTACGTGCC
AGCAGCCGCGGTAATACGTAGATGGCGAGCGTTGTCCGGAATTACTGGGTGTAAAGGGAGTGTAGGCGGGCTGGT
AAGTTGAATGTGAAACCTTCGGGCTCAACCCGGAGCGTGCGTTCAAAACTGCTGGTCTTGAGTGAAGTAGAGGCA
GGCGGAATTCCCGGTGTAGCGGTGGAATGCGTAGATATCGGGAGGAACACCAGTGGCGAAGGCGGCCTGCTGGGC
TTTTACTGACGCTGAGGCTCGAAAGCATGGGTAGCAAACAGGATTAGATACCCTGGTAGTCCATGCCGTAAACGA
TGATTACTAGGTGTGGGGGGATTGACCCCCTCCGTGCCGGAGTTAACACAATAAGTAATCCACCTGGGGAGTACG
ACCGCAAGGTTGAAACTCAAAGGAATTGACGGGGGCCCGCACAAGCAGTGGAGTATGTGGTTTAATTCGAAGCAA
CGCGAAAAACCTTACCAGGTCTTGACATCCATCGCCAGGCTAAGAGATTAGCTGTTCCCTCCGGGGACGATGAGA
CAGGTGGTGCATGGTTGTCGTCAGCTCGTGTCGTGAGATGTTGGGTTAAGTCCCGCAACGAGCGCAACCCTTACT
ATTAGTTGCTACGCAAGAGCACTCTAATGGGACTGCCGTTGACAAAACGGAGGAAGGTGGGGATGACGTCAAATC
ATCATGCCCCTTATGACCTGGGCTACACACGTACTACAATGGCCGTTAACAGAGAGCAGCGATACCGCGAGGTGG
AGCGAATCTAGAAAAACGGTCTCAGTTCGGATTGCAGGCTGAAACTCGCCTGCATGAAGTCGGAATTGCTAGTAA
TCGCGGATCAGCATGCCGCGGTGAATACGTTCCCGGGCCTTGTACACACCGCCCGTCACACCATGAGAGCCGGTA
ACACCCGAAGTCAGTAGCCTAACCGCAAGGAGGGCGCTGCCGAAGGTGGGGCTGGTAATTGGGGTGAAGTCGTAA
C
HMI_7 Clostridium xylanolyticum 16S rDNA sequence
(SEQ ID NO: 7)
GTAACGCGTGGGTAACCTGCCTCATACAGGGGGATAACAGTTAGAAATGACTGCTAATACCGCATAAGCGCACAG
GGTCGCATGACCTAGTGTGAAAAACTCCGGTGGTATGAGATGGACCCGCGTCTGATTAGCTAGTTGGTGAGGTAA
CGGCTCACCAAGGCGACGATCAGTAGCCGACCTGAGAGGGTGATCGGCCACATTGGGACTGAGACACGGCCCAAA
CTCCTACGGGAGGCAGCAGTGGGGAATATTGCACAATGGGGGAAACCCTGATGCAGCGACGCCGCGTGAAGGAAG
AAGTATTTCGGTATGTAAACTTCTATCAGCAGGGAAGAAAATGACGGTACCTGACTAAGAAGCCCCGGCTAACTA
CGTGCCAGCAGCCGCGGTAATACGTAGGGGGCAAGCGTTATCCGGATTTACTGGGTGTAAAGGGAGCGTAGGCGG
TCTGACAAGTCAGAAGTGAAAGCCCGGGGCTCAACTCCGGGACTGCTTTTGAAACTGCCGGACTAGATTGCAGGA
GAGGTAAGTGGAATTCCTAGTGTAGCGGTGAAATGCGTAGATATTAGGAGGAACACCAGTGGCGAAGGCGGCTTA
CTGGACTGTAAATGACGCTGAGGCTCGAAAGCGTGGGGAGCAAACAGGATTAGATACCCTGGTAGTCCACGCCGT
AAACGATGAATACTAGGTGTTGGGGAGCACAGCTCTTCGGTGCCGCAGCAAACGCAATAAGTATTCCACCTGGGG
AGTACGTTCGCAAGAATGAAACTCAAAGGAATTGACGGGGACCCGCACAAGCGGTGGAGCATGTGGTTTAATTCG
AAGCAACGCGAAGAACCTTACCAAGTCTTGACATCCCGATGACCGTCCCGTAACGGGGGCTTCTCTTCGGAGCAT
CGGTGACAGGTGGTGCATGGTTGTCGTCAGCTCGTGTCGTGAGATGTTGGGTTAAGTCCCGCAACGAGCGCAACC
CTTATCTTTAGTAGCCAGCGGTACGGCCGGGCACTCTAGAGAGACTGCCAGGGATAACCTGGAGGAAGGTGGGGA
TGACGTCAAATCATCATGCCCCTTATGATTTGGGCTACACACGTGCTACAATGGCGTAAACAAAGGGAAGCGAAA
CTGTGAAGTCTAGCAAATCTCAAAAATAACGTCTCAGTTCGGATTGTAGTCTGCAACTCGACTACATGAAGCTGG
AATCGCTAGTAATCGCGAATCAGCATGTCGCGGTGAATACGTTCCCGGGTCTTGTACACACCGCCCGTCACACCA
TGGGAGTTGGAAACGCCCGAAGTCAGTGACCCAACCGTAAGGAGGGAGCTGCCGAAGGCGGGTCTGATAACTGGG
GTGAAGTCGTAACAAGGTAACCG
HMI_8 Clostridium oroticum 16S rDNA sequence
(SEQ ID NO: 8)
TTTTGATTGATTTCTTCGGAAAGAGAGAGACTGTGACTGAGTGGCGGACGGGTGAGTAACGCGTGGGTAACCTGC
CTCATACAGGGGGATAACAGTTAGAAATGACTGCTAATACCGCATAAGCACACAGCTTCGCATGAAGCAGTGTGA
AAAACTCCGGTGGTATGAGATGGACCCGCGTCTGATTAGGTAGTTGGTGGGGTAACGGCCCACCAAGCCGACGAT
CAGTAGCCGACCTGAGAGGGTGACCGGCCACATTGGGACTGAGACACGGCCCAAACTCCTACGGGAGGCAGCAGT
GGGGAATATTGCACAATGGGGGAAACCCTGATGCAGCGACGCCGCGTGAAGGATGAAGTATTTCGGTATGTAAAC
TTCTATCAGCAGGGAAGAAAATGACGGTACCTGACTAAGAAGCCCCGGCTAACTACGTGCCAGCAGCCGCGGTAA
TACGTAGGGGGCAAGCGTTATCCGGATTTACTGGGTGTAAAGGGAGCGTAGACGGTAATGCAAGTCTGGAGTGAA
AACCCGGGGCTCAACCCCGGGACTGCTTTGGAAACTGTGTAACTAGAGTGTCGGAGAGGCAAGTGGAATTCCTAG
TGTAGCGGTGAAATGCGTAGATATTAGGAGGAACACCAGTGGCGAAGGCGGCTTGCTGGACGATGACTGACGTTG
AGGCTCGAAAGCGTGGGGAGCAAACAGGATTAGATACCCTGGTAGTCCACGCCGTAAACGATGACTACTAGGTGT
CGGTAAGCAAAGCTTATCGGTGCCGCAGCAAACGCAATAAGTAGTCCACCTGGGGAGTACGTTCGCAAGAATGAA
ACTCAAAGGAATTGACGGGGACCCGCACAAGCGGTGGAGCATGTGGTTTAATTCGAAGCAACGCGAAGAACCTTA
CCTGGTCTTGACATCCCTCTGACAGCTGAGTAATGTCGGTTTTCTTTCGGGACAGAGGAGACAGGTGGTGCATGG
TTGTCGTCAGCTCGTGTCGTGAGATGTTGGGTTAAGTCCCGCAACGAGCGCAACCCCTATCTTCAGTAGCCAGCA
TATGAGATGGGCACTCTGGAGAGACTGCCAGGGATAACCTGGAGGAAGGTGGGGATGACGTCAAATCATCATGCC
CCTTATGATCAGGGCTACACACGTGCTACAATGGCGTAAACAAAGGGAAGCGAGCCTGCGAGGGGGAGCAAATCC
CAAAAATAACGTCTCAGTTCGGATTGTAGTCTGCAACTCGACTACATGAAGCTGGAATCGCTAGTAATCGCGAAT
CAGAATGTCGCGGTGAATACGTTCCCGGGTCTTGTACACACCGCCCGTCACACCATGGGAGTCAGTAACGCCCGA
AGTCAGTGACCCAACCTTTCAGGAGGGAGCTGCCGAAGGCGGGACCGATAACTGGGGTGAAGTCGT
HMI_9 Eubacterium contortum 16S rDNA sequence
(SEQ ID NO: 9)
CTTAAGTTTGATTCTTCGGATGAAGACTTTTGTGACTGAGTGGCGGACGGGTGAGTAACGCGTGGGTAACCTGCC
TCATACAGGTGGATAACAGTTAGAAATGACTGCTAATACCGCATAAGACCACAGCACCGCATGGTGCAGGGGTAA
AAACTCCGGTGGTATGAGATGGACCCGCGTCTGATTAGCTAGTTGGTGGGGTAACGGCCTACCAAGGCGACGATC
AGTAGCCGACCTGAGAGGGTGACCGGCCACATTGGGACTGAGACACGGCCCAAACTCCTACGGGAGGCAGCAGTG
GGGAATATTGCACAATGGGGGAAACCCTGATGCAGCGACGCCGCGTGAAGGATGAAGTATTTCGGTATGTAAACT
TCTATCAGCAGGGAAGAAAATGACGGTACCTGACTAAGAAGCCCCGGCTAACTACGTGCCAGCAGCCGCGGTAAT
ACGTAGGGGGCAAGCGTTATCCGGATTTACTGGGTGTAAAGGGAGCGTAGACGGTATGGCAAGTCTGATGTGAAA
GGCCAGGGCTCAACCCTGGGACTGCATTGGAAACTGTCGAACTAGAGTGTCGGAGAGGCAAGTGGAATTCCTAGT
GTAGCGGTGAAATGCGTAGATATTAGGAGGAACACCAGTGGCGAAGGCGGCTTGCTGGACGATGACTGACGTTGA
GGCTCGAAAGCGTGGGGAGCAAACAGGATTAGATACCCTGGTAGTCCACGCCGTAAACGATGACTACTAGGTGTC
GGGTAGCAGAGCTATTCGGTGCCGCAGCCAACGCAATAAGTAGTCCACCTGGGGAGTACGTTCGCAAGAATGAAA
CTCAAAGGAATTGACGGGGACCCGCACAAGCGGTGGAGCATGTGGTTTAATTCGAAGCAACGCGAAGAACCTTAC
CTGCTCTTGACATCTCCCTGACCGGCAAGTAATGTTGCCTTTCCTTCGGGACAGGGATGACAGGTGGTGCATGGT
TGTCGTCAGCTCGTGTCGTGAGATGTTGGGTTAAGTCCCGCAACGAGCGCAACCCCTATCTTTAGTAGCCAGCGG
TTTGGCCGGGCACTCTAGAGAGACTGCCAGGGATAACCTGGAGGAAGGTGGGGATGACGTCAAATCATCATGCCC
CTTATGAGCAGGGCTACACACGTGCTACAATGGCGTAAACAAAGGGAAGCGAGCCTGCGAGGGTAAGCAAATCTC
AAAAATAACGTCTCAGTTCGGATTGTAGTCTGCAACTCGACTACATGAAGCTGGAATCGCTAGTAATCGCGAATC
AGAATGTCGCGGTGAATACGTTCCCGGGTCTTGTACACACCGCCCGTCACACCATGGGAGTTGGTAACGCCCGAA
GTCAGTGACCCAACCGCAAGGAGGGAGCTGCCGAAGGTGGGACCGATAACTGGGGTGAAGTCGTAACAAGGTAAC
CG
HMI_10 Clostridium oroticum 16S rDNA sequence
(SEQ ID NO: 10)
ACATGCAAGTCGAGCGAGCGCTTTAGTGGAATTCTACGGAAGGAAAGTGAAGTGACTGAGCGGCGGACGGGTGAG
TAACGCGTGGGTAACCTGCCTCATACAGGGGGATAACAGTTAGAAATGACTGCTAATACCGCATAAGACCCCAGT
ACCGCATGGTACAGAGGTAAAAACTGAGGTGGTATGAGATGGACCCGCGTCTGATTAGCTAGTTGGTGAGGTAGA
GGCTCACCAAGGCGACGATCAGTAGCCGACCTGAGAGGGTGACCGGCCACATTGGGACTGAGACACGGCCCAAAC
TCCTACGGGAGGCAGCAGTGGGGAATATTGCACAATGGGGGAAACCCTGATGCAGCGACGCCGCGTGAGCGAAGA
AGTATTTCGGTATGTAAAGCTCTATCAGCAGGGAAGAAAATGACGGTACCTGACTAAGAAGCACCGGCTAAATAC
GTGCCAGCAGCCGCGGTAATACGTATGGTGCAAGCGTTATCCGGATTTACTGGGTGTAAAGGGAGCGTAGACGGA
GCAGCAAGTCTGATGTGAAAACCCGGGGCTCAACCCCGGGAGTGCATTGGAAACTGTTGATCTAGAGTGCTGGAG
AGGTAAGTGGAATTCCTAGTGTAGCGGTGAAATGCGTAGATATTAGGAGGAACACCAGTGGCGAAGGCGGCTTAC
TGGACAGTGACTGACGTTGAGGCTCGAAAGCGTGGGGAGCAAACAGGATTAGATACCCTGGTAGTCCACGCCGTA
AACGATGACTACTAGGTGTCGGGTAGCAAAGCTATTCGGTGCCGCAGCCAACGCAATAAGTAGTCCACCTGGGGA
GTACGTTCGCAAGAATGAAACTCAAAGGAATTGACGGGGACCCGCACAAGCGGTGGAGCATGTGGTTTAATTCGA
AGCAACGCGAAGAACCTTACCTGCCCTTGACATCCGGGTGACCGGCGAGTAATGTCGCCTTCTCTTCGGAGCAGC
CGAGACAGGTGGTGCATGGTTGTCGTCAGCTCGTGTCGTGAGATGTTGGGTTAAGTCCCGCAACGAGCGCAACCC
TTATCTTTAGTAGCCAGCGGATAAGCCGGGGACTCTAGAGAGACTGCCAGGGATAACCTGGAGGAAGGTGGGGAT
GACGTCAAATCATCATGCCCCTTATGGGCAGGGCTACACACGTGCTACAATGGCGTAAACAAAGGGAAGCGAAGC
TGTGAAGCGGAGCGAATCTCAAAAATAACGTCTCAGTTCGGATTGTAGTCTGCAACTCGACTACATGAAGCTGGA
ATCGCTAGTAATCGCGAATCAGAATGTCGCGGTGAATACGTTCCCGGGTCTTGTACACACCGCCCGTCACACCAT
GGGAGTCAGTAACGCCCGAAGTCAGTGACCCAACCGTAAGGAGGGAGCTGCCGAAGGCGGGACGGATAACTGGGG
TGAAGTCGTAACAAGGTAACCG
HMI_11 Lachnospira pectinoschiza 16S rDNA sequence
(SEQ ID NO: 11)
AGTGGCGGACGGGTGAGTAACGCGTGGGTAACCTGCCCTGTACAGGGGGACAACAGCTGGAAACGGCTGCTAATA
CCGCATAAGCCCTTAGCACTGCATGGTGCATAGGGAAAAGGAGCAATCCGGTACAGGATGGACCCGCGTCTGATT
AGCCAGTTGGCAGGGTAACGGCCTACCAAAGCGACGATCAGTAGCCGATCTGAGAGGATGTACGGCCACATTGGG
ACTGAGACACGGCCCAGACTCCTACGGGAGGCAGCAGTGGGGAATATTGCACAATGGAGGAAACTCTGATGCAGC
GACGCCGCGTGAGTGAAGAAGTATTTCGGTATGTAAAGCTCTATCAGCAGGGAAGAAAATGACGGTACCTGACTA
AGAAGCACCGGCTAAATACGTGCCAGCAGCCGCGGTAATACGTATGGTGCAAGCGTTATCCGGATTTACTGGGTG
TAAAGGGAGCGTAGGTGGCAAGGCAAGCCAGAAGTGAAAACCCGGGGCTCAACCGCGGGATTGCTTTTGGAACTG
TCATGCTAGAGTGCAGGAGGGGTGAGCGGAATTCCTAGTGTAGCGGTGAAATGCGTAGATATTAGGAGGAACACC
GGAGGCGAAGGCGGCTCACTGGACTGTAACTGACACTGAGGCTCGAAAGCGTGGGGAGCAAACAGGATTAGATAC
CCTGGTAGTCCACGCGGTAAACGATGAATACTAGATGTCGGGTAGCAAAGCTACTCGGTGTCGTCGCAAACGCAA
TAAGTATTCCACCTGGGGAGTACGTTCGCAAGAATGAAACTCAAAGGAATTGACGGGGACCCGCACAAGCGGTGG
AGCATGTGGTTTAATTCGAAGCAACGCGAAGAACCTTACCTGCTCTTGACATCCCATTCGATAGAGGGTAATGCT
TCTAGCCCTTCGGGGGAATGGAGACAGGTGGTGCATGGTTGTCGTCAGCTCGTGTCGTGAGATGTTGGGTTAAGT
CCCGCAACGAGCGCAACCCTTATTGTCAGTAGCCAGCAGGTGAAGCTGGGCACTCTGATGAGACTGCCGGGGATA
ACCCGGAGGAAGGTGGGGATGACGTCAAATCATCATGCCCCTTATGAGCAGGGCTACACACGTGCTACAATGGCG
TAAACAGAGGGAAGCGAAGGAGTGATCTGGAGCAAATCTCAAAAATAACGTCTCAGTTCGGATTGTAGTCTGCAA
CTCGACTACATGAAGCTGGAATCGCTAGTAATCGCAGATCAAAATGCTGCGGTGAATACGTTCCCGGGTCTTGTA
CACACCGCCCGTCACACCATGGGAGTCGGTAATGCCCGAAGTCAGTGACTCAACCGAAAGGAAAAAGCTGCCGAA
GGCAGGACTGGTAACTGGGGTGAAGTCGT
HMI_12 Roseburia faecis 16S rDNA sequence
(SEQ ID NO: 12)
AGTCGAACGAAGCACTTTATTACGATTTCTTCGGAATGACGATTTAGTGACTGAGTGGCGGACGGGTGAGTAACG
CGTGGGTAACCTGCCTTATACAGGGGGATAACAGTTAGAAATGACTGCTAATACCGCATAAGCGCACAGGATTGC
ATGATCTGGTGTGAAAAACTCCGGTGGTATAAGATGGACCCGCGTCTGATTAGCTGGTTGGTGAGGTAACGGCCC
ACCAAGGCGACGATCAGTAGCCGACCTGAGAGGGTGACCGGCCACATTGGGACTGAGACACGGCCCAAACTCCTA
CGGGAGGCAGCAGTGGGGAATATTGCACAATGGGGGAAACCCTGATGCAGCGACGCCGCGTGAGCGAAGAAGTAT
TTCGGTATGTAAAGCTCTATCAGCAGGGAAGAAAAAAATGACGGTACCTGACTAAGAAGCCCCGGCTAACTACGT
GCCAGCAGCCGCGGTAATACGTAGGGGGCAAGCGTTATCCGGATTTACTGGGTGTAAAGGGAGCGCAGGCGGTGC
GGCAAGTCTGATGTGAAAGCCCGGGGCTCAACCCCGGGACTGCATTGGAAACTGTCGTACTTGAGTATCGGAGAG
GTAAGTGGAATTCCTAGTGTAGCGGTGAAATGCGTAGATATTAGGAGGAACACCAGTGGCGAAGGCGGCTTACTG
GACGATAACTGACGCTGAGGCTCGAAAGCGTGGGGAGCAAACAGGATTAGATACCCTGGTAGTCCACGCCGTAAA
CGATGAATACTAGGTGTCGGGGGACATAGTCCTTCGGTGCCGCAGCAAACGCAATAAGTATTCCACCTGGGGAGT
ACGTTCGCAAGAATGAAACTCAAAGGAATTGACGGGGACCCGCACAAGCGGTGGAGCATGTGGTTTAATTCGAAG
CAACGCGAAGAACCTTACCAAGTCTTGACATCCCGGTGACAAAGTATGTAATGTACTCTTTCTTCGGAACACCGG
TGACAGGTGGTGCATGGTTGTCGTCAGCTCGTGTCGTGAGATGTTGGGTTAAGTCCCGCAACGAGCGCAACCCCT
GTTCTTAGTAGCCAGCGGTTCGGCCGGGCACTCTAGGGAGACTGCCAGGGATAACCTGGAGGAAGGCGGGGATGA
CGTCAAATCATCATGCCCCTTATGACTTGGGCTACACACGTGCTACAATGGCGTAAACAAAGGGAAGCGAAAAGG
TGACTTCTAGCAAATCCCAAAAATAACGTCCCAGTTCGGACTGTAGTCTGCAACTCGACTACACGAAGCTGGAAT
CGCTAGTAATCGCGAATCAGAATGTCGCGGTGAATACGTTCCCGGGTCTTGTACACACCGCCCGTCACACCATGG
GAGTCGGGAATGCCCGAAGCCGGTGACTCAACCGAAAGGAGAGAGCCGTCGAAGGCAGGTCTGATAACTGGGGTG
AAGTCGTAACAAGGTAACC
HMI_14 Clostridium hathewayi 16S rDNA sequence
(SEQ ID NO: 13)
AGTCGACGGAGATGCGATGTGAGCGAGAGGTGCTTGCACTGATCAATCTTTTCGTATCTTAGTGGCGGACGGGTG
AGTAACGCGTGGGTAACCTGCCTTATACCGGGGGATAACACTTAGAAATAGGTGCTAATACCGCATAAGCGCACG
GTGTCGCATGACACAGTGTGAAAAACTCCGGTGGTATAAGATGGACCCGCGTCTGATTAGCCAGTTGGCAGGGTA
ACGGCCTACCAAAGCGACGATCAGTAGCCGGCCTGAGAGGGTGAACGGCCACATTGGGACTGAGACACGGCCCAA
ACTCCTACGGGAGGCAGCAGTGGGGAATATTGCACAATGGGGGAAACCCTGATGCAGCGACGCCGCGTGAGTGAA
GAAGTATTTCGGTATGTAAAGCTCTATCAGCAGGGAAGAAGAAATGACGGTACCTGACTAAGAAGCCCCGGCTAA
CTACGTGCCAGCAGCCGCGGTAATACGTAGGGGGCAAGCGTTATCCGGATTTACTGGGTGTAAAGGGAGCGTAGA
CGGTGAAGCAAGTCTGAAGTGAAAGGTTGGGGCTCAACCCCGAAACTGCTTTGGAAACTGTTTAACTGGAGTACA
GGAGAGGTAAGTGGAATTCCTAGTGTAGCGGTGAAATGCGTAGATATTAGGAGGAACACCAGTGGCGAAGGCGGC
TTACTGGACTGTAACTGACGTTGAGGCTCGAAAGCGTGGGGAGCAAACAGGATTAGATACCCTGGTAGTCCACGC
CGTAAACGATGATTACTAGGTGTTGGTGGATATGGATCCATCGGTGCCGCAGCAAACGCAATAAGTAATCCACCT
GGGGAGTACGTTCGCAAGAATGAAACTCAAAGGAATTGACGGGGACCCGCACAAGCGGTGGAGCATGTGGTTTAA
TTCGAAGCAACGCGAAGAACCTTACCTGATCTTGACATCCCTATGAATACAGGGTAATGCCTGTAGTACTTCGGT
ACATAGGAGACAGGTGGTGCATGGTTGTCGTCAGCTCGTGTCGTGAGATGTTGGGTTAAGTCCCGCAACGAGCGC
AACCCCTATCTTTAGTAGCCAGCAGTAAGATGGGCACTCTAGAGAGACTGCCGGGGATAACCCGGAGGAAGGTGG
GGATGACGTCAAATCATCATGCCCCTTATGACCAGGGCTACACACGTGCTACAATGGCGTAAACAGAGGGAAGCG
AAGTGGTGACATGGAGCAAATCCCAAAAATAACGTCCCAGTTCGGATTGCAGGCTGCAACTCGCCTGCATGAAGC
TGGAATCGCTAGTAATCGCAGATCAGAATGCTGCGGTGAATACGTTCCCGGGTCTTGTACACACCGCCCGTCACA
CCATGGGAGTAGGTAATGCCCGAAGTCGGTGACCTAACCGCAAGGAAGGAGCCGCCGAAGGCAGGACTTATAACT
GGGGTGAAGTCGTAACAAGGTAACCGT
HMI_15 Fusicatenibacter_saccharivorans 16S rDNA sequence
(SEQ ID NO: 14)
CGGATGGAATCGGTATAACTTAGTGGCGGACGGGTGAGTAACGCGTGGGAAACCTGCCCTGTACCGGGGGATAAC
ACTTAGAAATAGGTGCTAATACCGCATAAGCGCACGGAACTGCATGGTTCTGTGTGAAAAACTCCGGTGGTACAG
GATGGTCCCGCGTCTGATTAGCCAGTTGGCAGGGTAACGGCCTACCAAAGCGACGATCAGTAGCCGGCCTGAGAG
GGTGAACGGCCACATTGGGACTGAGACACGGCCCAAACTCCTACGGGAGGCAGCAGTGGGGAATATTGCACAATG
GGGGAAACCCTGATGCAGCGACGCCGCGTGAGTGAAGAAGTATTTCGGTATGTAAAGCTCTATCAGCAGGGAAGA
AAATGACGGTACCTGACTAAGAAGCCCCGGCTAACTACGTGCCAGCAGCCGCGGTAATACGTAGGGGGCAAGCGT
TATCCGGATTTACTGGGTGTAAAGGGAGCGTAGACGGCATGGCAAGCCAGATGTGAAAACCCAGGGCTCAACCTT
GGGATTGCATTTGGAACTGCCAGGCTGGAGTGCAGGAGAGGTAAGCGGAATTCCTAGTGTAGCGGTGAAATGCGT
AGATATTAGGAGGAACACCAGTGGCGAAGGCGGCTTACTGGACTGTAACTGACGTTGAGGCTCGAAAGCGTGGGG
AGCAAACAGGATTAGATACCCTGGTAGTCCACGCGGTAAACGATGATTGCTAGGTGTAGGTGGGTATGGACCCAT
CGGTGCCGCAGCTAACGCAATAAGCAATCCACCTGGGGAGTACGTTCGCAAGAATGAAACTCAAAGGAATTGACG
GGGACCCGCACAAGCGGTGGAGCATGTGGTTTAATTCGAAGCAACGCGAAGAACCTTACCAAGTCTTGACATCCC
AATGACGCACCTGTAAAGAGGTGTTCCCTTCGGGGCATTGGAGACAGGTGGTGCATGGTTGTCGTCAGCTCGTGT
CGTGAGATGTTGGGTTAAGTCCCGCAACGAGCGCAACCCTTATTCTTAGTAGCCAGCAGGTGAAGCTGGGCACTC
TAAGGAGACTGCCGGGGATAACCCGGAGGAAGGCGGGGATGACGTCAAATCATCATGCCCCTTATGATTTGGGCT
ACACACGTGCTACAATGGCGTAAACAAAGGGAAGCGAGACAGTGATGTGGAGCAAATCCCAGAAATAACGTCTCA
GTTCGGATTGTAGTCTGCAACTCGACTACATGAAGCTGGAATCGCTAGTAATCGCGAATCAGCATGTCGCGGTGA
ATACGTTCCCGGGTCTTGTACACACCGCCCGTCACACCATGGGAGTTGGAAATGCCCGAAGTCTGTGACCTAACC
GAAAGGGAGGAGCAGCCGAAGGCAGGTCTGATAACTGGGGTGAAGTCGTA
HMI_16 Clostridium_clostridioforme 16S rDNA sequence
(SEQ ID NO: 15)
CTGCTTTGATGAAGTTTTCGGATGGATTTAAAACAGCTTAGTGGCGGACGGGTGAGTAACGCGTGGGTAACCTGC
CTCACACTGGGGGATAACAGTTAGAAATAGCTGCTAATACCGCATAAGCGCACGGTTCCGCATGGAACAGTGTGA
AAAACTCCGGTGGTGTGAGATGGACCCGCGTCTGATTAGCCAGTTGGCGGGGTAACGGCCCACCAAAGCGACGAT
CAGTAGCCGGCCTGAGAGGGTGAACGGCCACATTGGGACTGAGACACGGCCCAAACTCCTACGGGAGGCAGCAGT
GGGGAATATTGCACAATGGGGGAAACCCTGATGCAGCGACGCCGCGTGAGTGAAGAAGTATTTCGGTATGTAAAG
CTCTATCAGCAGGGAAGAAAGTGACGGTACCTGAATAAGAAGCCCCGGCTAACTACGTGCCAGCAGCCGCGGTAA
TACGTAGGGGGCAAGCGTTATCCGGATTTACTGGGTGTAAAGGGAGCGTAGACGGCAAGGCAAGTCTGAAGTGAA
AGCCCGGTGCTTAACGCCGGGACTGCTTTGGAAACTGTTTAGCTGGAGTGCCGGAGAGGTAAGCGGAATTCCTAG
TGTAGCGGTGAAATGCGTAGATATTAGGAAGAACACCAGTGGCGAAGGCGGCTTACTGGACGGTAACTGACGTTG
AGGCTCGAAAGCGTGGGGAGCAAACAGGATTAGATACCCTGGTAGTCCACGCCGTAAACGATGATTGCTAGGTGT
AGGTGGGTATGGACCCATCGGTGCCGCAGCTAACGCAATAAGCAATCCACCTGGGGAGTACGTTCGCAAGAATGA
AACTCAAAGGAATTGACGGGGACCCGCACAAGCGGTGGAGCATGTGGTTTAATTCGAAGCAACGCGAAGAACCTT
ACCAGGTCTTGACATCCCGATGAAAAACCCGTAACGGGGTTCCCTCTTCGGAGCATCGGAGACAGGTGGTGCATG
GTTGTCGTCAGCTCGTGTCGTGAGATGTTGGGTTAAGTCCCGCAACGAGCGCAACCCTTATTCTTAGTAGCCAGC
AGGTAAGGCTGGGCACTCTAAGGAGACTGCCGGGGATAACCCGGAGGAAGGTGGGGATGACGTCAAATCATCATG
CCCCTTATGATCTGGGCTACACACGTGCTACAATGGCGTAACAAAGGGAAGCGAGCCTGCGAGGGTGAGCAAATC
CCAAAAATAACGTCCCAGTTCGGACTGTAGTCTGCAACCCGACTACACGAAGCTGGAATCGCTAGTAATCGCGAA
TCAGAATGTCGCGGTGAATACGTTCCCGGGTCTTGTACACACCGCCCGTCACACCATGGGAGTCGGAAATGCCCG
AAGTCTGTGACTCAACCGCAAGGAGAGAGCAGCCGAAGGCAGGTCTGATAACTGGGGTGAAGTCGT
HMI_17 Ruminococcus_torques 16S rDNA sequence
(SEQ ID NO: 16)
CGGTATGAGATGGACCCGCGTCTGATTAGCTAGTTGGTGGGGTAACGGCCTACCAAGGCG
ACGATCAGTAGCCGACCTGAGAGGGTGACCGGCCACATTGGGACTGAGACACGGCCCAAA
CTCCTACGGGAGGCAGCAGTGGGGAATATTGCACAATGGGGGAAACCCTGATGCAGCGAC
GCCGCGTGAGCGAAGAAGTATTTCGGTATGTAAAGCTCTATCAGCAGGGAAGAAAATGAC
GGTACCTGACTAAGAAGCCCCGGCTAACTACGTGCCAGCAGCCGCGGTAATACGTAGGGG
GCAAGCGTTATCCGGATTTACTGGGTGTAAAGGGAGCGTAGACGGCGAGGCAAGTCTGAT
GTGAAAACCCGGGGCTCAACCCCGTGACTGCATTGGAAACTGTTTTGCTTGAGTGCCGGA
GAGGTAAGCGGAATTCCTAGTGTAGCGGTGAAATGCGTAGATATTAGGAGGAACACCAGT
GGCGAAGGCGGCTTACTGGACGGCAACTGACGTTGAGGCTCGAAAGCGTGGGGAGCAAAC
AGGATTAGATACCCTGGTAGTCCACGCCGTAAACGATGAATACTAGGTGTCGGGGAGCAA
AGCTCTTCGGTGCCGCCGCAAACGCAATAAGTATTCCACCTGGGGAGTACGTTCGCAAGA
ATGAAACTCAAAGGAATTGACGGGGACCCGCACAAGCGGTGGAGCATGTGGTTTAATTCG
AAGCAACGCGAAGAACCTTACCAAGCCTTGACATCCCATTGACAGAGCATGTAATGTGCT
TTCCCTTCGGGGCAGTGGTGACAGGTGGTGCATGGTTGTCGTCAGCTCGTGTCGTGAGAT
GTTGGGTTAAGTCCCGCAACGAGCGCAACCCCTATCTTCAGTAGCCAGCGGTTTGGCCGG
GCACTCTGGAGAGACTGCCAGGGATAACCTGGAGGAAGGTGGGGATGACGTCAAATCATC
ATGCCCCTTATGGCTTGGGCTACACACGTGCTACAATGGCGTAAACAAAGGGAAGCGAGC
CTGCGAGGGGGAGCAAATCCCAAAAATAACGTCTCAGTTCGGATTGTAGTCTGCAACTCG
ACTACATGAAGCTGGAATCGCTAGTAATCGCGAATCAGAATGTCGCGGTGAATACGTTCC
CGGGTCTTGTACACACCGCCCGTCACACCATGGGAGTCGGCAACGCCCGAAGCCAGTGAC
CCAACCGAAAG
HMI_18 Clostridium_celerecrescens 16S rDNA sequence
(SEQ ID NO: 17)
AGTCGACGAGGTAATGAGATGAAGTTTTCGGATGGATTCTTATTTCCGAGTGGCGGACGGGTGAGTAACGCGTGG
GTAACCTGCCTCATACAGGGGGATAACGATTGGAAACGATTGCTAATACCGCATAAGCGCACAGTACCACATGGT
ACAGTGTGAAAAACTCCGGTGGTATGAGATGGACCCGCGTCTGATTAGCTAGTTGGTGAGGTAACGGCCCACCAA
GGCAACGATCAGTAGCCGACCTGAGAGGGTGACCGGCCACATTGGGACTGAGACACGGCCCAGACTCCTACGGGA
GGCAGCAGTGGGGGATATTGCACAATGGAGGAAACTCTGATGCAGCGACGCCGCGTGAGTGAAGAAGTATTTCGG
TATGTAAAGCTCTATCAGCAGGGAAGAAAATGACGGTACCTGACTAAGAAGCCCCGGCTAACTACGTGCCAGCAG
CCGCGGTAATACGTAGGGGGCAAGCGTTATCCGGATTTACTGGGTGTAAAGGGAGCGTAGACGGCGATGCAAGTC
TGAAGTGAAATACCCGGGCTCAACCTGGGAACTGCTTTGGAAACTGTATGGCTAGAGTGCTGGAGAGGTAAGCGG
AATTCCTAGTGTAGCGGTGAAATGCGTAGATATTAGGAAGAACACCAGTGGCGAAGGCGGCTTACTGGACAGTAA
CTGACGTTCAGGCTCGAAAGCGTGGGGAGCAAACAGGATTAGATACCCTGGTAGTCCACGCCGTAAACGATGAAT
ACTAGGTGTCGGGGGACAAAGTCTTTCGGTGCCGCCGCAAACGCAATAAGTATTCCACCTGGGGAGTACGTTCGC
AAGAATGAAACTCAAAGGAATTGACGGGGACCCGCACAAGCGGTGGAGCATGTGGTTTAATTCGAAGCAACGCGA
AGAACCTTACCAAATCTTGACATCCCTCTGAAAAGCCTTTAATCGAGCTCCTCCTTCGGGACAGAGGTGACAGGT
GGTGCATGGTTGTCGTCAGCTCGTGTCGTGAGATGTTGGGTTAAGTCCCGCAACGAGCGCAACCCCTATTGTCAG
TAGCCAGCAGGTAAAGCTGGGCACTCTGATGAGACTGCCAGGGATAACCTGGAGGAAGGTGGGGATGACGTCAAA
TCATCATGCCCCTTATGATTTGGGCTACACACGTGCTACAATGGCGTAAACAAAGAGAGGCGAAGCTGTGAGGCA
GAGCAAATCTCAAAAATAACGTCTCAGTTCGGATTGTAGTCTGCAACTCGACTACATGAAGCTGGAATCGCTAGT
AATCGCGGATCAGAATGCCGCGGTGAATACGTTCCCGGGTCTTGTACACACCGCCCGTCACACCATGGGAGTCGG
AAATGCCCGAAGCCAGTGACCCAAGCGAAAGCAGGGAGCTGTCGAAGGCAGGTCTGATAACTGGGGTGAAGTCGT
HMI_19 Clostridium_celerescens 16S rDNA sequence
(SEQ ID NO: 18)
TCGACGAGGTATTTTGATTGAAGTTTTCGGATGGATTTCAGATACCGAGTGGCGGACGGGTGAGTAACGCGTGGG
TAACCTGCCTCATACAGGGGGATAACGGTTAGAAATGACTGCTAATACCGCATAAGCGCACAGTACCGCATGGTA
CGGTGTGAAAAACTCCGGTGGTATGAGATGGACCCGCGTCTGATTAGCTAGTTGGTGGGGTAACGGCCCACCAAG
GCGACGATCAGTAGCCGACCTGAGAGGGTGACCGGCCACATTGGGACTGAGACACGGCCCAGACTCCTACGGGAG
GCAGCAGTGGGGGATATTGCACAATGGAGGAAACTCTGATGCAGCGACGCCGCGTGAGTGAAGAAGTATTTCGGT
ATGTAAAGCTCTATCAGCAGGGAAGAAAATGACGGTACCTGACTAAGAAGCCCCGGCTAACTACGTGCCAGCAGC
CGCGGTAATACGTAGGGGGCAAGCGTTATCCGGATTTACTGGGTGTAAAGGGAGCGTAGACGGCGACGCAAGTCT
GAAGTGAAATACCCGGGCTCAACCTGGGAACTGCTTTGGAAACTGTGTTGCTAGAGTGCTGGAGAGGTAAGCGGA
ATTCCTAGTGTAGCGGTGAAATGCGTAGATATTAGGAAGAACACCAGTGGCGAAGGCGGCTTACTGGACAGTAAC
TGACGTTGAGGCTCGAAAGCGTGGGGAGCAAACAGGATTAGATACCCTGGTAGTCCACGCCGTAAACGATGAATA
CTAGGTGTTGGTGAGCAAAGCTCATCGGTGCCGCCGCAAACGCAATAAGTATTCCACCTGGGGAGTACGTTCGCA
AGAATGAAACTCAAAGGAATTGACGGGGACCCGCACAAGCGGTGGAGCATGTGGTTTAATTCGAAGCAACGCGAA
GAACCTTACCAAATCTTGACATCCCTCTGAAACGCCCTTAATCGGGCTCCTCCTTCGGGACAGAGGTGACAGGTG
GTGCATGGTTGTCGTCAGCTCGTGTCGTGAGATGTTGGGTTAAGTCCCGCAACGAGCGCAACCCCTATTGTCAGT
AGCCAGCAGGTAAAGCTGGGCACTCTGATGAGACTGCCAGGGATAACCTGGAGGAAGGTGGGGATGACGTCAAAT
CATCATGCCCCTTATGATTTGGGCTACACACGTGCTACAATGGCGTAAACAAAGAGAAGCGAGCCTGCGAGGGGG
AGCAAATCTCAAAAATAACGTCTCAGTTCGGATTGTAGTCTGCAACTCGACTACATGAAGCTGGAATCGCTAGTA
ATCGCAGATCAGAATGCTGCGGTGAATACGTTCCCGGGTCTTGTACACACCGCCCGTCACACCATGGGAGTCGGA
AATGCCCGAAGCCAGTGACCCAAGCGAAAGCAGGGAGCTGTCGAAGGCAGGTCTGATAACTGGGGTGAAGTCGTA
ACAGGGTAACCG
HMI_20 Eubacterium_infirmum 16S rDNA sequence
(SEQ ID NO: 19)
GAGCTCATCACAGATGCTTCGGTTGAAGTGATGAGTGGAAAGCGGCGGACGGGTGAGTAA
CGCGTAGGCAACCTGCCCTTTGCAGAGGGATAGCCTCGGGAAACCGGGATTAAAACCTCA
TGACACCTCTTAAAGACATCTTTGAGAGGTCAAAGATTTATCGGCAGAGGATGGGCCTGC
GTCTGATTAGCTAGTTGGTGGGGTAACGGCCTACCAAGGCGACGATCAGTAGCCGACCTG
AGAGGGTGATCGGCCACATTGGAACTGAGACACGGTCCAAACTCCTACGGGAGGCAGCAG
TGGGGAATATTGCACAATGGGGGAAACCCTGATGCAGCAACGCCGCGTGAAGGAAGAAGG
CCTTTGGGTCGTAAACTTCTGTTCTAAGGGAAGATAATGACGGTACCTTAGGAGCAAGTC
CCGGCTAACTACGTGCCAGCAGCCGCGGTAATACGTAGGGGGCAAGCGTTATCCGGAATT
ATTGGGCGTAAAGAGTACGTAGGTGGTTACCTAAGCACGAGGTATAAGGCAATGGCTTAA
CCATTGTTCGCCTTGTGAACTGGGCTACTTGAGTGCAGGAGAGGAAAGCGGAATTCCTAG
TGTAGCGGTGAAATGCGTAGATATTAGGAGGAACACCAGTGGCGAAGGCGGCTTTCTGGA
CTGTAACTGACACTGAGGTACGAAAGCGTGGGGAGCAAACAGGATTAGATACCCTGGTAG
TCCACGCCGTAAACGATGAGCACTAGGTGTCGGGGTCGCAAGACTTCGGTGCCGCAGTTA
ACGCAATAAGTGCTCCGCCTGGGGAGTACGTTCGCAAGAATGAAACTCAAAGGAATTGAC
GGGGACCCGCACAAGCAGCGGAGCATGTGGTTTAATTCGAAGCAACGCGAAGAACCTTAC
CAGGACTTGACATCCCTCTGACAGCCTTTTAATCGAGGTTTTCTACGGACAGAGGAGACA
GGTGGTGCATGGTTGTCGTCAGCTCGTGTCGTGAGATGTTGGGTTAAGTCCCGCAACGAG
CGCAACCCTTGTCATTAGTTGCCAGCAGTAAGATGGGCACTCTAGTGAGACTGCCGGGGA
TAACTCGGAGGAAGGTGGGGATGACGTCAAATCATCATGCCCCTTATGTTCTGGGCTACA
CACGTGCTACAATGGCCGGTACAGAGAGAAAGCGAGACTGCGAAGTGGAGCGAAACTCAA
AAGCCGGTCCCAGTTCGGATTGCAGGCTGCAACTCGCCTGCATGAAGTCGGAGTTGCTAG
TAATCGCAGATCAGAATGCTGCGGTGAATGCGTTCCCGGGTCTTGTACACACCGCCCGTC
ACACCATGGAAGTTGGGGGCGCCCGAAGTTGGCAGATAAATATGTTACCTAAGGCGAAAT
CAATGACTGGGGTGAAGTCGT
HMI_21 Eubacterium_infirmum 16S rDNA sequence
(SEQ ID NO: 20)
TCGGTAAAGGGATATGGCGGAAAGCGGCGGACGGGTGAGTAACGCGTAGGCAACCTGCCC
CTTACAGAGGGATAGCCATTGGAAACGATGATTAAGACCTCATAACGCCTCCCTCCCACA
TGAGGGGGAGGCCAAAGATTCATCGGTAAGGGATGGGCCTGCGTCTGATTAGCTTGTTGG
CGGGGTAACGGCCCACCAAGGCGACGATCAGTAGCCGACCTGAGAGGGTGATCGGCCACA
TTGGAACTGAGACACGGTCCAAACTCCTACGGGAGGCAGCAGTGGGGAATATTGCACAAT
GGGCGGAAGCCTGATGCAGCAACGCCGCGTGAGGGATGAAGGCCTTCGGGTCGTAAACCT
CTGTCCTTGGGGAAGAAACAAATGACGGTACCCATGGAGGAAGCCCCGGCTAACTACGTG
CCAGCAGCCGCGGTAATACGTAGGGGGCGAGCGTTATCCGGAATTATTGGGCGTAAAGAG
TGCGTAGGTGGTTACCTAAGCGCAGGGTCTAAGGCAATGGCTCAACCATTGTTCGCCCTG
CGAACTGGGCTACTTGAGTGCAGGAGAGGAAAGCGGAATTCCTAGTGTAGCGGTGAAATG
CGTAGATATTAGGAGGAACACCAGTGGCGAAGGCGGCTTTCTGGACTGTTACTGACACTG
AGGCACGAAAGCGTGGGGAGCAAACAGGATTAGATACCCTGGTAGTCCACGCCGTAAACG
ATGAGCACTAGGTGTCGGGGCCGCAAGGCTTCGGTGCCGCAGTTAACGCATTAAGTGCTC
CGCCTGGGGAGTACGCACGCAAGTGTGAAACTCAAAGGAATTGACGGGGACCCGCACAAG
CAGCGGAGCATGTGGTTTAATTCGAAGCAACGCGAAGAACCTTACCAGGACTTGACATCC
CCCTGACAGATCCTTAACCGGATCCTTCTTCGGACAGGGGAGACAGGTGGTGCATGGTTG
TCGTCAGCTCGTGTCGTGAGATGTTGGGTTAAGTCCCGCAACGAGCGCAACCCTTGCCAT
TAGTTGCCATCATTCAGTTGGGCACTCTAATGGGACTGCCGGGGACAACTCGGAGGAAGG
TGGGGATGACGTCAAATCATCATGCCCCTTATGTTCTGGGCTACACACGTGCTACAATGG
CCGGTACAGCAGGAAGCGATCCCGCGAGGGGGAGCAAATCCCAAAAACCGGTCCCAGTTC
GGACTGCAGGCTGCAACCCGCCTGCACGAAGCCGGAGTTGCTAGTAATCGTGGATCAGAA
TGCCACGGTGAATGCGTTCCCGGGTCTTGTACACACCGCCCGTCACACCATGGAAGTTGG
GGGTGCCCGAAGCCGGCAGGGAGATATGCTGTCTAAGGCAAAACCAAT
HMI_22 Clostridium_thermocellum 16S rDNA sequence
(SEQ ID NO: 21)
GGATGAGGAAATGCTTCGGCATGGAGACATCCGATCTAGTGGCGGACGGGTGAGTAACGC
GTGAGCAACCTGTCCTGCACAGGGGGATAACACTGAGAAATCAGTGCTAATACCGCATGA
GACCACAGTATCACATGGTACAGGGGTCAAAGGAGAAATCCGGTGCAGGGTGGGCTCGCG
TCCCATTAGCTAGTTGGTAGGGTAAAGGCCTACCAAGGCGACGATGGGTAGCCGGACTGA
GAGGTTGGCCGGCCACACTGGGACTGAGACACGGCCCAGACTCCTACGGGAGGCAGCAGT
GGGGAATATTGGGCAATGGGCGAAAGCCTGACCCAGCAACGCCGCGTGAAGGAAGAAGGT
CTTTGGATTGTAAACTTTTGTCCTATGGGAAGAAGGAAGTGACGGTACCATGGGAGGAAG
CCCCGGCTAACTACGTGCCAGCAGCCGCGGTAATACGTAGGGGGCGAGCGTTGTCCGGAA
TTACTGGGCGTAAAGGGCGCGCAGGCGGCCGATCAAGTTAGATGTGAAATACCCGGGCTT
AACCTGGGAACTGCATTTAAAACTGGTTGGCTAGGAGTGCAGGAGAGGGAAGCGGAATTC
CTAGTGTAGCGGTGAAATGCGTAGATATTAGGAGGAACACCAGTGGCGAAGGCGGCTTTC
TGGACTGTAACTGACGCTGAGGCGCGAAAGCGTGGGGAGCGAACAGGATTAGATACCCTG
GTAGTCCACGCTGTAAACGATGAATACTAGGTGTAGGGGGTATCGACCCCCCCTGTGCCG
GAGCAAACGCAATAAGTATTCCGCCTGGGGAGTACGGCCGCAAGGTTGAAACTCAAAGGA
ATTGACGGGGGCCCGCACAAGCAGCGGAGCATGTGGTTTAATTCGAAGCAACGCGAAGAA
CCTTACCAGGTCTTGACATCCCTCGAAGTGCATAGAGATATGTACGTCCTTCGGGACGAG
GAGACAGGTGGTGCATGGTTGTCGTCAGCTCGTGTCGTGAGATGTTGGGTTAAGTCCCGC
AACGAGCGCAACCCCTACAGTTAGTTACCAGCGGGTAAAGCCGGGGACTCTAACAGGACT
GCCGTGGATAACACGGAGGAAGGTGGGGACGACGTCAAATCATCATGCTCCTTATGACCT
GGGCTACACACGTGCTACAATGGCCGGTACAAAGAGAAGCGAGACCGTAAGGTGGAGCGG
ATCTCAAAAAACCGGTCCCAGTTCGGATTGTGGGCTGCAACCCGCCCACATGAAGTTGGA
GTTGCTAGTAATCGCGAATCAGCATGTCGCGGTGAATGCGTTCCCGGGCCTTGTACACAC
CGCCCGTCACACCATGGGAGTTGGGAGCGCCCGAAGTCGTTGAGGTAACCCGCAAGGGAG
CCAGGCGCCGAAGGTGAGACCGATAACTGGGGTGAAGTCGT
HMI_23 Anaerovorax_odorimutans 16S rDNA sequence
(SEQ ID NO: 22)
AGAGTTTGATCCTGGCTCAGGATGAACGCTGGCGGCGTGCCTAACACATGCAAGTCGAGC
GGGAAATCTTGGAACGATACTTCGGTAAAGGGAAGAGATGGATAGCGGCGGACGGGTGAG
TAACGCGTAGGTAACCTGCCTCATGCAGAGGGATAGCCTCGGGAAACTGGGATTAATACC
TCATAATGCGGAGGAGTCACATGGCTCCATCGCCAAAGATTTATCGGCATGAGATGGACC
TGCGTCTGATTAGTTAGTTGGTGAGGTAACGGCTCACCAAGGCAGCGATCAGTAGCCGAC
CTGAGAGGGTAATCGGCCACATTGGAACTGAGACACGGTCCAAACTCCTACGGGAGGCAG
CAGTGGGGAATATTGCACAATGGGCGCAAGCCTGATGCAGCAACGCCGCGTGAGCGATGA
AGGTCTTCGGATCGTAAAGCTCTGTCCTAGGGGAAGAATATATGACGGTACCCTTGGAGG
AAGCCCCGGCTAACTACGTGCCAGCAGCCGCGGTAATACGTAGGGGGCGAGCGTTATCCG
GAATTATTGGGCGTAAAGAGTTCGTAGGTGGTTTTGTAAGCGCGGGGTTTAAGGCAACGG
CTCAACCGTTGTTCGCCTTGCGAACTGCAAGACTTGAGTGCGGGAGAGGAAAGTGGAATT
CCTAGTGTAGCGGTGAAATGCGTAGATATTAGGAGGAACACCAGTGGCGAAGGCGACTTT
CTGGACCGTAACTGACACTGAGGAACGAAAGCGTGGGGAGCAAACAGGATTAGATACCCT
GGTAGTCCACGCCGTAAACGATGAGCACTAGGTGTCGGGGCCGCAAGGTTTCGGTGCCGC
AGTTAACGCATTAAGTGCTCCGCCTGGGGAGTACGCACGCAAGTGTGAAACTCAAAGGAA
TTGACGGGGACCCGCACAAGCAGCGGAGCATGTGGTTTAATTCGAAGCAACGCGAAGAAC
CTTACCAGGGCTTGACATCCCGATGACCGGCGGGTAACGCCGCCTTCTCTTCGGAGCATC
GGTGACAGGTGGTGCATGGTTGTCGTCAGCTCGTGTCGTGAGATGTTGGGTTAAGTCCCG
CAACGAGCGCAACCCTTGTCATTAGTTGCCAGCAGTTCGGCTGGGCACTCTAGTGAGACT
GCCGGGGACAACTCGGAGGAAGGTGGGGATGACGTCAAATCATCATGCCCCTTATGTTCT
GGGCTACACACGTGCTACAATGGCCGGTACAGAGAGACGCAAGACTGTGAAGTGGAGCAA
AACTCTAAAACCGGTCCCAGTTCGGATTGTAGGCTGCAACTCGCCTACATGAAGTTGGAG
TTGCTAGTAATCGCGAATCAGAATGTCGCGGTGAATGCGTTCCCGGGTCTTGTACACACC
GCCCGTCACACCATGGAAGTTGGGGGCGCCCGAAGTTGGTCAACAAATCGATTACCTAAG
GCGAAACCAATGACTGGGGTGAAGTCGTAACAAGGTAGCCGTATCGGAAGGTGCGGCTGG
ATCACCT
HMI_24 Clostridium_saccharogumia 16S rDNA sequence
(SEQ ID NO: 23)
AGCCACCGGCTTCGGGTGTTATCAACTCTCATGGTGTGACGGGCGGTGTGTACAAGGCCC
GAGAACGTATTCACCGCGGCATGCTGATCCGCGATTACTAGCGATTCCATCTTCATGCAG
GCGAGTTGCAGCCTGCAATCCGAACTGAGAACGGGTTTTTGAGTTTCGCTCCAAGTCGCC
TCTTCGCTTCCCTTTGATCCGTCCATTGTAGCACGTGTGTAGCCCAGGTCATAAGGGGCA
TGATGATTTGACGTCATCCCCGCCTTCCTCCGGCTTGTCACCGGCTGTCTCGTTAGAGTC
CCCATCTTACTGCTGGTAACTAACGACAAGGGTTGCGCTCGTTGCGGGACTTAACCCAAC
ATCTCACGACACGAGCTGACGACAACCATGCACCACCTGTCTTGAGTATATCTATCCCTC
TATCTCTAGAGTCTTTACTCTGATGTCAAGACCTGGTAAGGTTCTTCGCGTTGCTTCGAA
TTAAACCACATGCTCCACCGCTTGTGCGGGCCCCCGTCAATTCCTTTGAGTTTCATTCTT
GCGAACGTACTACTCAGGCGGAGTACTTATTGCGTTAACTGCAGCACTGAGGCTTGTCCC
CCCAACACTTAGTACTCATCGTTTACGGCGTGGACTACTAGGGTATCTAATCCTATTTGC
TCCCCACGCTTTCGGGACTGAGCGTCAGTTACAGACCAGATCGTCGCCTTCGCCACTGGT
GTTCCTCCATATATCTACGCATTTCACCGCTACACATGGAATTCCACGATCCTCTTCTGC
ACTCTAGCTATTTGGTTTCCATGGCTTACTGAAGTTAAGCTTCAGCCTTTTACCACAGAC
CTCCATTGCCGCCTGCTCCCTCTTTACGCCCAATAATTCCGGATAACGCTTGCCACCTAC
GTATTACCGCGGCTGCTGGCACGTAGTTAGCCGTGGCTTCCTCACAAAGTACCGTCACTC
TAATACCATTCCCTGTATTAGTCGTTCTTCCTTTATAACAGAAGTTTACAACCCGAAGGC
CTTCTTCCTTCACGCGGCGTTGCTCGGTCAGGGTTCCCCCCATTGCCGAAAATTCCCTAC
TGCTGCCTCCCGTAGGAGTCTGGGCCGTGTCTCAGTCCCAGTGTGGCCGTTCACCCTCTC
AGGCCGGCTATGCATCGTCGCCTTGGTAGGCCGTTACCCCTCCAACTAGCTAATGCACCA
TAAGCCCATCTGTTCCCTATCCCTTAGGATATTTAACTTAGAGAAAATGCTTCCTCTAAG
CCTATGCGGTGTTAGCGCATGTTTCCACGCGTTATCCCCCTGGTACAGCCAGGTTGCTTA
TGTCTTACTCACCCGTTCGCCACTCATCACCGAAGTGATGCGTTCGACTTGCATGTAT
HMI_25 Clostridium_saccharogumia 16S rDNA sequence
(SEQ ID NO: 24)
GGCATCTACAGGGGGATAACTGATGGAAACGTCAGCTAAGACCGCATAGGTGTAGAGATC
GCATGAACTCTATATGAAAAGTGCTACGGGACTGGTAGATGATGGACTTATGGCGCATTA
GCTTGTTGGTAGGGTAACGGCCTACCAAGGCGACGATGCGTAGCCGACCTGAGAGGGTGA
CCGGCCACACTGGGACTGAGACACGGCCCAGACTCCTACGGGAGGCAGCAGTAGGGAATT
TTCGGCAATGGGGGAAACCCTGACCGAGCAACGCCGCGTGAAGGAAGAAGTAATTCGTTA
TGTAAACTTCTGTCATAGAGGAAGAACGGTGGATATAGGGAATGATATCCAAGTGACGGT
ACTCTATAAGAAAGCCACGGCTAACTACGTGCCAGCAGCCGCGGTAATACGTAGGTGGCG
AGCGTTATCCGGAATTATTGGGCGTAAAGAGGGAGCAGGCGGCACTAAGGGTCTGTGGTG
AAAGATCGAAGCTTAACTTCGGTAAGCCATGGAAACCGTAGAGCTAGAGTGTGTGAGAGG
ATCGTGGAATTCCATGTGTAGCGGTGAAATGCGTAGATATATGGAGGAACACCAGTGGCG
AAGGCGACGATCTGGCGCATAACTGACGCTCAGTCCCGAAAGCGTGGGGAGCAAATAGGA
TTAGATACCCTAGTAGTCCACGCCGTAAACGATGAGTACTAAGTGTTGGGTGTCAAAGCT
CAGTGCTGCAGTTAACGCAATAAGTACTCCGCCTGAGTAGTACGTTCGCAAGAATGAAAC
TCAAAGGAATTGACGGGGGCCCGCACAAGCGGTGGAGCATGTGGTTTAATTCGAAGCAAC
GCGAAGAACCTTACCAGGTCTTGACATCGATCTAAAGGCTCCAGAGATGGAGAGATAGCT
ATAGAGAAGACAGGTGGTGCATGGTTGTCGTCAGCTCGTGTCGTGAGATGTTGGGTTAAG
TCCCGCAACGAGCGCAACCCCTGTTGCCAGTTGCCAGCATTAAGTTGGGGACTCTGGCGA
GACTGCCGGTGACAAGCCGGAGGAAGGCGGGGATGACGTCAAATCATCATGCCCCTTATG
ACCTGGGCTACACACGTGCTACAATGGACAGAGCAGAGGGAAGCGAAGCCGCGAGGTGGA
GCGAAACCCATAAAACTGTTCTCAGTTCGGACTGCAGTCTGCAACTCGACTGCACGAAGA
TGGAATCGCTAGTAATCGCGAATCAGCATGTCGCGGTGAATACGTTCTCGGGCCTTGTAC
ACACCGCCCGTCACACCATGAGAGTCGGTAACACCCGAAGCCGGTGGCCTAACCGCAAGG
AAGGAGCTGTCTAAGGTGGGACTGATGATTGGGGTGAAGTCGTAACAAGGGTAACC
HMI_26 Blautia_luti 16S rDNA sequence
(SEQ ID NO: 25)
CGGGAATACTTTATTGAAACTTCGGTGGATTTAATTTATTTCTAGTGGCGGACGGGTGAGTAACGCGTGGGTAAC
CTGCCTTATACTGGGGGATAACAGCCAGAAATGACTGCTAATACCGCATAAGCGCACAGAACCGCATGGTTCCGT
GTGAAAAACTCCGGTGGTATAAGATGGACCCGCGTTGGATTAGCTAGTTGGCAGGGCAGCGGCCTACCAAGGCGA
CGATCCATAGCCGGCCTGAGAGGGTGAACGGCCACATTGGGACTGAGACACGGCCCAGACTCCTACGGGAGGCAG
CAGTGGGGAATATTGCACAATGGGGGAAACCCTGATGCAGCGACGCCGCGTGAAGGAAGAAGTATCTCGGTATGT
AAACTTCTATCAGCAGGGAAGATAATGACGGTACCTGACTAAGAAGCCCCGGCTAACTACGTGCCAGCAGCCGCG
GTAATACGTAGGGGGCAAGCGTTATCCGGATTTACTGGGTGTAAAGGGAGCGTAGACGGCGCAGCAAGTCTGATG
TGAAAGGCAGGGGCTTAACCCCTGGACTGCATTGGAAACTGCTGTGCTTGAGTGCCGGAGGGGTAAGCGGAATTC
CTAGTGTAGCGGTGAAATGCGTAGATATTAGGAGGAACACCAGTGGCGAAGGCGGCTTACTGGACGGTAACTGAC
GTTGAGGCTCGAAAGCGTGGGGAGCAAACAGGATTAGATACCCTGGTAGTCCACGCCGTAAACGATGAATACTAG
GTGTCAGGGAGCACAGCTCTTTGGTGCCGCCGCAAACGCATTAAGTATTCCACCTGGGGAGTACGTTCGCAAGAA
TGAAACTCAAAGGAATTGACGGGGACCCGCACAAGCGGTGGAGCATGTGGTTTAATTCGAAGCAACGCGAAGAAC
CTTACCAAATCTTGACATCCCTCTGACCGGGACTTAACCGTCCCTTTCCTTCGGGACAGGGGAGACAGGTGGTGC
ATGGTTGTCGTCAGCTCGTGTCGTGAGATGTTGGGTTAAGTCCCGCAACGAGCGCAACCCCTATCCTTAGTAGCC
AGCACGTAATGGTGGGCACTCTGAGGAGACTGCCAGGGATAACCTGGAGGAAGGCGGGGATGACGTCAAATCATC
ATGCCCCTTATGATTTGGGCTACACACGTGCTACAATGGCGTAAACAAAGGGAAGCGAACCCGCGAGGGTGGGCA
AATCTCAAAAATAACGTCCCAGTTCGGACTGCAGTCTGCAACTCGACTGCACGAAGCTGGAATCGCTAGTAATCG
CGGATCAGAATGCCGCGGTGAATACGTTCCCGGGTCTTGTACACACCGCCCGTCACACCATGGGAGTCAGTAACG
CCCGAAGTCAG
HMI_27 Clostridium_clostridioforme 16S rDNA sequence
(SEQ ID NO: 26)
TTGCGGTAGGTCACAGGCTTCGGGCATTTCCAACTCCCATGGTGTGACGGGCGGTGTGTA
CAAGACCCGGGAACGTATTCACCGCGACATGCTGATTCGCGATTACTAGCGATTCCAGCT
TCATGTAGTCGAGTTGCAGACTACAATCCGAACTGAGACGTTATTTCTGGGATTTGCTCA
ACATCACTGTCTCGCTTCCCTTTGTTTACGCCATTGTAGCACGTGTGTAGCCCAAATCAT
AAGGGGCATGATGATTTGACGTCATCCCCGCCTTCCTCCGGGTTATCCCCGGCAGTCTCC
CTAGAGTGCCCAGCTCTACCTGCTGGCTACTAAGGATAAGGGTTGCGCTCGTTGCGGGAC
TTAACCCAACATCTCACGACACGAGCTGACGACAACCATGCACCACCTGTCTCCAATGCT
CCGAAGAGAATGCCCCGTTACGGACACGTCATTGGGATGTCAAGACTTGGTAAGGTTCTT
CGCGTTGCTTCGAATTAAACCACATGCTCCACCGCTTGTGCGGGTCCCCGTCAATTCCTT
TGAGTTTCATTCTTGCGAACGTACTCCCCAGGTGGATTGCTTATTGCGTTAGCTGCGGCA
CCGATGGGTCCATACCCACCTACACCTAGCAATCATCGTTTACCGCGTGGACTACCAGGG
TATCTAATCCTGTTTGCTCCCCACGCTTTCGAGCCTCAACGTCAGTTACAGTCCAGTAAG
CCGCCTTCGCCACTGGTGTTCCTCCTAATATCTACGCATTTCACCGCTACACTAGGAATT
CCGCTTACCTCTCCTGCACTCCAGCCTGGCAGTTCCAAATGCAGTCCCAGGGTTGAGCCC
TGGGTTTTCACATCTGGCTTGTCATGCCGTCTACGCTCCCTTTACACCCAGTAAATCCGG
ATAACGCTTGCCCCCTACGTATTACCGCGGCTGCTGGCACGTAGTTAGCCGGGGCTTCTT
AGTCAGGTACCGTCATTTTCTTCCCTGCTGATAGAGCTTTACATACCGAAATACTTCTTC
ACTCACGCGGCGTCGCTGCATCAGGGTTTCCCCCATTGTGCAATATTCCCCACTGCTGCC
TCCCGTAGGAGTTTGGGCCGTGTCTCAGTCCCAATGTGGCCGTTCACCCTCTCAGGCCGG
CTACTGATCGTCGCTTTGGTAGGCCGTTACCCTGCCAACTGGCTAATCAGACGCGGGACC
ATCCTGTACCACCGGAGTTTTTCACACTGCCTCATGTGAAGCTGTGCGCTTATGCGGTAT
TAGCACCTATTTCTAAGTGTTATCCCCCGGTACAGGGCAGGTTTCCCACGCGTTACTCAC
CCGTCCGCCACTAAGTTACGCCGATTCCATCCGAAAACTTCCTCTGCATAACTCCGTCGA
CTG
HMI_28 Blautia_producta 16S rDNA sequence
(SEQ ID NO: 27)
CCGGTGGTCG CATCGGCGCT CCTCCTGTAG GTTGGGTCAC TGACTTCGGG
CGTTACTGAC TCCCATGGTG TGACGGGCGG TGTGTACAAG ACCCGGGAAC
GTATTCACCG CGACATTCTG ATTCGCGATT ACTAGCGATT CCAGCTTCGT
GCAGTCGAGT TGCAGACTGC AGTCCGAACT GGGACGTTAT TTTTGGGATT
TGCTCAACAT CGCTGTCTCG CTTCCCTTTG TTTACGCCAT TGTAGCACGT
GTGTAGCCCA AATCATAAGG GGCATGATGA TTTGACGTCG TCCCCGCCTT
CCTCCGGGTT ATCCCCGGCA GTCTCCCTAG AGTGCCCAGC TTCACCTGCT
GGCTACTAAG GATAGGGGTT GCGCTCGTTG CGGGACTTAA CCCAACATCT
CACGACACGA GCTGACGACA ACCATGCACC ACCTGTCTCC TCTGCCCCGA
AGGGAAGGCC CCGTTACGGG CCGGTCAGAG GGATGTCAAG ACTTGGTAAG
GTTCTTCGCG TTGCTTCGAA TTAAACCACA TGCTCCACCG CTTGTGCGGG
TCCCCGTCAA TTCCTTTGAG TTTCATTCTT GCGAACGTAC TCCCCAGGTG
GAATACTTAT TGCGTTTGCT GCGGCACCGA ATGGGCTTTG CCACCCGACA
CCTAGTATTC ATCGTTTACG GCGTGGACTA CCAGGGTATC TAATCCTGTT
TGCTCCCCAC GCTTTCGAGC CTCAACGTCA GTTACCGTCC AGAAAGCCGC
CTTCGCCACT GGTGTTCCTC CTAATATCTA CGCATTTCAC CGCTACACTA
GGAATTCCGC TTACCTCTCC GGCACTCTAG AAAAACAGTT TCCAATGCAG
TCCTGGGGTT AAGCCCCAGC CTTTCACATC AGACTTGCTC TTCCGTCTAC
GCTCCCTTTA CACCCAGTAA ATCCGGATAA CGCTTGCCCC CTACGTATTA
CCGCGGCTGA TGGCACGTAG TTAGCCGGGG CTTCTTAGTC AGGTACCGTC
ATTTTCTTCC CTGCTGATAG AAGTTTACAT ACCGAGATAC TTCTTCCTTC
ACGCGGCGTC GCTGCATCAG GGTTTCCCCC ATTGTGCAAT ATTCCCCACT
GCTGCCTCCC GTAGGAGTCT GGGCCGTGTC TCAGTCCCAA TGTGGCCGTT
CACCCTCTCA GGCCGGCTAC TGATCGTCGC CTTGGTGGGC CGTTACCCCT
CCAACTAGCT AATCAGACGC GGGTCCATCT CATACCACCG GAGTTTTTCA
CACCAGACCA TGCGGTCCTG TGCGCTTATG CGGTATTAGC AGCCATTTCT
AACTGTTATC CCCCTGTATG AGGCAGGTTA CCCACGCGTT ACTCAGCCCG
TCCGCCGCTC AGTCAAATAA GTTTCAATCC GAAGAGATCC ACTTAAGTGC
TTCGCTCGAC TTGCATGTGT TAAGCACGCC GCCAGCGTTC ATCCT
HMI_29 Blautia glucerasea 16S rDNA sequence
(SEQ ID NO: 28)
GCCTTCGGCAGCTCCGTCCTTTCGGTTCGGTCACTGACTTCGGGCGTTACTGACTCCCAT
GGTGTGACGGGCGGTGTGTACAAGACCCGGGAACGTATTCACCGCGGCATTCTGATCCGC
GATTACTAGCGATTCCAGCTTCGTGCAGTCGAGTTGCAGACTGCAGTCCGAACTGGGACG
TTATTTTTGGGATTTGCTTAAGCTCACACTCTCGCTTCCCTTTGTTTACGCCATTGTAGC
ACGTGTGTAGCCCAAATCATAAGGGGCATGATGATTTGACGTCATCCCCGCCTTCCTCCA
GGTTATCCCTGGCAGTCTCCTCAGAGTGCCCGGCCAAACCGCTGGCTACTAAGGATAGGG
GTTGCGCTCGTTGCGGGACTTAACCCAACATCTCACGACACGAGCTGACGACAACCATGC
ACCACCTGTCTCCGATGCTCCGAAGAAAAGGCGACGTTACTCGCCGGTCATAGGGATGTC
AAGACTTGGTAAGGTTCTTCGCGTTGCTTCGAATTAAACCACATGCTCCACCGCTTGTGC
GGGTCCCCGTCAATTCCTTTGAGTTTCATTCTTGCGAACGTACTCCCCAGGTGGAATACT
TACTGCGTTTGCTGCGGCACCGAATGGCTCTGCCACCCGACACCTAGTATTCATCGTTTA
CGGCGTGGACTACCAGGGTATCTAATCCTGTTTGCTCCCCACGCTTTCGAGCCTCAACGT
CAGTTACCGTCCAGTAAGCCGCCTTCGCCACTGGTGTTCCTCCTAATATCTACGCATTTC
ACCGCTACACTAGGAATTCCGCTTACCTCTCCGGTACTCAAGATCAACAGTTTCCAATGC
AGTCCGGGGGTTGAGCCCCCGCCTTTCACATCAGACTTGCTGCTCCGTCTACGCTCCCTT
TACACCCAGTAAATCCGGATAACGCTTGCCCCCTACGTATTACCGCGGCTGCTGGCACGT
AGTTAGCCGGGGCTTCTTAGTCAGGTACCGTCATTTTCTTCCCTGCTGATAGAAGTTTAC
ATACCGAGATACTTCTTCCTTCACGCGGCGTCGCTGCATCAGGGTTTCCCCCATTGTGCA
ATATTCCCCACTGCTGCCTCCCGTAGGAGTCTGGGCCGTGTCTCAGTCCCAATGTGGCCG
TCCACCCTCTCAGGCCGGCTATGGATCGTCGCTTTGGTAGGCCGTTACCCTGCCAACTGG
CTAATCCAACGCGGGTCCATCTCACACCACCGGAGTTTTTCACACTGGATCATGCAATCC
CGTGCGCTTATGCGGTATTAGCAGTCATTTCTGACTGTTATCCCCCAGTGTGAGGCAGGT
TACCCACGCGTTACTCACCCGTCCGCCACTAGGATTATAACGACTTCAACCGAAGTCTCT
GTCAAAATAATCCCCGTTCGACTTGCATGTGT
HMI_30 Clostridium_straminisolvens 16S rDNA sequence
(SEQ ID NO: 29)
AGCGGCGGACGGGTGAGTAACGCGTGAGTAACCTGCCTTTAGGAGGGGGACAACATTCCGAAAGGGATGCTAATA
CCGCATAAAATTATTGTATCGCATGGTATAATAATCAAAGATTTATCGCCTAAAGATGGACTCGCGTCCGATTAG
CTAGTTGGTGGGGTAAAAGCCTACCAAGGCGACGATCGGTAGCCGAACTGAGAGGTTGATCGGCCACATTGGGAC
TGAGACACGGCCCAGACTCCTACGGGAGGCAGCAGTGGGGGATATTGCGCAATGGGGGAAACCCTGACGCAGCAA
CGCCGCGTGAAGGAAGAAGGCCTTCGGGTTGTAAACTTCTTTAAGTGTGGAAGATAATGACGGTACACACAGAAT
AAGCCACGGCTAACTACGTGCCAGCAGCCGCGGTAATACGTAGGTGGCAAGCGTTGTCCGGATTTACTGGGTGTA
AAGGGCGTGTAGGCGGGTAGACAAGTCAGATGTGAAATACCGGGGCTCAACTCCGGGGCTGCATTTGAAACTGTA
TATCTTGAGTGTCGGAGAGGAAAGCGGAATTCCTAGTGTAGCGGTGAAATGCGTAGATATTAGGAGGAACACCAG
TGGCGAAGGCGGCTTTCTGGACGATAACTGACGCTGAGGCGCGAAAGCGTGGGGAGCAAACAGGATTAGATACCC
TGGTAGTCCACGCCGTAAACGATGGATACTAGGTGTAGGAGGTATCGACCCCTTCTGTGCCGCAGTTAACACAAT
AAGTATCCCACCTGGGGAGTACGGTCGCAAGATTGAAACTCAAAGGAATTGACGGGGGCCCGCACAAGCAGTGGA
GTATGTGGTTTAATTCGAAGCAACGCGAAGAACCTTACCAGGACTTGACATCCCACGCATAGCCTAGAGATAGGT
GAAGTCCTACGGGACGTGGAGACAGGTGGTGCATGGTTGTCGTCAGCTCGTGTCGTGAGATGTTGGGTTAAGTCC
CGCAACGAGCGCAACCCTTACTGTCAGTTACCATCATTAAGTTGGGGACTCTGGCAGGACTGCCGGTGACAAATC
GGAGGAAGGTGGGGACGACGTCAAATCATCATGCCCCTTATGTCCTGGGCTACACACGTACTACAATGGCTGTTA
ACAAAGTGAAGCAAAGCAGTGATGTGGAGCAAAACACAAAAAGCAGTCTCAGTTCAGATTGTAGGCTGAAACTCG
CCTATATGAAGTCGGAATTGCTAGTAATCGCAGATCAGCATGCTGCGGTGAATACGTTCCCGGGCCTTGTACACA
CCGCCCGTCACACCATGAGAGTCGATAACACCCGAAGCCTGT
HMI_31 Butyricoccus_pullicaecorum 16S rDNA sequence
(SEQ ID NO: 30)
AGTGGCGGACGGGTGAGTAACGCGTGAGCAATCTGCCTTTAAGAGGGGGATAACAGTCGGAAACGGCTGCTAATA
CCGCATAAAGCATCGAAACCGCATGATTTTGATGCCAAAGGAGCAATCCGCTTTTAGATGAGCTCGCGTCTGATT
AGCTGGTTGGCGGGGTAACGGCCCACCAAGGCGACGATCAGTAGCCGGACTGAGAGGTTGAACGGCCACATTGGG
ACTGAGACACGGCCCAGACTCCTACGGGAGGCAGCAGTGGGGAATATTGCGCAATGGGGGAAACCCTGACGCAGC
AACGCCGCGTGATTGAAGAAGGCCTTCGGGTTGTAAAGATCTTTAATCAGGGACGAAACAAATGACGGTACCTGA
AGAATAAGCTCCGGCTAACTACGTGCCAGCAGCCGCGGTAATACGTAGGGAGCAAGCGTTATCCGGATTTACTGG
GTGTAAAGGGCGCGCAGGCGGGCCGGTAAGTTGGAAGTGAAATCTATGGGCTTAACCCATAAACTGCTTTTCAAA
CTGCTGGTCTTGAGTGATGGAGAGGCAGGCGGAATTCCGTGTGTAGCGGTGAAATGCGTAGATATACGGAGGAAC
ACCAGTGGCGAAGGCGGCCTGCTGGACATTAACTGACGCTGAGGCGCGAAAGCGTGGGGAGCAAACAGGATTAGA
TACCCTGGTAGTCCACGCCGTAAACGATGGATACTAGGTGTGGGAGGTATTGACCCCTTCCGTGCCGCAGTTAAC
ACAATAAGTATCCCACCTGGGGAGTACGGCCGCAAGGTTGAAACTCAAAGGAATTGACGGGGGCCCGCACAAGCA
GTGGAGTATGTGGTTTAATTCGAAGCAACGCGAAGAACCTTACCAGGTCTTGACATCCCGATGACCGCCTCAGAG
ATGAGCCTTTTCTTCGGAACATCGGTGACAGGTGGTGCATGGTTGTCGTCAGCTCGTGTCGTGAGATGTTGGGTT
AAGTCCCGCAACGAGCGCAACCCTTACGGTTAGTTGATACGCAAGATCACTCTAGCCGGACTGCCGTTGACAAAA
CGGAGGAAGGTGGGGACGACGTCAAATCATCATGCCCCTTATGACCTGGGCTACACACGTACTACAATGGCAGTC
ATACAGAGGGAAGCAAAACCGCGAGGTGGAGCAAATCCCTAAAAGCTGTCCCAGTTCAGATTGCAGGCTGCAACC
CGCCTGCATGAAGTCGGAATTGCTAGTAATCGCGGATCAGCATGCCGCGGTGAATACGTTCCCGGGCCTTGTACA
CACCGCCCGTCACACCATGAGAGCCGTCAATACCCGAAGTCCGT
HMI_32 Clostridium_maritium 16S rDNA sequence
(SEQ ID NO: 31)
CTACACTGCA GTCGAGCGAT TTCTTCGGTA AGAGCGGCGG ACGGGTGAGT
AACGCGTGGG TAACCTGCCC TATACACACG GATAACATAC CGAAAGGTAT
GCTAATACGA GATAACATAA GAGATTCGCA TGGATTTCTT ATCAAAGCTT
TTGCGGTATA GGATGGACCC GCGTCTGATT AGCTAGTTGG TAAGGTAACG
GCTTACCAAG GCGACGATCA GTAGCCGACC TGAGAGGGTG ATCGGCCACA
TTGGAACTGA GACACGGTCC AAACTCCTAC GGGAGGCAGC AGTGGGGAAT
ATTGCACAAT GGGCGAAAGC CTGATGCAGC AACGCCGCGT GAGCGATGAA
GGCCTTCGGG TCGTAAAGCT CTGTCCTCAA GGAAGATAAT GACGGTACTT
GAGGAGGAAG CCCCGGCTAA CTACGTGCCA GCAGCCGCGG TAATACGTAG
GGGGCTAGCG TTATCCGGAA TTACTGGGCG TAAAGGGTGC GTAGGTGGTT
TCTTAAGTCA GAGGTGAAAG GCTACGGCTC AACCGTAGTA AGCCTTTGAA
ACTGAGAAAC TTGAGTGCAG GAGAGGAGAG TAGAATTCCT AGTGTAGCGG
TGAAATGCGT AGATATTAGG AGGAATACCA GTTGCGAAGG CGGCTCTCTG
GACTGTAACT GACACTGAGG CACGAAAGCG TGGGGAGCAA ACAGGATTAG
ATACCCTGGT AGTCCACGCC GTAAACGATG AGTACTAGCT GTCGGAGGTT
ACCCCCTTCG GTGGCGCAGC TAACGCATTA AGTACTCCGC C
HMI_33 Eubacterium fissicatens 16S rDNA sequence
(SEQ ID NO: 32)
AGTGGCGGAC GGGTGAGTAA CGCGTGGGTA ACCTGCCTTG TACAGGGGGA
TAACAGTTAG AAATGACTGC TAATACCGCA TAAGCGCACA GTATCGCATG
GTACAGTGTG AAAAACTCCG GTGGTACAAG ATGGACCCGC GTCTGATTAG
CTAGTTGGTA AGGTAACGGC TTACCAAGGC AACGATCAGT AGCCGACTTG
AGAGAGTGAT CGGCCACATT GGGACTGAGA CACGGCCCAA ACTCCTACGG
GAGGCAGCAG TGGGGAATAT TGCACAATGG GGGAAACCCT GATGCAGCGA
CGCCGCGTGA GTGAAGAAGT ATTTCGGTAT GTAAAACTCT ATCAGCAAGG
AAGATAATGA CGGTACTTGA CTAAGAAGCC CCGGCTAACT ACGTGCCAGC
AGCCGCGGTA ATACGTAGGG GGCAAGCGTT ATCCGGATTT ACTGGGTGTA
AAGGGAGCGT AGACGGTATG GTAAGTCAGA TGTGAAAGCC CGGGGCTTAA
CCCCGGAACT GCATTTGAAA CTATCAAACT AGAGTGTCGG AGAGGTAAGT
GGAATTCCTA GTGTAGCGGT GAAATGCGTA GATATTAGGA GGAACACCAG
TGGCGAAGGC GGCTTACTGG ACGATAACTG ACGTTGAGGC TCGAAAGCGT
GGGGAGCAAA CAGGATTAGA TACCCTGGTA GTCCACGCCG TAAACGATGA
ATACTAGGTG TCAGGGAACA ATAGTTCTTT GGTGCCGCAG CAAACGCATT
AAGTATTCCA CCTGGGGAGT ACGTTCGCAA GAATGAAACT CAAAGGAATT
GACGGGGACC CGCACAAGCG GTGGAGCATG TGGTTTAATT CGAAGCAACG
CGAAGAACCT TACCTGGTCT TGACATCCCA ATGACGCCTC TTTAATCGGA
GGTTTCCTTC GGGACATTGG AGACAGGTGG TGCATGGTTG TCGTCAGCTC
GTGTCGTGAG ATGTTGGGTT AAGTCCCGCA ACGAGCGCAA CCCTTATCTT
TAGTAGCCAG CAGTTCGGCT GGGCACTCTA GAGAGACTGC CAGGGATAAC
CTGGAGGAAG GTGGGGATGA CGTCAAATCA TCATGCCCCT TATGACCAGG
GCTACACACG TGCTACAATG GCGTAAACAA AGGGAAGCAA AACTGTGAGG
TTGAGCAAAT CCCAAAAATA ACGTCTCAGT TCGGATTGTA GTCTGCAACT
CGACTACATG AAGCTGGAAT CGCTAGTAAT CGCAGATCAG AATGCTGCGG
TGAATACGTT CCCGGGTCTT GTACACACCG CCCGTCACAC CATGGGAGTC
GGATATGCCC GAAGTCAGTG ACCCAACCGT AAGGAGGGAG CTGCCGAAGG
TGGAGCCGAT AACTGGGGTG AAGTCGT
HMI_34 Clostridium_saccharolyticum 16S rDNA sequence
(SEQ ID NO: 33)
AGCGGCGGAC GGGTGAGTAA CGCGTGGGTA ACCTGCCTCA TACAGGGGGA
TAACAGTTAG AAATGACTGC TAATACCGCA TAAGCGCACA GTGCTGCATG
GCACAGTGTG AAAAACTCCG GTGGTATGAG ATGGACCCGC GTTGGATTAG
GCAGTTGGCG GGGTAACGGC CCACCAAACC GACGATCCAT AGCCGGCCTG
AGAGGGTGAA CGGCCACATT GGGACTGAGA CACGGCCCAA ACTCCTACGG
GAGGCAGCAG TGGGGAATAT TGCACAATGG GGGAAACCCT GATGCAGCGA
CGCCGCGTGA GTGAAGAAGT AATTCGTTAT GTAAAGCTCT ATCAGCAGGG
AAGAAAATGA CGGTACCTGA CTAAGAAGCC CCGGCTAACT ACGTGCCAGC
AGCCGCGGTA ATACGTAGGG GGCAAGCGTT ATCCGGATTT ACTGGGTGTA
AAGGGAGCGT AGACGGCCGT GCAAGTCTGA TGTGAAAGGC TGGGGCTCAA
CCCCGGGACT GCATTGGAAA CTGTATGGCT GGAGTGCCGG AGAGGTAAGC
GGAATTCCTA GTGTAGCGGT GAAATGCGTA GATATTAGGA GGAACACCAG
TGGCGAAGGC GGCTTACTGG ACGGTAACTG ACGTTGAGGC TCGAAAGCGT
GGGGAGCAAA CAGGATTAGA TACCCTGGTA GTCCACGCCG TAAACGATGA
TTACTAGGTG TTGGGGGACA TGGTCCTTCG GTGCCGCCGC AAACGCAGTA
AGTAATCCAC CTGGGGAGTA CGTTCGCAAG AATGAAACTC AAAGGAATTG
ACGGGGACCC GCACAAGCGG TGGAGCATGT GGTTTAATTC GAAGCAACGC
GAAGAACCTT ACCAAGTCTT GACATCGAGA GGACAGAGTA TGTAATGTAC
TTTCCCTTCG GGGCCTCGAA GACAGGTGGT GCATGGTTGT CGTCAGCTCG
TGTCGTGAGA TGTTGGGTTA AGTCCCGCAA CGAGCGCAAC CCCTATCTTC
AGTAGCCAGC AATTCGGATG GGCACTCTGG AGAGACTGCC GGGGATAACC
CGGAGGAAGG CGGGGATGAC GTCAAATCAT CATGCCCCTT ATGACTTGGG
CTACACACGT GCTACAATGG CGTAAACAAA GGGAAGCGAG GGAGTGATCC
GGAGCAAATC CCAAAAATAA CGTCTCAGTT CGGATTGTAG TCTGCAACTC
GACTACATGA AGCTGGAATC GCTAGTAATC GCGAATCAGC ATGTCGCGGT
GAATACGTTC CCGGGTCTTG TACACACCGC CCGTCACACC ATGGGAGTCG
ATAACGCCCG AAGTCAGTGA CCCAACCGAA AGGAGGGAGC TGCCGAAGGC
GGGATTGGTA ACTGGGGTGA AGTCGT
HMI_35 Blautia_luti 16S rDNA sequence
(SEQ ID NO: 34)
AGTGGCGGAC GGGTGAGTAA CGCGTGGGTA ACCTGCCTTA TACTGGGGGA
TAACAGCCAG AAATGGCTGC TAATACCGCA TAAGCGCACG GGGCCGCATG
GTCCTGTGTG AAAAACTCCG GTGGTATAAG ATGGACCCGC GTTGGATTAG
CTAGTTGGCA GGGCAGCGGC CTACCAAGGC GACGATCCAT AGCCGGCCTG
AGAGGGTGAA CGGCCACATT GGGACTGAGA CACGGCCCAG ACTCCTACGG
GAGGCAGCAG TGGGGAATAT TGCACAATGG GGGAAACCCT GATGCAGCGA
CGCCGCGTGA AGGAAGAAGT ATCTCGGTAT GTAAACTTCT ATCAGCAGGG
AAGATAATGA CGGTACCTGA CTAAGAAGCC CCGGCTAACT ACGTGCCAGC
AGCCGCGGTA ATACGTAGGG GGCGAGCGTT ATCCGGATTT ACTGGGTGTA
AAGGGAGCGT AGACGGCGTA TCAAGTCTGA TGTGAAAGGC AGGGGCTTAA
CCCCTGGACT GCATTGGAAA CTGGTATGCT TGAGTGCCGG AGGGGTAAGC
GGAATTCCTA GTGTAGCGGT GAAATGCGTA GATATTAGGA GGAACACCAG
TGGCGAAGGC GGCTTACTGG ACGGTAACTG ACGTTGAGGC TCGAAAGCGT
GGGGAGCAAA CAGGATTAGA TACCCTGGTA GTCCACGCCG TAAACGATGA
ATACTAGGTG TCTGGGAGCA CAGCTCTTAG GTGCCGCCGC AAACGCATTA
AGTATTCCAC CTGGGGAGTA CGTTCGCAAG AATGAAACTC AAAGGAATTG
ACGGGGACCC GCACAAGCGG TGGAGCATGT GGTTTAATTC GAAGCAACGC
GAAGAACCTT ACCAAATCTT GACATCCCTC TGACAGAGTA TGTAATGTAC
TTTTCCTTCG GGACAGGGGA GACAGGTGGT GCATGGTTGT CGTCAGCTCG
TGTCGTGAGA TGTTGGGTTA AGTCCCGCAA CGAGCGCAAC CCCTATCCTT
AGTAGCCAGC AAGTAATGTT GGGCACTCTG AGGAGACTGC CAGGGATAAC
CTGGAGGAAG GCGGGGATGA CGTCAAATCA TCATGCCCCT TATGATTTGG
GCTACACACG TGCTACAATG GCGTAAACAA AGGGAAGCGA ACCTGTGAGG
GTGGGCAAAT CTCAAAAATA ACGTCCCAGT TCGGACTGCA GTCTGCAACT
CGACTGCACG AAGCTGGAAT CGCTAGTAAT CGCGGATCAG AATGCCGCGG
TGAATACGTT CCCGGGTCTT GTACACACCG CCCGTCACAC CATGGGAGTC
AGTAACGCCC GAAGTCAGTG ACCTAACCGT AAGGAAGGAG CTGCCGAAGG
CGGGACGGAT GACTGGGGTG AAGTCGT
HMI_36 Clostridium_methylpentosum 16S rDNA sequence
(SEQ ID NO: 35)
GGTTACCTTGTTACGACTTCACCCCAATCATCAACCCCACCTTCGACGACGTCCCCCTTG
CGGTTAGACTATCGGCTTCGGGTGTTGCCAACTCTCATGGTGTGACGGGCGGTGTGTACA
AGGCCCGGGAACGTATTCACCGCGGCATGCTGATCCGCGATTACTAGCAATTCCGGCTTC
ATGCAGGCGGGTTGCAGCCTGCAATCCGAACTGAGACTATTTTTAGGGGTTTGCTCCATG
TCACCATCTTGCTTCCCTCTGTTAATAGCCATTGTAGTACGTGTGTAGCCCAGGTCATAA
GGGGCATGATGATTTGACGTCATCCCCACCTTCCTCCGTTTTGTCAACGGCAGTCCGTCT
AGAGTGCTCTTGCGTAGCAACTAAACGTAAGGGTTGCGCTCGTTGCGGGACTTAACCCAA
CATCTCACGACACGAGCTGACGACAACCATGCACCACCTGTCTCGGTGCCCCGAAGGGCT
TCACCTATCTCTAGGCTATGCACCGGATGTCAAGACCTGGTAAGGTTCTTCGCGTTGCTT
CGAATTAAACCACATACTCCACTGCTTGTGCGGGCCCCCGTCAATTCCTTTGAGTTTCAA
CCTTGCGGTCGTACTCCCCAGGTGGATTACTTATTGTGTTAACTCCGGCACGGAAGGGGT
CAGTCCCCCCACACCTAGTAATCATCGTTTACAGCGTGGACTACCAGGGTATCTAATCCT
GTTTGCTACCCACGCTTTCGAGCCTCAGCGTCAGTTAAAGCCCAGCAGGCCGCCTTCGCC
ACTGGTGTTCCTCCTAATATTTACGCATTTCACCGCTACACTAGGAATTCCGCCTGCCTC
TACTTCACTCAAGAACTGCAGTTTTGAACGCGGCTATGGGTTGAGCCCATAGATTTAACA
TTCAACTTGCAATCCCGCCTACGCTCCCTTTACACCCAGTAATTCCGGACAACGCTCGCT
ACCTACGTATTACCGCGGCTGCTGGCACGTAGTTAGCCGTAGCTTCCTCCTTGGTTACCG
TCATTATCTTCACCAAGGACAGAGGTTTACAATCCGAAAACCTTCTTCCCTCACTCGGCG
TCGCTGCATCAGGGTTTCCCCCATTGTGCAATATTCCCCACTGCTGCCTCCCGTAGGAGT
CTGGGCCGTGTCTCAGTCCCAATGTGGCCGTTCAACCTCTCAGTCCGGCTACCAATCGTC
GCCTTGGTGGGCCGTTACCTCACCAACTAGCTAATTGGACGCGAGTCCATCTTTCAGCGG
ATTGCTCCTTTGATATCAGCTCCATGCGAAACCAATATGTTATGCGGTATTAGCGTCCGT
TTCCAGACGTTATCCCCCTCTGAAAGGCAGGTTACTCACGCGTTACTCACCCGTCCGCCA
CTAAGTTGAATCAAATTCCTTCCGAAGAATTCATTCAAAGCAACTTCGTCGACTTGCATG
TGTAAGGCGCGCCGACAGCGTTCGT
HMI_37 Clostridium_xylanolyticum 16S rDNA sequence
(SEQ ID NO: 36)
AGAGTTTGATCCTGGCTCAGGATGAACGCTGGCGGCGTGCCTAACACATGCAAGTCGAAC
GGAATTTACATGAAGCCTAGCGATTGTAAATTTAGTGGCGGACGGGTGAGTAACGCGTGG
GTAACCTGCCTTGTACTGGGGGACAACAGTTGGAAACGACTGCTAATACCGCATAAGCGC
ACAGCTTCGCATGAAGCAGTGTGAAAAACTCCGGTGGTACAAGATGGACCCGCGTCTGAT
TAGCTGGTTGGTGAGGTAACGGCCCACCAAGGCGACGATCAGTAGCCGGCCTGAGAGGGT
GAACGGCCACATTGGGACTGAGACACGGCCCAAACTCCTACGGGAGGCAGCAGTGGGGAA
TATTGCACAATGGGGGAAACCCTGATGCAGCAACGCCGCGTGAGTGAAGAAGTATTTCGG
TATGTAAAGCTCTATCAGCAGGAAAGAAAATGACGGTACCTGACTAAGAAGCCCCGGCTA
ACTACGTGCCAGCAGCCGCGGTAATACGTAGGGGGCAAGCGTTATCCGGATTTACTGGGT
GTAAAGGGAGCGTAGACGGTTTTGCAAGTCTGAAGTGAAAGCCCGGGGCTTAACCCCGGG
ACTGCTTTGGAAACTGTAGGACTAGAGTGCAGGAGAGGTAAGTGGAATTCCTAGTGTAGC
GGTGAAATGCGTAGATATTAGGAGGAACACCAGTGGCGAAGGCGGCTTACTGGACTGTAA
CTGACGTTGAGGCTCGAAAGCGTGGGGAGCAAACAGGATTAGATACCCTGGTAGTCCACG
CCGTAAACGATGATTACTAGGTGTTGGTGGGTACGACCCATCGGTGCCGCAGCAAACGCA
ATAAGTAATCCACCTGGGGAGTACGTTCGCAAGAATGAAACTCAAAGGAATTGACGGGGA
CCCGCACAAGCGGTGGAGCATGTGGTTTAATTCGAAGCAACGCGAAGAACCTTACCTGGT
CTTGACATCCCTATGAATAACGGGCAATGCCGTTAGTACTTCGGTACATAGGAGACAGGT
GGTGCATGGTTGTCGTCAGCTCGTGTCGTGAGATGTTGGGTTAAGTCCCGCAACGAGCGC
AACCCTTATCTTTAGTAGCCAGCAGTAAGATGGGCACTCTAGAGAGACTGCCGGGGATAA
CCCGGAGGAAGGTGGGGATGACGTCAAATCATCATGCCCCTTATGACCAGGGCTACACAC
GTGCTACAATGGCGTAAACAAAGAGAAGCGAAGTCGTGAGGCAGAGCGAATCTCAAAAAT
AACGTCTCAGTTCGGATTGTAGTCTGCAACTCGACTACATGAAGCTGGAATCGCTAGTAA
TCGCAGATCAGAATGCTGCGGTGAATACGTTCCCGGGTCTTGTACACACCGCCCGTCACA
CCATGGGAGTCGGAAATGCCCGAAGTCGGTGACCTAACCGAA
HMI_38 Oscillibacter_valericigenes 16S rDNA sequence
(SEQ ID NO: 37)
CTCAGGACGAACGCTGGCGGCGTGCTTAACACATGCAAGTCGAACGGAGCACCCTTGACT
GAGGTTTCGGCCAAATGATAGGAATGCTTAGTGGCGGACTGGTGAGTAACGCGTGAGGAA
CCTACCTTCCAGAGGGGGACAACAGTTGGAAACGACTGCTAATACCGCATGACGCATGAC
CGGGGCATCCCGGGCATGTCAAAGATTTTATCGCTGGAAGATGGCCTCGCGTCTGATTAG
CTAGATGGTGGGGTAACGGCCCACCATGGCGACGATCAGTAGCCGGACTGAGAGGTTGAC
CGGCCACATTGGGACTGAGATACGGCCCAGACTCCTACGGGAGGCAGCAGTGGGGAATAT
TGGGCAATGGACGCAAGTCTGACCCAGCAACGCCGCGTGAAGGAAGAAGGCTTTCGGGTT
GTAAACTTCTTTTGTCAGGGAAGAGTAGAAGACGGTACCTGACGAATAAGCCACGGCTAA
CTACGTGCCAGCAGCCGCGGTAATACGTAGGTGGCAAGCGTTGTCCGGATTTACTGGGTG
TAAAGGGCGTGCAGCCGGGCCGGCAAGTCAGATGTGAAATCTGGAGGCTTAACCTCCAAA
CTGCATTTGAAACTGTAGGTCTTGAGTACCGGAGAGGTTATCGGAATTCCTTGTGTAGCG
GTGAAATGCGTAGATATAAGGAAGAACACCAGTGGCGAAGGCGGATAACTGGACGGCAAC
TGACGGTGAGGCGCGAAAGCGTGGGGAGCAAACAGGATTAGATACCCTGGTAGTCCACGC
TGTAAACGATGGATACTAGGTGTGCGGGGACTGACCCCCTGCGTGCCGCAGTTAACACAA
TAAGTATCCCACCTGGGGAGTACGATCGCAAGGTTGAAACTCAAAGGAATTGACGGGGGC
CCGCACAAGCGGTGGATTATGTGGTTTAATTCGAAGCAACGCGAAGAACCTTACCAGGGC
TTGACATCCTACTAACGAAGTAGAGATACATCAGGTGCCCTTCGGGGAAAGTAGAGACAG
GTGGTGCATGGTTGTCGTCAGCTCGTGTCGTGAGATGTTGGGTTAAGTCCCGCAACGAGC
GCAACCCCTATTGTTAGTTGCTACGCAAGAGCACTCTAGCGAGACTGCCGTTGACAAAAC
GGAGGAAGGTGGGGACGACGTCAAATCATCATGCCCCTTATGTCCTGGGCTACACACGTA
ATACAATGGCGGTCAACAGAGGGAGGCAAAGCCGCGAGGCAGAGCAAACCCCCAAAAGCC
GTCCCAGTTCGGATCGCAGGCTGCAACCCGCCTGCGTGAAGTCGGAATCGCTAGTAATCG
CGGATCAGCATGCCGCGGTGAATACGTTCCCGGGCCTTGTACACACCGCCCGTCACACCA
TGAGAGTCGGGAACACCCGAAGTCCGTAGCCTAACCGCAAGGAGGGCGCGGCCGAAGGTG
GGTTCGATAATTGGGGTGAAGTCGTAACAAGGTAACCG
HMI_39 Ruminococcus_obeum 16S rDNA sequence
(SEQ ID NO: 38)
AGTCGAACGGGAACCTTTTATTGAAGCTTCGGCAGATTTAGCTGGTTTCTAGTGGCGGAC
GGGTGAGTAACGCGTGGGTAACCTGCCCTATACAGGGGGATAACAACCAGAAATGGTTGC
TAATACCGCATAAGCGCACAGGACCGCATGGTCCGGTGTGAAAAACTCCGGTGGTATAGG
ATGGACCCGCGTTGGATTAGCCAGTTGGCAGGGTAACGGCCTACCAAAGCGACGATCCAT
AGCCGGCCTGAGAGGGTGAACGGCCACATTGGGACTGAGACACGGCCCAGACTCCTACGG
GAGGCAGCAGTGGGGAATATTGCACAATGGGGGAAACCCTGATGCAGCGACGCCGCGTGA
AGGAAGAAGTATCTCGGTATGTAAACTTCTATCAGCAGGGAAGATAGTGACGGTACCTGA
CTAAGAAGCCCCGGCTAACTACGTGCCAGCAGCCGCGGTAATACGTAGGGGGCAAGCGTT
ATCCGGATTTACTGGGTGTAAAGGGAGCGTAGACGGATTAGCAAGTCTGATGTGAAAGGC
AGGGGCTCAACCCCTGGACTGCATTGGAAACTGCCAGTCTTGAGTGCCGGAGAGGTAAGC
GGAATTCCTAGTGTAGCGGTGAAATGCGTAGATATTAGGAGGAACACCAGTGGCGAAGGC
GGCTTACTGGACGGCAACTGACGTTGAGGCTCGAAAGCGTGGGGAGCAAACAGGATTAGA
TACCCTGGTAGTCCACGCCGTAAACGATGAATACTAGGTGTTGGGGAGCAAAGCTCTTCG
GTGCCGCCGCAAACGCATTAAGTATTCCACCTGGGGAGTACGTTCGCAAGAATGAAACTC
AAAGGAATTGACGGGGACCCGCACAAGCGGTGGAGCATGTGGTTTAATTCGAAGCAACGC
GAAGAACCTTACCAAGTCTTGACATCCCTCTGACGGACTCTTAACCGAGTCTTTCCTTCG
GGACAGAGGAGACAGGTGGTGCATGGTTGTCGTCAGCTCGTGTCGTGAGATGTTGGGTTA
AGTCCCGCAACGAGCGCAACCCCTATCCCCAGTAGCCAGCATTTCGGATGGGCACTCTGA
GGAGACTGCCAGGGATAACCTGGAGGAAGGCGGGGATGACGTCAAATCATCATGCCCCTT
ATGATTTGGGCTACACACGTGCTACAATGGCGTAAACAAAGGGAAGCGAGCCTGCGAGGG
TAAGCAAATCCCAAAAATAACGTCCCAGTTCGGACTGCAGTCTGCAACTCGACTGCACGA
AGCTGGAATCGCTAGTAATCGCGGATCAGAATGCCGCGGTGAATACGTTCCCGGGTCTTG
TACACACCGCCCGTCACACCATGGGAGTCAGTAACGCCCGAAGTCAGTGACCTAACCGCA
AGGGAGGAGCTGCCGAAGGCGGGACCGATGACTGGGGTGAAGTCGTAACAAGGTAACCGT
GACTACACGAAGCTGGAATCGCTAGTAATCGCGGATCAGAATGCCGCGGTGAATACGTTCC
CGGGTCTTGTACACACCGCCCGTCACACCATGGGAGTCAGTAACGCCCGAAGTCAGTGACC
TAACCGCAAGGAAGGAGCTGCCGAAGGCGGGACCGATGACTGGGGTGAAGTCGTAACA
HMI_40 Megasphaera elsdenii 16S rDNA sequence
(SEQ ID NO: 39)
ACGCGTAAGCAACCTGCCCTCCGGATGGGGACAACAGCTGGAAACGGCTGCTAATACCGA
ATACGTTTCCATTGCCGCATGGCAGTGGGAAGAAAGGTGGCCTCTGAATATGCTACCGCC
GGGGGAGGGGCTTGCGTCTGATTAGCTAGTTGGAGGGGTAACGGCCCACCAAGGCGACGA
TCAGTAGCCGGTCTGAGAGGATGAACGGCCACATTGGAACTGAGACACGGTCCAGACTCC
TACGGGAGGCAGCAGTGGGGAATCTTCCGCAATGGGCGAAAGCCTGACGGAGCAACGCCG
CGTGAGCGAAGACGGCCTTCGGGTTGTAAAGCTCTGTTATACGGGACGAACGGCTAGTGT
GCCAATACCACATTAGAATGACGGTACCGTAAGAGAAAGCCACGGCTAACTACGTGCCAG
CAGCCGCGGTAATACGTAGGTGGCAAGCGTTGTCCGGAATTATTGGGCGTAAAGGGCGCG
CAGGCGGTTTCATAAGTCTGTCTTAAAAGTGCGGGGCTTAACCCCGTGAGGGGACGGAAA
CTGTGAGACTGGAGTGTCGGAGAGGAAAGCGGAATTCCTAGTGTAGCGGTGAAATGCGTA
GATATTAGGAGGAACACCAGTGGCGAAAGCGGCTTTCTGGACGACAACTGACGCTGAGGC
GCGAAAGCCAGGGGAGCGAACGGGATTAGATACCCCGGTAGTCCTGGCCGTAAACGATGG
ATACTAGGTGTAGGGGGTATCGACCCCTCCTGTGCCGGAGTTAACGCAATAAGTATCCCG
CCTGGGGAGTACGGCCGCAAGGCTGAAACTCAAAGGAATTGACGGGGGCCCGCACAAGCG
GTGGAGTATGTGGTTTAATTCGACGCAACGCGAAGAACCTTACCAAGCCTTGACATTGAG
TGCTATCCTCAGAGATGAGGAGTTCTTCTTCGGAAGACGCGAAAACAGGTGGTGCACGGC
TGTCGTCAGCTCGTGTCGTGAGATGTTGGGTTAAGTCCCGCAACGAGCGCAACCCCTATC
TTCTGTTGCCAGCGCGTCATGGCGGGGACTCAGGAGAGACTGCCGCAGACAATGCGGAGG
AAGGCGGGGATGACGTCAAGTCATCATGCCCCTTATGGCTTGGGCTACACACGTACTACA
ATGGCTCTTAATAGAGGGAAGCGAAGGAGCGATCCGGAGCAAACCCCAAAAACAGAGTCC
CAGTTCGGATTGCAGGCTGCAACCCGCCTGCATGAAGCAGGAATCGCTAGTAATCGCAGG
TCAGCATACTGCGGTGAATACGTTCCCGGGCCTTGTACACACCGCCCGTCACACCACGAA
AGTCATTCACACCCGAAGCCGGTGAGGTAACCGTAAGGAGCCAGCCGTCGAAGGTGGGGG
CGATGATTGGGGTGAAGTCGTAA
HMI_41 Blautia_luti 16S rDNA sequence
(SEQ ID NO: 40)
GGTGAGTAACGCGTGGGTAACCTGCCTTATACAGGGGGATAACAGTCAGAAATGGCTGCT
AATACCGCATAAGCGCACAGGGCCGCATGGCCCGGTGTGAAAAACTGAGGTGGTATAAGA
TGGACCCGCGTTGGATTAGCCAGTTGGCAGGGTAACGGCCTACCAAAGCGACGATCCATA
GCCGGCCTGAGAGGGTGAACGGCCACATTGGGACTGAGACACGGCCCAGACTCCTACGGG
AGGCAGCAGTGGGGAATATTGCACAATGGGGGAAACCCTGATGCAGCGACGCCGCGTGAA
GGAAGAAGTATCTCGGTATGTAAACTTCTATCAGCAGGGAAGAAAATGACGGTACCTGAC
TAAGAAGCCCCGGCTAACTACGTGCCAGCAGCCGCGGTAATACGTAGGGGGCAAGCGTTA
TCCGGATTTACTGGGTGTAAAGGGAGCGTAGACGGCATAACAAGTCTGATGTGAAAGGCT
GGGGCTTAACCCCGGGACTGCATTGGAAACTGTTAAGCTTGAGTGCCGGAGGGGTAAGCG
GAATTCCTAGTGTAGCGGTGAAATGCGTAGATATTAGGAGGAACACCAGTGGCGAAGGCG
GCTTACTGGACGGTAACTGACGTTGAGGCTCGAAAGCGTGGGGAGCAAACAGGATTAGAT
ACCCTGGTAGTCCACGCCGTAAACGATGAATACTAGGTGTCGGGGAGCACAGCTCTTCGG
TGCCGCCGCAAACGCATTAAGTATTCCACCTGGGGAGTACGTTCGCAAGAATGAAACTCA
AAGGAATTGACGGGGACCCGCACAAGCGGTGGAGCATGTGGTTTAATTCGAAGCAACGCG
AAGAACCTTACCAAGTCTTGACATCTGCCTGACCGGTGAGTAACGTCACCTTTCCTTCGG
GACAGGCAAGACAGGTGGTGCATGGTTGTCGTCAGCTCGTGTCGTGAGATGTTGGGTTAA
GTCCCGCAACGAGCGCAACCCCTATCCCCAGTAGCCAGCATGTAAAGGTGGGCACTCTGA
GGAGACTGCCAGGGATAACCTGGAGGAAGGTGGGGATGACGTCAAATCATCATGCCCCTT
ATGATTTGGGCTACACACGTGCTACAATGGCGTAAACAGAGGGAAGCGAAAGGGTGACCT
GGAGCAAATCCCAAAAATAACGTCCCAGTTCGGACTGTAGTCTGCAACCCGACTACACGA
AGCTGGAATCGCTAGTAATCGCGGATCAGAATGCCGCGGTGAATACGTTCCCGGGTCTTG
TACACACCGCCCGTCACACCATGGGAGTCAGTAACGCCCGAAGTCAGTGACCTAACCGAA
AGGGAGGAGCTGCCGAAGGCGGGACGGATGACTGGGGTGAAGTCGTAAC
HMI_42 Bacteroides coprocola 16S rDNA sequence
(SEQ ID NO: 41)
GTATCCAACCTTCCGTTTACTCAGGGATAGCCTTTCGAAAGAAAGATTAATACCTGATAG
TATGGTAAGATTGCATGATAATACCATTAAAGATTCATCGGTAAACGATGGGGATGCGTT
CCATTAGGTAGTAGGCGGGGTAACGGCCCACCTAGCCGACGATGGATAGGGGTTCTGAGA
GGAAGGTCCCCCACATTGGAACTGAGACACGGTCCAAACTCCTACGGGAGGCAGCAGTGA
GGAATATTGGTCAATGGGCGAGAGCCTGAACCAGCCAAGTAGCGTGAAGGATGAAGGTTC
TATGGATTGTAAACTTCTTTTATAAGGGAATAAAGTGCTTTACGTGTAGAGTTTTGTATG
TACCTTATGAATAAGCATCGGCTAACTCCGTGCCAGCAGCCGCGGTAATACGGAGGATGC
GAGCGTTATCCGGATTTATTGGGTTTAAAGGGAGCGTAGACGGGATGTTAAGTCAGCTGT
GAAAGTTTGGGGCTCAACCTTAAAATTGCAGTTGAAACTGGCGTTCTTGAGTGCGGTAGA
GGCAGGCGGAATTCGTGGTGTAGCGGTGAAATGCTTAGATATCACGAAGAACCCCGATTG
CGAAGGCAGCTTGCTGGAGCGTAACTGACGTTGATGCTCGAAAGTGTGGGTATCAAACAG
GATTAGATACCCTGGTAGTCCACACGGTAAACGATGGATACTCGCTGTTGGCGATATACG
GTCAGCGGCCAAGCGAAAGCATTAAGTATCCCACCTGGGGAGTACGCCGGCAACGGTGAA
ACTCAAAGGAATTGACGGGGGCCCGCACAAGCGGAGGAACATGTGGTTTAATTCGATGAT
ACGCGAGGAACCTTACCCGGGCTTAAATTATGCATGAATGATCTGGAGACAGATCAGCCG
CAAGGCATGTATGAAGGTGCTGCATGGTTGTCGTCAGCTCGTGCCGTGAGGTGTCGGCTT
AAGTGCCATAACGAGCGCAACCCTTTCTGCCAGTTACTAACAGGCAATGCTGAGGACTCT
GGCGGTACTGCCATCGTAAGATGTGAGGAAGGTGGGGATGACGTCAAATCAGCACGGCCC
TTACGTCCGGGGCTACACACGTGTTACAATGGGGGGTACAGAAGGCAGCTTACCGGCGAC
GGTTGGCCAATCCCTAAAGCCCCTCTCAGTTCGGACTGGAGTCTGCAACCCGACTCCACG
AAGCTGGATTCGCTAGTAATCGCGCATCAGCCACGGCGCGGTGAATACGTTCCCGGGCCT
TGTACACACCGCCCGTCAAGCCATGAAAGCCGGGAGTACCTGAAGTGCGTAACCGCGAGG
AGCGCCCTAGGGTAACACTGGTAATTGGGGCTAAGTCGT
HMI_43 Bacteroides plebius 16S rDNA sequence
(SEQ ID NO: 42)
GGGGCAGCATGAACTTAGCTTGCTAAGTTCGATGGCGACCGGCGCACCGTTGAGTAACGC
GTATCCAACCTTCCGTACACTCAGGAATAGCCTTTCGAAAGAAAGATTAATACCTGATGG
TATGATGGGATTGCATGAAATCATCATTAAAGATTCATCGGTGTACGATGGGGATGCGTT
CCATTAGATAGTAGGCGGGGTAACGGCCCACCTAGTCGACGATGGATAGGGGTTCTGAGA
GGAAGGTCCCCCACATTGGAACTGAGACACGGTCCAAACTCCTACGGGAGGCAGCAGTGA
GGAATATTGGTCAATGGGCGCGAGCCTGAACCAGCCAAGTAGCGTGAAGGATGAAGGTCC
TACGGATTGTAAACTTCTTTTATAAGGGAATAAAGTCACCCACGTGTGGGTGTTTGTATG
TACCTTATGAATAAGCATCGGCTAACTCCGTGCCAGCAGCCGCGGTAATACGGAGGATGC
GAGCGTTATCCGGATTTATTGGGTTTAAAGGGAGCGTAGACGGGTCGTTAAGTCAGCTGT
GAAAGTTCGGGGCTCAACCTTGAAATTGCAGTTGATACTGGCGTCCTTGAGTACGGTTGA
GGCAGGCGGAATTCGTGGTGTAGCGGTGAAATGCTTAGATATCACGAAGAACCCCGATTG
CGAAGGCAGCCTGCTAAACCGCCACTGACGTTGAGGCTCGAAAGTGTGGGTATCAAACAG
GATTAGATACCCTGGTAGTCCACACGGTAAACGATGGATACTCGCTGTTGGCGATAGACT
GTCAGCGGCTTAGCGAAAGCGTTAAGTATCCCACCTGGGGAGTACGCCGGCAACGGTGAA
ACTCAAAGGAATTGACGGGGGCCCGCACAAGCGGAGGAACATGTGGTTTAATTCGATGAT
ACGCGAGGAACCTTACCCGGGCTTGAATTGCAGACGAATTGCTTGGAAACAGGCAAGCCG
CAAGGCGTCTGTGAAGGTGCTGCATGGTTGTCGTCAGCTCGTGCCGTGAGGTGTCGGCTT
AAGTGCCATAACGAGCGCAACCCTCGTGTCCAGTTGCTAGCAGGTAGTGCTGAGGACTCT
GGACAGACTGCCATCGTAAGATGTGAGGAAGGTGGGGATGACGTCAAATCAGCACGGCCC
TTACGTCCGGGGCTACACACGTGTTACAATGGGGGGTACAGCAGGCAGCTACCGGGCGAC
CGGATGCCAATCCCGAAAGCCTCTCTCAGTTCGGACTGGAGTCTGCAACCCGACTCCACG
AAGCTGGATTCGCTAGTAATCGCGCATCAGCCACGGCGCGGTGAATACGTTCCCGGGCCT
TGTACACACCGCCCGTCAAGCCATGAAAGCCGGGGGTACCTGAAGTGCGTAACCGCAAGG
AGCGCCCTAGGGTAAAACTGGTAATT
HMI_44 Roseburia inulinivorans 16S rDNA sequence
(SEQ ID NO: 43)
GCACTTTTGCCGATTTTCTTCGGAACTGAAGTAATAGTGACTGAGTGGCGGACGGGTGAG
TAACGCGTGGATAACCTGCCTCACACAGGGGGATAACAGTTAGAAATGACTGCTAATACC
GCATAAGCGCACAGTACCGCATGGTACAGTGTGAAAAACTCCGGTGGTGTGAGATGGATC
CGCGTCTGATTAGCCAGTTGGCGGGGTAACGGCCCACCAAAGCGACGATCAGTAGCCGGC
CTGAGAGGGCGACCGGCCACATTGGGACTGAGACACGGCCCAGACTCCTACGGGAGGCAG
CAGTGGGGAATATTGCACAATGGGGGAAACCCTGATGCAGCGACGCCGCGTGAGCGAAGA
AGTATTTCGGTATGTAAAGCTCTATCAGCAGGGAAGAAAATGACGGTACCTGACTAAGAA
GCTCCGGCTAAATACGTGCCAGCAGCCGCGGTAATACGTATGGAGCAAGCGTTATCCGGA
TTTACTGGGTGTAAAGGGAGCGCAGGCGGTATGACAAGTCTGATGTGAAAGGCTGGGGCT
CAACCCCAGGACTGCATTGGAAACTGTCAGACTAGAGTGTCGGAGAGGTAAGTGGAATTC
CTAGTGTAGCGGTGAAATGCGTAGATATTAGGAGGAACACCAGTGGCGAAGGCGGCTTAC
TGGACGACAACTGACGCTGAGGCTCGAAAGCGTGGGGAGCAAACAGGATTAGATACCCTG
GTAGTCCACGCCGTAAACGATGAATACTAGGTGTCGGGAGGCAGAGCCTTTCGGTGCCGC
AGCAAACGCAGTAAGTATTCCACCTGGGGAGTACGTTCGCAAGAATGAAACTCAAAGGAA
TTGACGGGGACCCGCACAAGCGGTGGAGCATGTGGTTTAATTCGAAGCAACGCGAAGAAC
CTTACCAGGCCTTGACATCCCCCTGACGGGACAGTAATGTGTCCGTTCCTTCGGGACAGA
GGAGACAGGTGGTGCATGGTTGTCGTCAGCTCGTGTCGTGAGATGTTGGGTTAAGTCCCG
CAACGAGCGCAACCCTTATCCTCAGTAGCCAGCGGATAAAGCCGGGCACTCTGTGGAGAC
TGCCAGGGACAACCTGGAGGAAGGTGGGGATGACGTCAAATCATCATGCCCCTTATGGCC
TGGGCTACACACGTGCTACAATGGCGTAAACAAAGGGAAGCGAAGCTGTGAAGTGAAGCA
AATCCCAAAAATAACGTCTCAGTTCGGATTGTAGTCTGCAACTCGACTACATGAAGCTGG
AATCGCTAGTAATCGCGAATCAGAATGTCGCGGTGAATACGTTCCCGGGTCTTGTACACA
CCGCCCGTCACACCATGGGAGTCGGGAATGCCCGAAGCCGGTGACCCAACCTTAAGGAGG
GAGCCGTCGAAGGCAGGCCTGATAACTGGGGTGAAGTCGT
HMI_45 Ruminococcus albus 16S rDNA sequence
(SEQ ID NO: 44)
CTGATCTAGTGGCGGACGGGTGAGTAACACGTGAGCAATCTGCCTTTCAGAGGGGGATAC
CGATTGGAAACGATCGTTAATACCGCATAACATAATTGAACCGCATGATTTGATTATCAA
AGATTTATCGCTGAAAGATGAGCTCGCGTCTGATTAGCTAGTTGGTAAGGTAACGGCTTA
CCAAGGCGACGATCAGTAGCCGGACTGAGAGGTTGATCGGCCACATTGGGACTGAGACAC
GGCCCAGACTCCTACGGGAGGCAGCAGTGGGGAATATTGCACAATGGAGGAAACTCTGAT
GCAGCGATGCCGCGTGAGGGAAGAAGGTTTTAGGATTGTAAACCTCTGTCTTCAGGGACG
AAAAAAGACGGTACCTGAGGAGGAAGCTCCGGCTAACTACGTGCCAGCAGCCGCGGTAAT
ACGTAGGGAGCGAGCGTTGTCCGGAATTACTGGGTGTAAAGGGAGCGTAGGCGGGATCGC
AAGTCAGATGTGAAAACTATGGGCTTAACCCATAAACTGCATTTGAAACTGTGGTTCTTG
AGTGAAGTAGAGGTAAGCGGAATTCCTAGTGTAGCGGTGAAATGCGTAGATATTAGGAGG
AACATCAGTGGCGAAGGCGGCTTACTGGGCTTTAACTGACGCTGAGGCTCGAAAGCGTGG
GGAGCAAACAGGATTAGATACCCTGGTAGTCCACGCCGTAAACGATGATTACTAGGTGTG
GGGGGACTGACCCCTTCCGTGCCGCAGCAAACGCAATAAGTAATCCACCTGGGGAGTACG
ACCGCAAGGTTGAAACTCAAAGGAATTGACGGGGGCCCGCACAAGCAGTGGAGTATGTGG
ATTAATTCGAAGCAACGCGAAGAACCTTACCAGGTCTTGACATCGTATGCATAGCTCAGA
GATGAGTGAAATCTCTTCGGAGACATATAGACAGGTGGTGCATGGTTGTCGTCAGCTCGT
GTCGTGAGATGTTGGGTTAAGTCCCGCAACGAGCGCAACCCTTACTGTTAGTTGCTACGC
AAGAGCACTCTAGCAGGACTGCCGTTGACAAAACGGAGGAAGGTGGGGATGACGTCAAAT
CATCATGCCCCTTATGACCTGGGCCTCACACGTACTACAATGGCTGTTAACAGAGGGATG
CAAAGCCGCGAGGTAGAGCGAACCCCTAAAAGCAGTCTTAGTTCGGATTGTAGGCTGCAA
CCCGCCTACATGAAGTCGGAATTGCTAGTAATCGCAGATCAGCATGCTGCGGTGAATACG
TTCCCGGGCCTTGTACACACCGCCCGTCACGCCATGGGAGTCGGTAACACCCGAAGCCTG
TAGTCTAACCGCAAGGAGGACGCAGTCGAAGGTGGGATTGATGACTGGGGTGAAGTCGTA
ACAGGGTAACCG
HMI_46 Blautia producta 16S rDNA sequence
(SEQ ID NO: 45)
TGGACAGATTCTTCGGATGAAGTCCTTAGTGACTGAGTGGCGGACGGGTGAGTAACGCGT
GGGTAACCTGCCTCATACAGGGGGATAACAGTTAGAAATGGCTGCTAATACCGCATAAGC
GCACGGTACTGCATGGTACAGTGTGAAAAACTCCGGTGGTATGAGATGGACCCGCGTTGG
ATTAGCTAGTTGGCAGGGTAACGGCCTACCAAGGCGACGATCCATAGCCGGCCTGAGAGG
GTGGACGGCCACATTGGGACTGAGACACGGCCCAGACTCCTACGGGAGGCAGCAGTGGGG
AATATTGCACAATGGGGGAAACCCTGATGCAGCGACGCCGCGTGAGCGAAGAAGTATTTC
GGTATGTAAAGCTCTATCAGCAGGGAAGAAAATGACGGTACCTGACTAAGAAGCCCCGGC
TAACTACGTGCCAGCAGCCGCGGTAATACGTAGGGGGCAAGCGTTATCCGGATTTACTGG
GTGTAAAGGGAGCGTAGACGGAATGGCAAGTCTGATGTGAAAGGCCGGGGCTCAACCCCG
GGACTGCATTGGAAACTGTCAATCTAGAGTACCGGAGGGGTAAGTGGAATTCCTAGTGTA
GCGGTGAAATGCGTAGATATTAGGAGGAACACCAGTGGCGAAGGCGGCTTACTGGACGGT
AACTGACGTTGAGGCTCGAAAGCGTGGGGAGCAAACAGGATTAGATACCCTGGTAGTCCA
CGCCGTAAACGATGAATACTAGGTGTTGGGGAGCAAAGCTCTTCGGTGCCGCAGCAAACG
CAATAAGTATTCCACCTGGGGAGTACGTTCGCAAGAATGAAACTCAAAGGAATTGACGGG
GACCCGCACAAGCGGTGGAGCATGTGGTTTAATTCGAAGCAACGCGAAGAACCTTACCAA
ATCTTGACATCGATCTGACCGGACTGTAATGAGTCCTTTCCCTTCGGGGACAGAGAAGAC
AGGTGGTGCATGGTTGTCGTCAGCTCGTGTCGTGAGATGTTGGGTTAAGTCCCGCAACGA
GCGCAACCCTTATCCTCAGTAGCCAGCAAGTGAAGTTGGGCACTCTGTGGAGACTGCCAG
GGATAACCTGGAGGAAGGTGGGGATGACGTCAAATCATCATGCCCCTTATGATTTGGGCT
ACACACGTGCTACAATGGCGTAAACAAAGGGAAGCGATCACGCGAGTGTGAGCAAATCTC
AAAAATAACGTCTCAGTTCGGATTGTAGTCTGCAACTCGACTACATGAAGCTGGAATCGC
TAGTAATCGCAGGTCAGCATACTGCGGTGAATACGTTCCCGGGTCTTGTACACACCGCCC
TCGAAGGTGGGACCGATAACTGGGGTGAAGTCGT
HMI_47 Clostridium nexile 16S rDNA sequence
(SEQ ID NO: 46)
GTTTGTGACTTAGTGGCGGACGGGTGAGTAACGCGTGGGTAACCTGCCTTATACAGGGGG
ATAACAGTTAGAAATGACTGCTAATACCGCATAAGCGCACAGTCTCGCATGGGACAGTGT
GAAAAACTAAGGTGGTATAAGATGGACCCGCGTCTGATTAGCTAGTTGGTGGGGTAAAGG
CCTACCAAGGCGACGATCAGTAGCCGACCTGAGAGGGTGATCGGCCACATTGGGACTGAG
ACACGGCCCAAACTCCTACGGGAGGCAGCAGTGGGGAATATTGCACAATGGGGGAAACCC
TGATGCAGCAACGCCGCGTGAAGGAAGAAGTATCTCGGTATGTAAACTTCTATCAGCAGG
GAAGAAAATGACGGTACCTGACTAAGAAGCTCCGGCTAAATACGTGCCAGCAGCCGCGGT
AATACGTATGGAGCAAGCGTTATCCGGATTTACTGGGTGTAAAGGGAGCGTAGGCGGTTA
TGCAAGTCAGATGTGAAAGCCCGGGGCTTAACCCCGGGACTGCATTTGAAACTGTGTAAC
TAGAGTGTCGGAGAGGTAAGTGGAATTCCTAGTGTAGCGGTGAAATGCGTAGATATTAGG
AGGAACACCAGTGGCGAAGGCGGCTTACTGGACGATAACTGACGCTGAGGCTCGAAAGCG
TGGGGAGCAAACAGGATTAGATACCCTGGTAGTCCACGCCGTAAACGATGAATACTAGGT
GTTGGGGAGCAAAGCTCTTCGGTGCCGCAGCAAACGCAATAAGTATTCCACCTGGGGAGT
ACGTTCGCAAGAATGAAACTCAAAGGAATTGACGGGGACCCGCACAAGCGGTGGAGCATG
TGGTTTAATTCGAAGCAACGCGAAGAACCTTACCAAGTCTTGACATCTGGATGACCGGAC
CGTAATGGGTCCTTTCCTTCGGGACATCCAAGACAGGTGGTGCATGGTTGTCGTCAGCTC
GTGTCGTGAGATGTTGGGTTAAGTCCCGCAACGAGCGCAACCCTTATCCTTAGTAGCCAG
CAGTAAGATGGGCACTCTAGGGAGACTGCCGGAGACAATCCGGAGGAAGGTGGGGATGAC
GTCAAATCATCATGCCCCTTATGACTTGGGCTACACACGTGCTACAATGGCGTAAACAAA
GGGAAGCGAGACCGCGAGGTTAAGCAAATCTCAAAAATAACGTCTCAGTTCGGATTGTAG
TCTGCAACTCGACTACATGAAGCTGGAATCGCTAGTAATCGCGAATCAGCATGTCGCGGT
GAATACGTTCCCGGGTCTTGTACACACCGCCCGTCACACCATGGGAGTCGATAACGCCCG
AAGCCGGTGACTCAACCGAAAGGAGAGAGCCGTCGAAGGCGGGATGGATAACTGGGGTGA
AGTCGTAAC
HMI_48 Butyricicoccus pullicaecorum 16S rDNA sequence
(SEQ ID NO: 47)
ATCTCTTCGGAGATGGAATTCTTAACCTAGTGGCGGACGGGTGAGTAACGCGTGAGCAAT
CTGCCTTTAGGAGGGGGATAACAGTCGGAAACGGCTGCTAATACCGCATAATACGTTTGG
GAGGCATCTCTTGAACGTCAAAGATTTTATCGCCTTTAGATGAGCTCGCGTCTGATTAGC
TGGTTGGCGGGGTAACGGCCCACCAAGGCGACGATCAGTAGCCGGACTGAGAGGTTGAAC
GGCCACATTGGGACTGAGACACGGCCCAGACTCCTACGGGAGGCAGCAGTGGGGAATATT
GCGCAATGGGGGAAACCCTGACGCAGCAACGCCGCGTGATTGAAGAAGGCCTTCGGGTTG
TAAAGATCTTTAATCAGGGACGAAAAATGACGGTACCTGAAGAATAAGCTCCGGCTAACT
ACGTGCCAGCAGCCGCGGTAATACGTAGGGAGCAAGCGTTATCCGGATTTACTGGGTGTA
AAGGGCGCGCAGGCGGGCCGGCAAGTTGGGAGTGAAATCCCGGGGCTTAACCCCGGAACT
GCTTTCAAAACTGCTGGTCTTGAGTGATGGAGAGGCAGGCGGAATTCCGTGTGTAGCGGT
GAAATGCGTAGATATACGGAGGAACACCAGTGGCGAAGGCGGCCTGCTGGACATTAACTG
ACGCTGAGGCGCGAAAGCGTGGGGAGCAAACAGGATTAGATACCCTGGTAGTCCACGCCG
TAAACGATGGATACTAGGTGTGGGAGGTATTGACCCCTTCCGTGCCGCAGTTAACACAAT
AAGTATCCCACCTGGGGAGTACGGCCGCAAGGTTGAAACTCAAAGGAATTGACGGGGGCC
CGCACAAGCAGTGGAGTATGTGGTTTAATTCGAAGCAACGCGAAGAACCTTACCAGGTCT
TGACATCCCGATGACCGGCGTAGAGATACGCCCTCTCTTCGGAGCATCGGTGACAGGTGG
TGCATGGTTGTCGTCAGCTCGTGTCGTGAGATGTTGGGTTAAGTCCCGCAACGAGCGCAA
CCCTTACGGTTAGTTGATACGCAAGATCACTCTAGCCGGACTGCCGTTGACAAAACGGAG
GAAGGTGGGGACGACGTCAAATCATCATGCCCCTTATGACCTGGGCTACACACGTACTAC
AATGGCAGTCATACAGAGGGAAGCAATACCGCGAGGTGGAGCAAATCCCTAAAAGCTGTC
CCAGTTCAGATTGCAGGCTGCAACCCGCCTGCATGAAGTCGGAATTGCTAGTAATCGCGG
ATCAGCATGCCGCGGTGAATACGTTCCCGGGCCTTGTACACACCGCCCGTCACACCATGA
GAGCCGTCAATACCCGAAGTCCGTAGCCTAACCGCAAGGGGGGCGCGGCCGAAGGTAGGG
GTGGTAATTAGGGTGAAGTCGTAC
HMI_49 Ruminococcus flavefaciens 16S rDNA sequence
(SEQ ID NO: 48)
AGTCGACGGACGAGGAGGAGCTTGCTTCTCCGAGTTAGTGGCGGACGGGTGAGTAACACG
TGAGCAACCTACCCTTGAGAGGGGGATAGCTTCTGGAAACGGATGGTAATACCCCATAAC
ATATATTTTAGGCATCTAAGATATATCAAAGAAATTCGCTCAAGGATGGGCTCGCGTCTG
ATTAGATAGTTGGTGAGGTAACGGCCCACCAAGTCGACGATCAGTAGCCGGACTGAGAGG
TTGAACGGCCACATTGGGACTGAGACACGGCCCAGACTCCTACGGGAGGCAGCAGTGGGG
AATATTGCACAATGGGGGGAACCCTGATGCAGCGATGCCGCGTGGAGGAAGAAGGTTTTC
GGATTGTAAACTCCTTTTAACAGGGACGATAATGACGGTACCTGAAGAAAAAGCTCCGGC
TAACTACGTGCCAGCAGCCGCGGTAATACGTAGGGAGCGAGCGTTGTCCGGAATTACTGG
GTGTAAAGGGAGCGTAGGCGGGACGGTAAGTCAGGTGTGAAATATACGTGCTCAACATGT
AGACTGCACTTGAAACTGCTGTTCTTGAGTGAAGTAGAGGTAAGCGGAATTCCTAGTGTA
GCGGTGAAATGCGTAGATATTAGGAGGAACATCGGTGGCGAAGGCGGCTTACTGGGCTTT
TACTGACGCTGAGGCTCGAAAGCGTGGGGAGCAAACAGGATTAGATACCCTGGTAGTCCA
CGCTGTAAACGATGATTACTAGGTGTGGGGGGACTGACCCCTTCCGTGCCGCAGTTAACA
CAATAAGTAATCCACCTGGGGAGTACGACCGCAAGGTTGAAACTCAAAGGAATTGACGGG
GGCCCGCACAAGCAGTGGAGTATGTGGTTTAATTCGAAGCAACGCGAAGAACCTTACCAG
GTCTTGACATCGTATGCATAGTCTAGAGATAGATGAAATCCCTTCGGGGACATATAGACA
GGTGGTGCATGGTTGTCGTCAGCTCGTGTCGTGAGATGTTGGGTTAAGTCCCGCAACGAG
CGCAACCCTTACCTTTAGTTGCTACGCAAGAGCACTCTAGAGGGACTGCCGTTGACAAAA
CGGAGGAAGGTGGGGATGACGTCAAATCATCATGCCCCTTATGACCTGGGCTACACACGT
ACTACAATGGCAATTAACAGAGGGAAGCAAAACAGCGATGTGGAGCAAATCCCGAAAAAT
TGTCCCAGTTCAGATTGCAGGCTGCAACTCGCCTGCATGAAGTCGGAATTGCTAGTAATC
GCAGATCAGAATGCTGCGGTGAATACGTTCCCGGGCCTTGTACACACCGCCCGTCACACC
ATGGGAGTCGGTAACACCCGAAGCCTGTAGTCTAACCTTATAGGAGGACGCAGTCGAAGG
TGGGATTGATGACTGGGGTGAAGTCGT
HMI_50 Clostridium orbiscindens 16S rDNA sequence
(SEQ ID NO: 49)
AAAGGGAATGCTTAGTGGCGGACGGGTGAGTAACGCGTGAGTAACCTGCCTTGGAGTGGG
GAATAACAGCCGGAAACGGCTGCTAATACCGCATGATGTATCTGGATCGCATGGTTCTGG
ATACCAAAGATTTATCGCTCTGAGATGGACTCGCGTCTGATTAGCTAGTTGGTGAGGTAA
CGGCTCACCAAGGCGACGATCAGTAGCCGGACTGAGAGGTTGGCCGGCCACATTGGGACT
GAGACACGGCCCAGACTCCTACGGGAGGCAGCAGTGGGGAATATTGGGCAATGGGCGAAA
GCCTGACCCAGCAACGCCGCGTGAAGGAAGAAGGCCCTCGGGTTGTAAACTTCTTTTGTC
AGGGACGAAGCAAGTGACGGTACCTGACGAATAAGCCACGGCTAACTACGTGCCAGCAGC
CGCGGTAATACGTAGGTGGCAAGCGTTATCCGGATTTACTGGGTGTAAAGGGCGTGTAGG
CGGGAGTGCAAGTCAGATGTGAAAACTATGGGCTCAACCCATAGCCTGCATTTGAAACTG
TACTTCTTGAGTGATGGAGAGGCAGGCGGAATTCCCTGTGTAGCGGTGAAATGCGTAGAT
ATAGGGAGGAACACCAGTGGCGAAGGCGGCCTGCTGGACATTAACTGACGCTGAGGCGCG
AAAGCGTGGGGAGCAAACAGGATTAGATACCCTGGTAGTCCACGCCGTAAACGATGGATA
CTAGGTGTGGGGGGTCTGACCCCCTCCGTGCCGCAGTTAACACAATAAGTATCCCACCTG
GGGAGTACGATCGCAAGGTTGAAACTCAAAGGAATTGACGGGGGCCCGCACAAGCGGTGG
AGTATGTGGTTTAATTCGAAGCAACGCGAAGAACCTTACCAGGACTTGACATCCTACTAA
CGAAGCAGAGATGCATAAGGTGCCCTTCGGGGAAAGTAGAGACAGGTGGTGCATGGTTGT
CGTCAGCTCGTGTCGTGAGATGTTGGGTTAAGTCCCGCAACGAGCGCAACCCTTATTGTT
AGTTGCTACGCAAGAGCACTCTAGCGAGACTGCCGTTGACAAAACGGAGGAAGGTGGGGA
CGACGTCAAATCATCATGCCCCTTATGTCCTGGGCCACACACGTACTACAATGGCGGTCA
ACAGAGGGAAGCAAAGCCGCGAGGTGGAGCAAATCCCTAAAAGCCGTCCCAGTTCGGATT
GCAGGCTGAAACTCGCCTGTATGAAGTCGGAATCGCTAGTAATCGCGGATCAGCATGCCG
CGGTGAATACGTTCCCGGGCCTTGTACACACCGCCCGTCACACCATGAGAGTCGGGAACA
CCCGAAGTCCGTAGCCTAACAGCAATGGG
HMI_51 Ruminococcus_bromii 16S rDNA sequence
(SEQ ID NO: 50)
ACGAAGCTTTGAGGAGCTTGCTTTTTAAGCTTAGTGGCGGACGGGTGAGTAACGCGTGAG
CAACCTGCCTCTCAGAGGGGAATAACGTTTTGAAAAGAACGCTAATACCGCATAACATAT
CGGAACCGCATGATTCTGATATCAAAGGAGCAATCCGCTGAGAGATGGGCTCGCGTCCGA
TTAGTTAGTTGGTGAGGTAACGGCTCACCAAGACTACGATCGGTAGCCGGACTGAGAGGT
TGATCGGCCACATTGGGACTGAGACACGGCCCAGACTCCTACGGGAGGCAGCAGTGGGGG
ATATTGCGCAATGGGGGAAACCCTGACGCAGCAACGCCGCGTGAAGGAAGAAGGTCTTCG
GATTGTAAACTTCTTTTGTCAGGGACGAAGAAAGTGACGGTACCTGACGAATAAGCTCCG
GCTAACTACGTGCCAGCAGCCGCGGTAATACGTAGGGAGCGAGCGTTGTCCGGATTTACT
GGGTGTAAAGGGTGCGTAGGCGGCCGAGCAAGTCAGTTGTGAAAACTATGGGCTTAACCC
ATAACGTGCAATTGAAACTGTCCGGCTTGAGTGAAGTAGAGGTAGGCGGAATTCCCGGTG
TAGCGGTGAAATGCGTAGAGATCGGGAGGAACACCAGTGGCGAAGGCGGCCTACTGGGCT
TTAACTGACGCTGAGGCACGAAAGCATGGGTAGCAAACAGGATTAGATACCCTGGTAGTC
CATGCCGTAAACGATGATTACTAGGTGTGGGGGGACTGACCCCTTCCGTGCCGCAGTTAA
CACAATAAGTAATCCACCTGGGGAGTACGGCCGCAAGGTTGAAACTCAAAGGAATTGACG
GGGGCCCGCACAAGCAGTGGAGTATGTGGTTTAATTCGAAGCAACGCGAAGAACCTTACC
AGGTCTTGACATCCTGAGAATCCTTAAGAGATTAGGGAGTGCCTTCGGGAACTCAGAGAC
AGGTGGTGCATGGTTGTCGTCAGCTCGTGTCGTGAGATGTTGGGTTAAGTCCCGCAACGA
GCGCAACCCTTGCTATTAGTTGCTACGCAAGAGCACTCTAATAGGACTGCCGTTGACAAA
ACGGAGGAAGGTGGGGACGACGTCAAATCATCATGCCCCTTATGACCTGGGCTACACACG
TACTACAATGGCCATTAACAGAGGGAAGCAAAACCGCGAGGCAGAGCAAACCCCTAAAAA
TGGTCCCAGTTCGGATTGTAGGCTGCAACCCGCCTACATGAAGTTGGAATTGCTAGTAAT
CGCGGATCAGCATGCCGCGGTGAATACGTTCCCGGGCCTTGTACACACCGCCCGTCACAC
CATGGGAGCCGGTAATACCCGAAGTCAGTAGTCTAACAGCAATGAGGACGCTGCCGAAGG
TAGGATTGGCGACTGGGGTGAAGTCGTAACAAGGTAACCG
HMI_52 Ruminococcus albus 926R 16S rDNA sequence
(SEQ ID NO: 51)
AGAGTTTGATCCTGGCTCAGGACGAACGCTGGCGGCACGCTTAACACATGCAAGTCGAAC
GAGAGAAGAGAAGCTTGCTTTTCTGATCTAGTGGCGGACGGGTGAGTAACACGTGAGCAA
TCTGCCTTTCAGAGGGGGATACCGATTGGAAACGATCGTTAATACCGCATAACATAATTG
AACCGCATGATTTGATTATCAAAGATTTATCGCTGAAAGATGAGCTCGCGTCTGATTAGC
TAGTTGGTAAGGTAACGGCTTACCAAGGCGACGATCAGTAGCCGGACTGAGAGGTTGATC
GGCCACATTGGGACTGAGACACGGCCCAGACTCCTACGGGAGGCAGCAGTGGGGAATATT
GCACAATGGAGGAAACTCTGATGCAGCGATGCCGCGTGAGGGAAGAAGGTTTTAGGATTG
TAAACCTCTGTCTTCAGGGACGAAAAAAAAGACGGTACCTGAGGAGGAAGCTCCGGCTAA
CTACGTGCCAGCAGCCGCGGTAATACGTAGGGAGCGAGCGTTGTCCGGAATTACTGGGTG
TAAAGGGAGCGTAGGCGGGATCGCAAGTCAGATGTGAAAACTATGGGCTTAACCCATAAA
CTGCATTTGAAACTGTGGTTCTTGAGTGAAGTAGAGGTAAGCGGAATTCCTAGTGTAGCG
GTGAAATGCGTAGATATTAGGAGGAACATCAGTGGCGAAGGCGGCTTACTGGGCTTTAAC
TGACGCTGAGGCTCGAAAGCGTGGGGAGCAAACAGGATTAGATACCCTGGTAGTCCACGC
CGTAAACGATGATTACTAGGTGTGGGGGGACTGACCCCTTCCGTGCCGCAGCAAACGCAA
TAAGTAATCCACCTGGGGAGTACGACCGCAAGGTTGAAACTCAAAGGAATTGACGGGGGC
CCGCACAAGCAGTGGAGTATGTGGATTAATTCGAAGCAACGCGAAGAACCTTACCAGGTC
TTGACATCGTATGCATAGCTCAGAGATGAGTGAAATCTCTTCGGAGACATATAGACAGGT
GGTGCATGGTTGTCGTCAGCTCGTGTCGTGAGATGTTGGGTTAAGTCCCGCAACGAGCGC
AACCCTTACTGTTAGTTGCTACGCAAGAGCACTCTAGCAGGACTGCCGTTGACAAAACGG
AGGAAGGTGGGGATGACGTCAAATCATCATGCCCCTTATGACCTGGGCCTCACACGTACT
ACAATGGCTGTCAACAGAGGGATGCAAAGCCGCGAGGTGGAGCGAACCCCTAAAAGCAGT
CTTAGTTCGGATTGTAGGCTGCAACCCGCCTACATGAAGTCGGAATTGCTAGTAATCGCA
GATCAGCATGCTGCGGTGAATACGTTCCCGGGCCTTGTACACACCGCCCGTCACGCCATG
GGAGTCGGTAACACCCGAAGCCTGTAGTCTAACCGCAAGGAGGACGCAGTCGAAGGTGGG
ATTGATGACTGGGGTGAAGTCGTAACAAGGTAGCCGTATCGGAAGGTGCG

### REFERENCES

All documents mentioned in this specification are incorporated herein by reference in their entirety.
Abecasis, A. B., M. Serrano, R. Alves, L. Quintais, J. B. Pereira-Leal and A. O. Henriques (2013). “A genomic signature and the identification of new sporulation genes.” J Bacteriol 195(9): 2101-2115.
Abujamel, T et al. (2013). “Defining the Vulnerable Period for Re-Establishment of Clostridium difficile Colonization after Treatment of C. difficile Infection with Oral Vancomycin or Metronidazole.” PLoS ONE 8(10): e76269.
Altschul, S. F., W. Gish, W. Miller, E. W. Myers and D. J. Lipman (1990). “Basic local alignment search tool.” Journal of Molecular Biology 215(3): 403-410.
Altschul, S. F., W. Gish, W. Miller, E. W. Myers and D. J. Lipman (1990). “Basic local alignment search tool.” J Mol Biol 215(3): 403-410.
Angriman, I., M. Scarpa and I. Castagliuolo (2014). “Relationship between pouch microbiota and pouchitis following restorative proctocolectomy for ulcerative colitis.” World J Gastroenterol 20(29): 9665-9674.
Atarashi, K., T. Tanoue, K. Oshima, W. Suda, Y. Nagano, H. Nishikawa, S. Fukuda, T. Saito, S. Narushima, K. Hase, S. Kim, J. V. Fritz, P. Wilmes, S. Ueha, K. Matsushima, H. Ohno, B.
Olle, S. Sakaguchi, T. Taniguchi, H. Morita, M. Hattori and K. Honda (2013). “T_{reg} induction by a rationally selected mixture of Clostridia strains from the human microbiota”. Nature 500(7461): 232-236.
Blanton, L. V., M. R. Charbonneau, T. Salih, M. J. Barratt, S. Venkatesh, O. Ilkaveya, S. Subramanian, M. J. Manary, I. Trehan, J. M. Jorgensen, Y. M. Fan, B. Henrissat, S. A. Leyn, D. A. Rodionov, A. L. Osterman, K. M. Maleta, C. B. Newgard, P. Ashorn, K. G. Dewey and J. I. Gordon (2016). “Gut bacteria that prevent growth impairments transmitted by microbiota from malnourished children.” Science 351(6275).
Bajaj, J. S. (2014). “The role of microbiota in hepatic encephalopathy.” Gut Microbes 5(3): 397-403.
Bajaj, J. S., P. B. Hylemon and Z. Younossi (2012). “The intestinal microbiota and liver disease.” Am J Gastroenterol. Suppl. 1:9-14
Bosshard, P. P., S. Abels, R. Zbinden, E. C. Bottger and M. Altwegg (2003). “Ribosomal DNA sequencing for identification of aerobic gram-positive rods in the clinical laboratory (an 18-month evaluation).” J Clin Microbiol 41(9): 4134-4140.
Britton, R. A. and V. B. Young (2014). “Role of the intestinal microbiota in resistance to colonization by Clostridium difficile.” Gastroenterology 146(6): 1547-1553.
Buffie, C. G., et al. (2015). “Precision microbiome reconstitution restores bile acid mediated resistance to Clostridium difficile.” Nature 517(7533): 205-208.
Chakraborty, A., S. Ghosh, G. Chowdhary, U. Maulik and S. Chakrabarti (2012). “DBETH: a Database of Bacterial Exotoxins for Human.” Nucleic Acids Res 40(Database issue): D615-620.
Clarridge, J. E., 3rd (2004). “Impact of 16S rRNA gene sequence analysis for identification of bacteria on clinical microbiology and infectious diseases.” Clin Microbiol Rev 17(4): 840-862, table of contents.
Cole, J. R., et al. (2014). “Ribosomal Database Project: data and tools for high throughput rRNA analysis.” Nucleic Acids Research 42(D1): D633-D642.
Collins, S. M. (2014). “A role for the gut microbiota in IBS.” Nat Rev Gastroenterol Hepatol 11(8): 497-505.
Comely, O. A., M. A. Miller, T. J. Louie, D. W. Crook and S. L. Gorbach (2012). “Treatment of First Recurrence of Clostridium difficile Infection: Fidaxomicin Versus Vancomycin.” Clinical Infectious Diseases: An Official Publication of the Infectious Diseases Society of America 55(Suppl 2): S154-S161.
Dominguez-Bello, M. G., E. K. Costello, M. Contreras, M. Magris, G. Hidalgo, N. Fierer and R. Knight (2010). “Delivery mode shapes the acquisition and structure of the initial microbiota across multiple body habitats in newborns.” Proc Natl Acad Sci USA 107(26): 11971-11975.
Duncan, S. H., G. L. Hold, H. J. Harmsen, C. S. Stewart and H. J. Flint (2002). “Growth requirements and fermentation products of Fusobacterium prausnitzii, and a proposal to reclassify it as Faecalibacterium prausnitzii gen. nov., comb. nov.” Int J Syst Evol Microbiol 52(Pt 6): 2141-2146.
Eckburg, P. B., E. M. Bik, C. N. Bernstein, E. Purdom, L. Dethlefsen, M. Sargent, S. R. Gill, K. E. Nelson and D. A. Relman (2005). “Diversity of the human intestinal microbial flora.” Science 308(5728): 1635-1638.
Fodor, A. A., T. Z. DeSantis, K. M. Wylie, J. H. Badger, Y. Ye, T. Hepburn, P. Hu, E. Sodergren, K. Liolios, H. Huot-Creasy, B. W. Birren and A. M. Earl (2012). “The “most wanted” taxa from the human microbiome for whole genome sequencing.” PLoS One 7(7): e41294.
Forster, S. C., H. P. Browne, N. Kumar, M. Hunt, H. Denise, A. Mitchell, R. D. Finn and T. D. Lawley (2015). “HPMCD: the database of human microbial communities from metagenomic datasets and microbial reference genomes.” Nucleic Acids Res.
Francis, M. B., C. A. Allen, R. Shrestha and J. A. Sorg (2013). “Bile acid recognition by the Clostridium difficile germinant receptor, CspC, is important for establishing infection.” PLoS Pathog 9(5): e1003356.
Galperin, M. Y., S. L. Mekhedov, P. Puigbo, S. Smirnov, Y. I. Wolf and D. J. Rigden (2012). “Genomic determinants of sporulation in Bacilli and Clostridia: towards the minimal set of sporulation-specific genes.” Environ Microbiol 14(11): 2870-2890.
Goodman, A. L., G. Kallstrom, J. J. Faith, A. Reyes, A. Moore, G. Dantas and J. I. Gordon (2011). “Extensive personal human gut microbiota culture collections characterized and manipulated in gnotobiotic mice.” Proc Natl Acad Sci USA 108(15): 6252-6257.
Hattori, M. and T. D. Taylor (2009). “The human intestinal microbiome: a new frontier of human biology.” DNA Res 16(1): 1-12.
Hold, G. L., M. Smith, C. Grange, E. R. Watt, E. M. EI-Omar and I. Mukhopadhya (2014). “Role of the gut microbiota in inflammatory bowel disease pathogenesis: what have we learnt in the past 10 years?” World J Gastroenterol 20(5): 1192-1210.
Hooper, L. V., D. R. Littman and A. J. Macpherson (2012). “Interactions between the microbiota and the immune system.” Science 336(6086): 1268-1273.
Hooper, L. V., M. H. Wong, A. Thelin, L. Hansson, P. G. Falk and J. I. Gordon (2001). “Molecular analysis of commensal host-microbial relationships in the intestine.” Science 291(5505): 881-884.
Huse, S. M., L. Dethlefsen, J. A. Huber, D. M. Welch, D. A. Relman and M. L. Sogin (2008). “Exploring Microbial Diversity and Taxonomy Using SSU rRNA Hypervariable Tag Sequencing.” PLoS Genet 4(11): el000255.
Janoir, C., C. Deneve, S. Bouttier, F. Barbut, S. Hoys, L. Caleechum, D. Chapeton-Montes, F. C. Pereira, A. O. Henriques, A. Collignon, M. Monot and B. Dupuy (2013). “Adaptive strategies and pathogenesis of Clostridium difficile from in vivo transcriptomics.” Infect Immun 81(10): 3757-3769.
Johnson, M., I. Zaretskaya, Y. Raytselis, Y. Merezhuk, S. McGinnis and T. L. Madden (2008). “NCBI BLAST: a better web interface.” Nucleic Acids Research 36(Web Server issue): W5-W9.
Jostins, L., et al. (2012). “Host-microbe interactions have shaped the genetic architecture of inflammatory bowel disease.” Nature 491(7422): 119-124.
Koenig, J. E., et al. (2011). “Succession of microbial consortia in the developing infant gut microbiome.” Proc Natl Acad Sci USA 108 Suppl 1: 4578-4585.
Kozich, J. J., S. L. Westcott, N. T. Baxter, S. K. Highlander and P. D. Schloss (2013). “Development of a dual-index sequencing strategy and curation pipeline for analyzing amplicon sequence data on the MiSeq Illumina sequencing platform.” Appl Environ Microbiol 79(17): 5112-5120.
Lagier, J. C., P. Hugon, S. Khelaifia, P. E. Fournier, B. La Scola and D. Raoult (2015). “The Rebirth of Culture in Microbiology through the Example of Culturomics To Study Human Gut Microbiota.” Clin Microbiol Rev 28(1): 237-264.
Landy, J., H. O. Al-Hassi, S. D. McLaughlin, A. W. Walker, P. J. Ciclitira, R. J. Nicholls, S. K. Clark and A. L. Hart (2011). “Review article: faecal transplantation therapy for gastrointestinal disease.” Alimentary Pharmacology & Therapeutics 34(4): 409-415.
Lawley, T. D., et al. (2009). “Antibiotic treatment of Clostridium difficile carrier mice triggers a supershedder state, spore-mediated transmission, and severe disease in immunocompromised hosts.” Infect Immun 77(9): 3661-3669.
Lawley, T. D., et al. (2012). “Targeted restoration of the intestinal microbiota with a simple, defined bacteriotherapy resolves relapsing Clostridium difficile disease in mice.” PLoS Pathog 8(10): e1002995.
Lawley, T. D. and A. W. Walker (2013). “Intestinal colonization resistance.” Immunology 138(1): 1-11.
Letunic, I. and P. Bork (2011). “Interactive Tree Of Life v2: online annotation and display of phylogenetic trees made easy.” Nucleic Acids Res 39(Web Server issue): W475-478.
Louis, P and H. J. Flint (2009). “Diversity, metabolism and microbial ecology of butyrate-producing bacteria from the human large intestine.” FEMS Microbiol. Lett. 294(1): 1-8.
Lozupone, C. and R. Knight (2005). “UniFrac: a New Phylogenetic Method for Comparing Microbial Communities.” Applied and Environmental Microbiology 71(12): 8228-8235.
Ludwig, W., et al. (2004). “ARB: a software environment for sequence data.” Nucleic Acids Res 32(4): 1363-1371.
McArthur, A. G. et al. (2013). “The comprehensive antibiotic resistance database.” Antimicrob Agents Chemother 57(7): 3348-3357.
Meehan, C. J. and R. G. Beiko (2014). “A phylogenomic view of ecological specialization in the Lachnospiraceae, a family of digestive tract-associated bacteria.” Genome Biol Evol 6(3): 703-713.
Nielsen, H. B., et al. (2014). “Identification and assembly of genomes and genetic elements in complex metagenomic samples without using reference genomes.” Nat Biotechnol 32(8): 822-828.
Ottman, N., H. Smidt, W. M. de Vos and C. Belzer (2012). “The function of our microbiota: who is out there and what do they do?” Front Cell Infect Microbiol 2: 104.
Perez Martinez, G., C. Bauerl and M. C. Collado (2014). “Understanding gut microbiota in elderly's health will enable intervention through probiotics.” Benef Microbes 5(3): 235-246. Petrof, E. O., G. B. Gloor, S. J. Vanner, S. J. Weese, D. Carter, M. C. Daigneault, E. M.
Brown, K. Schroeter and E. Allen-Vercoe (2013). “Stool substitute transplant therapy for the eradication of Clostridium difficile infection: ‘RePOOPulating’ the gut.” Microbiome 1(1): 3.
Price, M. N., P. S. Dehal and A. P. Arkin (2010). “FastTree 2—approximately maximum-likelihood trees for large alignments.” PLoS One 5(3): e9490.
Pruesse, E., C. Quast, K. Knittel, B. M. Fuchs, W. Ludwig, J. Peplies and F. O. Glöckner (2007). “SILVA: a comprehensive online resource for quality checked and aligned ribosomal RNA sequence data compatible with ARB.” Nucleic acids research 35(21): 7188-7196.
Qin, J., et al. (2010). “A human gut microbial gene catalogue established by metagenomic sequencing.” Nature 464(7285): 59-65.
Quince, C., A. Lanzen, R. Davenport and P. Turnbaugh (2011). “Removing Noise From Pyrosequenced Amplicons.” BMC Bioinformatics 12(1): 38.
Rajilic-Stojanovic, M. and W. M. de Vos (2014). “The first 1000 cultured species of the human gastrointestinal microbiota.” FEMS Microbiol Rev 38(5): 996-1047.
Riley, T. V., J. S. Brazier, H. Hassan, K. Williams and K. D. Phillips (1987). “Comparison of alcohol shock enrichment and selective enrichment for the isolation of Clostridium difficile.” Epidemiol Infect 99(2): 355-359.
Scheperjans, F., et al. (2015). “Gut microbiota are related to Parkinson's disease and clinical phenotype.” Mov Disord 30(3): 350-358.
Schloss, P. D., D. Gevers and S. L. Westcott (2011). “Reducing the Effects of PCR Amplification and Sequencing Artifacts on 16S rRNA-Based Studies.” PLoS ONE 6(12): e27310.
Schloss, P. D., et al. (2009). “Introducing mothur: Open-Source, Platform-Independent, Community-Supported Software for Describing and Comparing Microbial Communities.” Applied and Environmental Microbiology 75(23): 7537-7541.
Schloss, P. D., et al. (2009). “Introducing mothur: open-source, platform-independent, community-supported software for describing and comparing microbial communities.” Appl Environ Microbiol 75(23): 7537-7541.
Sekirov, I., S. L. Russell, L. C. Antunes and B. B. Finlay (2010). “Gut microbiota in health and disease.” Physiol Rev 90(3): 859-904.
Snyder, A (2014). “Genetic basis for clinical response to CTLA-4 blockade in melanoma”. N Engl J Med. 371(23):2189-99.
Stewart, E. J. (2012). “Growing unculturable bacteria.” J Bacteriol 194(16): 4151-4160. van Nood, E., A. Vrieze, M. Nieuwdorp, S. Fuentes, E. G. Zoetendal, W. M. de Vos, C. E. Visser, E. J. Kuijper, J. F. Bartelsman, J. G. Tijssen, P. Speelman, M. G. Dijkgraaf and J. J. Keller (2013). “Duodenal infusion of donor feces for recurrent Clostridium difficile.” N Engl J Med 368(5): 407-415.
Wang, Q., G. M. Garrity, J. M. Tiedje and J. R. Cole (2007). “Naive Bayesian classifier for rapid assignment of rRNA sequences into the new bacterial taxonomy.” Appl Environ Microbiol 73(16): 5261-5267.
Xu et al. (2015). “Fecal microbiota transplantation broadening its application beyond intestinal disorders”. World J Gastroenterol. 21(1): 102-111.
Ze, X., F. Le Mougen, S. H. Duncan, P. Louis and H. J. Flint (2013). “Some are more equal than others: the role of “keystone” species in the degradation of recalcitrant substrates.” Gut Microbes 4(3): 236-240.

1. A therapeutic composition comprising at least one isolated bacterium from the family Ruminococcaceae and a pharmaceutically acceptable excipient, wherein the bacterium from the family Ruminococcaceae comprises a gene encoding a 16S ribosomal RNA (rRNA) and said gene comprises a sequence with at least 90% sequence identity with the sequence set forth in any one of SEQ ID NOs 1, 2, 3, 4, 5, 6, 29, 30, 35, 37, 44, 47, 48, 49, 50, or 51.

2-52. (canceled)

53. The therapeutic composition according to claim 1, wherein the sequence identity is at least 91%, 92%, 93%, 94%, 95%, 96%, 97%, 98%, 98.7%, 99%, or 100%.

54-65. (canceled)

66. The therapeutic composition according to claim 1, wherein the isolated bacterium is a bacterium as deposited at the Leibniz-Institut DSMZ under accession number DSM32191, DSM32147, DSM32149, DSM32175, DSM32153, DSM32152, DSM32184, DSM32181, DSM32222, DSM32212, DSM32221, DSM32214, DSM32263, DSM32223, DSM32225, or DSM32265.

67. The therapeutic composition according to claim 1, wherein the composition comprises at least two distinct isolated bacteria, and wherein the bacteria are as defined in claim 1.

68. (canceled)

69. A method of treating a dysbiosis of the gastrointestinal tract in an individual, the method comprising administering a therapeutically effective amount of a therapeutic composition according to claim 1 to an individual in need thereof.

70. The method according to claim 69, wherein the dysbiosis is a dysbiosis associated with an enteric bacterial infection.

71. The method according to claim 70, wherein the enteric bacterial infection is an infection with a pathogenic bacterium.

72. The method according to claim 71, wherein the pathogenic bacterium is resistant to treatment with one or more antibiotics.

73. The method, according to claim 72, wherein the enteric bacterial infection is an infection with a pathogenic bacterium of the genus Clostridium, Escherichia, Enterococcus, Klebsiella, Enterobacter, Proteus, Salmonella, Shigella, Staphylococcus, Vibrio, Aeromonas, Campylobacter, Bacillus, Helicobacter, Listeria, Plesiomonas, or Yersinia.

74. The method according to claim 73, wherein the enteric bacterial infection is an infection with a pathogenic bacterium, and wherein the pathogenic bacterium is Clostridium difficile, or Escherichia coli.

75-76. (canceled)

77. The method according to claim 69, wherein the dysbiosis is a dysbiosis associated with inflammatory bowel disease (IBD) or pouchitis or hepatic encephalopathy.

78. The method according to claim 77, wherein the IBD is ulcerative colitis (UC) or Crohn's disease.

79. The method according to claim 69, wherein the dysbiosis is a dysbiosis associated with irritable bowel syndrome (IBS), a metabolic disease, a neuropsychiatric disorder, an autoimmune disease, an allergic disorder, or a cancer.

80. (canceled)

81. The method according to claim 79, wherein the metabolic disease is selected from the group consisting of: metabolic syndrome, obesity, type 2 diabetes mellitus, a cardiovascular disease, and non-alcoholic fatty liver, wherein the neuropsychiatric disorder is selected from the group consisting of: Parkinson's disease, Alzheimer's disease, multiple sclerosis, myoclonus dystonia, autism and chronic fatigue syndrome, wherein the neuropsychiatric disorder is selected from the group consisting of: Parkinson's disease, Alzheimer's disease, multiple sclerosis, myoclonus dystonia, autism and chronic fatigue syndrome, wherein the autoimmune disease is selected from the group consisting of: idiopathic thrombocytopenic purpura, arthritis, Sjógren's syndrome, systemic lupus erythematosus, and Hashimoto's thyroiditis, wherein the allergic disorder is selected from the group consisting of: atopy, and asthma, and wherein the cancer is selected from the group consisting of: colorectal cancer, extra-intestinal tumours, mammary tumours, hepatocellular carcinoma, lymphoma, melanoma, and lung cancer.

82-86. (canceled)

87. The therapeutic composition according to claim 1, wherein the isolated bacterium is a spore-forming bacterium.

88. The therapeutic composition according to claim 1, wherein the isolated bacterium is susceptible to treatment with one or more antibiotics.

89. The therapeutic composition according to claim 88, wherein the antibiotic is a beta-lactam, fusidic acid, elfamycin, aminoglycoside, fosfomycin, and/or tunicamycin.

90. The therapeutic composition according to claim 1, wherein at least one of bacteria in the composition is antagonistic towards a pathogenic intestinal bacterium, inhibits or prevents the growth of a pathogenic intestinal bacterium, or neutralizes or protects against a toxin produced by an intestinal bacterium.

91. The therapeutic composition according to claim 90, wherein the pathogenic bacterium is a pathogenic bacterium of the genus Clostridium, Escherichia, Enterococcus, Klebsiella, Enterobacter, Proteus, Salmonella, Shigella, Staphylococcus, Vibrio, Aeromonas, Campylobacter, Bacillus, Helicobacter, Listeria, Plesiomonas, or Yersinia.

92. The therapeutic composition according to claim 91, wherein the pathogenic bacterium is Clostridium difficile or Escherichia coli.

93. The therapeutic composition according to claim 1, wherein at least one of bacteria in the composition has immunomodulatory activity.

94. The therapeutic composition according to claim 1, wherein the composition further comprises a prebiotic.

95. The therapeutic composition according to claim 1, wherein the composition further comprises a carrier.

96-101. (canceled)

102. The therapeutic composition according to claim 1, wherein the composition is for oral administration and is lyophilised.

103. The therapeutic composition according to claim 102, wherein the composition further comprises a stabiliser and/or a cryoprotectant.

104. The therapeutic composition according to claim 1, wherein the composition is the form of a capsule, a tablet, or an enema.

105. The therapeutic composition according to claim 104, wherein the capsule or tablet is enteric-coated, pH dependent, slow-release, and/or gastro-resistant.

106. A method of preparing a therapeutic composition according to claim 1, wherein the method comprises the steps of:
(i) culturing an isolated bacterium as set out in claim 1; and
(ii) mixing the bacteria obtained in (i) with a pharmaceutically acceptable excipient.

107-108. (canceled)

109. A therapeutic composition comprising at least one isolated bacterium from the family Lachnospiraceae and a pharmaceutically acceptable excipient, wherein the bacterium from the family Lachnospiraceae comprises a gene encoding a 16S ribosomal RNA (rRNA) and said gene comprises a sequence with at least 90% sequence identity with the sequence set forth in any one of SEQ ID Nos 7, 8, 9, 10, 11, 12, 13, 14, 15, 16, 17, 18, 25, 26, 27, 28, 32, 33, 34, 36, 38, 40, 43, 45, or 46.

110. A method of treating a dysbiosis of the gastrointestinal tract in an individual, the method comprising administering a therapeutically effective amount of a therapeutic composition according to claim 109 to an individual in need thereof.


SEQUENCE LISTING (57 sequences)

SEQ ID NO: 1 - DNA, 1467 bp, Clostridium thermocellum
  source 1..1467: mol_type = genomic DNA
caggacgaacgctggcggcgtgcctaacacatgcaagtcgaacgagaatctttgaacagatcttttcggagtgacgttcaaagaggaaagtggcggacgggcgagtaacgcgtgagtaacctgcccataagagggggataatccatggaaacgtggactaataccgcatattgtagtcaagtcgcatgactagattatgaaagatttatcgcttatggatggactcgcgtcagattagatagttggtgaggtaacggctcaccaagtcaacgatctgtagccgaactgagaggttgatcggccgcattgggactgagacacggcccagactcctacgggaggcagcagtggggaatattgcgcaatgggggcaaccctgacgcagcaacgccgcgtgcaggaagaaggtcttcggattgtaaactgttgtcgcaagggaagaagacagtgacggtaccttgtgagaaagtcacggctaactacgtgccagcagccgcggtaatacgtaggtgacaagcgttgtccggatttactgggtgtaaagggcgcgtaggcggactgtcaagtcagtcgtgaaataccggggcttaaccccggggctgcgattgaaactgacagccttgagtatcggagaggaaagcggaattcctagtgtagcggtgaaatgcgtagatattaggaggaacaccagtggcgaaggcggctttctggacgacaactgacgctgaggcgcgaaagtgtggggagcaaacaggattagataccctggtagtccacaccgtaaacgatggatactaggtgtaggaggtatcgaccccttctgtgccgcagttaacacaataagtatcccacctggggagtacgaccgcaaggttgaaactcaaaggaattgacgggggcccgcacaagcagtggagtatgtggtttaattcgaagcaacgcgaagaaccttacctgggcttgacatccctggaatcgagtagagatacttgagtgccttcgggaatcaggtgacaggtggtgcatggttgtcgtcagctcgtgtcgtgagatgttgggttaagtcccgcaacgagcgcaacccctattgtcagttgccatcattaagttgggcactctggcgagactgccggtgacaaatcggaggaaggtggggacgacgtcaaatcatcatgccccttatgcccagggctacacacgtactacaatggccgataacaaagtgcagcgaaaccgtgaggtggagcgaatcacaaaactcggtctcagttcagattgcaggctgcaactcgcctgcatgaagttggaattgctagtaatcgcggatcagaatgccgcggtgaatacgttcccgggccttgtacacaccgcccgtcacaccatgagagtcgataacacccgaagcctgtgagctaacctattaggaggcagcagtcgaaggtggggttgatgattggggtgaagtcg

SEQ ID NO: 2 - DNA, 1419 bp, Flavonifractor pautii
  source 1..1419: mol_type = genomic DNA
gagtgctcatgacagaggattcgtccaatggagtgagttacttagtggcggacgggtgagtaacgcgtgagtaacctgccttggagtggggaataacaggtggaaacatctgctaataccgcatgatgcagttgggtcgcatggctctgactgccaaagatttatcgctctgagatggactcgcgtctgattagctggttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaagcaagtgacggtacctgaggaataagccacggctaactacgtgccagcagccgcggtaatacgtaggtggcgagcgttatccggatttactgggtgtaaagggcgtgtaggcgggactgcaagtcagatgtgaaaaccatgggctcaacctgtggcctgcatttgaaactgtagttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccggtgaccggtgtagagatacaccttcttcttcggaagcgccggtgacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggtcaacagagggaagcaagaccgcgaggtggagcaaacccctaaaagccatcccagttcggattgcaggctgcaactcgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgt

SEQ ID NO: 3 - DNA, 1398 bp, Flavonifractor plautii
  source 1..1398: mol_type = genomic DNA
tggctgtttagtggcggacgggtgagtaacgcgtgagtaacctgccttggagtggggaataacacagtgaaaactgtgctaataccgcatgacatattggtgtcgcatggcactgatatcaaagatttatcgctctgagatggactcgcgtctgattagatagttggcggggtaacggcccaccaagtcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttaacagggacgaagtaagtgacggtacctgttgaataagccacggctaactacgtgccagcagccgcggtaatacgtaggtggcaagcgttatccggatttactgggtgtaaagggcgtgtaggcgggactgcaagtcagatgtgaaaactatgggctcaacccatagcctgcatttgaaactgtagttcttgagtgtcggagaggcaatcggaattccgtgtgtagcggtgaaatgcgtagatatacggaggaacaccagtggcgaaggcggattgctggacgataactgacgctgaggcgcgaaagcgtggggagcaaacaggattagataccctggtagtccacgccgtaaacgatggatactaggtgtggggggtctgaccccctccgtgccgcagctaacgcaataagtatcccacctggggagtacgatcgcaaggttgaaactcaaaggaattgacgggggcccgcacaagcggtggagtatgtggtttaattcgaagcaacgcgaagaaccttaccagggcttgacatcctactaacgaaccagagatggattaggtgcccttcggggaaagtagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggcggttaacagagggaggcaaagccgcgaggcagagcaaacccctaaaagccgtcccagttcggattgcaggctgaaacccgcctgtatgaagtcggaatcgctagtaatcgcggatcagcatgccgcggtgaatacgttcccgggccttgtacacaccgcccgtcacaccatgagagtcgggaacacccgaagtccgtagcctaactgcaaagggggcgcggccgaaggtgggttcgataattggggtgaagtcgtaacagggtaaccg

SEQ ID NO: 4 - DNA, 1386 bp, Clostridium orbiscindens
  source 1..1386: mol_type = genomic DNA
tggcggacgggtgagtaacgcgtgaggaacctgcctcggagtggggaataacagaccgaaaggcctgctaataccgcatgatgcagttggaccgcatggtcctgactgccaaagatttatcgctctgagatggcctcgcgtctgattagcttgttggcggggtaatggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaacaaatgacggtacctgaggaataagccacggctaactacgtgccagcagccgcggtaatacgtaggtggcaagcgttatccggatttactgggtgtaaagggcgtgtaggcgggaaggcaagtcagatgtgaaaactatgggctcaacccatagcctgcatttgaaactgtttttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcctactaacgaagcagagatgcattaggtgcccttcggggaaagtagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggcggggacgacgtcaaatcatcatgccccttatgtcctgggctacacacgtactacaatggtggtaaacagagggaagcaagaccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagtctaaccgcaagggggacgcggccgaaggtgggttcgataattggggtgaagtcgtaacagggtaacc

SEQ ID NO: 5 - DNA, 1324 bp, Ruminococcus flavefaciens
  source 1..1324: mol_type = genomic DNA
cggatcagtggcggacgggtgagtaacacgtgagcaacctgcctttaagagggggataacgtttggaaacgaacgctaataccgcataacatagaagattcacatgtttcttctatcaaagatttatcgcttaaagatgggctcgcgtctgattagctagttggtgaggtaacggctcaccaaggcgacgatcagtagccgtactgagaggtagaacggccacattgggactgagacacggcccagactcctacgggaggcagcagtggggaatattgcacaatggaggaaactctgatgcagcgatgccgcgtgagggaagaaggttttcggattgtaaacctctgtcttcagggacgataatgacggtacctgaggaggaagctccggctaactacgtgccagcagccgcggtaatacgtagggagcgagcgttgtccggaattactgggtgtaaagggagcgtaggcgggatcttaagtcaggtgtgaaaactatgggctcaacccatagactgcacttgaaactgaggttcttgagtgaagtagaggcaggcggaattcctagtgtagcggtgaaatgcgtagatattaggaggaacatcagtggcgaaggcggcctgctgggcttttactgacgctgaggctcgaaagcgtggggagcaaacaggattagataccctggtagtccacgctgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcacgcgaagaaccttaccgggtcttgacatctacagaatcctttagagataagggagtgcccttcggggaactgtaagacaggtggtgcatggttgtcgtcagctcgtgtcgtgagatgttgggttaagtcccgcaacgagcgcaacccctatcattagttgctacgcaagagcactctaatgagactgccgttgacaaaacggaggaaggtggggatgacgtcaaatcatcatgccccttatgacccgggctacacacgtactacaatggcgtaacagagggaagcaacatcgcgaggtgaagcaaatctctaaaaaacgtcccagttcagattgcaggctgcaactcgcctgcatgaagacggaattgctagtaatcgcagatcagcatgctgcggtgaatacgttcccgggccttgtacacaccgcccgtcacaccatgggagtcggtaacacccgaagtcgcttgtctaa

SEQ ID NO: 6 - DNA, 1426 bp, Anaerotruncus colihominis
  source 1..1426: mol_type = genomic DNA
agtcgacggacacatccgacggaatagcttgctaggaagatggatgttgttagtggcggacgggtgagtaacacgtgagcaacctacctcagagtgggggacaacagttggaaacgactgctaataccgcataagatggcagggtcgcatggcctggtcataaaaggagcaattcgctctgagatgggctcgcgtctgattagctagttggtgaggtaacggctcaccaaggcaacgatcagtagccggactgagaggttgaacggccacattgggactgagacacggcccagactcctacgggaggcagcagtggggaatattgcacaatgggggaaaccctgatgcagcgacgccgcgtgagggaagacggttttcggattgtaaacctctgtcttgtgggacgatagtgacggtaccacaggaggaagccatggctaactacgtgccagcagccgcggtaatacgtagatggcgagcgttgtccggaattactgggtgtaaagggagtgtaggcgggctggtaagttgaatgtgaaaccttcgggctcaacccggagcgtgcgttcaaaactgctggtcttgagtgaagtagaggcaggcggaattcccggtgtagcggtggaatgcgtagatatcgggaggaacaccagtggcgaaggcggcctgctgggcttttactgacgctgaggctcgaaagcatgggtagcaaacaggattagataccctggtagtccatgccgtaaacgatgattactaggtgtggggggattgaccccctccgtgccggagttaacacaataagtaatccacctggggagtacgaccgcaaggttgaaactcaaaggaattgacgggggcccgcacaagcagtggagtatgtggtttaattcgaagcaacgcgaaaaaccttaccaggtcttgacatccatcgccaggctaagagattagctgttccctccggggacgatgagacaggtggtgcatggttgtcgtcagctcgtgtcgtgagatgttgggttaagtcccgcaacgagcgcaacccttactattagttgctacgcaagagcactctaatgggactgccgttgacaaaacggaggaaggtggggatgacgtcaaatcatcatgccccttatgacctgggctacacacgtactacaatggccgttaacagagagcagcgataccgcgaggtggagcgaatctagaaaaacggtctcagttcggattgcaggctgaaactcgcctgcatgaagtcggaattgctagtaatcgcggatcagcatgccgcggtgaatacgttcccgggccttgtacacaccgcccgtcacaccatgagagccggtaacacccgaagtcagtagcctaaccgcaaggagggcgctgccgaaggtggggctggtaattggggtgaagtcgtaac

SEQ ID NO: 7 - DNA, 1373 bp, Clostridium xylanolyticum
  source 1..1373: mol_type = genomic DNA
gtaacgcgtgggtaacctgcctcatacagggggataacagttagaaatgactgctaataccgcataagcgcacagggtcgcatgacctagtgtgaaaaactccggtggtatgagatggacccgcgtctgattagctagttggtgaggtaacggctcaccaaggcgacgatcagtagccgacctgagagggtgatcggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtaggcggtctgacaagtcagaagtgaaagcccggggctcaactccgggactgcttttgaaactgccggactagattgcaggagaggtaagtggaattcctagtgtagcggtgaaatgcgtagatattaggaggaacaccagtggcgaaggcggcttactggactgtaaatgacgctgaggctcgaaagcgtggggagcaaacaggattagataccctggtagtccacgccgtaaacgatgaatactaggtgttggggagcacagctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccgatgaccgtcccgtaacgggggcttctcttcggagcatcggtgacaggtggtgcatggttgtcgtcagctcgtgtcgtgagatgttgggttaagtcccgcaacgagcgcaacccttatctttagtagccagcggtacggccgggcactctagagagactgccagggataacctggaggaaggtggggatgacgtcaaatcatcatgccccttatgatttgggctacacacgtgctacaatggcgtaaacaaagggaagcgaaactgtgaagtctagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaacgcccgaagtcagtgacccaaccgtaaggagggagctgccgaaggcgggtctgataactggggtgaagtcgtaacaaggtaaccg

SEQ ID NO: 8 - DNA, 1416 bp, Clostridium oroticum
  source 1..1416: mol_type = genomic DNA
ttttgattgatttcttcggaaagagagagactgtgactgagtggcggacgggtgagtaacgcgtgggtaacctgcctcatacagggggataacagttagaaatgactgctaataccgcataagcacacagcttcgcatgaagcagtgtgaaaaactccggtggtatgagatggacccgcgtctgattaggtagttggtggggtaacggcccaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggtaatgcaagtctggagtgaaaacccggggctcaaccccgggactgctttggaaactgtgtaactagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcggtaagcaaagcttatcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctggtcttgacatccctctgacagctgagtaatgtcggttttctttcgggacagaggagacaggtggtgcatggttgtcgtcagctcgtgtcgtgagatgttgggttaagtcccgcaacgagcgcaacccctatcttcagtagccagcatatgagatgggcactctggagagactgccagggataacctggaggaaggtggggatgacgtcaaatcatcatgccccttatgatcagggctacacacgtgctacaatggcgtaaacaaagggaagcgagcctgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaacctttcaggagggagctgccgaaggcgggaccgataactggggtgaagtcgt

SEQ ID NO: 9 - DNA, 1427 bp, Eubacterium contortum
  source 1..1427: mol_type = genomic DNA
cttaagtttgattcttcggatgaagacttttgtgactgagtggcggacgggtgagtaacgcgtgggtaacctgcctcatacaggtggataacagttagaaatgactgctaataccgcataagaccacagcaccgcatggtgcaggggtaaaaactccggtggtatgagatggacccgcgtctgattagctagttggtggggtaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggtatggcaagtctgatgtgaaaggccagggctcaaccctgggactgcattggaaactgtcgaactagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcgggtagcagagctattcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatctccctgaccggcaagtaatgttgcctttccttcgggacagggatgacaggtggtgcatggttgtcgtcagctcgtgtcgtgagatgttgggttaagtcccgcaacgagcgcaacccctatctttagtagccagcggtttggccgggcactctagagagactgccagggataacctggaggaaggtggggatgacgtcaaatcatcatgccccttatgagcagggctacacacgtgctacaatggcgtaaacaaagggaagcgagcctgcgagggtaagcaaatctcaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagttggtaacgcccgaagtcagtgacccaaccgcaaggagggagctgccgaaggtgggaccgataactggggtgaagtcgtaacaaggtaaccg

SEQ ID NO: 10 - DNA, 1447 bp, Clostridium oroticum
  source 1..1447: mol_type = genomic DNA
acatgcaagtcgagcgagcgctttagtggaattctacggaaggaaagtgaagtgactgagcggcggacgggtgagtaacgcgtgggtaacctgcctcatacagggggataacagttagaaatgactgctaataccgcataagaccccagtaccgcatggtacagaggtaaaaactgaggtggtatgagatggacccgcgtctgattagctagttggtgaggtagaggctcaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggagcagcaagtctgatgtgaaaacccggggctcaaccccgggagtgcattggaaactgttgatctagagtgctggagaggtaagtggaattcctagtgtagcggtgaaatgcgtagatattaggaggaacaccagtggcgaaggcggcttactggacagtgactgacgttgaggctcgaaagcgtggggagcaaacaggattagataccctggtagtccacgccgtaaacgatgactactaggtgtcgggtagcaaagctattcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgcccttgacatccgggtgaccggcgagtaatgtcgccttctcttcggagcagccgagacaggtggtgcatggttgtcgtcagctcgtgtcgtgagatgttgggttaagtcccgcaacgagcgcaacccttatctttagtagccagcggataagccggggactctagagagactgccagggataacctggaggaaggtggggatgacgtcaaatcatcatgccccttatgggcagggctacacacgtgctacaatggcgtaaacaaagggaagcgaagctgtgaagcggagcgaatctcaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggacggataactggggtgaagtcgtaacaaggtaaccg

SEQ ID NO: 11 - DNA, 1379 bp, Lachnospira pectinoschiza
  source 1..1379: mol_type = genomic DNA
agtggcggacgggtgagtaacgcgtgggtaacctgccctgtacagggggacaacagctggaaacggctgctaataccgcataagcccttagcactgcatggtgcatagggaaaaggagcaatccggtacaggatggacccgcgtctgattagccagttggcagggtaacggcctaccaaagcgacgatcagtagccgatctgagaggatgtacggccacattgggactgagacacggcccagactcctacgggaggcagcagtggggaatattgcacaatggaggaaactctgatgcagcgacgccgcgtgagtgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtaggtggcaaggcaagccagaagtgaaaacccggggctcaaccgcgggattgcttttggaactgtcatgctagagtgcaggaggggtgagcggaattcctagtgtagcggtgaaatgcgtagatattaggaggaacaccggaggcgaaggcggctcactggactgtaactgacactgaggctcgaaagcgtggggagcaaacaggattagataccctggtagtccacgcggtaaacgatgaatactagatgtcgggtagcaaagctactcggtgtcgtcgcaaacgcaataagtattccacctggggagtacgttcgcaagaatgaaactcaaaggaattgacggggacccgcacaagcggtggagcatgtggtttaattcgaagcaacgcgaagaaccttacctgctcttgacatcccattcgatagagggtaatgcttctagcccttcgggggaatggagacaggtggtgcatggttgtcgtcagctcgtgtcgtgagatgttgggttaagtcccgcaacgagcgcaacccttattgtcagtagccagcaggtgaagctgggcactctgatgagactgccggggataacccggaggaaggtggggatgacgtcaaatcatcatgccccttatgagcagggctacacacgtgctacaatggcgtaaacagagggaagcgaaggagtgatctggagcaaatctcaaaaataacgtctcagttcggattgtagtctgcaactcgactacatgaagctggaatcgctagtaatcgcagatcaaaatgctgcggtgaatacgttcccgggtcttgtacacaccgcccgtcacaccatgggagtcggtaatgcccgaagtcagtgactcaaccgaaaggaaaaagctgccgaaggcaggactggtaactggggtgaagtcgt

SEQ ID NO: 12 - DNA, 1444 bp, Roseburia faecis
  source 1..1444: mol_type = genomic DNA
agtcgaacgaagcactttattacgatttcttcggaatgacgatttagtgactgagtggcggacgggtgagtaacgcgtgggtaacctgccttatacagggggataacagttagaaatgactgctaataccgcataagcgcacaggattgcatgatctggtgtgaaaaactccggtggtataagatggacccgcgtctgattagctggttggtgaggtaacggcccaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaaaaatgacggtacctgactaagaagccccggctaactacgtgccagcagccgcggtaatacgtagggggcaagcgttatccggatttactgggtgtaaagggagcgcaggcggtgcggcaagtctgatgtgaaagcccggggctcaaccccgggactgcattggaaactgtcgtacttgagtatcggagaggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgtcgggggacatagtccttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccggtgacaaagtatgtaatgtactctttcttcggaacaccggtgacaggtggtgcatggttgtcgtcagctcgtgtcgtgagatgttgggttaagtcccgcaacgagcgcaacccctgttcttagtagccagcggttcggccgggcactctagggagactgccagggataacctggaggaaggcggggatgacgtcaaatcatcatgccccttatgacttgggctacacacgtgctacaatggcgtaaacaaagggaagcgaaaaggtgacttctagcaaatcccaaaaataacgtcccagttcggactgtagtctgcaactcgactacacgaagctggaatcgctagtaatcgcgaatcagaatgtcgcggtgaatacgttcccgggtcttgtacacaccgcccgtcacaccatgggagtcgggaatgcccgaagccggtgactcaaccgaaaggagagagccgtcgaaggcaggtctgataactggggtgaagtcgtaacaaggtaacc

SEQ ID NO: 13 - DNA, 1452 bp, Clostridium hathewayi
  source 1..1452: mol_type = genomic DNA
agtcgacggagatgcgatgtgagcgagaggtgcttgcactgatcaatcttttcgtatcttagtggcggacgggtgagtaacgcgtgggtaacctgccttataccgggggataacacttagaaataggtgctaataccgcataagcgcacggtgtcgcatgacacagtgtgaaaaactccggtggtataagatggacccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaagaaatgacggtacctgactaagaagccccggctaactacgtgccagcagccgcggtaatacgtagggggcaagcgttatccggatttactgggtgtaaagggagcgtagacggtgaagcaagtctgaagtgaaaggttggggctcaaccccgaaactgctttggaaactgtttaactggagtacaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgattactaggtgttggtggatatggatccatcggtgccgcagcaaacgcaataagtaatccacctggggagtacgttcgcaagaatgaaactcaaaggaattgacggggacccgcacaagcggtggagcatgtggtttaattcgaagcaacgcgaagaaccttacctgatcttgacatccctatgaatacagggtaatgcctgtagtacttcggtacataggagacaggtggtgcatggttgtcgtcagctcgtgtcgtgagatgttgggttaagtcccgcaacgagcgcaacccctatctttagtagccagcagtaagatgggcactctagagagactgccggggataacccggaggaaggtggggatgacgtcaaatcatcatgccccttatgaccagggctacacacgtgctacaatggcgtaaacagagggaagcgaagtggtgacatggagcaaatcccaaaaataacgtcccagttcggattgcaggctgcaactcgcctgcatgaagctggaatcgctagtaatcgcagatcagaatgctgcggtgaatacgttcccgggtcttgtacacaccgcccgtcacaccatgggagtaggtaatgcccgaagtcggtgacctaaccgcaaggaaggagccgccgaaggcaggacttataactggggtgaagtcgtaacaaggtaaccgt

SEQ ID NO: 14 - DNA, 1400 bp, Fusicatenibacter saccharivorans
  source 1..1400: mol_type = genomic DNA
cggatggaatcggtataacttagtggcggacgggtgagtaacgcgtgggaaacctgccctgtaccgggggataacacttagaaataggtgctaataccgcataagcgcacggaactgcatggttctgtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatcccaatgacgcacctgtaaagaggtgttcccttcggggcattggagacaggtggtgcatggttgtcgtcagctcgtgtcgtgagatgttgggttaagtcccgcaacgagcgcaacccttattcttagtagccagcaggtgaagctgggcactctaaggagactgccggggataacccggaggaaggcggggatgacgtcaaatcatcatgccccttatgatttgggctacacacgtgctacaatggcgtaaacaaagggaagcgagacagtgatgtggagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagtctgtgacctaaccgaaagggaggagcagccgaaggcaggtctgataactggggtgaagtcgta

SEQ ID NO: 15 - DNA, 1416 bp, Clostridium clostridioforme
  source 1..1416: mol_type = genomic DNA
ctgctttgatgaagttttcggatggatttaaaacagcttagtggcggacgggtgagtaacgcgtgggtaacctgcctcacactgggggataacagttagaaatagctgctaataccgcataagcgcacggttccgcatggaacagtgtgaaaaactccggtggtgtgagatggacccgcgtctgattagccagttggcggggtaacggcccaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaagtgacggtacctgaataagaagccccggctaactacgtgccagcagccgcggtaatacgtagggggcaagcgttatccggatttactgggtgtaaagggagcgtagacggcaaggcaagtctgaagtgaaagcccggtgcttaacgccgggactgctttggaaactgtttagctggagtgccggagaggtaagcggaattcctagtgtagcggtgaaatgcgtagatattaggaagaacaccagtggcgaaggcggcttactggacggtaactgacgttgaggctcgaaagcgtggggagcaaacaggattagataccctggtagtccacgccgtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaggtcttgacatcccgatgaaaaacccgtaacggggttccctcttcggagcatcggagacaggtggtgcatggttgtcgtcagctcgtgtcgtgagatgttgggttaagtcccgcaacgagcgcaacccttattcttagtagccagcaggtaaggctgggcactctaaggagactgccggggataacccggaggaaggtggggatgacgtcaaatcatcatgccccttatgatctgggctacacacgtgctacaatggcgtaacaaagggaagcgagcctgcgagggtgagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcggaaatgcccgaagtctgtgactcaaccgcaaggagagagcagccgaaggcaggtctgataactggggtgaagtcgt

SEQ ID NO: 16 - DNA, 1211 bp, Rumincoccus torques
  source 1..1211: mol_type = genomic DNA
cggtatgagatggacccgcgtctgattagctagttggtggggtaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaggcaagtctgatgtgaaaacccggggctcaaccccgtgactgcattggaaactgttttgcttgagtgccggagaggtaagcggaattcctagtgtagcggtgaaatgcgtagatattaggaggaacaccagtggcgaaggcggcttactggacggcaactgacgttgaggctcgaaagcgtggggagcaaacaggattagataccctggtagtccacgccgtaaacgatgaatactaggtgtcggggagcaaagctcttcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaagccttgacatcccattgacagagcatgtaatgtgctttcccttcggggcagtggtgacaggtggtgcatggttgtcgtcagctcgtgtcgtgagatgttgggttaagtcccgcaacgagcgcaacccctatcttcagtagccagcggtttggccgggcactctggagagactgccagggataacctggaggaaggtggggatgacgtcaaatcatcatgccccttatggcttgggctacacacgtgctacaatggcgtaaacaaagggaagcgagcctgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcggcaacgcccgaagccagtgacccaaccgaaag

SEQ ID NO: 17 - DNA, 1425 bp, Closstridium celerecrescens
  source 1..1425: mol_type = genomic DNA
agtcgacgaggtaatgagatgaagttttcggatggattcttatttccgagtggcggacgggtgagtaacgcgtgggtaacctgcctcatacagggggataacgattggaaacgattgctaataccgcataagcgcacagtaccacatggtacagtgtgaaaaactccggtggtatgagatggacccgcgtctgattagctagttggtgaggtaacggcccaccaaggcaacgatcagtagccgacctgagagggtgaccggccacattgggactgagacacggcccagactcctacgggaggcagcagtgggggatattgcacaatggaggaaactctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgatgcaagtctgaagtgaaatacccgggctcaacctgggaactgctttggaaactgtatggctagagtgctggagaggtaagcggaattcctagtgtagcggtgaaatgcgtagatattaggaagaacaccagtggcgaaggcggcttactggacagtaactgacgttcaggctcgaaagcgtggggagcaaacaggattagataccctggtagtccacgccgtaaacgatgaatactaggtgtcgggggacaaagtctttcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaaaagcctttaatcgagctcctccttcgggacagaggtgacaggtggtgcatggttgtcgtcagctcgtgtcgtgagatgttgggttaagtcccgcaacgagcgcaacccctattgtcagtagccagcaggtaaagctgggcactctgatgagactgccagggataacctggaggaaggtggggatgacgtcaaatcatcatgccccttatgatttgggctacacacgtgctacaatggcgtaaacaaagagaggcgaagctgtgaggcagagcaaatctcaaaaataacgtctcagttcggattgtagtctgcaactcgactacatgaagctggaatcgctagtaatcgcggatcagaatgccgcggtgaatacgttcccgggtcttgtacacaccgcccgtcacaccatgggagtcggaaatgcccgaagccagtgacccaagcgaaagcagggagctgtcgaaggcaggtctgataactggggtgaagtcgt

SEQ ID NO: 18 - DNA, 1437 bp, Clostridium celerescens
  source 1..1437: mol_type = genomic DNA
tcgacgaggtattttgattgaagttttcggatggatttcagataccgagtggcggacgggtgagtaacgcgtgggtaacctgcctcatacagggggataacggttagaaatgactgctaataccgcataagcgcacagtaccgcatggtacggtgtgaaaaactccggtggtatgagatggacccgcgtctgattagctagttggtggggtaacggcccaccaaggcgacgatcagtagccgacctgagagggtgaccggccacattgggactgagacacggcccagactcctacgggaggcagcagtgggggatattgcacaatggaggaaactctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctgaagtgaaatacccgggctcaacctgggaactgctttggaaactgtgttgctagagtgctggagaggtaagcggaattcctagtgtagcggtgaaatgcgtagatattaggaagaacaccagtggcgaaggcggcttactggacagtaactgacgttgaggctcgaaagcgtggggagcaaacaggattagataccctggtagtccacgccgtaaacgatgaatactaggtgttggtgagcaaagctcatcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatccctctgaaacgcccttaatcgggctcctccttcgggacagaggtgacaggtggtgcatggttgtcgtcagctcgtgtcgtgagatgttgggttaagtcccgcaacgagcgcaacccctattgtcagtagccagcaggtaaagctgggcactctgatgagactgccagggataacctggaggaaggtggggatgacgtcaaatcatcatgccccttatgatttgggctacacacgtgctacaatggcgtaaacaaagagaagcgagcctgcgagggggagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcggaaatgcccgaagccagtgacccaagcgaaagcagggagctgtcgaaggcaggtctgataactggggtgaagtcgtaacagggtaaccg

SEQ ID NO: 19 - DNA, 1401 bp, Eubacterium infirmum
  source 1..1401: mol_type = genomic DNA
gagctcatcacagatgcttcggttgaagtgatgagtggaaagcggcggacgggtgagtaacgcgtaggcaacctgccctttgcagagggatagcctcgggaaaccgggattaaaacctcatgacacctcttaaagacatctttgagaggtcaaagatttatcggcagaggatgggcctgcgtctgattagctagttggtggggtaacggcctaccaaggcgacgatcagtagccgacctgagagggtgatcggccacattggaactgagacacggtccaaactcctacgggaggcagcagtggggaatattgcacaatgggggaaaccctgatgcagcaacgccgcgtgaaggaagaaggcctttgggtcgtaaacttctgttctaagggaagataatgacggtaccttaggagcaagtcccggctaactacgtgccagcagccgcggtaatacgtagggggcaagcgttatccggaattattgggcgtaaagagtacgtaggtggttacctaagcacgaggtataaggcaatggcttaaccattgttcgccttgtgaactgggctacttgagtgcaggagaggaaagcggaattcctagtgtagcggtgaaatgcgtagatattaggaggaacaccagtggcgaaggcggctttctggactgtaactgacactgaggtacgaaagcgtggggagcaaacaggattagataccctggtagtccacgccgtaaacgatgagcactaggtgtcggggtcgcaagacttcggtgccgcagttaacgcaataagtgctccgcctggggagtacgttcgcaagaatgaaactcaaaggaattgacggggacccgcacaagcagcggagcatgtggtttaattcgaagcaacgcgaagaaccttaccaggacttgacatccctctgacagccttttaatcgaggttttctacggacagaggagacaggtggtgcatggttgtcgtcagctcgtgtcgtgagatgttgggttaagtcccgcaacgagcgcaacccttgtcattagttgccagcagtaagatgggcactctagtgagactgccggggataactcggaggaaggtggggatgacgtcaaatcatcatgccccttatgttctgggctacacacgtgctacaatggccggtacagagagaaagcgagactgcgaagtggagcgaaactcaaaagccggtcccagttcggattgcaggctgcaactcgcctgcatgaagtcggagttgctagtaatcgcagatcagaatgctgcggtgaatgcgttcccgggtcttgtacacaccgcccgtcacaccatggaagttgggggcgcccgaagttggcagataaatatgttacctaaggcgaaatcaatgactggggtgaagtcgt

SEQ ID NO: 20 - DNA, 1368 bp, Eubacterium infirmum
  source 1..1368: mol_type = genomic DNA
tcggtaaagggatatggcggaaagcggcggacgggtgagtaacgcgtaggcaacctgccccttacagagggatagccattggaaacgatgattaagacctcataacgcctccctcccacatgagggggaggccaaagattcatcggtaagggatgggcctgcgtctgattagcttgttggcggggtaacggcccaccaaggcgacgatcagtagccgacctgagagggtgatcggccacattggaactgagacacggtccaaactcctacgggaggcagcagtggggaatattgcacaatgggcggaagcctgatgcagcaacgccgcgtgagggatgaaggccttcgggtcgtaaacctctgtccttggggaagaaacaaatgacggtacccatggaggaagccccggctaactacgtgccagcagccgcggtaatacgtagggggcgagcgttatccggaattattgggcgtaaagagtgcgtaggtggttacctaagcgcagggtctaaggcaatggctcaaccattgttcgccctgcgaactgggctacttgagtgcaggagaggaaagcggaattcctagtgtagcggtgaaatgcgtagatattaggaggaacaccagtggcgaaggcggctttctggactgttactgacactgaggcacgaaagcgtggggagcaaacaggattagataccctggtagtccacgccgtaaacgatgagcactaggtgtcggggccgcaaggcttcggtgccgcagttaacgcattaagtgctccgcctggggagtacgcacgcaagtgtgaaactcaaaggaattgacggggacccgcacaagcagcggagcatgtggtttaattcgaagcaacgcgaagaaccttaccaggacttgacatccccctgacagatccttaaccggatccttcttcggacaggggagacaggtggtgcatggttgtcgtcagctcgtgtcgtgagatgttgggttaagtcccgcaacgagcgcaacccttgccattagttgccatcattcagttgggcactctaatgggactgccggggacaactcggaggaaggtggggatgacgtcaaatcatcatgccccttatgttctgggctacacacgtgctacaatggccggtacagcaggaagcgatcccgcgagggggagcaaatcccaaaaaccggtcccagttcggactgcaggctgcaacccgcctgcacgaagccggagttgctagtaatcgtggatcagaatgccacggtgaatgcgttcccgggtcttgtacacaccgcccgtcacaccatggaagttgggggtgcccgaagccggcagggagatatgctgtctaaggcaaaaccaat

SEQ ID NO: 21 - DNA, 1421 bp, Clostridium thermocellum
  source 1..1421: mol_type = genomic DNA
ggatgaggaaatgcttcggcatggagacatccgatctagtggcggacgggtgagtaacgcgtgagcaacctgtcctgcacagggggataacactgagaaatcagtgctaataccgcatgagaccacagtatcacatggtacaggggtcaaaggagaaatccggtgcagggtgggctcgcgtcccattagctagttggtagggtaaaggcctaccaaggcgacgatgggtagccggactgagaggttggccggccacactgggactgagacacggcccagactcctacgggaggcagcagtggggaatattgggcaatgggcgaaagcctgacccagcaacgccgcgtgaaggaagaaggtctttggattgtaaacttttgtcctatgggaagaaggaagtgacggtaccatgggaggaagccccggctaactacgtgccagcagccgcggtaatacgtagggggcgagcgttgtccggaattactgggcgtaaagggcgcgcaggcggccgatcaagttagatgtgaaatacccgggcttaacctgggaactgcatttaaaactggttggctaggagtgcaggagagggaagcggaattcctagtgtagcggtgaaatgcgtagatattaggaggaacaccagtggcgaaggcggctttctggactgtaactgacgctgaggcgcgaaagcgtggggagcgaacaggattagataccctggtagtccacgctgtaaacgatgaatactaggtgtagggggtatcgaccccccctgtgccggagcaaacgcaataagtattccgcctggggagtacggccgcaaggttgaaactcaaaggaattgacgggggcccgcacaagcagcggagcatgtggtttaattcgaagcaacgcgaagaaccttaccaggtcttgacatccctcgaagtgcatagagatatgtacgtccttcgggacgaggagacaggtggtgcatggttgtcgtcagctcgtgtcgtgagatgttgggttaagtcccgcaacgagcgcaacccctacagttagttaccagcgggtaaagccggggactctaacaggactgccgtggataacacggaggaaggtggggacgacgtcaaatcatcatgctccttatgacctgggctacacacgtgctacaatggccggtacaaagagaagcgagaccgtaaggtggagcggatctcaaaaaaccggtcccagttcggattgtgggctgcaacccgcccacatgaagttggagttgctagtaatcgcgaatcagcatgtcgcggtgaatgcgttcccgggccttgtacacaccgcccgtcacaccatgggagttgggagcgcccgaagtcgttgaggtaacccgcaagggagccaggcgccgaaggtgagaccgataactggggtgaagtcgt

SEQ ID NO: 22 - DNA, 1507 bp, Anaerovorax odorimutans
  source 1..1507: mol_type = genomic DNA
agagtttgatcctggctcaggatgaacgctggcggcgtgcctaacacatgcaagtcgagcgggaaatcttggaacgatacttcggtaaagggaagagatggatagcggcggacgggtgagtaacgcgtaggtaacctgcctcatgcagagggatagcctcgggaaactgggattaatacctcataatgcggaggagtcacatggctccatcgccaaagatttatcggcatgagatggacctgcgtctgattagttagttggtgaggtaacggctcaccaaggcagcgatcagtagccgacctgagagggtaatcggccacattggaactgagacacggtccaaactcctacgggaggcagcagtggggaatattgcacaatgggcgcaagcctgatgcagcaacgccgcgtgagcgatgaaggtcttcggatcgtaaagctctgtcctaggggaagaatatatgacggtacccttggaggaagccccggctaactacgtgccagcagccgcggtaatacgtagggggcgagcgttatccggaattattgggcgtaaagagttcgtaggtggttttgtaagcgcggggtttaaggcaacggctcaaccgttgttcgccttgcgaactgcaagacttgagtgcgggagaggaaagtggaattcctagtgtagcggtgaaatgcgtagatattaggaggaacaccagtggcgaaggcgactttctggaccgtaactgacactgaggaacgaaagcgtggggagcaaacaggattagataccctggtagtccacgccgtaaacgatgagcactaggtgtcggggccgcaaggtttcggtgccgcagttaacgcattaagtgctccgcctggggagtacgcacgcaagtgtgaaactcaaaggaattgacggggacccgcacaagcagcggagcatgtggtttaattcgaagcaacgcgaagaaccttaccagggcttgacatcccgatgaccggcgggtaacgccgccttctcttcggagcatcggtgacaggtggtgcatggttgtcgtcagctcgtgtcgtgagatgttgggttaagtcccgcaacgagcgcaacccttgtcattagttgccagcagttcggctgggcactctagtgagactgccggggacaactcggaggaaggtggggatgacgtcaaatcatcatgccccttatgttctgggctacacacgtgctacaatggccggtacagagagacgcaagactgtgaagtggagcaaaactctaaaaccggtcccagttcggattgtaggctgcaactcgcctacatgaagttggagttgctagtaatcgcgaatcagaatgtcgcggtgaatgcgttcccgggtcttgtacacaccgcccgtcacaccatggaagttgggggcgcccgaagttggtcaacaaatcgattacctaaggcgaaaccaatgactggggtgaagtcgtaacaaggtagccgtatcggaaggtgcggctggatcacct

SEQ ID NO: 23 - DNA, 1378 bp, Clostridium saccharogumia
  source 1..1378: mol_type = genomic DNA
agccaccggcttcgggtgttatcaactctcatggtgtgacgggcggtgtgtacaaggcccgagaacgtattcaccgcggcatgctgatccgcgattactagcgattccatcttcatgcaggcgagttgcagcctgcaatccgaactgagaacgggtttttgagtttcgctccaagtcgcctcttcgcttccctttgatccgtccattgtagcacgtgtgtagcccaggtcataaggggcatgatgatttgacgtcatccccgccttcctccggcttgtcaccggctgtctcgttagagtccccatcttactgctggtaactaacgacaagggttgcgctcgttgcgggacttaacccaacatctcacgacacgagctgacgacaaccatgcaccacctgtcttgagtatatctatccctctatctctagagtctttactctgatgtcaagacctggtaaggttcttcgcgttgcttcgaattaaaccacatgctccaccgcttgtgcgggcccccgtcaattcctttgagtttcattcttgcgaacgtactactcaggcggagtacttattgcgttaactgcagcactgaggcttgtccccccaacacttagtactcatcgtttacggcgtggactactagggtatctaatcctatttgctccccacgctttcgggactgagcgtcagttacagaccagatcgtcgccttcgccactggtgttcctccatatatctacgcatttcaccgctacacatggaattccacgatcctcttctgcactctagctatttggtttccatggcttactgaagttaagcttcagccttttaccacagacctccattgccgcctgctccctctttacgcccaataattccggataacgcttgccacctacgtattaccgcggctgctggcacgtagttagccgtggcttcctcacaaagtaccgtcactctaataccattccctgtattagtcgttcttcctttataacagaagtttacaacccgaaggccttcttccttcacgcggcgttgctcggtcagggttccccccattgccgaaaattccctactgctgcctcccgtaggagtctgggccgtgtctcagtcccagtgtggccgttcaccctctcaggccggctatgcatcgtcgccttggtaggccgttacccctccaactagctaatgcaccataagcccatctgttccctatcccttaggatatttaacttagagaaaatgcttcctctaagcctatgcggtgttagcgcatgtttccacgcgttatccccctggtacagccaggttgcttatgtcttactcacccgttcgccactcatcaccgaagtgatgcgttcgacttgcatgtat

SEQ ID NO: 24 - DNA, 1376 bp, Clostridium saccharogumia
  source 1..1376: mol_type = genomic DNA
ggcatctacagggggataactgatggaaacgtcagctaagaccgcataggtgtagagatcgcatgaactctatatgaaaagtgctacgggactggtagatgatggacttatggcgcattagcttgttggtagggtaacggcctaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaagtaattcgttatgtaaacttctgtcatagaggaagaacggtggatatagggaatgatatccaagtgacggtactctataagaaagccacggctaactacgtgccagcagccgcggtaatacgtaggtggcgagcgttatccggaattattgggcgtaaagagggagcaggcggcactaagggtctgtggtgaaagatcgaagcttaacttcggtaagccatggaaaccgtagagctagagtgtgtgagaggatcgtggaattccatgtgtagcggtgaaatgcgtagatatatggaggaacaccagtggcgaaggcgacgatctggcgcataactgacgctcagtcccgaaagcgtggggagcaaataggattagataccctagtagtccacgccgtaaacgatgagtactaagtgttgggtgtcaaagctcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatcgatctaaaggctccagagatggagagatagctatagagaagacaggtggtgcatggttgtcgtcagctcgtgtcgtgagatgttgggttaagtcccgcaacgagcgcaacccctgttgccagttgccagcattaagttggggactctggcgagactgccggtgacaagccggaggaaggcggggatgacgtcaaatcatcatgccccttatgacctgggctacacacgtgctacaatggacagagcagagggaagcgaagccgcgaggtggagcgaaacccataaaactgttctcagttcggactgcagtctgcaactcgactgcacgaagatggaatcgctagtaatcgcgaatcagcatgtcgcggtgaatacgttctcgggccttgtacacaccgcccgtcacaccatgagagtcggtaacacccgaagccggtggcctaaccgcaaggaaggagctgtctaaggtgggactgatgattggggtgaagtcgtaacaagggtaacc

SEQ ID NO: 25 - DNA, 1361 bp, Blautia luti
  source 1..1361: mol_type = genomic DNA
cgggaatactttattgaaacttcggtggatttaatttatttctagtggcggacgggtgagtaacgcgtgggtaacctgccttatactgggggataacagccagaaatgactgctaataccgcataagcgcacagaaccgcatggttccgtgtgaaaaactccggtggtataagatggacccgcgttggattagctagttggcagggcagcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcagggagcacagctctttggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggacttaaccgtccctttccttcgggacaggggagacaggtggtgcatggttgtcgtcagctcgtgtcgtgagatgttgggttaagtcccgcaacgagcgcaacccctatccttagtagccagcacgtaatggtgggcactctgaggagactgccagggataacctggaggaaggcggggatgacgtcaaatcatcatgccccttatgatttgggctacacacgtgctacaatggcgtaaacaaagggaagcgaacccgcgagggtgggcaaatctcaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcag

SEQ ID NO: 26 - DNA, 1383 bp, Clostridium clostridioforme
  source 1..1383: mol_type = genomic DNA
ttgcggtaggtcacaggcttcgggcatttccaactcccatggtgtgacgggcggtgtgtacaagacccgggaacgtattcaccgcgacatgctgattcgcgattactagcgattccagcttcatgtagtcgagttgcagactacaatccgaactgagacgttatttctgggatttgctcaacatcactgtctcgcttccctttgtttacgccattgtagcacgtgtgtagcccaaatcataaggggcatgatgatttgacgtcatccccgccttcctccgggttatccccggcagtctccctagagtgcccagctctacctgctggctactaaggataagggttgcgctcgttgcgggacttaacccaacatctcacgacacgagctgacgacaaccatgcaccacctgtctccaatgctccgaagagaatgccccgttacggacacgtcattgggatgtcaagacttggtaaggttcttcgcgttgcttcgaattaaaccacatgctccaccgcttgtgcgggtccccgtcaattcctttgagtttcattcttgcgaacgtactccccaggtggattgcttattgcgttagctgcggcaccgatgggtccatacccacctacacctagcaatcatcgtttaccgcgtggactaccagggtatctaatcctgtttgctccccacgctttcgagcctcaacgtcagttacagtccagtaagccgccttcgccactggtgttcctcctaatatctacgcatttcaccgctacactaggaattccgcttacctctcctgcactccagcctggcagttccaaatgcagtcccagggttgagccctgggttttcacatctggcttgtcatgccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcactcacgcggcgtcgctgcatcagggtttcccccattgtgcaatattccccactgctgcctcccgtaggagtttgggccgtgtctcagtcccaatgtggccgttcaccctctcaggccggctactgatcgtcgctttggtaggccgttaccctgccaactggctaatcagacgcgggaccatcctgtaccaccggagtttttcacactgcctcatgtgaagctgtgcgcttatgcggtattagcacctatttctaagtgttatcccccggtacagggcaggtttcccacgcgttactcacccgtccgccactaagttacgccgattccatccgaaaacttcctctgcataactccgtcgactg

SEQ ID NO: 27 - DNA, 1445 bp, Blautia producta
  source 1..1445: mol_type = genomic DNA
ccggtggtcgcatcggcgctcctcctgtaggttgggtcactgacttcgggcgttactgactcccatggtgtgacgggcggtgtgtacaagacccgggaacgtattcaccgcgacattctgattcgcgattactagcgattccagcttcgtgcagtcgagttgcagactgcagtccgaactgggacgttatttttgggatttgctcaacatcgctgtctcgcttccctttgtttacgccattgtagcacgtgtgtagcccaaatcataaggggcatgatgatttgacgtcgtccccgccttcctccgggttatccccggcagtctccctagagtgcccagcttcacctgctggctactaaggataggggttgcgctcgttgcgggacttaacccaacatctcacgacacgagctgacgacaaccatgcaccacctgtctcctctgccccgaagggaaggccccgttacgggccggtcagagggatgtcaagacttggtaaggttcttcgcgttgcttcgaattaaaccacatgctccaccgcttgtgcgggtccccgtcaattcctttgagtttcattcttgcgaacgtactccccaggtggaatacttattgcgtttgctgcggcaccgaatgggctttgccacccgacacctagtattcatcgtttacggcgtggactaccagggtatctaatcctgtttgctccccacgctttcgagcctcaacgtcagttaccgtccagaaagccgccttcgccactggtgttcctcctaatatctacgcatttcaccgctacactaggaattccgcttacctctccggcactctagaaaaacagtttccaatgcagtcctggggttaagccccagcctttcacatcagacttgctcttccgtctacgctccctttacacccagtaaatccggataacgcttgccccctacgtattaccgcggctgatggcacgtagttagccggggcttcttagtcaggtaccgtcattttcttccctgctgatagaagtttacataccgagatacttcttccttcacgcggcgtcgctgcatcagggtttcccccattgtgcaatattccccactgctgcctcccgtaggagtctgggccgtgtctcagtcccaatgtggccgttcaccctctcaggccggctactgatcgtcgccttggtgggccgttacccctccaactagctaatcagacgcgggtccatctcataccaccggagtttttcacaccagaccatgcggtcctgtgcgcttatgcggtattagcagccatttctaactgttatccccctgtatgaggcaggttacccacgcgttactcagcccgtccgccgctcagtcaaataagtttcaatccgaagagatccacttaagtgcttcgctcgacttgcatgtgttaagcacgccgccagcgttcatcct

SEQ ID NO: 28 - DNA, 1412 bp, Blautia glucerasea
  source 1..1412: mol_type = genomic DNA
gccttcggcagctccgtcctttcggttcggtcactgacttcgggcgttactgactcccatggtgtgacgggcggtgtgtacaagacccgggaacgtattcaccgcggcattctgatccgcgattactagcgattccagcttcgtgcagtcgagttgcagactgcagtccgaactgggacgttatttttgggatttgcttaagctcacactctcgcttccctttgtttacgccattgtagcacgtgtgtagcccaaatcataaggggcatgatgatttgacgtcatccccgccttcctccaggttatccctggcagtctcctcagagtgcccggccaaaccgctggctactaaggataggggttgcgctcgttgcgggacttaacccaacatctcacgacacgagctgacgacaaccatgcaccacctgtctccgatgctccgaagaaaaggcgacgttactcgccggtcatagggatgtcaagacttggtaaggttcttcgcgttgcttcgaattaaaccacatgctccaccgcttgtgcgggtccccgtcaattcctttgagtttcattcttgcgaacgtactccccaggtggaatacttactgcgtttgctgcggcaccgaatggctctgccacccgacacctagtattcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacctctccggtactcaagatcaacagtttccaatgcagtccgggggttgagcccccgcctttcacatcagacttgctgctccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattccccactgctgcctcccgtaggagtctgggccgtgtctcagtcccaatgtggccgtccaccctctcaggccggctatggatcgtcgctttggtaggccgttaccctgccaactggctaatccaacgcgggtccatctcacaccaccggagtttttcacactggatcatgcaatcccgtgcgcttatgcggtattagcagtcatttctgactgttatcccccagtgtgaggcaggttacccacgcgttactcacccgtccgccactaggattataacgacttcaaccgaagtctctgtcaaaataatccccgttcgacttgcatgtgt

SEQ ID NO: 29 - DNA, 1317 bp, Clostridium straminisolvens
  source 1..1317: mol_type = genomic DNA
agcggcggacgggtgagtaacgcgtgagtaacctgcctttaggagggggacaacattccgaaagggatgctaataccgcataaaattattgtatcgcatggtataataatcaaagatttatcgcctaaagatggactcgcgtccgattagctagttggtggggtaaaagcctaccaaggcgacgatcggtagccgaactgagaggttgatcggccacattgggactgagacacggcccagactcctacgggaggcagcagtgggggatattgcgcaatgggggaaaccctgacgcagcaacgccgcgtgaaggaagaaggccttcgggttgtaaacttctttaagtgtggaagataatgacggtacacacagaataagccacggctaactacgtgccagcagccgcggtaatacgtaggtggcaagcgttgtccggatttactgggtgtaaagggcgtgtaggcgggtagacaagtcagatgtgaaataccggggctcaactccggggctgcatttgaaactgtatatcttgagtgtcggagaggaaagcggaattcctagtgtagcggtgaaatgcgtagatattaggaggaacaccagtggcgaaggcggctttctggacgataactgacgctgaggcgcgaaagcgtggggagcaaacaggattagataccctggtagtccacgccgtaaacgatggatactaggtgtaggaggtatcgaccccttctgtgccgcagttaacacaataagtatcccacctggggagtacggtcgcaagattgaaactcaaaggaattgacgggggcccgcacaagcagtggagtatgtggtttaattcgaagcaacgcgaagaaccttaccaggacttgacatcccacgcatagcctagagataggtgaagtcctacgggacgtggagacaggtggtgcatggttgtcgtcagctcgtgtcgtgagatgttgggttaagtcccgcaacgagcgcaacccttactgtcagttaccatcattaagttggggactctggcaggactgccggtgacaaatcggaggaaggtggggacgacgtcaaatcatcatgccccttatgtcctgggctacacacgtactacaatggctgttaacaaagtgaagcaaagcagtgatgtggagcaaaacacaaaaagcagtctcagttcagattgtaggctgaaactcgcctatatgaagtcggaattgctagtaatcgcagatcagcatgctgcggtgaatacgttcccgggccttgtacacaccgcccgtcacaccatgagagtcgataacacccgaagcctgt

SEQ ID NO: 30 - DNA, 1319 bp, Butyricoccus pullicaecorum
  source 1..1319: mol_type = genomic DNA
agtggcggacgggtgagtaacgcgtgagcaatctgcctttaagagggggataacagtcggaaacggctgctaataccgcataaagcatcgaaaccgcatgattttgatgccaaaggagcaatccgcttttagatgagctcgcgtctgattagctggttggcggggtaacggcccaccaaggcgacgatcagtagccggactgagaggttgaacggccacattgggactgagacacggcccagactcctacgggaggcagcagtggggaatattgcgcaatgggggaaaccctgacgcagcaacgccgcgtgattgaagaaggccttcgggttgtaaagatctttaatcagggacgaaacaaatgacggtacctgaagaataagctccggctaactacgtgccagcagccgcggtaatacgtagggagcaagcgttatccggatttactgggtgtaaagggcgcgcaggcgggccggtaagttggaagtgaaatctatgggcttaacccataaactgcttttcaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggatactaggtgtgggaggtattgaccccttccgtgccgcagttaacacaataagtatcccacctggggagtacggccgcaaggttgaaactcaaaggaattgacgggggcccgcacaagcagtggagtatgtggtttaattcgaagcaacgcgaagaaccttaccaggtcttgacatcccgatgaccgcctcagagatgagccttttcttcggaacatcggtgacaggtggtgcatggttgtcgtcagctcgtgtcgtgagatgttgggttaagtcccgcaacgagcgcaacccttacggttagttgatacgcaagatcactctagccggactgccgttgacaaaacggaggaaggtggggacgacgtcaaatcatcatgccccttatgacctgggctacacacgtactacaatggcagtcatacagagggaagcaaaaccgcgaggtggagcaaatccctaaaagctgtcccagttcagattgcaggctgcaacccgcctgcatgaagtcggaattgctagtaatcgcggatcagcatgccgcggtgaatacgttcccgggccttgtacacaccgcccgtcacaccatgagagccgtcaatacccgaagtccgt

SEQ ID NO: 31 - DNA, 791 bp, Clostridium maritium
  source 1..791: mol_type = genomic DNA
ctacactgcagtcgagcgatttcttcggtaagagcggcggacgggtgagtaacgcgtgggtaacctgccctatacacacggataacataccgaaaggtatgctaatacgagataacataagagattcgcatggatttcttatcaaagcttttgcggtataggatggacccgcgtctgattagctagttggtaaggtaacggcttaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggtttcttaagtcagaggtgaaaggctacggctcaaccgtagtaagcctttgaaactgagaaacttgagtgcaggagaggagagtagaattcctagtgtagcggtgaaatgcgtagatattaggaggaataccagttgcgaaggcggctctctggactgtaactgacactgaggcacgaaagcgtggggagcaaacaggattagataccctggtagtccacgccgtaaacgatgagtactagctgtcggaggttacccccttcggtggcgcagctaacgcattaagtactccgcc

SEQ ID NO: 32 - DNA, 1377 bp, Eubacterium fissicatens
  source 1..1377: mol_type = genomic DNA
agtggcggacgggtgagtaacgcgtgggtaacctgccttgtacagggggataacagttagaaatgactgctaataccgcataagcgcacagtatcgcatggtacagtgtgaaaaactccggtggtacaagatggacccgcgtctgattagctagttggtaaggtaacggcttaccaaggcaacgatcagtagccgacttgagagagtgatcggccacattgggactgagacacggcccaaactcctacgggaggcagcagtggggaatattgcacaatgggggaaaccctgatgcagcgacgccgcgtgagtgaagaagtatttcggtatgtaaaactctatcagcaaggaagataatgacggtacttgactaagaagccccggctaactacgtgccagcagccgcggtaatacgtagggggcaagcgttatccggatttactgggtgtaaagggagcgtagacggtatggtaagtcagatgtgaaagcccggggcttaaccccggaactgcatttgaaactatcaaactagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatactaggtgtcagggaacaatagttctttggtgccgcagcaaacgcattaagtattccacctggggagtacgttcgcaagaatgaaactcaaaggaattgacggggacccgcacaagcggtggagcatgtggtttaattcgaagcaacgcgaagaaccttacctggtcttgacatcccaatgacgcctctttaatcggaggtttccttcgggacattggagacaggtggtgcatggttgtcgtcagctcgtgtcgtgagatgttgggttaagtcccgcaacgagcgcaacccttatctttagtagccagcagttcggctgggcactctagagagactgccagggataacctggaggaaggtggggatgacgtcaaatcatcatgccccttatgaccagggctacacacgtgctacaatggcgtaaacaaagggaagcaaaactgtgaggttgagcaaatcccaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcggatatgcccgaagtcagtgacccaaccgtaaggagggagctgccgaaggtggagccgataactggggtgaagtcgt

SEQ ID NO: 33 - DNA, 1376 bp, Clostridium saccharolyticum
  source 1..1376: mol_type = genomic DNA
agcggcggacgggtgagtaacgcgtgggtaacctgcctcatacagggggataacagttagaaatgactgctaataccgcataagcgcacagtgctgcatggcacagtgtgaaaaactccggtggtatgagatggacccgcgttggattaggcagttggcggggtaacggcccaccaaaccgacgatccatagccggcctgagagggtgaacggccacattgggactgagacacggcccaaactcctacgggaggcagcagtggggaatattgcacaatgggggaaaccctgatgcagcgacgccgcgtgagtgaagaagtaattcgttatgtaaagctctatcagcagggaagaaaatgacggtacctgactaagaagccccggctaactacgtgccagcagccgcggtaatacgtagggggcaagcgttatccggatttactgggtgtaaagggagcgtagacggccgtgcaagtctgatgtgaaaggctggggctcaaccccgggactgcattggaaactgtatggctggagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgattactaggtgttgggggacatggtccttcggtgccgccgcaaacgcagtaagtaatccacctggggagtacgttcgcaagaatgaaactcaaaggaattgacggggacccgcacaagcggtggagcatgtggtttaattcgaagcaacgcgaagaaccttaccaagtcttgacatcgagaggacagagtatgtaatgtactttcccttcggggcctcgaagacaggtggtgcatggttgtcgtcagctcgtgtcgtgagatgttgggttaagtcccgcaacgagcgcaacccctatcttcagtagccagcaattcggatgggcactctggagagactgccggggataacccggaggaaggcggggatgacgtcaaatcatcatgccccttatgacttgggctacacacgtgctacaatggcgtaaacaaagggaagcgagggagtgatccggagcaaatcccaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagtcgataacgcccgaagtcagtgacccaaccgaaaggagggagctgccgaaggcgggattggtaactggggtgaagtcgt

SEQ ID NO: 34 - DNA, 1377 bp, Blautia luti
  source 1..1377: mol_type = genomic DNA
agtggcggacgggtgagtaacgcgtgggtaacctgccttatactgggggataacagccagaaatggctgctaataccgcataagcgcacggggccgcatggtcctgtgtgaaaaactccggtggtataagatggacccgcgttggattagctagttggcagggcagcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcgagcgttatccggatttactgggtgtaaagggagcgtagacggcgtatcaagtctgatgtgaaaggcaggggcttaacccctggactgcattggaaactggtatgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtctgggagcacagctcttaggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgacagagtatgtaatgtacttttccttcgggacaggggagacaggtggtgcatggttgtcgtcagctcgtgtcgtgagatgttgggttaagtcccgcaacgagcgcaacccctatccttagtagccagcaagtaatgttgggcactctgaggagactgccagggataacctggaggaaggcggggatgacgtcaaatcatcatgccccttatgatttgggctacacacgtgctacaatggcgtaaacaaagggaagcgaacctgtgagggtgggcaaatctcaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgtaaggaaggagctgccgaaggcgggacggatgactggggtgaagtcgt

SEQ ID NO: 35 - DNA, 1465 bp, Clostridium metylpentosum
  source 1..1465: mol_type = genomic DNA
ggttaccttgttacgacttcaccccaatcatcaaccccaccttcgacgacgtcccccttgcggttagactatcggcttcgggtgttgccaactctcatggtgtgacgggcggtgtgtacaaggcccgggaacgtattcaccgcggcatgctgatccgcgattactagcaattccggcttcatgcaggcgggttgcagcctgcaatccgaactgagactatttttaggggtttgctccatgtcaccatcttgcttccctctgttaatagccattgtagtacgtgtgtagcccaggtcataaggggcatgatgatttgacgtcatccccaccttcctccgttttgtcaacggcagtccgtctagagtgctcttgcgtagcaactaaacgtaagggttgcgctcgttgcgggacttaacccaacatctcacgacacgagctgacgacaaccatgcaccacctgtctcggtgccccgaagggcttcacctatctctaggctatgcaccggatgtcaagacctggtaaggttcttcgcgttgcttcgaattaaaccacatactccactgcttgtgcgggcccccgtcaattcctttgagtttcaaccttgcggtcgtactccccaggtggattacttattgtgttaactccggcacggaaggggtcagtccccccacacctagtaatcatcgtttacagcgtggactaccagggtatctaatcctgtttgctacccacgctttcgagcctcagcgtcagttaaagcccagcaggccgccttcgccactggtgttcctcctaatatttacgcatttcaccgctacactaggaattccgcctgcctctacttcactcaagaactgcagttttgaacgcggctatgggttgagcccatagatttaacattcaacttgcaatcccgcctacgctccctttacacccagtaattccggacaacgctcgctacctacgtattaccgcggctgctggcacgtagttagccgtagcttcctccttggttaccgtcattatcttcaccaaggacagaggtttacaatccgaaaaccttcttccctcactcggcgtcgctgcatcagggtttcccccattgtgcaatattccccactgctgcctcccgtaggagtctgggccgtgtctcagtcccaatgtggccgttcaacctctcagtccggctaccaatcgtcgccttggtgggccgttacctcaccaactagctaattggacgcgagtccatctttcagcggattgctcctttgatatcagctccatgcgaaaccaatatgttatgcggtattagcgtccgtttccagacgttatccccctctgaaaggcaggttactcacgcgttactcacccgtccgccactaagttgaatcaaattccttccgaagaattcattcaaagcaacttcgtcgacttgcatgtgtaaggcgcgccgacagcgttcgt

SEQ ID NO: 36 - DNA, 1422 bp, Clostridium xylanolyticum
  source 1..1422: mol_type = genomic DNA
agagtttgatcctggctcaggatgaacgctggcggcgtgcctaacacatgcaagtcgaacggaatttacatgaagcctagcgattgtaaatttagtggcggacgggtgagtaacgcgtgggtaacctgccttgtactgggggacaacagttggaaacgactgctaataccgcataagcgcacagcttcgcatgaagcagtgtgaaaaactccggtggtacaagatggacccgcgtctgattagctggttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcaacgccgcgtgagtgaagaagtatttcggtatgtaaagctctatcagcaggaaagaaaatgacggtacctgactaagaagccccggctaactacgtgccagcagccgcggtaatacgtagggggcaagcgttatccggatttactgggtgtaaagggagcgtagacggttttgcaagtctgaagtgaaagcccggggcttaaccccgggactgctttggaaactgtaggactagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgattactaggtgttggtgggtacgacccatcggtgccgcagcaaacgcaataagtaatccacctggggagtacgttcgcaagaatgaaactcaaaggaattgacggggacccgcacaagcggtggagcatgtggtttaattcgaagcaacgcgaagaaccttacctggtcttgacatccctatgaataacgggcaatgccgttagtacttcggtacataggagacaggtggtgcatggttgtcgtcagctcgtgtcgtgagatgttgggttaagtcccgcaacgagcgcaacccttatctttagtagccagcagtaagatgggcactctagagagactgccggggataacccggaggaaggtggggatgacgtcaaatcatcatgccccttatgaccagggctacacacgtgctacaatggcgtaaacaaagagaagcgaagtcgtgaggcagagcgaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcggaaatgcccgaagtcggtgacctaaccgaa

SEQ ID NO: 37 - DNA, 1478 bp, Oscillibacter valericigenes
  source 1..1478: mol_type = genomic DNA
ctcaggacgaacgctggcggcgtgcttaacacatgcaagtcgaacggagcacccttgactgaggtttcggccaaatgataggaatgcttagtggcggactggtgagtaacgcgtgaggaacctaccttccagagggggacaacagttggaaacgactgctaataccgcatgacgcatgaccggggcatcccgggcatgtcaaagattttatcgctggaagatggcctcgcgtctgattagctagatggtggggtaacggcccaccatggcgacgatcagtagccggactgagaggttgaccggccacattgggactgagatacggcccagactcctacgggaggcagcagtggggaatattgggcaatggacgcaagtctgacccagcaacgccgcgtgaaggaagaaggctttcgggttgtaaacttcttttgtcagggaagagtagaagacggtacctgacgaataagccacggctaactacgtgccagcagccgcggtaatacgtaggtggcaagcgttgtccggatttactgggtgtaaagggcgtgcagccgggccggcaagtcagatgtgaaatctggaggcttaacctccaaactgcatttgaaactgtaggtcttgagtaccggagaggttatcggaattccttgtgtagcggtgaaatgcgtagatataaggaagaacaccagtggcgaaggcggataactggacggcaactgacggtgaggcgcgaaagcgtggggagcaaacaggattagataccctggtagtccacgctgtaaacgatggatactaggtgtgcggggactgaccccctgcgtgccgcagttaacacaataagtatcccacctggggagtacgatcgcaaggttgaaactcaaaggaattgacgggggcccgcacaagcggtggattatgtggtttaattcgaagcaacgcgaagaaccttaccagggcttgacatcctactaacgaagtagagatacatcaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtaatacaatggcggtcaacagagggaggcaaagccgcgaggcagagcaaacccccaaaagccgtcccagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtaaccg

SEQ ID NO: 38 - DNA, 1620 bp, Ruminococcus obeum
  source 1..1620: mol_type = genomic DNA
agtcgaacgggaaccttttattgaagcttcggcagatttagctggtttctagtggcggacgggtgagtaacgcgtgggtaacctgccctatacagggggataacaaccagaaatggttgctaataccgcataagcgcacaggaccgcatggtccggtgtgaaaaactccggtggtataggatggacccgcgttggattagccagttggcagggtaacggcctaccaaagcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggattagcaagtctgatgtgaaaggcaggggctcaacccctggactgcattggaaactgccagtcttgagtgccggagaggtaagcggaattcctagtgtagcggtgaaatgcgtagatattaggaggaacaccagtggcgaaggcggcttactggacggcaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgacggactcttaaccgagtctttccttcgggacagaggagacaggtggtgcatggttgtcgtcagctcgtgtcgtgagatgttgggttaagtcccgcaacgagcgcaacccctatccccagtagccagcatttcggatgggcactctgaggagactgccagggataacctggaggaaggcggggatgacgtcaaatcatcatgccccttatgatttgggctacacacgtgctacaatggcgtaaacaaagggaagcgagcctgcgagggtaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgcaagggaggagctgccgaaggcgggaccgatgactggggtgaagtcgtaacaaggtaaccgtgactacacgaagctggaatcgctagtaatcgcggatcagaatgccgcggtgaatacgttcccgggtcttgtacacaccgcccgtcacaccatgggagtcagtaacgcccgaagtcagtgacctaaccgcaaggaaggagctgccgaaggcgggaccgatgactggggtgaagtcgtaaca

SEQ ID NO: 39 - DNA, 1403 bp, Megasphaera elsdenii
  source 1..1403: mol_type = genomic DNA
acgcgtaagcaacctgccctccggatggggacaacagctggaaacggctgctaataccgaatacgtttccattgccgcatggcagtgggaagaaaggtggcctctgaatatgctaccgccgggggaggggcttgcgtctgattagctagttggaggggtaacggcccaccaaggcgacgatcagtagccggtctgagaggatgaacggccacattggaactgagacacggtccagactcctacgggaggcagcagtggggaatcttccgcaatgggcgaaagcctgacggagcaacgccgcgtgagcgaagacggccttcgggttgtaaagctctgttatacgggacgaacggctagtgtgccaataccacattagaatgacggtaccgtaagagaaagccacggctaactacgtgccagcagccgcggtaatacgtaggtggcaagcgttgtccggaattattgggcgtaaagggcgcgcaggcggtttcataagtctgtcttaaaagtgcggggcttaaccccgtgaggggacggaaactgtgagactggagtgtcggagaggaaagcggaattcctagtgtagcggtgaaatgcgtagatattaggaggaacaccagtggcgaaagcggctttctggacgacaactgacgctgaggcgcgaaagccaggggagcgaacgggattagataccccggtagtcctggccgtaaacgatggatactaggtgtagggggtatcgacccctcctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgagtgctatcctcagagatgaggagttcttcttcggaagacgcgaaaacaggtggtgcacggctgtcgtcagctcgtgtcgtgagatgttgggttaagtcccgcaacgagcgcaacccctatcttctgttgccagcgcgtcatggcggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaacccgcctgcatgaagcaggaatcgctagtaatcgcaggtcagcatactgcggtgaatacgttcccgggccttgtacacaccgcccgtcacaccacgaaagtcattcacacccgaagccggtgaggtaaccgtaaggagccagccgtcgaaggtgggggcgatgattggggtgaagtcgtaa

SEQ ID NO: 40 - DNA, 1369 bp, Blautia luti
  source 1..1369: mol_type = genomic DNA
ggtgagtaacgcgtgggtaacctgccttatacagggggataacagtcagaaatggctgctaataccgcataagcgcacagggccgcatggcccggtgtgaaaaactgaggtggtataagatggacccgcgttggattagccagttggcagggtaacggcctaccaaagcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcataacaagtctgatgtgaaaggctggggcttaaccccgggactgcattggaaactgttaagcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagcacagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatctgcctgaccggtgagtaacgtcacctttccttcgggacaggcaagacaggtggtgcatggttgtcgtcagctcgtgtcgtgagatgttgggttaagtcccgcaacgagcgcaacccctatccccagtagccagcatgtaaaggtgggcactctgaggagactgccagggataacctggaggaaggtggggatgacgtcaaatcatcatgccccttatgatttgggctacacacgtgctacaatggcgtaaacagagggaagcgaaagggtgacctggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaaccgaaagggaggagctgccgaaggcgggacggatgactggggtgaagtcgtaac

SEQ ID NO: 41 - DNA, 1359 bp, Bacteroides coprocola
  source 1..1359: mol_type = genomic DNA
gtatccaaccttccgtttactcagggatagcctttcgaaagaaagattaatacctgatagtatggtaagattgcatgataataccattaaagattcatcggtaaacgatggggatgcgttccattaggtagtaggcggggtaacggcccacctagccgacgatggataggggttctgagaggaaggtcccccacattggaactgagacacggtccaaactcctacgggaggcagcagtgaggaatattggtcaatgggcgagagcctgaaccagccaagtagcgtgaaggatgaaggttctatggattgtaaacttcttttataagggaataaagtgctttacgtgtagagttttgtatgtaccttatgaataagcatcggctaactccgtgccagcagccgcggtaatacggaggatgcgagcgttatccggatttattgggtttaaagggagcgtagacgggatgttaagtcagctgtgaaagtttggggctcaaccttaaaattgcagttgaaactggcgttcttgagtgcggtagaggcaggcggaattcgtggtgtagcggtgaaatgcttagatatcacgaagaaccccgattgcgaaggcagcttgctggagcgtaactgacgttgatgctcgaaagtgtgggtatcaaacaggattagataccctggtagtccacacggtaaacgatggatactcgctgttggcgatatacggtcagcggccaagcgaaagcattaagtatcccacctggggagtacgccggcaacggtgaaactcaaaggaattgacgggggcccgcacaagcggaggaacatgtggtttaattcgatgatacgcgaggaaccttacccgggcttaaattatgcatgaatgatctggagacagatcagccgcaaggcatgtatgaaggtgctgcatggttgtcgtcagctcgtgccgtgaggtgtcggcttaagtgccataacgagcgcaaccctttctgccagttactaacaggcaatgctgaggactctggcggtactgccatcgtaagatgtgaggaaggtggggatgacgtcaaatcagcacggcccttacgtccggggctacacacgtgttacaatggggggtacagaaggcagcttaccggcgacggttggccaatccctaaagcccctctcagttcggactggagtctgcaacccgactccacgaagctggattcgctagtaatcgcgcatcagccacggcgcggtgaatacgttcccgggccttgtacacaccgcccgtcaagccatgaaagccgggagtacctgaagtgcgtaaccgcgaggagcgccctagggtaacactggtaattggggctaagtcgt

SEQ ID NO: 42 - DNA, 1406 bp, Bacteroides plebius
  source 1..1406: mol_type = genomic DNA
ggggcagcatgaacttagcttgctaagttcgatggcgaccggcgcaccgttgagtaacgcgtatccaaccttccgtacactcaggaatagcctttcgaaagaaagattaatacctgatggtatgatgggattgcatgaaatcatcattaaagattcatcggtgtacgatggggatgcgttccattagatagtaggcggggtaacggcccacctagtcgacgatggataggggttctgagaggaaggtcccccacattggaactgagacacggtccaaactcctacgggaggcagcagtgaggaatattggtcaatgggcgcgagcctgaaccagccaagtagcgtgaaggatgaaggtcctacggattgtaaacttcttttataagggaataaagtcacccacgtgtgggtgtttgtatgtaccttatgaataagcatcggctaactccgtgccagcagccgcggtaatacggaggatgcgagcgttatccggatttattgggtttaaagggagcgtagacgggtcgttaagtcagctgtgaaagttcggggctcaaccttgaaattgcagttgatactggcgtccttgagtacggttgaggcaggcggaattcgtggtgtagcggtgaaatgcttagatatcacgaagaaccccgattgcgaaggcagcctgctaaaccgccactgacgttgaggctcgaaagtgtgggtatcaaacaggattagataccctggtagtccacacggtaaacgatggatactcgctgttggcgatagactgtcagcggcttagcgaaagcgttaagtatcccacctggggagtacgccggcaacggtgaaactcaaaggaattgacgggggcccgcacaagcggaggaacatgtggtttaattcgatgatacgcgaggaaccttacccgggcttgaattgcagacgaattgcttggaaacaggcaagccgcaaggcgtctgtgaaggtgctgcatggttgtcgtcagctcgtgccgtgaggtgtcggcttaagtgccataacgagcgcaaccctcgtgtccagttgctagcaggtagtgctgaggactctggacagactgccatcgtaagatgtgaggaaggtggggatgacgtcaaatcagcacggcccttacgtccggggctacacacgtgttacaatggggggtacagcaggcagctaccgggcgaccggatgccaatcccgaaagcctctctcagttcggactggagtctgcaacccgactccacgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaactggtaatt

SEQ ID NO: 43 - DNA, 1420 bp, Roseburia inulinivorans
  source 1..1420: mol_type = genomic DNA
gcacttttgccgattttcttcggaactgaagtaatagtgactgagtggcggacgggtgagtaacgcgtggataacctgcctcacacagggggataacagttagaaatgactgctaataccgcataagcgcacagtaccgcatggtacagtgtgaaaaactccggtggtgtgagatggatccgcgtctgattagccagttggcggggtaacggcccaccaaagcgacgatcagtagccggcctgagagggcgaccggccacattgggactgagacacggcccagactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagctccggctaaatacgtgccagcagccgcggtaatacgtatggagcaagcgttatccggatttactgggtgtaaagggagcgcaggcggtatgacaagtctgatgtgaaaggctggggctcaaccccaggactgcattggaaactgtcagactagagtgtcggagaggtaagtggaattcctagtgtagcggtgaaatgcgtagatattaggaggaacaccagtggcgaaggcggcttactggacgacaactgacgctgaggctcgaaagcgtggggagcaaacaggattagataccctggtagtccacgccgtaaacgatgaatactaggtgtcgggaggcagagcctttcggtgccgcagcaaacgcagtaagtattccacctggggagtacgttcgcaagaatgaaactcaaaggaattgacggggacccgcacaagcggtggagcatgtggtttaattcgaagcaacgcgaagaaccttaccaggccttgacatccccctgacgggacagtaatgtgtccgttccttcgggacagaggagacaggtggtgcatggttgtcgtcagctcgtgtcgtgagatgttgggttaagtcccgcaacgagcgcaacccttatcctcagtagccagcggataaagccgggcactctgtggagactgccagggacaacctggaggaaggtggggatgacgtcaaatcatcatgccccttatggcctgggctacacacgtgctacaatggcgtaaacaaagggaagcgaagctgtgaagtgaagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcgggaatgcccgaagccggtgacccaaccttaaggagggagccgtcgaaggcaggcctgataactggggtgaagtcgt

SEQ ID NO: 44 - DNA, 1392 bp, Ruminococcus albus
  source 1..1392: mol_type = genomic DNA
ctgatctagtggcggacgggtgagtaacacgtgagcaatctgcctttcagagggggataccgattggaaacgatcgttaataccgcataacataattgaaccgcatgatttgattatcaaagatttatcgctgaaagatgagctcgcgtctgattagctagttggtaaggtaacggcttaccaaggcgacgatcagtagccggactgagaggttgatcggccacattgggactgagacacggcccagactcctacgggaggcagcagtggggaatattgcacaatggaggaaactctgatgcagcgatgccgcgtgagggaagaaggttttaggattgtaaacctctgtcttcagggacgaaaaaagacggtacctgaggaggaagctccggctaactacgtgccagcagccgcggtaatacgtagggagcgagcgttgtccggaattactgggtgtaaagggagcgtaggcgggatcgcaagtcagatgtgaaaactatgggcttaacccataaactgcatttgaaactgtggttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttccgtgccgcagcaaacgcaataagtaatccacctggggagtacgaccgcaaggttgaaactcaaaggaattgacgggggcccgcacaagcagtggagtatgtggattaattcgaagcaacgcgaagaaccttaccaggtcttgacatcgtatgcatagctcagagatgagtgaaatctcttcggagacatatagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggcctcacacgtactacaatggctgttaacagagggatgcaaagccgcgaggtagagcgaacccctaaaagcagtcttagttcggattgtaggctgcaacccgcctacatgaagtcggaattgctagtaatcgcagatcagcatgctgcggtgaatacgttcccgggccttgtacacaccgcccgtcacgccatgggagtcggtaacacccgaagcctgtagtctaaccgcaaggaggacgcagtcgaaggtgggattgatgactggggtgaagtcgtaacagggtaaccg

SEQ ID NO: 45 - DNA, 1414 bp, Blautia producta
  source 1..1414: mol_type = genomic DNA
tggacagattcttcggatgaagtccttagtgactgagtggcggacgggtgagtaacgcgtgggtaacctgcctcatacagggggataacagttagaaatggctgctaataccgcataagcgcacggtactgcatggtacagtgtgaaaaactccggtggtatgagatggacccgcgttggattagctagttggcagggtaacggcctaccaaggcgacgatccatagccggcctgagagggtggacggccacattgggactgagacacggcccagactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggaatggcaagtctgatgtgaaaggccggggctcaaccccgggactgcattggaaactgtcaatctagagtaccggaggggtaagtggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaaatcttgacatcgatctgaccggactgtaatgagtcctttcccttcggggacagagaagacaggtggtgcatggttgtcgtcagctcgtgtcgtgagatgttgggttaagtcccgcaacgagcgcaacccttatcctcagtagccagcaagtgaagttgggcactctgtggagactgccagggataacctggaggaaggtggggatgacgtcaaatcatcatgccccttatgatttgggctacacacgtgctacaatggcgtaaacaaagggaagcgatcacgcgagtgtgagcaaatctcaaaaataacgtctcagttcggattgtagtctgcaactcgactacatgaagctggaatcgctagtaatcgcaggtcagcatactgcggtgaatacgttcccgggtcttgtacacaccgcccgtcacaccatgggagtcagtaacacccgaagccggtgacctaaccgaaaggaaggagccgtcgaaggtgggaccgataactggggtgaagtcgt

SEQ ID NO: 46 - DNA, 1389 bp, Clostridium nexile
  source 1..1389: mol_type = genomic DNA
gtttgtgacttagtggcggacgggtgagtaacgcgtgggtaacctgccttatacagggggataacagttagaaatgactgctaataccgcataagcgcacagtctcgcatgggacagtgtgaaaaactaaggtggtataagatggacccgcgtctgattagctagttggtggggtaaaggcctaccaaggcgacgatcagtagccgacctgagagggtgatcggccacattgggactgagacacggcccaaactcctacgggaggcagcagtggggaatattgcacaatgggggaaaccctgatgcagcaacgccgcgtgaaggaagaagtatctcggtatgtaaacttctatcagcagggaagaaaatgacggtacctgactaagaagctccggctaaatacgtgccagcagccgcggtaatacgtatggagcaagcgttatccggatttactgggtgtaaagggagcgtaggcggttatgcaagtcagatgtgaaagcccggggcttaaccccgggactgcatttgaaactgtgtaactagagtgtcggagaggtaagtggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttggggagcaaagctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatctggatgaccggaccgtaatgggtcctttccttcgggacatccaagacaggtggtgcatggttgtcgtcagctcgtgtcgtgagatgttgggttaagtcccgcaacgagcgcaacccttatccttagtagccagcagtaagatgggcactctagggagactgccggagacaatccggaggaaggtggggatgacgtcaaatcatcatgccccttatgacttgggctacacacgtgctacaatggcgtaaacaaagggaagcgagaccgcgaggttaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagtcgataacgcccgaagccggtgactcaaccgaaaggagagagccgtcgaaggcgggatggataactggggtgaagtcgtaac

SEQ ID NO: 47 - DNA, 1404 bp, Butyricicoccus pullicaecorum
  source 1..1404: mol_type = genomic DNA
atctcttcggagatggaattcttaacctagtggcggacgggtgagtaacgcgtgagcaatctgcctttaggagggggataacagtcggaaacggctgctaataccgcataatacgtttgggaggcatctcttgaacgtcaaagattttatcgcctttagatgagctcgcgtctgattagctggttggcggggtaacggcccaccaaggcgacgatcagtagccggactgagaggttgaacggccacattgggactgagacacggcccagactcctacgggaggcagcagtggggaatattgcgcaatgggggaaaccctgacgcagcaacgccgcgtgattgaagaaggccttcgggttgtaaagatctttaatcagggacgaaaaatgacggtacctgaagaataagctccggctaactacgtgccagcagccgcggtaatacgtagggagcaagcgttatccggatttactgggtgtaaagggcgcgcaggcgggccggcaagttgggagtgaaatcccggggcttaaccccggaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggatactaggtgtgggaggtattgaccccttccgtgccgcagttaacacaataagtatcccacctggggagtacggccgcaaggttgaaactcaaaggaattgacgggggcccgcacaagcagtggagtatgtggtttaattcgaagcaacgcgaagaaccttaccaggtcttgacatcccgatgaccggcgtagagatacgccctctcttcggagcatcggtgacaggtggtgcatggttgtcgtcagctcgtgtcgtgagatgttgggttaagtcccgcaacgagcgcaacccttacggttagttgatacgcaagatcactctagccggactgccgttgacaaaacggaggaaggtggggacgacgtcaaatcatcatgccccttatgacctgggctacacacgtactacaatggcagtcatacagagggaagcaataccgcgaggtggagcaaatccctaaaagctgtcccagttcagattgcaggctgcaacccgcctgcatgaagtcggaattgctagtaatcgcggatcagcatgccgcggtgaatacgttcccgggccttgtacacaccgcccgtcacaccatgagagccgtcaatacccgaagtccgtagcctaaccgcaaggggggcgcggccgaaggtaggggtggtaattagggtgaagtcgtac

SEQ ID NO: 48 - DNA, 1407 bp, Ruminococcus flavefaciens
  source 1..1407: mol_type = genomic DNA
agtcgacggacgaggaggagcttgcttctccgagttagtggcggacgggtgagtaacacgtgagcaacctacccttgagagggggatagcttctggaaacggatggtaataccccataacatatattttaggcatctaagatatatcaaagaaattcgctcaaggatgggctcgcgtctgattagatagttggtgaggtaacggcccaccaagtcgacgatcagtagccggactgagaggttgaacggccacattgggactgagacacggcccagactcctacgggaggcagcagtggggaatattgcacaatggggggaaccctgatgcagcgatgccgcgtggaggaagaaggttttcggattgtaaactccttttaacagggacgataatgacggtacctgaagaaaaagctccggctaactacgtgccagcagccgcggtaatacgtagggagcgagcgttgtccggaattactgggtgtaaagggagcgtaggcgggacggtaagtcaggtgtgaaatatacgtgctcaacatgtagactgcacttgaaactgctgttcttgagtgaagtagaggtaagcggaattcctagtgtagcggtgaaatgcgtagatattaggaggaacatcggtggcgaaggcggcttactgggcttttactgacgctgaggctcgaaagcgtggggagcaaacaggattagataccctggtagtccacgctgtaaacgatgattactaggtgtggggggactgaccccttccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcgtatgcatagtctagagatagatgaaatcccttcggggacatatagacaggtggtgcatggttgtcgtcagctcgtgtcgtgagatgttgggttaagtcccgcaacgagcgcaacccttacctttagttgctacgcaagagcactctagagggactgccgttgacaaaacggaggaaggtggggatgacgtcaaatcatcatgccccttatgacctgggctacacacgtactacaatggcaattaacagagggaagcaaaacagcgatgtggagcaaatcccgaaaaattgtcccagttcagattgcaggctgcaactcgcctgcatgaagtcggaattgctagtaatcgcagatcagaatgctgcggtgaatacgttcccgggccttgtacacaccgcccgtcacaccatgggagtcggtaacacccgaagcctgtagtctaaccttataggaggacgcagtcgaaggtgggattgatgactggggtgaagtcgt

SEQ ID NO: 49 - DNA, 1349 bp, Clostridium orbiscindens
  source 1..1349: mol_type = genomic DNA
aaagggaatgcttagtggcggacgggtgagtaacgcgtgagtaacctgccttggagtggggaataacagccggaaacggctgctaataccgcatgatgtatctggatcgcatggttctggataccaaagatttatcgctctgagatggactcgcgtctgattagctagttggtgaggtaacggctcaccaaggcgacgatcagtagccggactgagaggttggccggccacattgggactgagacacggcccagactcctacgggaggcagcagtggggaatattgggcaatgggcgaaagcctgacccagcaacgccgcgtgaaggaagaaggccctcgggttgtaaacttcttttgtcagggacgaagcaagtgacggtacctgacgaataagccacggctaactacgtgccagcagccgcggtaatacgtaggtggcaagcgttatccggatttactgggtgtaaagggcgtgtaggcgggagtgcaagtcagatgtgaaaactatgggctcaacccatagcctgcatttgaaactgtacttcttgagtgatggagaggcaggcggaattccctgtgtagcggtgaaatgcgtagatatagggaggaacaccagtggcgaaggcggcctgctggacattaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccaggacttgacatcctactaacgaagcagagatgcataaggtgcccttcggggaaagtagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggcggtcaacagagggaagcaaagccgcgaggtggagcaaatccctaaaagccgtcccagttcggattgcaggctgaaactcgcctgtatgaagtcggaatcgctagtaatcgcggatcagcatgccgcggtgaatacgttcccgggccttgtacacaccgcccgtcacaccatgagagtcgggaacacccgaagtccgtagcctaacagcaatggg

SEQ ID NO: 50 - DNA, 1420 bp, Ruminococcus bromii
  source 1..1420: mol_type = genomic DNA
acgaagctttgaggagcttgctttttaagcttagtggcggacgggtgagtaacgcgtgagcaacctgcctctcagaggggaataacgttttgaaaagaacgctaataccgcataacatatcggaaccgcatgattctgatatcaaaggagcaatccgctgagagatgggctcgcgtccgattagttagttggtgaggtaacggctcaccaagactacgatcggtagccggactgagaggttgatcggccacattgggactgagacacggcccagactcctacgggaggcagcagtgggggatattgcgcaatgggggaaaccctgacgcagcaacgccgcgtgaaggaagaaggtcttcggattgtaaacttcttttgtcagggacgaagaaagtgacggtacctgacgaataagctccggctaactacgtgccagcagccgcggtaatacgtagggagcgagcgttgtccggatttactgggtgtaaagggtgcgtaggcggccgagcaagtcagttgtgaaaactatgggcttaacccataacgtgcaattgaaactgtccggcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccctggtagtccatgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcctgagaatccttaagagattagggagtgccttcgggaactcagagacaggtggtgcatggttgtcgtcagctcgtgtcgtgagatgttgggttaagtcccgcaacgagcgcaacccttgctattagttgctacgcaagagcactctaataggactgccgttgacaaaacggaggaaggtggggacgacgtcaaatcatcatgccccttatgacctgggctacacacgtactacaatggccattaacagagggaagcaaaaccgcgaggcagagcaaacccctaaaaatggtcccagttcggattgtaggctgcaacccgcctacatgaagttggaattgctagtaatcgcggatcagcatgccgcggtgaatacgttcccgggccttgtacacaccgcccgtcacaccatgggagccggtaatacccgaagtcagtagtctaacagcaatgaggacgctgccgaaggtaggattggcgactggggtgaagtcgtaacaaggtaaccg

SEQ ID NO: 51 - DNA, 1490 bp, Ruminococcus albus
  source 1..1490: mol_type = genomic DNA
agagtttgatcctggctcaggacgaacgctggcggcacgcttaacacatgcaagtcgaacgagagaagagaagcttgcttttctgatctagtggcggacgggtgagtaacacgtgagcaatctgcctttcagagggggataccgattggaaacgatcgttaataccgcataacataattgaaccgcatgatttgattatcaaagatttatcgctgaaagatgagctcgcgtctgattagctagttggtaaggtaacggcttaccaaggcgacgatcagtagccggactgagaggttgatcggccacattgggactgagacacggcccagactcctacgggaggcagcagtggggaatattgcacaatggaggaaactctgatgcagcgatgccgcgtgagggaagaaggttttaggattgtaaacctctgtcttcagggacgaaaaaaaagacggtacctgaggaggaagctccggctaactacgtgccagcagccgcggtaatacgtagggagcgagcgttgtccggaattactgggtgtaaagggagcgtaggcgggatcgcaagtcagatgtgaaaactatgggcttaacccataaactgcatttgaaactgtggttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttccgtgccgcagcaaacgcaataagtaatccacctggggagtacgaccgcaaggttgaaactcaaaggaattgacgggggcccgcacaagcagtggagtatgtggattaattcgaagcaacgcgaagaaccttaccaggtcttgacatcgtatgcatagctcagagatgagtgaaatctcttcggagacatatagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggcctcacacgtactacaatggctgtcaacagagggatgcaaagccgcgaggtggagcgaacccctaaaagcagtcttagttcggattgtaggctgcaacccgcctacatgaagtcggaattgctagtaatcgcagatcagcatgctgcggtgaatacgttcccgggccttgtacacaccgcccgtcacgccatgggagtcggtaacacccgaagcctgtagtctaaccgcaaggaggacgcagtcgaaggtgggattgatgactggggtgaagtcgtaacaaggtagccgtatcggaaggtgcg

SEQ ID NO: 52 - DNA, 20 bp, synthetic construct
  source 1..20: mol_type = other DNA; note = 16S rRNA forward primer
agagtttgatymtggctcag

SEQ ID NO: 53 - DNA, 21 bp, synthetic construct
  source 1..21: mol_type = other DNA; note = 1510R reverse primer
acggytaccttgttacgactt

SEQ ID NO: 54 - DNA, 61 bp, synthetic construct
  source 1..61: mol_type = other DNA; note = 27F primer
aatgatacggcgaccaccgagatctacactatggtaattccagmgttygatymtggctcag

SEQ ID NO: 55 - DNA, 66 bp, synthetic construct
  source 1..66: mol_type = other DNA; note = 338R primer
caagcagaagacggcatacgagatacgagactgattagtcagtcagaagctgcctcccgtaggagt

SEQ ID NO: 56 - DNA, 20 bp, synthetic construct
  source 1..20: mol_type = other DNA; note = 338F primer
actcctacgggaggcagcag

SEQ ID NO: 57 - DNA, 18 bp, synthetic construct
  source 1..18: mol_type = other DNA; note = 926R primer
ccgtcaattcmtttragt